(19) United States
(12) Patent Application Publication
(10) Pub. No.: US 2026/0242391 A1
(43) Pub. Date: Aug. 20, 2026

(54) ANTIMALARIAL ENDOPEROXIDE DERIVATIVES FOR THE TREATMENT OF MYELODYPLASTIC SYNDROME

(71) Applicant: THE SCRIPPS RESEARCH INSTITUTE, La Jolla, CA (US)

(72) Inventors: Michael J. BOLLONG, San Diego, CA (US); Priscilla DO, San Diego, CA (US); Yuyin LIU, San Diego, CA (US); Arnab K. CHATTERJEE, San Diego, CA (US)

(73) Assignee: THE SCRIPPS RESEARCH INSTITUTE, La Jolla, CA (US)

(21) Appl. No.: 19/523,271

(22) PCT Filed: Aug. 5, 2024
(86) PCT No.: PCT/US2024/040900

§ 371 Date: Mar. 26, 2026

Related U.S. Application Data
(60) Provisional application No. 63/517,427, filed on Aug. 3, 2023.

(51) Int. Cl. C07D 493/18; A61K 31/36; A61K 31/38; A61K 31/397; A61K 31/4025; A61K 31/404; A61K 31/4155; A61K 31/4178; A61K 31/4192; A61K 31/4245; A61K 31/4433; A61K 31/453; A61K 31/4535; A61K 31/4725; A61K 31/496; A61K 31/69; A61K 45/06; A61P 35/00; A61P 35/02; C07F 5/02

(52) U.S. Cl. CPC C07D 493/18; A61K 31/36; A61K 31/38; A61K 31/397; A61K 31/4025; A61K 31/404; A61K 31/4155; A61K 31/4178; A61K 31/4192; A61K 31/4245; A61K 31/4433; A61K 31/453; A61K 31/4535; A61K 31/4725; A61K 31/496; A61K 31/69; A61K 45/06; A61P 35/00; A61P 35/02; C07F 5/025

(57) ABSTRACT

Disclosed herein are methods for using an antimalarial endoperoxide compound, such as artemisinin, intreating a subject suffering from myelodysplastic syndromes (MDS), and in slowing or preventing the progression of MDS in the subject to development of acute myeloid leukemia (AML).

### CROSS REFERENCE TO RELATED APPLICATION

This application claims priority to U.S. provisional patent application No. 63/517,427 which was filed on Aug. 3, 2023, and which is hereby incorporated by reference in its entirety.

### FIELD OF THE INVENTION

The application relates to methods for using an antimalarial endoperoxide compound, such as artemisinin, in treating a subject suffering from myelodysplastic syndromes (MDS), and in slowing or preventing the progression of MDS in the subject to development of acute myeloid leukemia (AML).

### BACKGROUND OF THE INVENTION

Myelodysplastic syndromes (MDS) are clonal stem-cell disorders characterized by ineffective hemopoiesis leading to cytopenias and increased blast cell count. About one-third of MDS patients subsequently progress to acute myeloid leukemia (AML). Advances over the past few decades have uncovered the genetic cause of MDS: it is now understood that somatic mutations (e.g., TET2, RUNX1, DNMT3A) in hematopoietic progenitor cells (HPCs) lead to aberrant regulation of hemopoiesis. To date, the only FDA-approved medicines for treating MDS are repurposed AML drugs, including Azacitidine and Decitabine, which promote epigenetic changes in cells leading to cytotoxicity.

Anemia affects the majority of MDS patients, leading to fatigue, poor quality of life and destabilization of underlying cardiovascular disease. As a result, chronic blood transfusions are typically necessary for MDS patients. However, iron overloading due to transfusion dependence is also associated with decreased survival and increased progression to acute myeloid leukemia.

The last U.S. FDA approval of a drug for MDS occurred in 2006 with the approval of lenalidomide for low- or intermediate-risk MDS with 5q deletion. Before that, azacitidine and decitabine were approved in 2004 and 2005, respectively. Although these drugs were breakthroughs and have helped extend the lives of the MDS patient population, they are not curative for most patients, for whom relapsed disease is almost certain. The only known cure for MDS is an allogeneic hematopoietic stem cell transplant, but its use is limited by the older age and attendant comorbidities of typical MDS patients.

### SUMMARY OF THE INVENTION

The present disclosure overcomes these shortcomings of MDS therapies by providing, in an embodiment, a method for treating a myelodysplastic syndrome (MDS) in a subject suffering therefrom. The method comprises administering to the subject a therapeutically effective amount of an antimalarial endoperoxide compound or a pharmaceutically acceptable salt thereof.

In another embodiment, the present disclosure provides a method for slowing or preventing the development of leukemia in a subject who suffers from a myelodysplastic syndrome (MDS). The method comprises administering to the subject a therapeutically effective amount of an antimalarial endoperoxide compound or a pharmaceutically acceptable salt thereof.

The present disclosure also provides, in an additional embodiment, an antimalarial endoperoxide compound or a pharmaceutically acceptable salt thereof for treating a myelodysplastic syndrome (MDS) in a subject suffering therefrom.

In still another embodiment, the present disclosure provides an antimalarial endoperoxide compound or a pharmaceutically acceptable salt thereof for slowing or preventing the development of leukemia in a subject who suffers from a myelodysplastic syndrome (MDS).

The application further provides a compound of any one of Formulae I-V

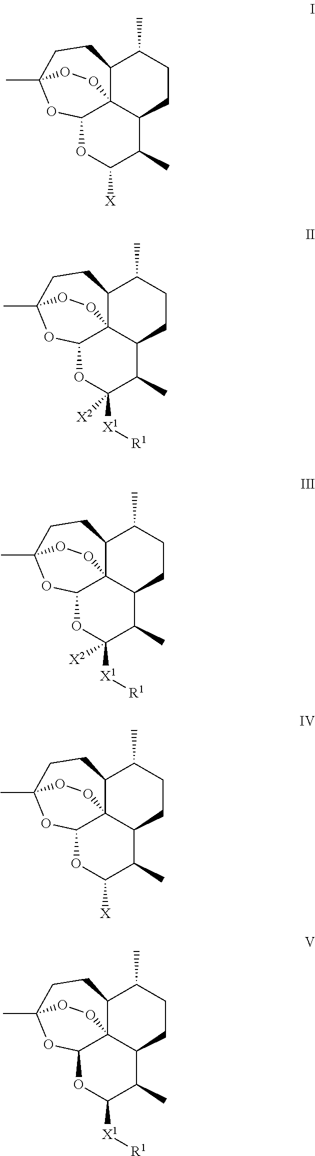

wherein
X is X¹R¹ or Y;
X¹ is —O—, —OC(═O)—, —OCH₂—, —OCH₂C(═O)—, —OCH(CH₃)—, —OCH₂C(═O)NH—, —CH₂—, —N(CH₃)—, —NH—, —NHCH₂—, —N(CH₃)CH₂—, —NHCH(CH₃)— or —S—;
R¹ is —(C₁-C₆) alkyl, —(C₁-C₆) heteroalkyl, —(C₁-C₆) hydroxyalkyl, —(C₆-C₁₀) aryl, —(C₁-C₆) alkyl (C₆-C₁₀) aryl, —(C₆-C₁₀) aryl-(C₁-C₆) alkyl, —NH(C₆-C₁₀) aryl, —(C₅-C₁₀) heteroaryl, —(C₁-C₆) alkyl (C₅-C₁₀) heteroaryl, —(C₃-C₁₀) cycloalkyl, —(C₁-C₆) alkyl (C₃-C₁₀) cycloalkyl, —(C₂-C₆) alkenyl (C₃-C₁₀) cycloalkyl, spiro —(C₃-C₁₀) cycloalkyl, —(C₃-C₁₀) heterocycloalkyl, —(C₁-C₆) alkyl (C₃-C₁₀) heterocycloalkyl, —S(═O)₂(C₁-C₆) alkyl, —O—S(═O)₂(C₁-C₆) alkyl, —(C₁-C₆) alkyl-S(═O)₂(C₁-C₆) alkyl, —(C₁-C₆) alkyl-S(═O)₂NH(C₁-C₆) alkyl), —(C₁-C₆) alkyl-S(═O)₂N((C₁-C₆) alkyl)₂, —(C₁-C₆) alkyl-S(═O)₂NH₂, —(C₁-C₆) alkyl-NHS(═O)₂(C₁-C₆) alkyl, —(C₁-C₆) alkyl) C(═O)NH(C₁-C₆) alkyl, —(C₁-C₆) alkyl) C(═O)NH₂, —C(═O)OH, —(C₁-C₆) alkyl-C(═O)OH, 2,3-dihydrobenzofuran, 2,3-dihydro-1H-indene, 3,4-dihydroisoquinolin-1(2H)-one, —B(OH)₂, —(C₁-C₆) alkyl-C(═O)O(C₁-C₆) alkyl, or partially unsaturated fused bicyclic (C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, —(C₁-C₆) heteroalkyl, or partially unsaturated fused bicyclic (C₃-C₁₀) heterocycloalkyl may optionally be substituted with one or more R¹′;
each R¹′ is independently —OH, halo, oxo, —CN, —NH₂, —(C₁-C₆) alkyl, —(C₁-C₆) halo alkyl, —O—(C₁-C₆) halo alkyl, —(C₁-C₆) alkyl-OH, —CH₂C(═O)O(C₁-C₆) alkyl, —C(═O)O(C₁-C₆) alkyl, —(C₁-C₆) alkyl-C(═O)OH, —CH(CH₃)C(═O)OH, —C(═O)OH, —C(═O)C(═O)OH, —(C₁-C₆) heteroalkyl, —O—(C₁-C₆) alkyl, —S(═O)₂(C₁-C₆) alkyl, —C(═O)NHOH, —C(═O)NH(C₁-C₆) alkyl, —(C₁-C₆) alkyl-C(═O)NH(C₁-C₆) alkyl, —NHS(═O)₂(C₁-C₆) alkyl, —S(═O)₂NH₂, —S(═O)₂NH(C₁-C₆) alkyl, —S(═O)₂N((C₁-C₆) alkyl)₂, —S(═O)₂NH—(C₃-C₁₀) cycloalkyl, —(C₁-C₆) alkyl-S(═O)₂NH—(C₃-C₁₀) cycloalkyl, —S(═O)₂CF₃, —C(═O)NH₂, —(C₃-C₁₀) cycloalkyl, —O—(C₃-C₁₀) cycloalkyl, —(C₃-C₁₀) heterocycloalkyl, —(C₅-C₁₀) heteroaryl, —(C₁-C₆) alkyl (C₅-C₁₀) heteroaryl, —C(═O)NH—(C₃-C₁₀) cycloalkyl, —NHC(═O)(C₃-C₁₀) cycloalkyl, —(C₁-C₆) alkyl-NHC(═O)(C₃-C₁₀) cycloalkyl, —(C₁-C₆) alkyl-C(═O)(C₃-C₁₀) heterocycloalkyl, —C(═O)N(CH₃)—(C₃-C₁₀) cycloalkyl, —(C₁-C₆) alkyl-C(═O)NH—(C₃-C₁₀) cycloalkyl, —C(═O)NH(C₁-C₆) alkyl-(C₃-C₁₀) cycloalkyl, —(C₁-C₆) alkyl-C(═O)NH(C₁-C₆) alkyl-(C₃-C₈) cycloalkyl, —C(═O)NH—(C₃-C₁₀) heterocycloalkyl, —C(═O)NH(C₅-C₁₀) heteroaryl, —(C₁-C₆) alkyl C(═O)NH(C₅-C₁₀) heteroaryl, C(═O)NH-thietanyl 1,1-dioxide, —(C₁-C₆) alkyl-C(═O)OH, —(C₂-C₆) alkenyl-C(═O)OH, —(C₂-C₆) alkynyl-C(═O)OH, —CH₂C(═O)NH₂, —C(═O)NH(C₁-C₆)alkyl), —C(═O)N((C₁-C₆)alkyl)₂, —(C₁-C₆) alkyl-C(═O)OH, —(C₆-C₁₀) aryl, —C(═O)NH(C₆-C₁₀) aryl, —C(═O)NH—(C₁-C₆) alkyl(C₆-C₁₀) aryl, —C(═O)NH—(C₁-C₆) alkyl-P⁺(Ph)₃, —P⁺(Ph)₃, —B(OH)₂, —NHC(═O)—C(═O)OH, or —C(═O)(C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, or —(C₁-C₆) heteroalkyl may optionally be substituted with one or more R¹″;
   or two R¹′ together form —(C₃-C₁₀) cycloalkyl or (C₃-C₁₀) heterocycloalkyl, optionally substituted with one or more R¹″;
each R¹″ is independently —OH, —NH₂, —(C₁-C₆) alkyl, —C(═O)OH, —(C₁-C₆) alkyl-C(═O)OH, —C(═O)O(C₁-C₆) alkyl, —CH₂C(═O)O(C₁-C₆) alkyl, —O(C₁-C₆) alkyl, —(C₁-C₆) halo alkyl, —O—(C₁-C₆) halo alkyl, —NHC(═O)OH, —NHC(═O)—(C₁-C₆) alkyl, —S(═O)₂NH₂, —S(═O)₂NH(C₁-C₆) alkyl, —S(═O)₂N((C₁-C₆) alkyl)₂, —S(═O)₂CF₃, —C(═O)NH₂, —(C₃-C₁₀) cycloalkyl, —(C₃-C₁₀) heterocycloalkyl, —(C₅-C₁₀) heteroaryl, oxo, or halo; and
Y is —(C₃-C₈) heterocycloalkyl, optionally substituted with one or more R¹′;
including enantiomers, racemic and scalemic mixtures, and pharmaceutically acceptable salts thereof.

### BRIEF DESCRIPTION OF THE DRAWINGS

FIG. 1. Schematic depicting the differentiation roadblock observed in myelodysplastic syndromes, and the effect of endoperoxide compounds on the same.

FIG. 2. High throughput multi-parametric flow cytometry-based assay for identifying inhibitors of MDS differentiation using MDS-L cells.

FIG. 3. Exemplary antimalarial endoperoxide compounds are efficacious differentiation-inducing agents in MDS-L cells.

FIG. 4A-FIG. 4C. Heme depletion induces terminal differentiation of MDS-L cells. A: Relative induction of GlyA positive MDS-L cells in response to treatment for 96 hours with the indicated concentrations of NMPP. B: Heme levels of MDS-L cells treated with the indicated compounds. C: Relative induction of GlyA positive cells after 96-hour treatment with artemisone and hemin.

### DETAILED DESCRIPTION OF THE INVENTION

Patent application WO2022/251788 is hereby incorporated herein by reference in its entirety. The present disclosure relates, in part, to repurposed drugs that correct the differentiation roadblock observed in MDS disease, allowing for the appropriate replenishment of the downstream blood lineages, an effect unachievable by standard chemotherapy drugs. More specifically, the present disclosure relates to therapeutic uses of known antimalarial medications as efficacious inducers of differentiation in MDS blast-like cells. Though the present disclosure is not bound by any particular theory, it is believed that induction occurs through a mechanism involving heme depletion.

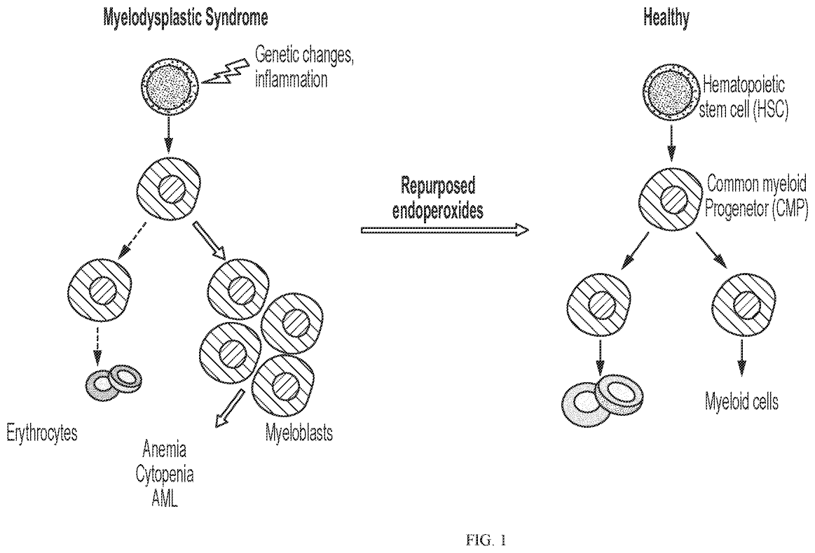

Ineffective erythropoiesis in MDS patients primarily results from aberrant regulation of terminal erythrocyte differentiation and maturation. A characteristic repertoire of MDS-associated mutations in HPCs induce a “differentiation roadblock” at later stages of the hierarchical differentiation tree. Re-stimulation of terminal erythrocyte differentiation and maturation in response to erythropoiesis-inducing stimuli (such as erythropoietin (EPO)) in erythrocyte progenitors underlies a functional cure for MDS. To this end, the present disclosure identifies repurposed drugs from high-throughput phenotypic screening for the functional rescue of erythropoiesis in patients with MDS (FIG. 1).

### EMBODIMENTS

Embodiment 1. A compound of any one of Formulae I-V

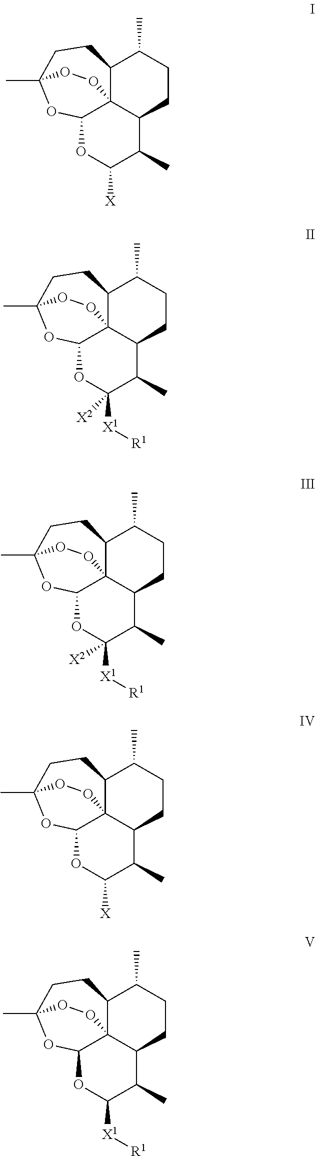

wherein
X is X¹R¹ or Y;
X¹ is —O—, —OC(═O)—, —OCH₂—, —OCH₂C(═O)—, —OCH(CH₃)—, —OCH₂C(═O)NH—, —CH₂—, —N(CH₃)—, —NH—, —NHCH₂—, —N(CH₃)CH₂—, —NHCH(CH₃)— or —S—;
R¹ is —(C₁-C₆) alkyl, —(C₁-C₆) heteroalkyl, —(C₁-C₆) hydroxyalkyl, —(C₆-C₁₀) aryl, —(C₁-C₆) alkyl (C₆-C₁₀) aryl, —(C₆-C₁₀) aryl-(C₁-C₆) alkyl, —NH(C₆-C₁₀) aryl, —(C₅-C₁₀) heteroaryl, —(C₁-C₆) alkyl (C₅-C₁₀) heteroaryl, —(C₃-C₁₀) cycloalkyl, —(C₁-C₆) alkyl (C₃-C₁₀) cycloalkyl, —(C₂-C₆) alkenyl (C₃-C₁₀) cycloalkyl, spiro —(C₃-C₁₀) cycloalkyl, —(C₃-C₁₀) heterocycloalkyl, —(C₁-C₆) alkyl (C₃-C₁₀) heterocycloalkyl, —S(═O)₂(C₁-C₆) alkyl, —O—S(═O)₂(C₁-C₆) alkyl, —(C₁-C₆) alkyl-S(═O)₂(C₁-C₆) alkyl, —(C₁-C₆) alkyl-S(═O)₂NH(C₁-C₆) alkyl), —(C₁-C₆) alkyl-S(═O)₂N((C₁-C₆) alkyl)₂, —(C₁-C₆) alkyl-S(═O)₂NH₂, —(C₁-C₆) alkyl-NHS(═O)₂(C₁-C₆) alkyl, —(C₁-C₆) alkyl) C(═O)NH(C₁-C₆) alkyl, —(C₁-C₆) alkyl) C(═O)NH₂, —C(═O)OH, —(C₁-C₆) alkyl-C(═O)OH, 2,3-dihydrobenzofuran, 2,3-dihydro-1H-indene, 3,4-dihydroisoquinolin-1(2H)-one, —B(OH)₂, —(C₁-C₆) alkyl-C(═O)O(C₁-C₆) alkyl, or partially unsaturated fused bicyclic (C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, —(C₁-C₆) heteroalkyl, or partially unsaturated fused bicyclic (C₃-C₁₀) heterocycloalkyl may optionally be substituted with one or more R¹′;
each R¹′ is independently —OH, halo, oxo, —CN, —NH₂, —(C₁-C₆) alkyl, —(C₁-C₆) halo alkyl, —O—(C₁-C₆) halo alkyl, —(C₁-C₆) alkyl-OH, —CH₂C(═O)O(C₁-C₆) alkyl, —C(═O)O(C₁-C₆) alkyl, —(C₁-C₆) alkyl-C(═O)OH, —CH(CH₃)C(═O)OH, —C(═O)OH, —C(═O)C(═O)OH, —(C₁-C₆) heteroalkyl, —O—(C₁-C₆) alkyl, —S(═O)₂(C₁-C₆) alkyl, —C(═O)NHOH, —C(═O)NH(C₁-C₆) alkyl, —(C₁-C₆) alkyl-C(═O)NH(C₁-C₆) alkyl, —NHS(═O)₂(C₁-C₆) alkyl, —S(═O)₂NH₂, —S(═O)₂NH(C₁-C₆) alkyl, —S(═O)₂N((C₁-C₆) alkyl)₂, —S(═O)₂NH—(C₃-C₁₀) cycloalkyl, —(C₁-C₆) alkyl-S(═O)₂NH—(C₃-C₁₀) cycloalkyl, —S(═O)₂CF₃, —C(═O)NH₂, —(C₃-C₁₀) cycloalkyl, —O—(C₃-C₁₀) cycloalkyl, —(C₃-C₁₀) heterocycloalkyl, —(C₅-C₁₀) heteroaryl, —(C₁-C₆) alkyl (C₅-C₁₀) heteroaryl, —C(═O)NH—(C₃-C₁₀) cycloalkyl, —NHC(═O)(C₃-C₁₀) cycloalkyl, —(C₁-C₆) alkyl-NHC(═O)(C₃-C₁₀) cycloalkyl, —(C₁-C₆) alkyl-C(═O)(C₃-C₁₀) heterocycloalkyl, —C(═O)N(CH₃)—(C₃-C₁₀) cycloalkyl, —(C₁-C₆) alkyl-C(═O)NH—(C₃-C₁₀) cycloalkyl, —C(═O)NH(C₁-C₆) alkyl-(C₃-C₁₀) cycloalkyl, —(C₁-C₆) alkyl-C(═O)NH(C₁-C₆) alkyl-(C₃-C₈) cycloalkyl, —C(═O)NH—(C₃-C₁₀) heterocycloalkyl, —C(═O)NH(C₅-C₁₀) heteroaryl, —(C₁-C₆) alkyl C(═O)NH(C₅-C₁₀) heteroaryl, C(═O)NH-thietanyl 1,1-dioxide, —(C₁-C₆) alkyl-C(═O)OH, —(C₂-C₆) alkenyl-C(═O)OH, —(C₂-C₆) alkynyl-C(═O)OH, —CH₂C(═O)NH₂, —C(═O)NH(C₁-C₆)alkyl), —C(═O)N((C₁-C₆)alkyl)₂, —(C₁-C₆) alkyl-C(═O)OH, —(C₆-C₁₀) aryl, —C(═O)NH(C₆-C₁₀) aryl, —C(═O)NH—(C₁-C₆) alkyl(C₆-C₁₀) aryl, —C(═O)NH—(C₁-C₆) alkyl-P⁺(Ph)₃, —P⁺(Ph)₃, —B(OH)₂, —NHC(═O)—C(═O)OH, or —C(═O)(C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, or —(C₁-C₆) heteroalkyl may optionally be substituted with one or more R¹′;
   or two R¹′ together form —(C₃-C₁₀) cycloalkyl or (C₃-C₁₀) heterocycloalkyl, optionally substituted with one or more R¹″;
each R¹″ is independently —OH, —NH₂, —(C₁-C₆) alkyl, —C(═O)OH, —(C₁-C₆) alkyl-C(═O)OH, —C(═O)O(C₁-C₆) alkyl, —CH₂C(═O)O(C₁-C₆) alkyl, —O(C₁-C₆) alkyl, —(C₁-C₆) halo alkyl, —O—(C₁-C₆) halo alkyl, —NHC(═O)OH, —NHC(═O)—(C₁-C₆) alkyl, —S(═O)₂NH₂, —S(═O)₂NH(C₁-C₆) alkyl, —S(═O)₂N((C₁-C₆) alkyl)₂, —S(═O)₂CF₃, —C(═O)NH₂, —(C₃-C₁₀) cycloalkyl, —(C₃-C₁₀) heterocycloalkyl, —(C₅-C₁₀) heteroaryl, oxo, or halo; and
Y is —(C₃-C₈) heterocycloalkyl, optionally substituted with one or more R¹′;
including enantiomers, racemic and scalemic mixtures, and pharmaceutically acceptable salts thereof.

Embodiment 2. The compound of Embodiment 1, wherein X² is H.

Embodiment 3. The compound of Embodiment 2, wherein X is X¹R¹.

Embodiment 4. The compound of Embodiment 3, wherein X¹ is —O—, —O—CH₂—, —O—CH₂C(═O)—, or —O—CH₂C(═O)NH—.

Embodiment 5. The compound of Embodiment 4, wherein R¹ is Ph, —(C₅-C₆) heteroaryl, or —(C₃-C₇) heterocycloalkyl optionally substituted with one or more R¹′.

Embodiment 6. The compound of Embodiment 5, wherein at least one R¹′ is —C(═O)OCH₃, —(C₁-C₆) alkyl-C(═O)OH, or —C(═O)OH.

Embodiment 7. The compound of Embodiment 5, wherein at least one R¹′ is (C₁-C₆) alkyl or is —(C₁-C₆) heteroalkyl.

Embodiment 8. The compound of Embodiment 5, wherein at least one R¹′ is —C(═O)NH(C₁-C₆) alkyl.

Embodiment 9. The compound of Embodiment 5, wherein at least one R¹′ is —OH, halo (C₁-C₆) alkyl, or CN.

Embodiment 10. The compound of Embodiment 5, wherein at least one R¹′ is —(C₃-C₁₀) cycloalkyl, —(C₃-C₁₀) heterocycloalkyl, or —(C₅-C₁₀) heteroaryl.

Embodiment 11. The compound of Embodiment 5, wherein at least one R¹″ is —C(═O)OH or —(C₁-C₆) alkyl-C(═O)OH and at least one R¹′ is halo, halo (C₁-C₆) alkyl, or —(C₁-C₆) alkyl.

Embodiment 12. The compound of Embodiment 5, wherein at least one R¹″ is —C(═O)OH.

Embodiment 13. A compound selected from the group consisting of:
(3R,5aS,6R,8aS,9R,10S,12R,12aR)-10-((2,2-difluorobenzo[d][1,3]dioxol-5-yl)methoxy)-3,6,9-trimethyldecahydro-12H-3,12-epoxy[1,2]dioxepino[4,3-i]isochromene;
5-(4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)phenyl)-2H-tetrazole;
2-(3-methoxy-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
3-(methylsulfonyl)-1-((3R,5aS,6R,8aS,9R,10R,12R,12aR)-3,6,9-trimethyldecahydro-12H-3,12-epoxy[1,2]dioxepino[4,3-i]isochromen-10-yl)azetidine;
N-hydroxy-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamide;
(3R,5aS,6R,8aS,9R,10R,12R,12aR)-10-((2,2-difluorobenzo[d][1,3]dioxol-5-yl)methoxy)-3,6,9-trimethyldecahydro-12H-3,12-epoxy[1,2]dioxepino[4,3-i]isochromene;
4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzonitrile;
2-(3-methyl-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
2-(3-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
4-(3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)propyl)benzoic acid;
(1R,4s)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
2-(4-(((3R,5aS,6R,8aS,9R,10R,12R,12aR)-3,6,9-trimethyldecahydro-12H-3,12-epoxy[1,2]dioxepino[4,3-i]isochromen-10-yl)oxy)phenyl)acetic acid;
5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)picolinic acid;
2-(2,6-difluoro-4-(((3R,5aS,6R,8aS,9R,10R,12R,12aR)-3,6,9-trimethyldecahydro-12H-3,12-epoxy[1,2]dioxepino[4,3-i]isochromen-10-yl)oxy)phenyl)acetic acid;
4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)picolinic acid;
2-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
(3R,5aS,6R,8aS,9R,10R,12R,12aR)—N,3,6,9-tetramethyl-N-(2-(methylsulfonyl)ethyl)decahydro-12H-3,12-epoxy[1,2]dioxepino[4,3-i]isochromen-10-amine;
4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzenesulfonamide;
4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzonitrile;
2-(3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
4-(3-(((3R,5aS,6R,8aS,9R,10R,12R,12aR)-3,6,9-trimethyldecahydro-12H-3,12-epoxy[1,2]dioxepino[4,3-i]isochromen-10-yl)oxy)propyl)benzoic acid;
2-(2,6-difluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
2-(2,3-difluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
2-fluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
2-fluoro-4-(((3R,5aS,6R,8aS,9R,10S,12S,12aR)-3,6,9-trimethyldecahydro-12H-3,12-epoxy[1,2]dioxepino[4,3-i]isochromen-10-yl)oxy)benzoic acid;
2-(2-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
2-(2-fluoro-5-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
(3R,5aS,6R,8aS,9R,10R,12R,12aR)-3,6,9-trimethyl-N-(2-((trifluoromethyl)sulfonyl)ethyl)decahydro-12H-3,12-epoxy[1,2]dioxepino[4,3-i]isochromen-10-amine;
2,6-difluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamide;
2,3-difluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)furan-2-carboxylic acid;
2-chloro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
2-(3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)acetic acid;
2-methoxy-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
4-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)ethyl)benzoic acid;
(3R,5aS,6R,8aS,9R,10R,12R,12aR)—N-((2,6-dimethylpyridin-4-yl)methyl)-3,6,9-trimethyldecahydro-12H-3,12-epoxy[1,2]dioxepino[4,3-i]isochromen-10-amine;
5-(4-((((3R,5aS,6R,8aS,9R,0S,12R,12aR)-3,6,9-trimethyldecahydro-12H-3,12-epoxy[1,2]dioxepino[4,3-i]isochromen-10-yl)oxy)methyl)phenyl)-2H-tetrazole;
2-fluoro-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
(1R,4r)-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)cyclohexane-1-carboxylic acid;
2-methyl-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
(1S,4s)-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)cyclohexane-1-carboxylic acid;
(1S,4r)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
3-methyl-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
3-fluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
(3R,5aS,6R,8aS,9R,10R,12R,12aR)-3,6,9-trimethyl-N-((1-methyl-1H-pyrazol-4-yl)methyl)decahydro-12H-3,12-epoxy[1,2]dioxepino[4,3-i]isochromen-10-amine;
2-((diethylamino)methyl)-5-(1-(((3R,5aS,6R,8aS,9R,10R,12R,12aR)-3,6,9-trimethyldecahydro-12H-3,12-epoxy[1,2]dioxepino[4,3-i]isochromen-10-yl)oxy)ethyl)phenol;
(3R,5aS,6R,8aS,9R,10R,12R,12aR)—N-((2-fluoropyridin-4-yl)methyl)-3,6,9-trimethyldecahydro-12H-3,12-epoxy[1,2]dioxepino[4,3-i]isochromen-10-amine;
4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzenesulfonamide;
(3R,5aS,6R,8aS,9R,10R,12R,12aR)—N-((2-methoxypyridin-4-yl)methyl)-3,6,9-trimethyldecahydro-12H-3,12-epoxy[1,2]dioxepino[4,3-i]isochromen-10-amine;
2-methyl-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
3-(((3R,5aS,6R,8aS,9R,10R,12R,12aR)-3,6,9-trimethyldecahydro-12H-3,12-epoxy[1,2]dioxepino[4,3-i]isochromen-10-yl)amino)thietane 1,1-dioxide;
(3R,5aS,6R,8aS,9R,10R,12R,12aR)-3,6,9-trimethyl-10-(2-(methylsulfonyl)ethoxy)decahydro-12H-3,12-epoxy[1,2]dioxepino[4,3-i]isochromene;
2-methoxy-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)bicyclo[1.1.1]pentane-1-carboxylic acid;
(3R,5aS,6R,8aS,9R,10S,12R,12aR)—N-(3-fluorophenyl)-3,6,9-trimethyldecahydro-12H-3,12-epoxy[1,2]dioxepino[4,3-i]isochromen-10-amine;
(3R,5aS,6R,8aS,9R,10R,12R,12aR)-3,6,9-trimethyl-N—((S)-1-(4-(methylsulfonyl)phenyl)ethyl)decahydro-12H-3,12-epoxy[1,2]dioxepino[4,3-i]isochromen-10-amine;
(3R,5aS,6R,8aS,9R,10R,12R,12aR)-3,6,9-trimethyl-N-(4-(methylsulfonyl)benzyl)decahydro-12H-3,12-epoxy[1,2]dioxepino[4,3-i]isochromen-10-amine;
N,N-dimethyl-2-(((3R,5aS,6R,8aS,9R,10R,12R,12aR)-3,6,9-trimethyldecahydro-12H-3,12-epoxy[1,2]dioxepino[4,3-i]isochromen-10-yl)amino)ethane-1-sulfonamide;
N-(oxetan-3-yl)-1-((3R,5aS,6R,8aS,9R,10R,12R,12aR)-3,6,9-trimethyldecahydro-12H-3,12-epoxy[1,2]dioxepino[4,3-i]isochromen-10-yl)piperidine-4-carboxamide;
5-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)furan-2-carboxylic acid;
N-(2-((3R,5aS,6R,8aS,9R,10S,12R,12aR)-3,6,9-trimethyldecahydro-12H-3,12-epoxy[1,2]dioxepino[4,3-i]isochromen-10-yl)ethyl)methanesulfonamide;
1-((3R,5aS,6R,8aS,9R,10R,12R,12aR)-3,6,9-trimethyldecahydro-12H-3,12-epoxy[1,2]dioxepino[4,3-i]isochromen-10-yl)piperidine;
1-((3R,5aS,6R,8aS,9R,10R,12R,12aR)-3,6,9-trimethyldecahydro-12H-3,12-epoxy[1,2]dioxepino[4,3-i]isochromen-10-yl)piperidine-4-carboxylic acid;
N-(1,1-dioxidothietan-3-yl)-1-((3R,5aS,6R,8aS,9R,10R,12R,12aR)-3,6,9-trimethyldecahydro-12H-3,12-epoxy[1,2]dioxepino[4,3-i]isochromen-10-yl)piperidine-4-carboxamide;
1-(2-((3R,5aS,6R,8aS,9R,10R,12R,12aR)-3,6,9-trimethyldecahydro-12H-3,12-epoxy[1,2]dioxepino[4,3-i]isochromen-10-yl)ethyl)piperidine-4-carboxylic acid;
N-methyl-4-((3R,5aS,6R,8aS,9R,10R,12R,12aR)-3,6,9-trimethyldecahydro-12H-3,12-epoxy[1,2]dioxepino[4,3-i]isochromen-10-yl)piperazine-1-carboxamide;
1-(2-((3R,5aS,6R,8aS,9R,10R,12R,12aR)-3,6,9-trimethyldecahydro-12H-3,12-epoxy[1,2]dioxepino[4,3-i]isochromen-10-yl)ethyl)azetidine-3-carboxylic acid;
(R)-1-((3R,5aS,6R,8aS,9R,10R,12R,12aR)-3,6,9-trimethyldecahydro-12H-3,12-epoxy[1,2]dioxepino[4,3-i]isochromen-10-yl)pyrrolidine-3-carboxylic acid;
methyl (R)-1-(2-((3R,5aS,6R,8aS,9R,10R,12R,12aR)-3,6,9-trimethyldecahydro-12H-3,12-epoxy[1,2]dioxepino[4,3-i]isochromen-10-yl)ethyl)pyrrolidine-3-carboxylate;
(1R,4s)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxylic acid;
5-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)pentanoic acid;
5-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)-2H-tetrazole;
3-methyl-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)bicyclo[2.2.2]octane-1-carboxylic acid;
3-methoxy-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
3-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanoic acid;
2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamide;
2-((1S,4s)-4-(((3R,5aS,6R,8aS,9R,10R,12R,12aR)-3,6,9-trimethyldecahydro-12H-3,12-epoxy[1,2]dioxepino[4,3-i]isochromen-10-yl)oxy)cyclohexyl)acetic acid;
6-(((3R,5aS,6R,8aS,9R,10R,12R,12aR)-3,6,9-trimethyldecahydro-12H-3,12-epoxy[1,2]dioxepino[4,3-i]isochromen-10-yl)oxy)spiro[3.3]heptane-2-carboxylic acid;
6-(((3R,5aS,6R,8aS,9R,10S,12R,12aR)-3,6,9-trimethyldecahydro-12H-3,12-epoxy[1,2]dioxepino[4,3-i]isochromen-10-yl)oxy)spiro[3.3]heptane-2-carboxylic acid;
2-((1R,4s)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexyl)acetic acid;
(3R,5aS,6R,8aS,9R,10R,12R,12aR)-3,6,9-trimethyldecahydro-12H-3,12-epoxy[1,2]dioxepino[4,3-i]isochromen-10-yl 4-(dimethylcarbamoyl)benzoate;
(R)-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanoic acid;
(3R,5aS,6R,8aS,9R,10S,12R,12aR)-10-(4-fluorophenoxy)-3,6,9-trimethyldecahydro-12H-3,12-epoxy[1,2]dioxepino[4,3-i]isochromene;
2-((1R,3S)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexyl)acetic acid;
2-fluoro-4-(3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)propyl)benzoic acid;
2-fluoro-4-(3-(((3R,5aS,6R,8aS,9R,10R,12R,12aR)-3,6,9-trimethyldecahydro-12H-3,12-epoxy[1,2]dioxepino[4,3-i]isochromen-10-yl)oxy)propyl)benzoic acid;
2-methoxy-4-(3-(((3R,5aS,6R,8aS,9R,10R,12R,12aR)-3,6,9-trimethyldecahydro-12H-3,12-epoxy[1,2]dioxepino[4,3-i]isochromen-10-yl)oxy)propyl)benzoic acid;
(3R,5aS,6R,8aS,9R,10S,12R,12aR)-3,6,9-trimethyl-10-(2-(methylsulfonyl)ethoxy)decahydro-12H-3,12-epoxy[1,2]dioxepino[4,3-i]isochromene;
3-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
2-chloro-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
(3R,5aS,6R,8aS,9R,10R,12R,12aR)-3,6,9-trimethyldecahydro-12H-3,12-epoxy[1,2]dioxepino[4,3-i]isochromen-10-yl 4-(4-chlorophenyl)-4-hydroxybutanoate;
(3R,5aS,6R,8aS,9R,10R,12R,12aR)-3,6,9-trimethyl-N-phenethyldecahydro-12H-3,12-epoxy[1,2]dioxepino[4,3-i]isochromen-10-amine;
N-methyl-3-(((3R,5aS,6R,8aS,9R,10R,12R,12aR)-3,6,9-trimethyldecahydro-12H-3,12-epoxy[1,2]dioxepino[4,3-i]isochromen-10-yl)amino)propenamide;
3-(((3R,5aS,6R,8aS,9R,10R,12R,12aR)-3,6,9-trimethyldecahydro-12H-3,12-epoxy[1,2]dioxepino[4,3-i]isochromen-10-yl)amino)propenamide;
N-methyl-2-(((3R,5aS,6R,8aS,9R,10R,12R,12aR)-3,6,9-trimethyldecahydro-12H-3,12-epoxy[1,2]dioxepino[4,3-i]isochromen-10-yl)amino)ethane-1-sulfonamide;
(3R,5aS,6R,8aS,9R,10R,12R,12aR)-3,6,9-trimethyl-N-(pyridin-4-ylmethyl)decahydro-12H-3,12-epoxy[1,2]dioxepino[4,3-i]isochromen-10-amine;
(3R,5aS,6R,8aS,9R,10R,12R,12aR)-3,6,9-trimethyl-N-(2-(methylsulfonyl)ethyl)decahydro-12H-3,12-epoxy[1,2]dioxepino[4,3-i]isochromen-10-amine;
(3R,5aS,6R,8aS,9R,10R,12R,12aR)-3,6,9-trimethyl-N-(3-(methylsulfonyl)propyl)decahydro-12H-3,12-epoxy[1,2]dioxepino[4,3-i]isochromen-10-amine;
(3R,5aS,6R,8aS,9R,10R,12R,12aR)—N-benzyl-3,6,9-trimethyldecahydro-12H-3,12-epoxy[1,2]dioxepino[4,3-i]isochromen-10-amine;
4-((methyl((3R,5aS,6R,8aS,9R,10R,12R,12aR)-3,6,9-trimethyldecahydro-12H-3,12-epoxy[1,2]dioxepino[4,3-i]isochromen-10-yl)amino)methyl)benzoic acid;
3-(methyl((3R,5aS,6R,8aS,9R,10R,12R,12aR)-3,6,9-trimethyldecahydro-12H-3,12-epoxy[1,2]dioxepino[4,3-i]isochromen-10-yl)amino)bicyclo[1.1.1]pentane-1-carboxylic acid;
N-(tert-butyl)-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamide;
(3R,5aS,6R,8aS,9R,10R,12R,12aR)-10-((4,4-difluorocyclohexyl)methoxy)-3,6,9-trimethyldecahydro-12H-3,12-epoxy[1,2]dioxepino[4,3-i]isochromene;
(3R,5aS,6R,8aS,9R,10S,12R,12aR)-10-((4,4-difluorocyclohexyl)methoxy)-3,6,9-trimethyldecahydro-12H-3,12-epoxy[1,2]dioxepino[4,3-i]isochromene;
4′-(((3R,5aS,6R,8aS,9R,10S,12R,12aR)-3,6,9-trimethyldecahydro-12H-3,12-epoxy[1,2]dioxepino[4,3-i]isochromen-10-yl)oxy)-[1,1′-biphenyl]-4-carboxylic acid;
((R)-3-fluoropyrrolidin-1-yl)(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)methanone;
3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)bicyclo[1.1.1]pentane-1-carboxylic acid;
(1R,3s)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclobutane-1-carboxylic acid;
2-(3-fluoro-4-(((3R,5aS,6R,8aS,9R,10R,12R,12aR)-3,6,9-trimethyldecahydro-12H-3,12-epoxy[1,2]dioxepino[4,3-i]isochromen-10-yl)oxy)phenyl)propanoic acid;
(3R,5aS,6R,8aS,9R,10S,12R,12aR)-10-((1,3-dihydroisobenzofuran-5-yl)oxy)-3,6,9-trimethyldecahydro-12H-3,12-epoxy[1,2]dioxepino[4,3-i]isochromene;
2-(3-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanoic acid;
(1S,3r)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclobutane-1-carboxylic acid;
3-methyl-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
3-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)bicyclo[1.1.1]pentane-1-carboxylic acid;
4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzonitrile;
2-methyl-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)pyridine;
3-chloro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
2-methyl-4-(3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)propyl)benzoic acid;
2-methoxy-4-(3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)propyl)benzoic acid;
2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propan-2-ol;
4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)bicyclo[2.2.1]heptane-1-carboxylic acid;
1-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)cyclopropane-1-carboxylic acid;
1-(4-(((3R,5aS,6R,8aS,9R,10R,12R,12aR)-3,6,9-trimethyldecahydro-12H-3,12-epoxy[1,2]dioxepino[4,3-i]isochromen-10-yl)oxy)phenyl)cyclopropane-1-carboxylic acid;
2-methyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanoic acid;
1-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclobutane-1-carboxylic acid;
2-oxo-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
2-oxo-2-(4-(((3R,5aS,6R,8aS,9R,10R,12R,12aR)-3,6,9-trimethyldecahydro-12H-3,12-epoxy[1,2]dioxepino[4,3-i]isochromen-10-yl)oxy)phenyl)acetic acid;
2-((1R,3S)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclopentyl)acetic acid;
(1R,4s)-4-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)propan-2-yl)cyclohexane-1-carboxylic acid;
tert-butyl 2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetate;
3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propiolic acid;
(1R,3 S)-3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)cyclopentane-1-carboxylic acid;
(1R,3 S)-3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
(1R,3 S)-3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclopentane-1-carboxylic acid;
3,5-difluoro-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
(1S,3R)-3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
3-(4-(((3R,5aS,6R,8aS,9R,10R,12R,12aR)-3,6,9-trimethyldecahydro-12H-3,12-epoxy[1,2]dioxepino[4,3-i]isochromen-10-yl)oxy)phenyl)propiolic acid;
(1S,4r)-4-fluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)isophthalic acid;
(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)glycine;
(1R,4s)-N-cyclopropyl-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamide;
(2S,5R)-1-methyl-5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)pyrrolidine-2-carboxylic acid;
(3R,5aS,6R,8aS,9R,10R,12R,12aR)-3,6,9-trimethyldecahydro-12H-3,12-epoxy[1,2]dioxepino[4,3-i]isochromen-10-yl 2-(3-(trifluoromethyl)-1H-pyrazol-1-yl)acetate;
3-methyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)butanoic acid;
(1R,3S,5r)-5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1,3-dicarboxylic acid;
1-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetyl)piperidine-4-carboxylic acid;
1-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)azetidine-3-carboxylic acid;
(2S,4R)-4-fluoro-1-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetyl)pyrrolidine-2-carboxylic acid;
(1R,2S,5S)-6,6-dimethyl-3-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetyl)-3-azabicyclo[3.1.0]hexane-2-carboxylic acid;
(1R,3S)-3-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetamido)cyclopentane-1-carboxylic acid;
(1R,4s)-N-(tert-butyl)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamide;
6-(((3R,5aS,6R,8aS,9R,10S,12R,12aR)-3,6,9-trimethyldecahydro-12H-3,12-epoxy[1,2]dioxepino[4,3-i]isochromen-10-yl)oxy)-2,3-dihydrobenzofuran-2-carboxylic acid;
2-(trifluoromethyl)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
((1R,4s)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexyl)methanol;
(1R,5R)-6-(((3R,5aS,6R,8aS,9R,10S,12R,12aR)-3,6,9-trimethyldecahydro-12H-3,12-epoxy[1,2]dioxepino[4,3-i]isochromen-10-yl)oxy)bicyclo[3.2.0]heptane-3-carboxylic acid;
6-(((3R,5aS,6R,8aS,9R,10S,12R,12aR)-3,6,9-trimethyldecahydro-12H-3,12-epoxy[1,2]dioxepino[4,3-i]isochromen-10-yl)oxy)bicyclo[3.1.0]hexane-3-carboxylic acid;
(2S,4R)-4-fluoro-1-(2-(((3R,5aS,6R,8aS,9R,10R,12R,12aR)-3,6,9-trimethyldecahydro-12H-3,12-epoxy[1,2]dioxepino[4,3-i]isochromen-10-yl)oxy)acetyl)pyrrolidine-2-carboxylic acid;
(2S,4S)-4-hydroxy-1-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetyl)pyrrolidine-2-carboxylic acid;
(R)-1-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetyl)pyrrolidine-3-carboxylic acid;
(1R,2S,5S)-6,6-dimethyl-3-(2-(((3R,5aS,6R,8aS,9R,10R,12R,12aR)-3,6,9-trimethyldecahydro-12H-3,12-epoxy[1,2]dioxepino[4,3-i]isochromen-10-yl)oxy)acetyl)-3-azabicyclo[3.1.0]hexane-2-carboxylic acid;
(R)-1-(2-(((3R,5aS,6R,8aS,9R,10R,12R,12aR)-3,6,9-trimethyldecahydro-12H-3,12-epoxy[1,2]dioxepino[4,3-i]isochromen-10-yl)oxy)acetyl)pyrrolidine-3-carboxylic acid;
1-(2-(((3R,5aS,6R,8aS,9R,10R,12R,12aR)-3,6,9-trimethyldecahydro-12H-3,12-epoxy[1,2]dioxepino[4,3-i]isochromen-10-yl)oxy)acetyl)piperidine-4-carboxylic acid;
3-fluoro-4-(3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)prop-1-yn-1-yl)benzoic acid;
3-fluoro-4-(3-(((3R,5aS,6R,8aS,9R,10R,12R,12aR)-3,6,9-trimethyldecahydro-12H-3,12-epoxy[1,2]dioxepino[4,3-i]isochromen-10-yl)oxy)prop-1-yn-1-yl)benzoic acid;
4-chloro-3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
4-chloro-3-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)boronic acid;
1-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)-2-oxabicyclo[2.1.1]hexane-4-carboxylic acid;
2,2-difluoro-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
2-phenyl-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)propanoic acid;
(1R,3S)-3-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)cyclohexane-1-carboxylic acid;
3-fluoro-4-(3-(((3R,5aS,6R,8aS,9R,10R,12R,12aR)-3,6,9-trimethyldecahydro-12H-3,12-epoxy[1,2]dioxepino[4,3-i]isochromen-10-yl)oxy)propyl)benzoic acid;
3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)cyclobutane-1-carboxylic acid;
(4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)phenyl)boronic acid;
3,5-dichloro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
3-chloro-5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
3-chloro-4-(1-(((3R,5aS,6R,8aS,9R,10S,12R,12aR)-3,6,9-trimethyldecahydro-12H-3,12-epoxy[1,2]dioxepino[4,3-i]isochromen-10-yl)oxy)ethyl)benzoic acid;
2-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)-2-methylpropanoic acid;
3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)bicyclo[3.1.0]hexane-6-carboxylic acid;
(1R,3s)-3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)cyclobutane-1-carboxylic acid;
2-(3-(trifluoromethyl)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
(S)-3,3-dimethyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)butanoic acid;
(S)-2-cyclobutyl-2-((1R,4R)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)acetic acid;
2-(3-chloro-5-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
(1R,2S)-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)cyclopentane-1-carboxylic acid;
(S)-3,3-dimethyl-2-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetamido)butanoic acid;
(S)-2-phenyl-2-((1R,4R)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)acetic acid;
(1R,2S,5S)-6,6-dimethyl-3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)-3-azabicyclo[3.1.0]hexane-2-carboxylic acid;
(1S,3aR,6aS)-2-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetyl)octahydrocyclopenta[c]pyrrole-1-carboxylic acid;
2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)cyclopropane-1-carboxylic acid;
(1R,4s)-4-methoxy-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
(S)-2-amino-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
3-(trifluoromethyl)-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
3-chloro-4-((((3R,5aS,6R,8aS,9R,10R,12R,12aR)-3,6,9-trimethyl-10-(trifluoromethyl)decahydro-12H-3,12-epoxy[1,2]dioxepino[4,3-i]isochromen-10-yl)oxy)methyl)benzoic acid;
3-(trifluoromethyl)-4-((((3R,5aS,6R,8aS,9R,12R,12aR)-3,6,9-trimethyldecahydro-12H-3,12-epoxy[1,2]dioxepino[4,3-i]isochromen-10-yl)oxy)methyl)benzoic acid;
N—((S)-2,6-dioxopiperidin-3-yl)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamide;
(1S,4r)-4-methoxy-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
(1R,4s)-4-fluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
(1R,3S)-3-((1R,3R)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclobutane-1-carboxamido)cyclopentane-1-carboxylic acid;
(1R,3S)-3-((1R,4R)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)cyclopentane-1-carboxylic acid;
2-oxo-2-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)piperidin-1-yl)acetic acid;
(1S,4s)-4-((((3R,5aS,6R,8aS,9R,10R,12R,12aR)-3,6,9-trimethyl-10-(trifluoromethyl)decahydro-12H-3,12-epoxy[1,2]dioxepino[4,3-i]isochromen-10-yl)oxy)methyl)cyclohexane-1-carboxylic acid;
(S)-3-cyclobutyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)propanoic acid;
(1R,3s)-3-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)propan-2-yl)cyclobutane-1-carboxylic acid;
(2S,3aS,6aS)-1-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)octahydrocyclopenta[b]pyrrole-2-carboxylic acid;
5-(((3R,5aS,6R,8aS,9R,10S,12R,12aR)-3,6,9-trimethyldecahydro-12H-3,12-epoxy[1,2]dioxepino[4,3-i]isochromen-10-yl)oxy)-1H-indole-2-carboxylic acid;
5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)tetrahydrofuran-2-carboxylic acid;
(3R)-3-fluoro-3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclopentane-1-carboxylic acid;
4-hydroxy-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
(6R)-6-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)tetrahydro-2H-pyran-3-carboxylic acid;
4-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetamido)benzoic acid;
6-(((3R,5aS,6R,8aS,9R,10S,12R,12aR)-3,6,9-trimethyldecahydro-12H-3,12-epoxy[1,2]dioxepino[4,3-i]isochromen-10-yl)oxy)-2-naphthoic acid;
3-chloro-5-fluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
3-chloro-5-(methylsulfonyl)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
3-chloro-5-(methylsulfonyl)-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
2-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)acetic acid;
2-((4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzyl)oxy)acetic acid;
2-(3-fluoro-5-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)acetic acid;
2-methyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)propanoic acid;
3-fluoro-3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
2-(3-chloro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenoxy)acetic acid;
2-(((1R,4s)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexyl)oxy)acetic acid;
1-(((3R,5aS,6R,8aS,9R,10R,12R,12aR)-3,6,9-trimethyldecahydro-12H-3,12-epoxy[1,2]dioxepino[4,3-i]isochromen-10-yl)oxy)-2,3-dihydro-1H-indene-5-carboxylic acid;
1-(((3R,5aS,6R,8aS,9R,10S,12R,12aR)-3,6,9-trimethyldecahydro-12H-3,12-epoxy[1,2]dioxepino[4,3-i]isochromen-10-yl)oxy)-2,3-dihydro-1H-indene-5-carboxylic acid;
3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)cyclopentane-1-carboxylic acid;
(1S,3s)-3-((1R,4S)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)cyclobutane-1-carboxylic acid;
(S)-3,3-dimethyl-2-((1R,4R)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)butanoic acid;
2-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)acetic acid;
(S)-2-cyclohexyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)acetic acid;
(1R,3S)-3-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)cyclopentane-1-carboxylic acid;
(1R,3s)-3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)cyclobutane-1-carboxylic acid;
(S)-2-phenyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)acetic acid;
3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoic acid;
(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)-L-valine;
(S)-4,4,4-trifluoro-2-((1R,4R)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)butanoic acid;
2-(3,5-difluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)acetic acid;
(1R,3S)-3-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)cyclopentane-1-carboxylic acid;
(S)-4,4,4-trifluoro-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)butanoic acid;
(1S,3s)-3-((1R,4S)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)cyclobutane-1-carboxylic acid;
(S)-3,3-dimethyl-2-((1R,4R)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)butanoic acid;
(1R,4s)-4-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)cyclohexane-1-carboxylic acid;
1-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)azetidine-3-carboxylic acid;
1-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzyl)-1H-1,2,3-triazole-4-carboxylic acid;
4-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)benzoic acid;
(R)-1-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)pyrrolidine-3-carboxylic acid;
O-methyl-N-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)-L-threonine;
(S)-3-hydroxy-3-methyl-2-((1R,4R)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)butanoic acid;
3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)propanoic acid;
(S)-2-cyclobutyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)acetic acid;
(S)-2-cyclobutyl-2-((1R,4R)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)acetic acid;
(2S,3S,5S,6S,7S,8S)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cubane-1-carboxylic acid;
(S)-3-hydroxy-3-methyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)butanoic acid;
4-((1R,3s)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclobutane-1-carboxamido)cubane-1-carboxylic acid;
(S)-4,4,4-trifluoro-2-((1R,4R)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)butanoic acid;
(S)-2-(4-(trifluoromethyl)phenyl)-2-((1R,3R)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclobutane-1-carboxamido)acetic acid;
(1S,3S,5S)-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)-2-azabicyclo[3.1.0]hexane-3-carboxylic acid;
1-((1R,4s)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carbonyl)azetidine-3-carboxylic acid;
(2R,3R,5R,6R,7R,8R)-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)cubane-1-carboxylic acid;
(S)-3-methyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)butanoic acid;
1-((1R,4s)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)cyclopropane-1-carboxylic acid;
(R)-1-((1R,4S)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carbonyl)pyrrolidine-3-carboxylic acid;
1-(2-bromo-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzyl)-1H-1,2,3-triazole-4-carboxylic acid;
3-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)oxetane-3-carboxylic acid;
2-(3-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)-3-methylbutanoic acid;
(1R,3S)-3-((1R,4R)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)cyclopentane-1-carboxylic acid;
1-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)cyclopropane-1-carboxylic acid;
(1S,3R)-3-((E)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)prop-1-en-1-yl)cyclohexane-1-carboxylic acid;
2-(3-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)-2-methylpropanoic acid;
(1S,4r)-4-fluoro-4-((E)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)prop-1-en-1-yl)cyclohexane-1-carboxylic acid;
(1R,3S)-3-(N-methyl-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)cyclopentane-1-carboxylic acid;
3-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)bicyclo[1.1.1]pentane-1-carboxylic acid;
2-(4-((((3S,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenoxy)acetic acid;
(1R,3S)-3-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclopentane-1-carboxylic acid;
(2S,4S)-4-fluoro-1-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoyl)pyrrolidine-2-carboxylic acid;
1-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoyl)piperidine-4-carboxylic acid;
(1R,3s)-3-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclobutane-1-carboxylic acid;
(R)-2-(3-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanoic acid;
3-chloro-5-methyl-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
(S)-3,3-dimethyl-2-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)butanoic acid;
(S)-2-(3-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanoic acid;
3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)oxetane-3-carboxylic acid;
2-methyl-2-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)propanoic acid;
(2-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)acetyl)-L-valine;
1-((4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)methyl)cyclobutane-1-carboxylic acid;
4-((1R,4s)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)benzoic acid;
(1S,3R,4R)-3-hydroxy-4-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclopentane-1-carboxylic acid;
(1S,3R)-3-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)cyclopentane-1-carboxylic acid;
(1R,3R)-3-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclohexane-1-carboxylic acid;
6-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)spiro[3.3]heptane-2-carboxylic acid;
(1R,3s)-3-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)cyclobutane-1-carboxylic acid;
4-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)bicyclo[2.1.1]hexane-1-carboxylic acid;
(1R,3S)-3-((4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)carbamoyl)cyclopentane-1-carboxylic acid;
4-((1R,4s)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)bicyclo[2.1.1]hexane-1-carboxylic acid;
2-(5,5-dimethyl-2,4-dioxo-3-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)imidazolidin-1-yl)acetic acid;
1-(((1R,4s)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)methyl)cyclobutane-1-carboxylic acid;
2-(trifluoromethoxy)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
(1S,3R,4R)-3-hydroxy-4-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)cyclopentane-1-carboxylic acid;
4-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)bicyclo[2.1.1]hexane-1-carboxylic acid;
(S)-2-cyclopropyl-2-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)acetic acid;
(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetyl)-L-valine;
3-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)bicyclo[1.1.1]pentane-1-carboxylic acid;
2-(5-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)-1,3,4-oxadiazol-2-yl)acetic acid;
(1S,3R)-3-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclopentane-1-carboxylic acid;
(1R,3S)-3-(2-(3-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanamido)cyclopentane-1-carboxylic acid;
(1S,3S)-3-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)cyclopentane-1-carboxylic acid;
(1R,4s)-4-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)cyclohexane-1-carboxylic acid;
(1R,3S)-3-(3,5-difluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclopentane-1-carboxylic acid;
2-(trifluoromethoxy)-5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
(1R,3R)-3-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)cyclopentane-1-carboxylic acid;
2-(1-methyl-1H-pyrazol-4-yl)-2-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)acetic acid;
(1S,3R)-3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)cyclopentane-1-carboxylic acid;
(S)-2-acetamido-3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanoic acid;
(1S,2R,3S,4R)-2,3-dihydroxy-4-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclopentane-1-carboxylic acid;
(1R,3S)-3-((4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzyl)carbamoyl)cyclopentane-1-carboxylic acid;
(1R,3S)-3-(3-chloro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclopentane-1-carboxylic acid;
2-(4-(4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)phenyl)-1H-1,2,3-triazol-1-yl)acetic acid;
2-(4-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)-1H-1,2,3-triazol-1-yl)acetic acid;
3-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)propanoic acid;
2-(2-(trifluoromethoxy)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
2-(2-(difluoromethoxy)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
(1R,2S,4R,5S)-4-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)bicyclo[3.1.0]hexane-2-carboxylic acid;
(2S,4R)-4-hydroxy-1-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoyl)pyrrolidine-2-carboxylic acid;
(1R,3S)-3-(((4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)methyl)sulfonamido)cyclopentane-1-carboxylic acid;
(1R,3S)-3-((4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)sulfonamido)cyclopentane-1-carboxylic acid;
6-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)spiro[3.3]heptane-2-carboxylic acid;
(1R,3s)-3-((4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)carbamoyl)cyclobutane-1-carboxylic acid;
(S)-1-oxo-7-(((3R,5aS,6R,8aS,9R,10S,12R,12aR)-3,6,9-trimethyldecahydro-12H-3,12-epoxy[1,2]dioxepino[4,3-i]isochromen-10-yl)oxy)-1,2,3,4-tetrahydroisoquinoline-3-carboxylic acid; and
(1R,5S,6S)-3-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetyl)-3-azabicyclo[3.1.0]hexane-6-carboxylic acid.

Embodiment 14. A pharmaceutical composition comprising the compound of any one of Embodiments 1-13 admixed with a pharmaceutically acceptable carrier, diluent, or excipient.

Embodiment 15. The pharmaceutical composition of Embodiment 14, further comprising one or more therapeutic compounds or compositions.

Embodiment 16. The pharmaceutical composition of Embodiment 15, wherein the one or more therapeutic compounds or compositions is a second anticancer compound or composition.

Embodiment 17. A method of preventing, ameliorating, or treating a myelodysplastic syndrome (MDS), comprising administering to a subject in need thereof a therapeutically effective amount of the compound of either Embodiment 1 or Embodiment 13.

Embodiment 18. A method of preventing, ameliorating, or treating a myelodysplastic syndrome (MDS), comprising administering to a subject in need thereof a therapeutically effective amount of the pharmaceutical composition of Embodiment 14.

Embodiment 19. A method of preventing, ameliorating, or treating a myelodysplastic syndrome (MDS), comprising administering to a subject in need thereof a therapeutically effective amount of the pharmaceutical composition of Embodiment 15.

Embodiment 20. A method of preventing, ameliorating, or treating a myelodysplastic syndrome (MDS), comprising administering to a subject in need thereof a therapeutically effective amount of the pharmaceutical composition of Embodiment 16.

Embodiment 21. A method for slowing or preventing the development of leukemia in a subject, comprising administering to the subject a therapeutically effective amount of the compound of either Embodiment 1 or Embodiment 13.

Embodiment 22. A method for slowing or preventing the development of leukemia in a subject, comprising administering to the subject a therapeutically effective amount of the pharmaceutical composition of Embodiment 14.

Embodiment 23. A method for slowing or preventing the development of leukemia in a subject, comprising administering to the subject a therapeutically effective amount of the pharmaceutical composition of Embodiment 15.

Embodiment 24. A method for slowing or preventing the development of leukemia in a subject, comprising administering to the subject a therapeutically effective amount of the pharmaceutical composition of Embodiment 16.

Embodiment 25. The method of Embodiment 17, wherein the MDS is primary MDS.

Embodiment 26. The method of Embodiment 17, wherein the MDS is secondary MDS.

Embodiment 27. The method of Embodiment 17, wherein the MDS is one or more subtypes chosen from refractory anemia (RA), refractory anemia with ringed sideroblasts (RARS), refractory cytopenia with multilineage dysplasia (RCMD), refractory cytopenia with multilineage dysplasia and ringed sideroblasts (RCMD-RS), refractory anemia with excess blasts (RAEB), myelodysplastic syndrome unclassified (MDS-U), and MDS associated with isolated del(5q).

Embodiment 28. A compound of Formula I

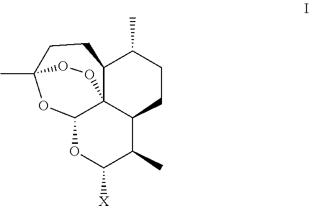

wherein
X is X¹R¹ or Y;
X¹ is —O—, —OC(═O)—, —OCH₂—, —OCH₂C(═O)—, —OCH(CH₃)—, —OCH₂C(═O)NH—, —CH₂—, —N(CH₃)—, —NH—, —NHCH₂—, —N(CH₃)CH₂—, —NHCH(CH₃)— or —S—;
R¹ is —(C₁-C₆) alkyl, —(C₁-C₆) heteroalkyl, —(C₁-C₆) hydroxyalkyl, —(C₆-C₁₀) aryl, —(C₁-C₆) alkyl (C₆-C₁₀) aryl, —(C₆-C₁₀) aryl-(C₁-C₆) alkyl, —NH(C₆-C₁₀) aryl, —(C₅-C₁₀) heteroaryl, —(C₁-C₆) alkyl (C₅-C₁₀) heteroaryl, —(C₃-C₁₀) cycloalkyl, —(C₁-C₆) alkyl (C₃-C₁₀) cycloalkyl, —(C₂-C₆) alkenyl (C₃-C₁₀) cycloalkyl, spiro —(C₃-C₁₀) cycloalkyl, —(C₃-C₁₀) heterocycloalkyl, —(C₁-C₆) alkyl (C₃-C₁₀) heterocycloalkyl, —S(═O)₂(C₁-C₆) alkyl, —O—S(═O)₂(C₁-C₆) alkyl, —(C₁-C₆) alkyl-S(═O)₂(C₁-C₆) alkyl, —(C₁-C₆) alkyl-S(═O)₂NH(C₁-C₆) alkyl), —(C₁-C₆) alkyl-S(═O)₂N((C₁-C₆) alkyl)₂, —(C₁-C₆) alkyl-S(═O)₂NH₂, —(C₁-C₆) alkyl-NHS(═O)₂(C₁-C₆) alkyl, —(C₁-C₆) alkyl) C(═O)NH(C₁-C₆) alkyl, —(C₁-C₆) alkyl) C(═O)NH₂, —C(═O)OH, —(C₁-C₆) alkyl-C(═O)OH, 2,3-dihydrobenzofuran, 2,3-dihydro-1H-indene, 3,4-dihydroisoquinolin-1(2H)-one, —B(OH)₂, —(C₁-C₆) alkyl-C(═O)O(C₁-C₆) alkyl, or partially unsaturated fused bicyclic (C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, —(C₁-C₆) heteroalkyl, or partially unsaturated fused bicyclic (C₃-C₁₀) heterocycloalkyl may optionally be substituted with one or more R¹′;
each R¹′ is independently —OH, halo, oxo, —CN, —NH₂, —(C₁-C₆) alkyl, —(C₁-C₆) halo alkyl, —O—(C₁-C₆) halo alkyl, —(C₁-C₆) alkyl-OH, —CH₂C(═O)O(C₁-C₆) alkyl, —C(═O)O(C₁-C₆) alkyl, —(C₁-C₆) alkyl-C(═O)OH, —CH(CH₃)C(═O)OH, —C(═O)OH, —C(═O)C(═O)OH, —(C₁-C₆) heteroalkyl, —O—(C₁-C₆) alkyl, —S(═O)₂(C₁-C₆) alkyl, —C(═O)NHOH, —C(═O)NH(C₁-C₆) alkyl, —(C₁-C₆) alkyl-C(═O)NH(C₁-C₆) alkyl, —NHS(═O)₂(C₁-C₆) alkyl, —S(═O)₂NH₂, —S(═O)₂NH(C₁-C₆) alkyl, —S(═O)₂N((C₁-C₆) alkyl)₂, —S(═O)₂NH—(C₃-C₁₀) cycloalkyl, —(C₁-C₆) alkyl-S(═O)₂NH—(C₃-C₁₀) cycloalkyl, —S(═O)₂CF₃, —C(═O)NH₂, —(C₃-C₁₀) cycloalkyl, —O—(C₃-C₁₀) cycloalkyl, —(C₃-C₁₀) heterocycloalkyl, —(C₅-C₁₀) heteroaryl, —(C₁-C₆) alkyl (C₅-C₁₀) heteroaryl, —C(═O)NH—(C₃-C₁₀) cycloalkyl, —NHC(═O)(C₃-C₁₀) cycloalkyl, —(C₁-C₆) alkyl-NHC(═O)(C₃-C₁₀) cycloalkyl, —(C₁-C₆) alkyl-C(═O)(C₃-C₁₀) heterocycloalkyl, —C(═O)N(CH₃)—(C₃-C₁₀) cycloalkyl, —(C₁-C₆) alkyl-C(═O)NH—(C₃-C₁₀) cycloalkyl, —C(═O)NH(C₁-C₆) alkyl-(C₃-C₁₀) cycloalkyl, —(C₁-C₆) alkyl-C(═O)NH(C₁-C₆) alkyl-(C₃-C₈) cycloalkyl, —C(═O)NH—(C₃-C₁₀) heterocycloalkyl, —C(═O)NH(C₅-C₁₀) heteroaryl, —(C₁-C₆) alkyl C(═O)NH(C₅-C₁₀) heteroaryl, C(═O)NH-thietanyl 1,1-dioxide, —(C₁-C₆) alkyl-C(═O)OH, —(C₂-C₆) alkenyl-C(═O)OH, —(C₂-C₆) alkynyl-C(═O)OH, —CH₂C(═O)NH₂, —C(═O)NH(C₁-C₆)alkyl), —C(═O)N((C₁-C₆)alkyl)₂, —(C₁-C₆) alkyl-C(═O)OH, —(C₆-C₁₀) aryl, —C(═O)NH(C₆-C₁₀) aryl, —C(═O)NH—(C₁-C₆) alkyl(C₆-C₁₀) aryl, —C(═O)NH—(C₁-C₆) alkyl-P⁺(Ph)₃, —P⁺(Ph)₃, —B(OH)₂, —NHC(═O)—C(═O)OH, or —C(═O)(C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, or —(C₁-C₆) heteroalkyl may optionally be substituted with one or more R¹″;
   or two R¹′ together form —(C₃-C₁₀) cycloalkyl or (C₃-C₁₀) heterocycloalkyl, optionally substituted with one or more R¹″;
each R¹″ is independently —OH, —NH₂, —(C₁-C₆) alkyl, —C(═O)OH, —(C₁-C₆) alkyl-C(═O)OH, —C(═O)O(C₁-C₆) alkyl, —CH₂C(═O)O(C₁-C₆) alkyl, —O(C₁-C₆) alkyl, —(C₁-C₆) halo alkyl, —O—(C₁-C₆) halo alkyl, —NHC(═O)OH, —NHC(═O)—(C₁-C₆) alkyl, —S(═O)₂NH₂, —S(═O)₂NH(C₁-C₆) alkyl, —S(═O)₂N((C₁-C₆) alkyl)₂, —S(═O)₂CF₃, —C(═O)NH₂, —(C₃-C₁₀) cycloalkyl, —(C₃-C₁₀) heterocycloalkyl, —(C₅-C₁₀) heteroaryl, oxo, or halo; and
Y is —(C₃-C₈) heterocycloalkyl, optionally substituted with one or more R¹′; including enantiomers, racemic and scalemic mixtures, and pharmaceutically acceptable salts thereof.

Embodiment 29. A compound of Formula II

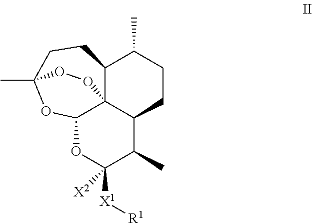

wherein
X¹ is —O—, —OC(═O)—, —OCH₂—, —OCH₂C(═O)—, —OCH(CH₃)—, —OCH₂C(═O)NH—, —CH₂—, —N(CH₃)—, —NH—, —NHCH₂—, —N(CH₃)CH₂—, —NHCH(CH₃)— or —S—;
R¹ is —(C₁-C₆) alkyl, —(C₁-C₆) heteroalkyl, —(C₁-C₆) hydroxyalkyl, —(C₆-C₁₀) aryl, —(C₁-C₆) alkyl (C₆-C₁₀) aryl, —(C₆-C₁₀) aryl-(C₁-C₆) alkyl, —NH(C₆-C₁₀) aryl, —(C₅-C₁₀) heteroaryl, —(C₁-C₆) alkyl (C₅-C₁₀) heteroaryl, —(C₃-C₁₀) cycloalkyl, —(C₁-C₆) alkyl (C₃-C₁₀) cycloalkyl, —(C₂-C₆) alkenyl (C₃-C₁₀) cycloalkyl, spiro —(C₃-C₁₀) cycloalkyl, —(C₃-C₁₀) heterocycloalkyl, —(C₁-C₆) alkyl (C₃-C₁₀) heterocycloalkyl, —S(═O)₂(C₁-C₆) alkyl, —O—S(═O)₂(C₁-C₆) alkyl, —(C₁-C₆) alkyl-S(═O)₂(C₁-C₆) alkyl, —(C₁-C₆) alkyl-S(═O)₂NH(C₁-C₆) alkyl), —(C₁-C₆) alkyl-S(═O)₂N((C₁-C₆) alkyl)₂, —(C₁-C₆) alkyl-S(═O)₂NH₂, —(C₁-C₆) alkyl-NHS(═O)₂(C₁-C₆) alkyl, —(C₁-C₆) alkyl) C(═O)NH(C₁-C₆) alkyl, —(C₁-C₆) alkyl) C(═O)NH₂, —C(═O)OH, —(C₁-C₆) alkyl-C(═O)OH, 2,3-dihydrobenzofuran, 2,3-dihydro-1H-indene, 3,4-dihydroisoquinolin-1(2H)-one, —B(OH)₂, —(C₁-C₆) alkyl-C(═O)O(C₁-C₆) alkyl, or partially unsaturated fused bicyclic (C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, —(C₁-C₆) heteroalkyl, or partially unsaturated fused bicyclic (C₃-C₁₀) heterocycloalkyl may optionally be substituted with one or more R¹′;
each R¹′ is independently —OH, halo, oxo, —CN, —NH₂, —(C₁-C₆) alkyl, —(C₁-C₆) halo alkyl, —O—(C₁-C₆) halo alkyl, —(C₁-C₆) alkyl-OH, —CH₂C(═O)O(C₁-C₆) alkyl, —C(═O)O(C₁-C₆) alkyl, —(C₁-C₆) alkyl-C(═O)OH, —CH(CH₃)C(═O)OH, —C(═O)OH, —C(═O)C(═O)OH, —(C₁-C₆) heteroalkyl, —O—(C₁-C₆) alkyl, —S(═O)₂(C₁-C₆) alkyl, —C(═O)NHOH, —C(═O)NH(C₁-C₆) alkyl, —(C₁-C₆) alkyl-C(═O)NH(C₁-C₆) alkyl, —NHS(═O)₂(C₁-C₆) alkyl, —S(═O)₂NH₂, —S(═O)₂NH(C₁-C₆) alkyl, —S(═O)₂N((C₁-C₆) alkyl)₂, —S(═O)₂NH—(C₃-C₁₀) cycloalkyl, —(C₁-C₆) alkyl-S(═O)₂NH—(C₃-C₁₀) cycloalkyl, —S(═O)₂CF₃, —C(═O)NH₂, —(C₃-C₁₀) cycloalkyl, —O—(C₃-C₁₀) cycloalkyl, —(C₃-C₁₀) heterocycloalkyl, —(C₅-C₁₀) heteroaryl, —(C₁-C₆) alkyl (C₅-C₁₀) heteroaryl, —C(═O)NH—(C₃-C₁₀) cycloalkyl, —NHC(═O)(C₃-C₁₀) cycloalkyl, —(C₁-C₆) alkyl-NHC(═O)(C₃-C₁₀) cycloalkyl, —(C₁-C₆) alkyl-C(═O)(C₃-C₁₀) heterocycloalkyl, —C(═O)N(CH₃)—(C₃-C₁₀) cycloalkyl, —(C₁-C₆) alkyl-C(═O)NH—(C₃-C₁₀) cycloalkyl, —C(═O)NH(C₁-C₆) alkyl-(C₃-C₁₀) cycloalkyl, —(C₁-C₆) alkyl-C(═O)NH(C₁-C₆) alkyl-(C₃-C₈) cycloalkyl, —C(═O)NH—(C₃-C₁₀) heterocycloalkyl, —C(═O)NH(C₅-C₁₀) heteroaryl, —(C₁-C₆) alkyl C(═O)NH(C₅-C₁₀) heteroaryl, C(═O)NH-thietanyl 1,1-dioxide, —(C₁-C₆) alkyl-C(═O)OH, —(C₂-C₆) alkenyl-C(═O)OH, —(C₂-C₆) alkynyl-C(═O)OH, —CH₂C(═O)NH₂, —C(═O)NH(C₁-C₆)alkyl), —C(═O)N((C₁-C₆)alkyl)₂, —(C₁-C₆) alkyl-C(═O)OH, —(C₆-C₁₀) aryl, —C(═O)NH(C₆-C₁₀) aryl, —C(═O)NH—(C₁-C₆) alkyl(C₆-C₁₀) aryl, —C(═O)NH—(C₁-C₆) alkyl-P⁺(Ph)₃, —P⁺(Ph)₃, —B(OH)₂, —NHC(═O)—C(═O)OH, or —C(═O)(C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, or —(C₁-C₆) heteroalkyl may optionally be substituted with one or more R¹″;
   or two R¹′ together form —(C₃-C₁₀) cycloalkyl or (C₃-C₁₀) heterocycloalkyl, optionally substituted with one or more R¹″;
each R¹″ is independently —OH, —NH₂, —(C₁-C₆) alkyl, —C(═O)OH, —(C₁-C₆) alkyl-C(═O)OH, —C(═O)O(C₁-C₆) alkyl, —CH₂C(═O)O(C₁-C₆) alkyl, —O(C₁-C₆) alkyl, —(C₁-C₆) halo alkyl, —O—(C₁-C₆) halo alkyl, —NHC(═O)OH, —NHC(═O)—(C₁-C₆) alkyl, —S(═O)₂NH₂, —S(═O)₂NH(C₁-C₆) alkyl, —S(═O)₂N((C₁-C₆) alkyl)₂, —S(═O)₂CF₃, —C(═O)NH₂, —(C₃-C₁₀) cycloalkyl, —(C₃-C₁₀) heterocycloalkyl, —(C₅-C₁₀) heteroaryl, oxo, or halo; and
X² is H, —OH, halo, —(C₁-C₆) alkyl, or —(C₁-C₆) halo alkyl; including enantiomers, racemic and scalemic mixtures, and pharmaceutically acceptable salts thereof.

Embodiment 30. A compound of Formula III

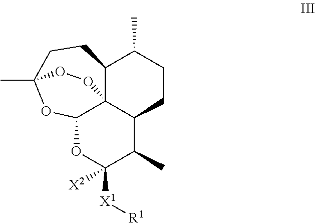

wherein
X¹ is —O—, —OC(═O)—, —OCH₂—, —OCH₂C(═O)—, —OCH(CH₃)—, —OCH₂C(═O)NH—, —CH₂—, —N(CH₃)—, —NH—, —NHCH₂—, —N(CH₃)CH₂—, —NHCH(CH₃)— or —S—;
R¹ is —(C₁-C₆) alkyl, —(C₁-C₆) heteroalkyl, —(C₁-C₆) hydroxyalkyl, —(C₆-C₁₀) aryl, —(C₁-C₆) alkyl (C₆-C₁₀) aryl, —(C₆-C₁₀) aryl-(C₁-C₆) alkyl, —NH(C₆-C₁₀) aryl, —(C₅-C₁₀) heteroaryl, —(C₁-C₆) alkyl (C₅-C₁₀) heteroaryl, —(C₃-C₁₀) cycloalkyl, —(C₁-C₆) alkyl (C₃-C₁₀) cycloalkyl, —(C₂-C₆) alkenyl (C₃-C₁₀) cycloalkyl, spiro —(C₃-C₁₀) cycloalkyl, —(C₃-C₁₀) heterocycloalkyl, —(C₁-C₆) alkyl (C₃-C₁₀) heterocycloalkyl, —S(═O)₂(C₁-C₆) alkyl, —O—S(═O)₂(C₁-C₆) alkyl, —(C₁-C₆) alkyl-S(═O)₂(C₁-C₆) alkyl, —(C₁-C₆) alkyl-S(═O)₂NH(C₁-C₆) alkyl), —(C₁-C₆) alkyl-S(═O)₂N((C₁-C₆) alkyl)₂, —(C₁-C₆) alkyl-S(═O)₂NH₂, —(C₁-C₆) alkyl-NHS(═O)₂(C₁-C₆) alkyl, —(C₁-C₆) alkyl) C(═O)NH(C₁-C₆) alkyl, —(C₁-C₆) alkyl) C(═O)NH₂, —C(═O)OH, —(C₁-C₆) alkyl-C(═O)OH, 2,3-dihydrobenzofuran, 2,3-dihydro-1H-indene, 3,4-dihydroisoquinolin-1(2H)-one, —B(OH)₂, —(C₁-C₆) alkyl-C(═O)O(C₁-C₆) alkyl, or partially unsaturated fused bicyclic (C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, —(C₁-C₆) heteroalkyl, or partially unsaturated fused bicyclic (C₃-C₁₀) heterocycloalkyl may optionally be substituted with one or more R¹′;
each R¹′ is independently —OH, halo, oxo, —CN, —NH₂, —(C₁-C₆) alkyl, —(C₁-C₆) halo alkyl, —O—(C₁-C₆) halo alkyl, —(C₁-C₆) alkyl-OH, —CH₂C(═O)O(C₁-C₆) alkyl, —C(═O)O(C₁-C₆) alkyl, —(C₁-C₆) alkyl-C(═O)OH, —CH(CH₃)C(═O)OH, —C(═O)OH, —C(═O)C(═O)OH, —(C₁-C₆) heteroalkyl, —O—(C₁-C₆) alkyl, —S(═O)₂(C₁-C₆) alkyl, —C(═O)NHOH, —C(═O)NH(C₁-C₆) alkyl, —(C₁-C₆) alkyl-C(═O)NH(C₁-C₆) alkyl, —NHS(═O)₂(C₁-C₆) alkyl, —S(═O)₂NH₂, —S(═O)₂NH(C₁-C₆) alkyl, —S(═O)₂N((C₁-C₆) alkyl)₂, —S(═O)₂NH—(C₃-C₁₀) cycloalkyl, —(C₁-C₆) alkyl-S(═O)₂NH—(C₃-C₁₀) cycloalkyl, —S(═O)₂CF₃, —C(═O)NH₂, —(C₃-C₁₀) cycloalkyl, —O—(C₃-C₁₀) cycloalkyl, —(C₃-C₁₀) heterocycloalkyl, —(C₅-C₁₀) heteroaryl, —(C₁-C₆) alkyl (C₅-C₁₀) heteroaryl, —C(═O)NH—(C₃-C₁₀) cycloalkyl, —NHC(═O)(C₃-C₁₀) cycloalkyl, —(C₁-C₆) alkyl-NHC(═O)(C₃-C₁₀) cycloalkyl, —(C₁-C₆) alkyl-C(═O)(C₃-C₁₀) heterocycloalkyl, —C(═O)N(CH₃)—(C₃-C₁₀) cycloalkyl, —(C₁-C₆) alkyl-C(═O)NH—(C₃-C₁₀) cycloalkyl, —C(═O)NH(C₁-C₆) alkyl-(C₃-C₁₀) cycloalkyl, —(C₁-C₆) alkyl-C(═O)NH(C₁-C₆) alkyl-(C₃-C₈) cycloalkyl, —C(═O)NH—(C₃-C₁₀) heterocycloalkyl, —C(═O)NH(C₅-C₁₀) heteroaryl, —(C₁-C₆) alkyl C(═O)NH(C₅-C₁₀) heteroaryl, C(═O)NH-thietanyl 1,1-dioxide, —(C₁-C₆) alkyl-C(═O)OH, —(C₂-C₆) alkenyl-C(═O)OH, —(C₂-C₆) alkynyl-C(═O)OH, —CH₂C(═O)NH₂, —C(═O)NH(C₁-C₆)alkyl), —C(═O)N((C₁-C₆)alkyl)₂, —(C₁-C₆) alkyl-C(═O)OH, —(C₆-C₁₀) aryl, —C(═O)NH(C₆-C₁₀) aryl, —C(═O)NH—(C₁-C₆) alkyl(C₆-C₁₀) aryl, —C(═O)NH—(C₁-C₆) alkyl-P⁺(Ph)₃, —P⁺(Ph)₃, —B(OH)₂, —NHC(═O)—C(═O)OH, or —C(═O)(C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, or —(C₁-C₆) heteroalkyl may optionally be substituted with one or more R¹″;
   or two R¹′ together form —(C₃-C₁₀) cycloalkyl or (C₃-C₁₀) heterocycloalkyl, optionally substituted with one or more R¹″;
each R¹″ is independently —OH, —NH₂, —(C₁-C₆) alkyl, —C(═O)OH, —(C₁-C₆) alkyl-C(═O)OH, —C(═O)O(C₁-C₆) alkyl, —CH₂C(═O)O(C₁-C₆) alkyl, —O(C₁-C₆) alkyl, —(C₁-C₆) halo alkyl, —O—(C₁-C₆) halo alkyl, —NHC(═O)OH, —NHC(═O)—(C₁-C₆) alkyl, —S(═O)₂NH₂, —S(═O)₂NH(C₁-C₆) alkyl, —S(═O)₂N((C₁-C₆) alkyl)₂, —S(═O)₂CF₃, —C(═O)NH₂, —(C₃-C₁₀) cycloalkyl, —(C₃-C₁₀) heterocycloalkyl, —(C₅-C₁₀) heteroaryl, oxo, or halo; and
X² is H, —OH, halo, —(C₁-C₆) alkyl, or —(C₁-C₆) halo alkyl; including enantiomers, racemic and scalemic mixtures, and pharmaceutically acceptable salts thereof.

Embodiment 31. A compound of the Formula IV

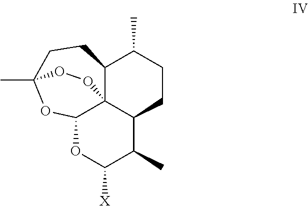

wherein
X is X¹R¹ or Y;
X¹ is —O—, —OC(═O)—, —OCH₂—, —OCH₂C(═O)—, —OCH(CH₃)—, —OCH₂C(═O)NH—, —CH₂—, —N(CH₃)—, —NH—, —NHCH₂—, —N(CH₃)CH₂—, —NHCH(CH₃)— or —S—;
R¹ is —(C₁-C₆) alkyl, —(C₁-C₆) heteroalkyl, —(C₁-C₆) hydroxyalkyl, —(C₆-C₁₀) aryl, —(C₁-C₆) alkyl (C₆-C₁₀) aryl, —(C₆-C₁₀) aryl-(C₁-C₆) alkyl, —NH(C₆-C₁₀) aryl, —(C₅-C₁₀) heteroaryl, —(C₁-C₆) alkyl (C₅-C₁₀) heteroaryl, —(C₃-C₁₀) cycloalkyl, —(C₁-C₆) alkyl (C₃-C₁₀) cycloalkyl, —(C₂-C₆) alkenyl (C₃-C₁₀) cycloalkyl, spiro —(C₃-C₁₀) cycloalkyl, —(C₃-C₁₀) heterocycloalkyl, —(C₁-C₆) alkyl (C₃-C₁₀) heterocycloalkyl, —S(═O)₂(C₁-C₆) alkyl, —O—S(═O)₂(C₁-C₆) alkyl, —(C₁-C₆) alkyl-S(═O)₂(C₁-C₆) alkyl, —(C₁-C₆) alkyl-S(═O)₂NH(C₁-C₆) alkyl), —(C₁-C₆) alkyl-S(═O)₂N((C₁-C₆) alkyl)₂, —(C₁-C₆) alkyl-S(═O)₂NH₂, —(C₁-C₆) alkyl-NHS(═O)₂(C₁-C₆) alkyl, —(C₁-C₆) alkyl) C(═O)NH(C₁-C₆) alkyl, —(C₁-C₆) alkyl) C(═O)NH₂, —C(═O)OH, —(C₁-C₆) alkyl-C(═O)OH, 2,3-dihydrobenzofuran, 2,3-dihydro-1H-indene, 3,4-dihydroisoquinolin-1(2H)-one, —B(OH)₂, —(C₁-C₆) alkyl-C(═O)O(C₁-C₆) alkyl, or partially unsaturated fused bicyclic (C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, —(C₁-C₆) heteroalkyl, or partially unsaturated fused bicyclic (C₃-C₁₀) heterocycloalkyl may optionally be substituted with one or more R¹′;
each R¹′ is independently —OH, halo, oxo, —CN, —NH₂, —(C₁-C₆) alkyl, —(C₁-C₆) halo alkyl, —O—(C₁-C₆) halo alkyl, —(C₁-C₆) alkyl-OH, —CH₂C(═O)O(C₁-C₆) alkyl, —C(═O)O(C₁-C₆) alkyl, —(C₁-C₆) alkyl-C(═O)OH, —CH(CH₃)C(═O)OH, —C(═O)OH, —C(═O)C(═O)OH, —(C₁-C₆) heteroalkyl, —O—(C₁-C₆) alkyl, —S(═O)₂(C₁-C₆) alkyl, —C(═O)NHOH, —C(═O)NH(C₁-C₆) alkyl, —(C₁-C₆) alkyl-C(═O)NH(C₁-C₆) alkyl, —NHS(═O)₂(C₁-C₆) alkyl, —S(═O)₂NH₂, —S(═O)₂NH(C₁-C₆) alkyl, —S(═O)₂N((C₁-C₆) alkyl)₂, —S(═O)₂NH—(C₃-C₁₀) cycloalkyl, —(C₁-C₆) alkyl-S(═O)₂NH—(C₃-C₁₀) cycloalkyl, —S(═O)₂CF₃, —C(═O)NH₂, —(C₃-C₁₀) cycloalkyl, —O—(C₃-C₁₀) cycloalkyl, —(C₃-C₁₀) heterocycloalkyl, —(C₅-C₁₀) heteroaryl, —(C₁-C₆) alkyl (C₅-C₁₀) heteroaryl, —C(═O)NH—(C₃-C₁₀) cycloalkyl, —NHC(═O)(C₃-C₁₀) cycloalkyl, —(C₁-C₆) alkyl-NHC(═O)(C₃-C₁₀) cycloalkyl, —(C₁-C₆) alkyl-C(═O)(C₃-C₁₀) heterocycloalkyl, —C(═O)N(CH₃)—(C₃-C₁₀) cycloalkyl, —(C₁-C₆) alkyl-C(═O)NH—(C₃-C₁₀) cycloalkyl, —C(═O)NH(C₁-C₆) alkyl-(C₃-C₁₀) cycloalkyl, —(C₁-C₆) alkyl-C(═O)NH(C₁-C₆) alkyl-(C₃-C₈) cycloalkyl, —C(═O)NH—(C₃-C₁₀) heterocycloalkyl, —C(═O)NH(C₅-C₁₀) heteroaryl, —(C₁-C₆) alkyl C(═O)NH(C₅-C₁₀) heteroaryl, C(═O)NH-thietanyl 1,1-dioxide, —(C₁-C₆) alkyl-C(═O)OH, —(C₂-C₆) alkenyl-C(═O)OH, —(C₂-C₆) alkynyl-C(═O)OH, —CH₂C(═O)NH₂, —C(═O)NH(C₁-C₆)alkyl), —C(═O)N((C₁-C₆)alkyl)₂, —(C₁-C₆) alkyl-C(═O)OH, —(C₆-C₁₀) aryl, —C(═O)NH(C₆-C₁₀) aryl, —C(═O)NH—(C₁-C₆) alkyl(C₆-C₁₀) aryl, —C(═O)NH—(C₁-C₆) alkyl-P⁺(Ph)₃, —P⁺(Ph)₃, —B(OH)₂, —NHC(═O)—C(═O)OH, or —C(═O)(C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, or —(C₁-C₆) heteroalkyl may optionally be substituted with one or more R¹″;
   or two R¹′ together form —(C₃-C₁₀) cycloalkyl or (C₃-C₁₀) heterocycloalkyl, optionally substituted with one or more R¹″;
each R¹″ is independently —OH, —NH₂, —(C₁-C₆) alkyl, —C(═O)OH, —(C₁-C₆) alkyl-C(═O)OH, —C(═O)O(C₁-C₆) alkyl, —CH₂C(═O)O(C₁-C₆) alkyl, —O(C₁-C₆) alkyl, —(C₁-C₆) halo alkyl, —O—(C₁-C₆) halo alkyl, —NHC(═O)OH, —NHC(═O)—(C₁-C₆) alkyl, —S(═O)₂NH₂, —S(═O)₂NH(C₁-C₆) alkyl, —S(═O)₂N((C₁-C₆) alkyl)₂, —S(═O)₂CF₃, —C(═O)NH₂, —(C₃-C₁₀) cycloalkyl, —(C₃-C₁₀) heterocycloalkyl, —(C₅-C₁₀) heteroaryl, oxo, or halo; and
Y is —(C₃-C₈) heterocycloalkyl, optionally substituted with one or more R¹′; including enantiomers, racemic and scalemic mixtures, and pharmaceutically acceptable salts thereof.

Embodiment 32. A compound of the Formula V

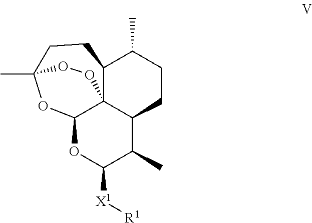

wherein
X¹ is —O—, —OC(═O)—, —OCH₂—, —OCH₂C(═O)—, —OCH(CH₃)—, —OCH₂C(═O)NH—, —CH₂—, —N(CH₃)—, —NH—, —NHCH₂—, —N(CH₃)CH₂—, —NHCH(CH₃)— or —S—;
R¹ is —(C₁-C₆) alkyl, —(C₁-C₆) heteroalkyl, —(C₁-C₆) hydroxyalkyl, —(C₆-C₁₀) aryl, —(C₁-C₆) alkyl (C₆-C₁₀) aryl, —(C₆-C₁₀) aryl-(C₁-C₆) alkyl, —NH(C₆-C₁₀) aryl, —(C₅-C₁₀) heteroaryl, —(C₁-C₆) alkyl (C₅-C₁₀) heteroaryl, —(C₃-C₁₀) cycloalkyl, —(C₁-C₆) alkyl (C₃-C₁₀) cycloalkyl, —(C₂-C₆) alkenyl (C₃-C₁₀) cycloalkyl, spiro —(C₃-C₁₀) cycloalkyl, —(C₃-C₁₀) heterocycloalkyl, —(C₁-C₆) alkyl (C₃-C₁₀) heterocycloalkyl, —S(═O)₂(C₁-C₆) alkyl, —O—S(═O)₂(C₁-C₆) alkyl, —(C₁-C₆) alkyl-S(═O)₂(C₁-C₆) alkyl, —(C₁-C₆) alkyl-S(═O)₂NH(C₁-C₆) alkyl), —(C₁-C₆) alkyl-S(═O)₂N((C₁-C₆) alkyl)₂, —(C₁-C₆) alkyl-S(═O)₂NH₂, —(C₁-C₆) alkyl-NHS(═O)₂(C₁-C₆) alkyl, —(C₁-C₆) alkyl) C(═O)NH(C₁-C₆) alkyl, —(C₁-C₆) alkyl) C(═O)NH₂, —C(═O)OH, —(C₁-C₆) alkyl-C(═O)OH, 2,3-dihydrobenzofuran, 2,3-dihydro-1H-indene, 3,4-dihydroisoquinolin-1(2H)-one, —B(OH)₂, —(C₁-C₆) alkyl-C(═O)O(C₁-C₆) alkyl, or partially unsaturated fused bicyclic (C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, —(C₁-C₆) heteroalkyl, or partially unsaturated fused bicyclic (C₃-C₁₀) heterocycloalkyl may optionally be substituted with one or more R¹′;
each R₁′ is independently —OH, halo, oxo, —CN, —NH₂, —(C₁-C₆) alkyl, —(C₁-C₆) halo alkyl, —O—(C₁-C₆) halo alkyl, —(C₁-C₆) alkyl-OH, —CH₂C(═O)O(C₁-C₆) alkyl, —C(═O)O(C₁-C₆) alkyl, —(C₁-C₆) alkyl-C(═O)OH, —CH(CH₃)C(═O)OH, —C(═O)OH, —C(═O)C(═O)OH, —(C₁-C₆) heteroalkyl, —O—(C₁-C₆) alkyl, —S(═O)₂(C₁-C₆) alkyl, —C(═O)NHOH, —C(═O)NH(C₁-C₆) alkyl, —(C₁-C₆) alkyl-C(═O)NH(C₁-C₆) alkyl, —NHS(═O)₂(C₁-C₆) alkyl, —S(═O)₂NH₂, —S(═O)₂NH(C₁-C₆) alkyl, —S(═O)₂N((C₁-C₆) alkyl)₂, —S(═O)₂NH—(C₃-C₁₀) cycloalkyl, —(C₁-C₆) alkyl-S(═O)₂NH—(C₃-C₁₀) cycloalkyl, —S(═O)₂CF₃, —C(═O)NH₂, —(C₃-C₁₀) cycloalkyl, —O—(C₃-C₁₀) cycloalkyl, —(C₃-C₁₀) heterocycloalkyl, —(C₅-C₁₀) heteroaryl, —(C₁-C₆) alkyl (C₅-C₁₀) heteroaryl, —C(═O)NH—(C₃-C₁₀) cycloalkyl, —NHC(═O)(C₃-C₁₀) cycloalkyl, —(C₁-C₆) alkyl-NHC(═O)(C₃-C₁₀) cycloalkyl, —(C₁-C₆) alkyl-C(═O)(C₃-C₁₀) heterocycloalkyl, —C(═O)N(CH₃)—(C₃-C₁₀) cycloalkyl, —(C₁-C₆) alkyl-C(═O)NH—(C₃-C₁₀) cycloalkyl, —C(═O)NH(C₁-C₆) alkyl-(C₃-C₁₀) cycloalkyl, —(C₁-C₆) alkyl-C(═O)NH(C₁-C₆) alkyl-(C₃-C₈) cycloalkyl, —C(═O)NH—(C₃-C₁₀) heterocycloalkyl, —C(═O)NH(C₅-C₁₀) heteroaryl, —(C₁-C₆) alkyl C(═O)NH(C₅-C₁₀) heteroaryl, C(═O)NH-thietanyl 1,1-dioxide, —(C₁-C₆) alkyl-C(═O)OH, —(C₂-C₆) alkenyl-C(═O)OH, —(C₂-C₆) alkynyl-C(═O)OH, —CH₂C(═O)NH₂, —C(═O)NH(C₁-C₆)alkyl), —C(═O)N((C₁-C₆)alkyl)₂, —(C₁-C₆) alkyl-C(═O)OH, —(C₆-C₁₀) aryl, —C(═O)NH(C₆-C₁₀) aryl, —C(═O)NH—(C₁-C₆) alkyl(C₆-C₁₀) aryl, —C(═O)NH—(C₁-C₆) alkyl-P⁺(Ph)₃, —P⁺(Ph)₃, —B(OH)₂, —NHC(═O)—C(═O)OH, or —C(═O)(C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, or —(C₁-C₆) heteroalkyl may optionally be substituted with one or more R¹″;
   or two R¹′ together form —(C₃-C₁₀) cycloalkyl or (C₃-C₁₀) heterocycloalkyl, optionally substituted with one or more R¹″; and
each R¹″ is independently —OH, —NH₂, —(C₁-C₆) alkyl, —C(═O)OH, —(C₁-C₆) alkyl-C(═O)OH, —C(═O)O(C₁-C₆) alkyl, —CH₂C(═O)O(C₁-C₆) alkyl, —O(C₁-C₆) alkyl, —(C₁-C₆) halo alkyl, —O—(C₁-C₆) halo alkyl, —NHC(═O)OH, —NHC(═O)—(C₁-C₆) alkyl, —S(═O)₂NH₂, —S(═O)₂NH(C₁-C₆) alkyl, —S(═O)₂N((C₁-C₆) alkyl)₂, —S(═O)₂CF₃, —C(═O)NH₂, —(C₃-C₁₀) cycloalkyl, —(C₃-C₁₀) heterocycloalkyl, —(C₅-C₁₀) heteroaryl, oxo, or halo; including enantiomers, racemic and scalemic mixtures, and pharmaceutically acceptable salts thereof.

Embodiment 33. The compound of either Embodiment 28 or Embodiment 31 wherein X is X¹R¹.

Embodiment 34. The compound of any one of Embodiments 28-33, wherein X¹ is —O—, —NH—, —NHCH₂—, or —OCH₂—.

Embodiment 35. The compound of any one of Embodiments 28-34, wherein R¹ is Ph optionally substituted with one or more R¹′.

Embodiment 36. The compound of any one of Embodiments 28-34, wherein R¹ is —(C₅-C₆) heteroaryl optionally substituted with one or more R¹′.

Embodiment 37. The compound of any one of Embodiments 28-34, wherein R¹ is (C₃-C₇) heterocycloalkyl optionally substituted with one or more R¹′.

Embodiment 38. The compound of any one of Embodiments 28-37, wherein at least one R¹′ is —C(═O)O(C₁-C₆) alkyl.

Embodiment 39. The compound of any one of Embodiments 28-37, wherein at least one R¹′ is (C₁-C₆) alkyl.

Embodiment 40. The compound of any one of Embodiments 28-37, wherein at least one R¹′ is —(C₁-C₆) heteroalkyl.

Embodiment 41. The compound of Embodiment 40, wherein —(C₁-C₆) heteroalkyl is —OMe.

Embodiment 42. The compound of any one of Embodiments 28-37, wherein at least one R¹′ is —(C₁-C₆) alkyl-C(═O)OH.

Embodiment 43. The compound of Embodiment 42, wherein —(C₁-C₆) alkyl-C(═O)OH is —CH₂C(═O)OH.

Embodiment 44. The compound of any one of Embodiments 28-37, wherein at least one R¹′ is CN.

Embodiment 45. The compound of any one of Embodiments 28-37, wherein at least one R¹′ is halo.

Embodiment 46. The compound of Embodiment 45, wherein halo is F.

Embodiment 47. The compound of Embodiment 45, wherein halo is C₁.

Embodiment 48. The compound of any one of Embodiments 28-37, wherein at least one R¹′ is —(C₅-C₆) heteroaryl.

Embodiment 49. The compound of any one of Embodiments 28-37, wherein at least one R¹′ is —OH.

Embodiment 50. The compound of any one of Embodiments 28-37, wherein at least one R¹′ is —S(═O)₂(C₁-C₆) alkyl or —(C₁-C₆) alkyl-S(═O)₂(C₁-C₆) alkyl.

Embodiment 51. The compound of any one of Embodiments 28-50, wherein X is X¹R¹ and X¹ is or —OCH₂—.

Embodiment 52. The compound of any one of Embodiments 28-50, wherein X is X¹R¹ and X¹ is —O—.

Embodiment 53. The compound of any one of Embodiments 28-50, wherein X is X¹R¹ and X¹ is —NH— or —NHCH₂—.

Embodiment 54. A pharmaceutical composition comprising the compound of any one of Embodiments 13, or 28-53 admixed with a pharmaceutically acceptable carrier, diluent, or excipient.

Embodiment 55. The pharmaceutical composition of Embodiment 54, further comprising one or more therapeutic compounds or compositions.

Embodiment 56. The pharmaceutical composition of Embodiment 55, wherein the one or more therapeutic compounds or compositions is a second anticancer compound or composition.

Embodiment 57. A method of preventing, ameliorating, or treating a myelodysplastic syndrome (MDS), comprising administering to a subject in need thereof a therapeutically effective amount of the compound of any one of Embodiments 13, or 28-53 or the pharmaceutical compositions of any one of Embodiments 54-56.

Embodiment 58. A method for slowing or preventing the development of leukemia in a subject, comprising administering to the subject a therapeutically effective amount of the compound of any one of Embodiments 13, or 28-53 or the pharmaceutical compositions of any one of Embodiments 54-56.

Embodiment 59. The method according to embodiment 57, wherein the MDS is primary MDS.

Embodiment 60. The method according to embodiment 57, wherein the MDS is secondary MDS.

Embodiment 61. The method according to embodiment 57, wherein the MDS is one or more subtypes chosen from refractory anemia (RA), refractory anemia with ringed sideroblasts (RARS), refractory cytopenia with multilineage dysplasia (RCMD), refractory cytopenia with multilineage dysplasia and ringed sideroblasts (RCMD-RS), refractory anemia with excess blasts (RAEB), myelodysplastic syndrome unclassified (MDS-U), and MDS associated with isolated del(5q).

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention, the Detailed Description of the Invention, the Experimentals, or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which “R” appears twice and is defined as “independently selected from” means that each instance of that R group is separately identified as one member of the set which follows in the definition of that R group. For example, “each R¹ and R² is independently selected from carbon and nitrogen” means that both R¹ and R² can be carbon, both R¹ and R² can be nitrogen, or R¹ or R² can be carbon and the other nitrogen or vice versa.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or a line drawn through a bond or “~~~” drawn through a bond each refer to the point of attachment of a functional group or other chemical moiety to the rest of the molecule of which it is a part.

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the “optionally substituted” moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds disclosed herein may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “alkyl” as used herein denotes an unbranched or branched chain, saturated, monovalent hydrocarbon residue containing 1 to 12 carbon atoms. The term “lower alkyl” or “C₁-C₆ alkyl” as used herein denotes a straight or branched chain hydrocarbon residue containing 1 to 6 carbon atoms. “C₁₋₁₂ alkyl” as used herein refers to an alkyl composed of 1 to 12 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁-s alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like.

“Alkenyl” or “olefin” refers to a radical of a straight-chain or branched hydrocarbon group having from 2 to 10 carbon atoms and 1, 2, 3, or 4 carbon-carbon double bonds (“C₂-10 alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂-s alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR′S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N, O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (0-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents wherein a moiety is “optionally substituted” as used herein means the moiety may be substituted with any additional moiety selected from, but not limited to, the group consisting of halogen, —CN, —NO₂, —N₃, —SO₂H, —SO₃H, —OH, —OR^{aa}, —N(R^{bb})₂, —N(OR^{cc})R^{bb}, —SH, —SR^{aa}, —C(═O)R^{aa}, —CO₂H, —CHO, —CO₂R^{aa}, OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —S(═O)R^{aa}, —OS(═O)R^{aa}, —B(OR^{cc})₂, C₁₋₁₀ alkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group ═O; each instance of R^{aa} is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from the group consisting of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —SO₂N(R^{cc})₂, —SOR^{aa}, C₁₋₁₀ alkyl, C₁₋₁₀ perhaloalkyl, C₂₋₁₀ alkenyl, C₂₋₁₀ alkynyl, C₃-14 carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from the group consisting of hydrogen, C₁₋₁₀ alkyl, C₁₋₁₀ perhaloalkyl, C₂₋₁₀ alkenyl, C₂₋₁₀ alkynyl, C₃-14 carbocyclyl, 3- to 14-membered heterocyclyl, C₆-1₄ aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and each instance of R^{dd} is, independently, selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(OC₁₋₆ alkyl)(C₁₋₆ alkyl), —N(OH)(C₁₋₆ alkyl), —NH(OH), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —B(OH)₂, —B(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 5- to 10-membered heteroaryl; or two geminal R^{dd} substituents on a carbon atom may be joined to form ═O.

“Halo” or “halogen” refers to fluorine (fluoro, —F), chlorine (chloro, —Cl), bromine (bromo, —Br), or iodine (iodo, —I).

As used herein, the term “composition” is intended to encompass a product comprising the specified ingredients, as well as any product which results, directly or indirectly, from combination of the specified ingredients.

“Salt” includes any and all salts. “Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

In this disclosure, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

Unless otherwise indicated, compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC). Compounds described herein can be in the form of individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, replacement of a carbon by a ¹³C- or ¹⁴C-enriched carbon, and/or replacement of an oxygen atom with ¹⁸O, are within the scope of the disclosure. Other examples of isotopes include ⁵N, ¹⁸O, ¹⁷O, ³¹P ³²P, ³⁵S, ¹⁸F, ³⁶Cl and ¹²³I. Compounds with such isotopically enriched atoms are useful, for example, as analytical tools or probes in biological assays.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability.

Certain isotopically-labelled compounds of Formula (I) can be useful for medical imaging purposes, for example, those labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements), and hence, may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half-lives (t_{1/2}>1 day), can generally be prepared by following procedures analogous to those disclosed in the Schemes and/or in the Examples herein below, by substituting an appropriate isotopically labeled reagent for a non-isotopically labeled reagent.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

### Methods of Use

In some embodiments, the present disclosure provides a method for treating a myelodysplastic syndrome (MDS) in a subject suffering therefrom. The method comprises administering to the subject a therapeutically effective amount of an antimalarial endoperoxide compound or a pharmaceutically acceptable salt thereof.

As illustrated by the appended examples, screening hits identified endoperoxide compounds that, although heretofore unknown to possess any therapeutic activity in an MDS context, have demonstrated therapeutic but pathologically unrelated antimalarial uses. Thus, in accordance with some embodiments, the endoperoxide compound is the naturally-occurring artemisinin, derived from the plant Artemisia annua, or any known artemisinin derivatives, including semisynthetic derivatives. Non-limiting examples of these compounds, per various embodiments, are shown in Table 1 and Table 2.

TABLE 1
Artemisinin, some its known derivatives, and novel derivatives of Formulae I-V.

| | | | | | | HL-60 | |
|---|---|---|---|---|---|---|---|
| | | | | | | differ- | HL-60 |
| | | | | | | entia- | cytotoxi- |
| | | | | Mass | Mass | tion | city |
| | | | Data of ¹HNMR | value | value | potency | IC₅₀ |
| # | Structure | Name | spectrum | (M + 1) | (M + 23) | (uM) | (uM) |
| | 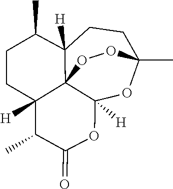 [SMILES: C[C@@H]1CC[C@H]2[C@@H](C)C(=O)O[C@@H]3O[C@@]4(C)CC[C@@H]1[C@]32OO4] | artemisinin | | | | | |
| | 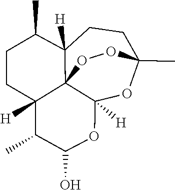 | dihydro- artemisinin | | | | | |
| | 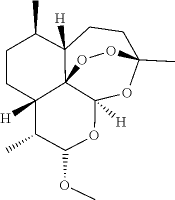 | artemether | | | | | |
| | 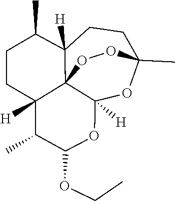 | arteether | | | | | |
| | 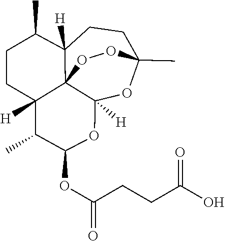 [SMILES: C[C@H]1[C@H](OC(=O)CCC(=O)O)O[C@@H]2O[C@@]3(C)CC[C@H]4[C@H](C)CC[C@@H]1[C@@]24OO3] | artesunate | | | | | |
| | 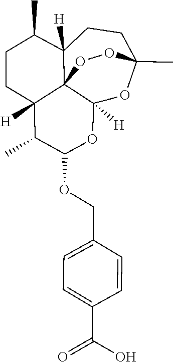 | artelinate | | | | | |
| 1 | 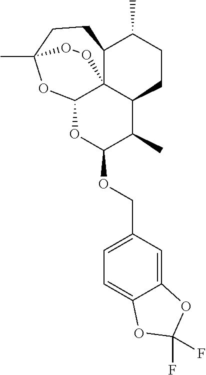 [SMILES: C[C@H]1[C@@H](OCc2ccc3c(c2)OC(F)(F)O3)O[C@@H]2O[C@@]3(C)CC[C@H]4[C@H](C)CC[C@@H]1[C@@]24OO3] | (3R,5aS,6R,8aS, 9R,10S,12R, 12aR)-10-((2,2- difluorobenzo[d] [1,3]dioxol-5- yl)methoxy)- 3,6,9- trimethyldeca- hydro-12H-3,12- epoxy[1,2] dioxepino[4,3- i]isochromene | ¹H NMR (400 MHz, DMSO) δ 7.39 (d, J = 8.3 Hz, 1H), 7.37 (s, 1H), 7.22-7.15 (m, 1H), 5.41 (s, 1H), 4.81 (d, J = 3.6 Hz, 1H), 4.76 (d, J = 12.3 Hz, 1H), 4.43 (d, J = 12.3 Hz, 1H), 2.45 (dt, J = 7.8, 4.2 Hz, 0H), 2.19 (td, J = 13.9, 3.9 Hz, 1H), 2.00 (dt, J = 14.2, 4.2 Hz, 1H), 1.80 (ddd, J = 13.4, 5.8, 2.4 Hz, 1H), 1.76-1.63 (m, 2H), 1.54 (dd, J = 13.2, 3.4 Hz, 1H), 1.45- 1.33 (m, 1H), 1.16 (dt, J = 11.4, 5.7 Hz, 1H), 0.91-0.81 (m, 7H). | | 477.34 | 0.011092 | 0.01061 |
| 2 | 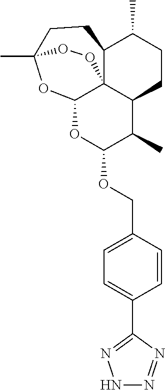 [SMILES: C[C@H]1[C@H](OCc2ccc(-c3nn[nH]n3)cc2)O[C@@H]2O[C@@]3(C)CC[C@H]4[C@H](C)CC[C@@H]1[C@@]24OO3] | 5-(4- ((((3R,5aS,6R, 8aS,9R,10R,12R, 12aR)-3,6,9- trimethyldeca- hydro-12H- 3,12-epoxy[1,2] dioxepino[4,3-i] isochromen-10- yl)oxy)methyl) phenyl)-2H- tetrazole | | 443.13 | | 0.0413 | 0.05854 |
| 3 | 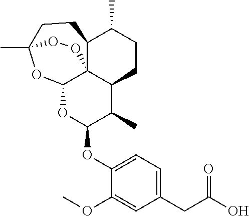 [SMILES: COc1cc(CC(=O)O)ccc1O[C@H]1O[C@@H]2O[C@@]3(C)CC[C@H]4[C@H](C)CC[C@@H]([C@H]1C)[C@@]24OO3] | 2-(3-methoxy- 4- (((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 12.29 (s, 1H), 7.07 (d, J = 8.1 Hz, 1H), 6.91 (s, 1H), 6.78 (d, J = 8.2 Hz, 1H), 5.54 (s, 1H), 5.41 (s, 1H), 3.74 (d, J = 2.4 Hz, 3H), 3.51 (s, 2H), 2.17 (d, J = 14.8 Hz, 1H), 2.09 (d, J = 13.7 Hz, 1H), 1.98 (d, J = 14.7 Hz, 1H), 1.85-1.73 (m, 3H), 1.64 (d, J = | 449.31 | | 0.0423 | >20 |
| | | | 13.1 Hz, 1H), 1.49 | | | | |
| | | | (d, J = 13.3 Hz, 1H), | | | | |
| | | | 1.38 (d, J = 12.9 | | | | |
| | | | Hz, 3H), 1.25 (s, | | | | |
| | | | 4H), 1.20 (d, J = 7.5 | | | | |
| | | | Hz, 1H), 1.01 (d, J = | | | | |
| | | | 7.4 Hz, 3H), 0.93 (d, | | | | |
| | | | J = 6.6 Hz, 4H). | | | | |
| 4 | 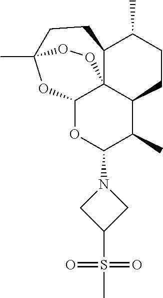 | 3- (methylsulfonyl)- 1-((3R,5aS,6R, 8aS,9R,10R, 12R,12aR)-3,6,9- trimethyldeca- hydro-1H-3,12- epoxy[1,2] dioxepino[4,3-i] isochromen- 10-yl)azetidine | ¹H NMR (400 MHz, CDCl₃) δ 5.33 (s, 1H), 4.04- 3.77 (m, 6H), 2.37 (ddd, J = 14.5, 13.4, 4.0 Hz, 1H), 2.17 (ddd, J = 10.1, 7.2, 4.3 Hz, 1H), 2.09- 1.95 (m, 2H), 1.88 (ddd, J = 13.1, 6.5, 3.4 Hz, 1H), 1.78- 1.63 (m, 2H), 1.62- 1.43 (m, 2H), 1.39 (s, 3H), 1.31 (dd, J = 8.2, 3.0 Hz, 0H), 1.30-1.15 (m, 2H), 1.14-0.97 (m, 1H), 0.95 (d, J = 6.1 Hz, | 402.35 | | 0.0428 | 0.101 |
| | | | 3H), 0.81 (d, J = | | | | |
| | | | 7.1 Hz, 3H). | | | | |
| 5 | 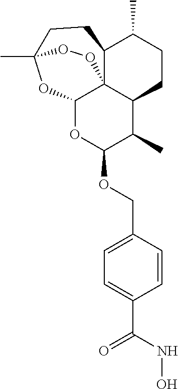 [SMILES: C[C@H]1[C@@H](OCc2ccc(C(=O)NO)cc2)O[C@@H]2O[C@@]3(C)CC[C@H]4[C@H](C)CC[C@@H]1[C@@]24OO3] | N-hydroxy-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamide | ¹H NMR (400 MHz, CDCl₃) δ 7.75 (d, J = 8.0 Hz, 2H), 7.41 (d, J = 8.0 Hz, 2H), 5.44 (s, 1H), 4.95-4.82 (m, 2H), 4.57 (d, J = 13.4 Hz, 1H), 2.70 (s, 1H), 2.38 (t, J = 13.9 Hz, 1H), 2.05 (d, J = 14.7 Hz, 1H), 1.88 (m, 2H), 1.86- 1.75 (m, 3H), 1.63 (d, J = 13.0 Hz, 1H), 1.52 (s, 1H), 1.34- 1.22 (m, 3H), 0.95 (t, J = 6.5 Hz, 6H). | 434.36 | | 0.0522 | 2.88261 |
| 6 | 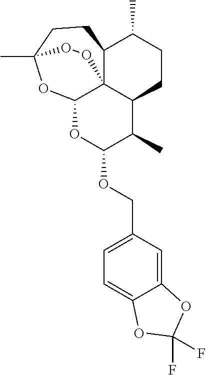 [SMILES: C[C@H]1[C@H](OCc2ccc3c(c2)OC(F)(F)O3)O[C@@H]2O[C@@]3(C)CC[C@H]4[C@H](C)CC[C@@H]1[C@@]24OO3] | (3R,5aS,6R,8aS, 9R,10R,12R, 12aR)-10-((2,2- difluorobenzo [d][1,3]dioxol-5- yl)methoxy)-3,6, 9-trimethyldeca- hydro-12H-3,12- epoxy[1,2] dioxepino[4,3-i] isochromene | ¹H NMR (400 MHz, CDCl₃) δ 7.13 (s, 1H), 7.04 (d, J = 8.1 Hz, 1H), 6.99 (d, J = 8.2 Hz, 1H), 5.34 (s, 1H), 4.93 (d, J = 12.4 Hz, 1H), 4.59 (d, J = 12.4 Hz, 1H), 4.50 (d, J = 9.2 Hz, 1H), 2.58-2.45 (m, 1H), 2.43-2.35 (m, 1H), 2.04 (dq, J = 8.0, 4.1 Hz, 1H), 1.93-1.87 (m, 1H), 1.75-1.65 (m, 2H), 1.59-1.49 (m, 3H), 1.35-1.16 (m, 5H), 1.09-1.00 (m, 1H), 0.95 (d, J = 5.7 Hz, 3H) 0.91 (d, J = 7.2 Hz, 3H). | | 477.41 | 0.053234 | 0.04997 |
| 7 | 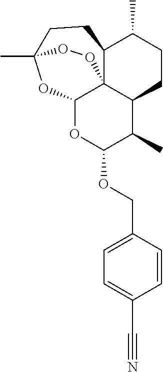 [SMILES: C[C@H]1[C@H](OCc2ccc(C#N)cc2)O[C@@H]2O[C@@]3(C)CC[C@H]4[C@H](C)CC[C@@H]1[C@@]24OO3] | 4-((((3R,5aS,6R, 8aS,9R,10R,12R, 12aR)-3,6,9- trimethyldeca- hydro-12H-3,12- epoxy[1,2] dioxepino[4,3-i] isochromen-10- yl)oxy)methyl) benzonitrile | ¹H NMR (400 MHz, CDCl₃) δ 7.62 (d, J = 7.9 Hz, 2H), 7.47 (d, J = 7.9 Hz, 2H), 5.35 (s, 1H), 5.04 (d, J = 13.5 Hz, 1H), 4.66 (d, J = 13.5 Hz, 1H), 4.53 (d, J = 9.2 Hz, 1H), 2.53 (d, J = 7.2 Hz, 1H), 2.38 (dd, J = 13.8, 4.0 Hz, 1H), 2.04 (dt, J = 15.1, 4.3 Hz, 1H), 1.90 (d, J = 13.9 Hz, 1H), 1.75 (dd, J = 13.5, 3.8 Hz, 1H), 1.72-1.65 (m, 1H), 1.61-1.52 (m, , 2H), 1.45 (s, 3H), 1.36-1.16 (m, 3H), 1.0-0.9 (m, 1H), 0.96-0.93 (m, 6 H) | | 422.09 | 0.0546 | 0.19205 |
| 8 | 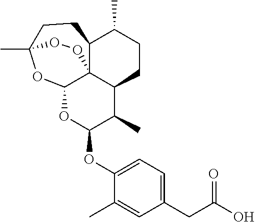 [SMILES: Cc1cc(CC(=O)O)ccc1O[C@H]1O[C@@H]2O[C@@]3(C)CC[C@H]4[C@H](C)CC[C@@H]([C@H]1C)[C@@]24OO3] | 2-(3-methyl-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 12.20 (s, 1H), 7.08- 7.03 (m, 3H), 5.52 (d, J = 2.9 Hz, 1H), 5.42 (s, 1H), 3.45 (s, 2H), 2.23-2.16 (m, 1H), 2.15 (s, 3H) 2.01 (d, J = 14.2 Hz, 1H), 1.95-1.77 (m, 3H), 1.64 (d, J = 13.2 Hz, 1H), 1.56- 1.50 (m, 1H), 1.42- 1.34 (m, 2H), 1.30 | | 455.43 | 0.057704 | >20 |
| | | | (s, 3H), 1.26-1.14 | | | | |
| | | | (m, 1H), 0.98 (d, J = | | | | |
| | | | 7.5 Hz, 3H), 0.93- | | | | |
| | | | 0.84 (m, 4H). | | | | |
| 9 | 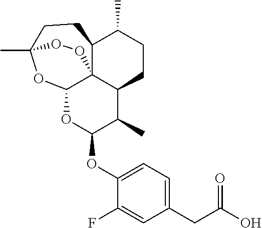 [SMILES: C[C@H]1[C@@H](Oc2ccc(CC(=O)O)cc2F)O[C@@H]2O[C@@]3(C)CC[C@H]4[C@H](C)CC[C@@H]1[C@@]24OO3] | 2-(3-fluoro-4- (((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 12.36 (s, 1H), 7.26 (t, J = 8.5 Hz, 1H), 7.14 (d, J = 12.0 Hz, 1H), 7.03 (d, J = 8.4 Hz, 1H), 5.53 (s, 1H), 5.47 (s, 1H), 3.55 (s, 2H), 2.60 (s, 1H), 2.23-2.16 (m, 1H), 2.00 (d, J = 15.1 Hz, 1H), 1.95- 1.88 (m, 1H), 1.87- 1.74 (m, H), 1.63 (d, | | 459.31 | 0.059261 | >20 |
| | | | J = 13.1 Hz, 1H), | | | | |
| | | | 1.54 (dd, J = 11.5, | | | | |
| | | | 6.4 Hz, 1H), 1.45- | | | | |
| | | | 1.31 (m, 2H), 1.27 | | | | |
| | | | (s, 3H), 1.21 (dq, J = | | | | |
| | | | 11.9, 6.2 Hz, 1H), | | | | |
| | | | 1.00 (d, J = 7.3 Hz, | | | | |
| | | | 3H), 0.96 (s, 1H), | | | | |
| | | | 0.92 (d, J = 6.4 Hz, | | | | |
| | | | 3H). | | | | |
| 10 | 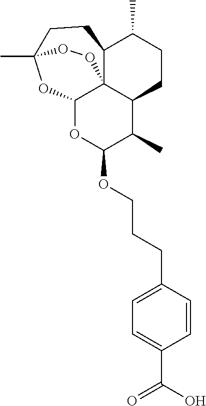 [SMILES: C[C@H]1[C@@H](OCCCc2ccc(C(=O)O)cc2)O[C@@H]2O[C@@]3(C)CC[C@H]4[C@H](C)CC[C@@H]1[C@@]24OO3] | 4-(3- (((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)propyl) benzoic acid | ¹H NMR (400 MHz, CDCl₃) δ 8.02 (d, J = 8.4 Hz, 2H), 7.28 (d, J = 8.5 Hz, 2H), 5.39 (s, 1H), 4.79 (d, J = 3.6 Hz, 1H), 3.88 (dt, J = 9.8, 6.2 Hz, 1H), 3.41 (dt, J = 9.9, 6.3 Hz, 1H), 2.84-2.71 (m, 2H), 2.64 (dt, J = 7.7, 4.0 Hz, 1H), 2.47-2.28 (m, 1H), 2.04 (ddd, J = 14.6, 4.9, 3.0 Hz, 1H), 1.97-1.85 (m, 3H), 1.83-1.75 (m, 2H), 1.66-1.62 (m, 1H), 1.56-1.45 (m, 2H), 1.44 (s, 3H), 1.39- 1.29 (m, 1H), 1.28- 1.23 (m, 2H), 0.96 (d, J = 6.2 Hz, 3H), 0.94-0.87 (m, 4H). | 447.22 | | 0.0638 | >10 |
| 11 | 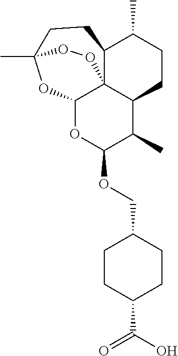 [SMILES: C[C@H]1[C@@H](OC[C@H]2CC[C@@H](C(=O)O)CC2)O[C@@H]2O[C@@]3(C)CC[C@H]4[C@H](C)CC[C@@H]1[C@@]24OO3] | (1R,4s)-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxylic acid | ¹H NMR (400 MHz, DMSO) δ 12.07 (s, 1H), 5.26 (s, 1H), 4.64 (d, J = 3.5 Hz, 1H), 3.53 (dd, J = 9.4, 6.7 Hz, 1H), 3.13 (dd, J = 9.4, 6.2 Hz, 1H), 2.39 (dt, J = 7.9, 4.2 Hz, 1H), 2.17 (td, J = 13.8, 3.8 Hz, 1H), 1.99 (dt, J = 14.0, 4.1 Hz, 1H), 1.92-1.76 (m, 3H), 1.72 (dd, J = 13.6, 3.3 Hz, 1H), 1.68- 1.57 (m, 3H), 1.56- 1.44 (m, 5H), 1.39- 1.35 (m, 2H), 1.28 (s, 3H), 1.23 (d, J = 12.0 Hz, 3H), 1.18- 1.10 (m, 1H), 0.92- 0.80 (m, 7H). | | 447.15 | 0.0874 | >20 |
| 12 | 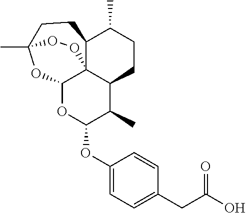 [SMILES: C[C@H]1[C@H](Oc2ccc(CC(=O)O)cc2)O[C@@H]2O[C@@]3(C)CC[C@H]4[C@H](C)CC[C@@H]1[C@@]24OO3] | 2-(4-(((3R,5aS, 6R,8aS,9R,10R, 12R,12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 7.17 (d, J = 8.4 Hz, 2H), 7.02 (d, J = 8.4, Hz, 2H), 5.68 (s, 1H), 5.2 (d, J = 9.2 Hz, 1H), 3.50 (s, 2H), 2.42- 2.35 (m, 1H), 2.25- 2.15 (m, 1H), 2.05- 1.95 (m, 1H), 1.85- 1.75 (m, 1H), 1.66- 1.56 (m, 4H), 1.45- 1.30 (m, 2H), 1.26 | | 441.05 | 0.0924 | >20 |
| | | | (s, 3H), 1.25-1.10 | | | | |
| | | | (m, 1H), 0.95-0.85 | | | | |
| | | | (m, 1H), 0.90-0.88 | | | | |
| | | | (m, 6H). | | | | |
| 13 | 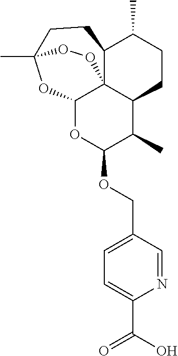 [SMILES: C[C@H]1[C@@H](OCc2ccc(C(=O)O)nc2)O[C@@H]2O[C@@]3(C)CC[C@H]4[C@H](C)CC[C@@H]1[C@@]24OO3] | 5-((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) picolinic acid | ¹H NMR (400 MHz, CDCl₃) δ 8.60-8.55 (m, 1H), 8.22 (dd, J = 8.0, 0.8 Hz, 1H), 7.91-7.85 (m, 1H), 5.43 (s, 1H), 5.04 (d, J = 13.5 Hz, 1H), 4.94 (d, J = 3.6 Hz, 1H), 4.65 (d, J = 13.5 Hz, 1H), 2.73 (dt, J = 7.9, 4.1 Hz, 1H), 2.39 (ddd, J = 14.6, 13.4, 3.9 Hz, 1H), 2.10-2.00 (m, 1H), 1.94-1.78 (m, 2H), 1.74 (dd, J = 13.4, 3.4 Hz, 1H), 1.66 (ddt, J = 13.4, 6.6, 3.3 Hz, 1H), 1.52 (dt, J = 12.1, 4.7 Hz, 1H), 1.43 (d, J = 3.7 Hz, 1H), | 420.15 | | 0.0889 | 1.423067 |
| | | | 1.35-1.22 (m, 2H), | | | | |
| | | | 1.02-0.90 (m, 7H). | | | | |
| 14 | 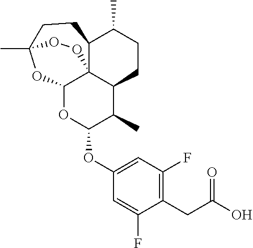 [SMILES: C[C@H]1[C@H](Oc2cc(F)c(CC(=O)O)c(F)c2)O[C@@H]2O[C@@]3(C)CC[C@H]4[C@H](C)CC[C@@H]1[C@@]24OO3] | 2-(2,6-difluoro- 4-(((3R,5aS,6R, 8aS,9R,10R,12R, 12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 12.64 (s, 1H), 6.92 (m, d, J = 9.3 Hz, 2H), 5.77 (s, 1H), 5.28 (d, J = 9.2 Hz, 1H), 3.54 (s, 2H), 2.39 (dq, J = 9.9, 5.0 Hz, 1H), 2.19 (td, J = 14.0, 3.9 Hz, 1H), 2.00 (d, J = 13.9 Hz, 1H), 1.89-1.79 (m, 1H), 1.67-1.55 (m, 4H), 1.42-1.32 (m, 2H), 1.26 (s, 3H), 1.25- 1.14 (m, 1H), 1.02- 0.95 (m, 3H), 0.92 | | | 0.090931 | 20 |
| | | | (d, J = 6.3 Hz, 3H), | | | | |
| | | | 0.88 (d, J = 7.1 Hz, | | | | |
| | | | 3H). | | | | |
| 15 | 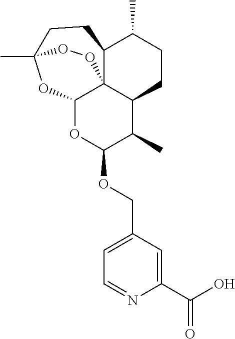 [SMILES: C[C@H]1[C@@H](OCc2ccnc(C(=O)O)c2)O[C@@H]2O[C@@]3(C)CC[C@H]4[C@H](C)CC[C@@H]1[C@@]24OO3] | 4-((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) picolinic acid | ¹H NMR (400 MHz, CDCl₃) δ 8.59 (s, 1H), 8.17 (s, 1H), 7.52 (s, 1H), 5.42 (s, 1H), 5.06-4.98 (m, 1H), 4.94 (d, J = 3.5 Hz, 1H), 4.64 (d, J = 14.6 Hz, 1H), 2.74 (s, 1H), 2.38 (td, J = 13.9, 3.8 Hz, 1H), 2.04 (d, J = 14.9 Hz, H), 1.88 (d, J = 12.0 Hz, 2H), 1.79 (d, J = 13.3 Hz, 1H), 1.67 (d, J = 12.3 Hz, 1H), 1.53 (dd, J = 12.6, 7.1 Hz, 2H), 1.44 (s, 3H), 1.33-1.23 (m, 2H), 1.02 (d, J = 7.4 Hz, 3H), 0.97-0.85 (m, 4H). | 420.15 | | 0.0931 | 2.552 |
| 16 | 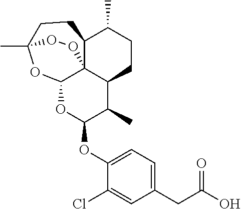 [SMILES: C[C@H]1[C@@H](Oc2ccc(CC(=O)O)cc2Cl)O[C@@H]2O[C@@]3(C)CC[C@H]4[C@H](C)CC[C@@H]1[C@@]24OO3] | 2-(3-chloro-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 7.34 (s, 1H), 7.29 (d, J = 8.6 Hz, 1H), 7.19 (d, J = 8.7 Hz, 1H), 5.65 (s, 1H), 5.47 (s, 1H), 3.53 (s, 2H), 2.67 (s, 1H), 2.25-2.16 (m, 1H), 2.08-1.98 (m, 2H), 1.85-1.78 (m, 2H), 1.67-1.62 (m, 1H), 1.58-1.52 (m, 1H), 1.41-1.31 (m, 2H), 1.28 (s, 3H), 1.25-1.19 (m, 1H), | | 475.38 | 0.0948 | >10 |
| | | | 1.01 (d, J = 7.2 Hz, | | | | |
| | | | 3H), 0.97-0.95 (s, | | | | |
| | | | 1H), 0.91 (d, J = 8.0 | | | | |
| | | | Hz, 3H). | | | | |
| 17 | 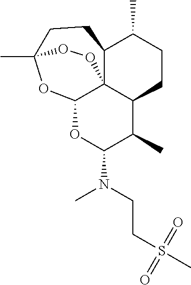 | (3R,5aS,6R,8aS, 9R,10R,12R, 12aR)-N,3,6,9- tetramethyl-N-(2- (methylsulfonyl) ethyl)decahydro- 12H-3,12-epoxy [1,2]dioxepino [4,3-i] isochromen-10- amine | ¹H NMR (400 MHz, CDCl₃) δ 5.29 (s, 1H), 4.13 (s, 1H), 3.61 (s, 1H), 3.39 (s, 1H), 3.24 (d, J = 16.0 Hz, 2H), 2.96 (s, 3H), 2.63-2.43 (m, 4H), 2.35 (td, J = 13.9, 4.0 Hz, 1H), 2.03-2.00 (m, 1H), 1.88 (dd, J = 11.2, 4.7 Hz, 1H), 1.72 (d, J = 12.8 Hz, 2H), 1.53-1.40 (m, 1H), 1.38 (s, 3H), 1.35-1.20 (m, 3H), 1.02 (t, J = 11.5 Hz, 1H), 0.96 | 404.08 | | 0.0955 | 0.2434 |
| | | | (s, 3H), 0.95 (s, 3H). | | | | |
| 18 | 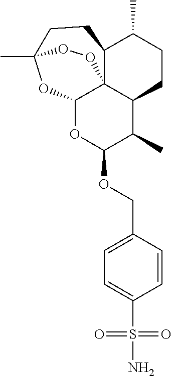 [SMILES: C[C@H]1[C@@H](OCc2ccc(S(N)(=O)=O)cc2)O[C@@H]2O[C@@]3(C)CC[C@H]4[C@H](C)CC[C@@H]1[C@@]24OO3] | 4-((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) benzene- sulfonamide | ¹H NMR (400 MHz, CDCl₃) δ 7.91 (d, J = 8.1 Hz, 2H), 7.46 (d, J = 8.0 Hz, 2H), 5.44 (s, 1H), 4.97 (d, J = 13.3 Hz, 1H), 4.92 (d, J = 3.5 Hz, 1H), 4.79 (s, 2H), 4.59 (d, J = 13.3 Hz, 1H), 2.71 (d, J = 8.0 Hz, 1H), 2.45-2.32 (m, 1H), 2.09-2.00 (m, 1H), 1.93-1.85 (m, 1H), 1.83-1.75 (m, 2H), 1.71-1.60 (m, 1H), 1.54-1.48 (m, 2H), 1.36-1.22 (m, 2H), 1.00-0.86 (m, 7H). | | 476.75 | 0.0990 | 2.253 |
| 19 | 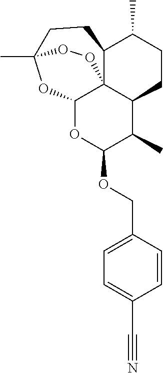 [SMILES: C[C@H]1[C@@H](OCc2ccc(C#N)cc2)O[C@@H]2O[C@@]3(C)CC[C@H]4[C@H](C)CC[C@@H]1[C@@]24OO3] | 4-((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) benzonitrile | ¹H NMR (400 MHz, CDCl₃) δ 7.63 (d, J = 8.4 Hz, 1H), 7.41 (d, J = 8.0 Hz, 1H), 5.42 (s, 1H), 4.95 (d, J = 13.5 Hz, 1H), 4.91 (d, J = 3.5 Hz, 1H), 4.57 (d, J = 13.5 Hz, 1H), 2.70 (dqd, J = 11.0, 7.5, 4.1 Hz, 1H), 2.38 (ddd, J = 14.6, 13.4, 4.0 Hz, 1H), 2.04 (ddd, J = 14.6, 5.0, 3.0 Hz, 1H), 1.89 (ddt, J = 13.7, 6.9, 3.7 Hz, 0H), 1.83-1.78 (m, 1H), 1.64 (dq, J = 13.6, 3.4 Hz, 2H), 1.55-1.47 (m, 2H), 1.45 (s, 3H), 1.29 (ddd, J = 17.0, | 400.20 | | 0.1042 | 0.1379 |
| | | | 10.2, 6.1 Hz, 2H), | | | | |
| | | | 0.98-0.94 (m, 7H). | | | | |
| 20 | 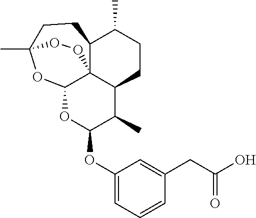 [SMILES: C[C@H]1[C@@H](Oc2cccc(CC(=O)O)c2)O[C@@H]2O[C@@]3(C)CC[C@H]4[C@H](C)CC[C@@H]1[C@@]24OO3] | 2-(3- (((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 12.33 (s, 1H), 7.24 (t, J = 7.8 Hz, 1H), 6.98 (dd, J = 8.1, 2.4 Hz, 1H), 6.94 (s, 1H), 6.90 (d, J = 7.6 Hz, 1H), 5.53 (d, J = 3.4 Hz, 1H), 5.43 (s, 1H), 3.54 (s, 2H), 2.59 (s, 1H), 2.26-2.13 (m, 1H), 2.01-1.98 (m, 1H), 1.90 (td, J = 13.3, 3.3 Hz, 1H), | | 441.35 | 0.1165 | 8.326 |
| | | | 1.82 (dt, J = 14.3, | | | | |
| | | | 4.9 Hz, 2H), 1.64 | | | | |
| | | | (dd, J = 13.5, 3.4 | | | | |
| | | | Hz, 1H), 1.52 (dt, | | | | |
| | | | J = 13.1, 4.7 Hz, | | | | |
| | | | 1H), 1.45-1.30 (m, | | | | |
| | | | 2H), 1.29 (s, 3H), | | | | |
| | | | 1.24-1.14 (m, 1H), | | | | |
| | | | 0.96 (d, J = 7.3 Hz, | | | | |
| | | | 3H), 0.91 (d, J = 6.4 | | | | |
| | | | Hz, 3H). | | | | |
| 21 | 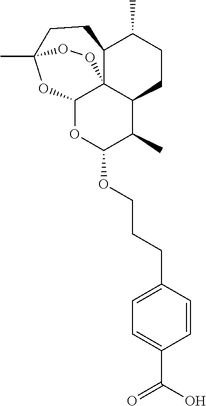 [SMILES: C[C@H]1[C@H](OCCCc2ccc(C(=O)O)cc2)O[C@@H]2O[C@@]3(C)CC[C@H]4[C@H](C)CC[C@@H]1[C@@]24OO3] | 4-(3- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)propyl) benzoic acid | ¹H NMR (400 MHz, CDCl₃) δ 8.01 (d, J = 7.9 Hz, 2H), 7.30 (d, J = 8.0 Hz, 2H), 5.33 (s, 1H), 4.41 (d, J = 9.3 Hz, 1H), 4.05-3.95 (m, 1H), 3.41 (q, J = 7.5 Hz, 1H), 2.79 (q, J = 8.3 Hz, 2H), 2.49-2.32 (m, 2H), 2.07-1.85 (m, 4H), 1.83-1.72 (m, 1H), 1.69 (dd, J = 12.7, 3.0 Hz, 1H), 1.61- 1.51 (m, 1H), 1.45 (s, 3H), 1.30 (ddd, J = 16.1, 11.8, 5.0 Hz, 4H), 1.00 (m, 1H), 0.96 (d, J = 5.8 Hz, 3H), 0.92 (d, J = 7.0 Hz, 3H). | 464.22 (M + 18) | | 0.1226 | >20 |
| 22 | 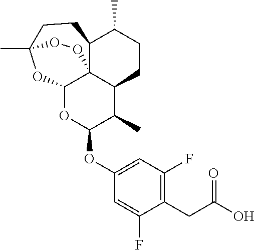 [SMILES: C[C@H]1[C@@H](Oc2cc(F)c(CC(=O)O)c(F)c2)O[C@@H]2O[C@@]3(C)CC[C@H]4[C@H](C)CC[C@@H]1[C@@]24OO3] | 2-(2,6-difluoro-4- (((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 12.61 (s, 1H), 6.87 (d, J = 9.2 Hz, 2H), 5.66 (d, J = 3.4 Hz, 1H), 5.44 (s, 1H), 3.55 (s, 2H), 2.61 (tq, J = 11.3, 5.6 Hz, 1H), 2.20 (td, J = 14.0, 3.9 Hz, 1H), 2.00 (dt, J = 14.5, 4.1 Hz, 1H), 1.87-1.79 (m, 3H), 1.63 (dd, J = 13.3, 3.4 Hz, 1H), 1.53 (dt, J = 12.6, 5.0 Hz, 1H), 1.48-1.33 (m, 2H), 1.30 (s, 3H), | | | 0.130611 | 20 |
| | | | 1.20 (td, J = 11.3, | | | | |
| | | | 6.4 Hz, 1H), 1.04- | | | | |
| | | | 0.83 (m, 7H). | | | | |
| 23 | 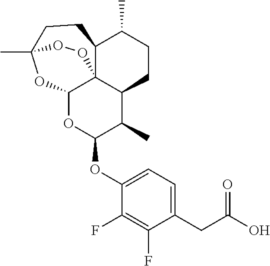 [SMILES: C[C@H]1[C@@H](Oc2ccc(CC(=O)O)c(F)c2F)O[C@@H]2O[C@@]3(C)CC[C@H]4[C@H](C)CC[C@@H]1[C@@]24OO3] | 2-(2,3-difluoro-4- (((3R,5aS,6R,8aS, 9R,10S,12R, 12aR)-3,6,9- trimethyldeca- hydro-12H-3,12- epoxy[1,2] dioxepino[4,3- i]isochromen-10- yl)oxy)phenyl) acetic acid | ¹H NMR (400 MHz, DMSO) δ 12.54 (s, 1H), 7.12 (q, J = 7.6 Hz, 2H), 5.60 (d, J = 3.3 Hz, 1H), 5.47 (s, 1H), 3.62 (s, 2H), 2.63 (qt, J = 7.5, 3.9 Hz, 1H), 2.19 (td, J = 14.0, 3.8 Hz, 1H), 1.99 (dt, J = 14.5, 4.0 Hz, 1H), 1.93-1.75 (m, 3H), 1.67-1.59 (m, 1H), 1.56-1.50 (m, 1H), 1.43-1.32 (m, 2H), 1.28 (s, 3H), 1.23-1.15 (m, 1H), | | | 0.191719 | 15 |
| | | | 1.00 (d, J = 7.3 Hz, | | | | |
| | | | 3H), 0.95-0.89 (d, | | | | |
| | | | J = 6.4 Hz, 4H). | | | | |
| 24 | 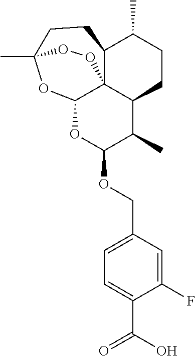 [SMILES: C[C@H]1[C@@H](OCc2ccc(C(=O)O)c(F)c2)O[C@@H]2O[C@@]3(C)CC[C@H]4[C@H](C)CC[C@@H]1[C@@]24OO3] | 2-fluoro-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, CDCl₃) δ 8.00 (t, J = 7.7 Hz, 1H), 7.15 (t, J = 10.4 Hz, 2H), 5.44 (s, 1H), 4.96 (d, J = 13.7 Hz, 1H), 4.92 (d, J = 3.5 Hz, 1H), 4.58 (d, J = 13.8 Hz, 1H), 2.75-2.68 (m, 1H), 2.39 (td, J = 14.0, 4.0 Hz, 1H), 2.05 (d, J = 14.6 Hz, 1H), 1.93- 1.72 (m, 3H), 1.70- 1.63 (m, 1H), 1.51 (dd, J = 14.2, 8.9 Hz, 2H), 1.39-1.27 (m, 2H), 1.25 (s, 3H), 0.99 (d, J = 7.3 Hz, 3H), 0.95 (d, J = 6.0 Hz, 3H), 0.91- | | 459.19 | 0.2097 | >20 |
| | | | 0.79 (m, 1H). | | | | |
| 25 | 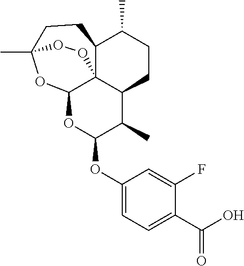 [SMILES: C[C@H]1[C@@H](Oc2ccc(C(=O)O)c(F)c2)O[C@H]2O[C@@]3(C)CC[C@H]4[C@H](C)CC[C@@H]1[C@@]24OO3] | 2-fluoro-4- (((3R,5aS,6R,8aS, 9R,10S,12S, 12aR)-3,6,9- trimethyldeca- hydro-12H-3,12- epoxy[1,2] dioxepino[4,3-i] isochromen-10- yl)oxy)benzoic acid | ¹H NMR (400 MHz, DMSO) δ 12.99 (s, 1H), 7.85 (t, J = 8.9 Hz, 1H), 5.73 (s, 1H), 5.41 (s, 1H), 2.19 (t, J = 13.8 Hz, 1H), 2.04-1.96 (m, 1H), 1.92-1.75 (m, 3H), 1.63 (d, J = 13.0 Hz, 1H), 1.59- 1.50 (m, 1H), 1.42 (s, 2H), 1.37-1.32 (m, 1H), 1.30 (s, 3H), 1.25-1.14 (m, 1H), 1.05-0.81 (m, 7H). | 423.42 | | 0.2170 | >20 |
| 26 | 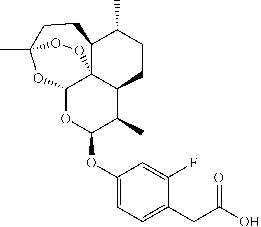 [SMILES: C[C@H]1[C@@H](Oc2ccc(CC(=O)O)c(F)c2)O[C@@H]2O[C@@]3(C)CC[C@H]4[C@H](C)CC[C@@H]1[C@@]24OO3] | 2-(2-fluoro-4- (((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 12.24 (s, 1H), 7.24 (t, J = 8.6 Hz, 1H), 6.93 (d, J = 11.7 Hz, 1H), 6.86 (dd, J = 8.4, 2.4 Hz, 1H), 5.58 (d, J = 3.4 Hz, 1H), 5.42 (s, 1H), 3.54 (s, 2H), 2.59 (qt, J = 7.3, 3.8 Hz, 1H), 2.19 (td, J = 13.9, 3.8 Hz, 1H), 1.99 (dt, J = 14.7, 3.9 Hz, 1H), | | | 0.224887 | >20 |
| | | | 1.86-1.74 (m, 3H), | | | | |
| | | | 1.67-1.58 (m, 1H), | | | | |
| | | | 1.52 (dt, J = 12.9, | | | | |
| | | | 4.8 Hz, 1H), 1.44- | | | | |
| | | | 1.34 (m, 2H), 1.29 | | | | |
| | | | (s, 3H), 1.25-1.13 | | | | |
| | | | (m, 1H), 1.00-0.83 | | | | |
| | | | (m, 7H). | | | | |
| 27 | 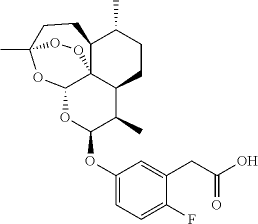 [SMILES: C[C@H]1[C@@H](Oc2ccc(F)c(CC(=O)O)c2)O[C@@H]2O[C@@]3(C)CC[C@H]4[C@H](C)CC[C@@H]1[C@@]24OO3] | 2-(2-fluoro-5- (((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 12.48 (s, 1H), 7.12 (t, J = 9.0 Hz, 1H), 7.03 (d, J = 5.4 Hz, 2H), 5.50- 5.43 (m, 2H), 3.59 (s, 2H), 2.19 (t, J = 13.7 Hz, 1H), 2.00 (d, J = 14.6 Hz, 1H), 1.89 (d, J = 13.1 Hz, 1H), 1.85-1.74 (m, 2H), 1.63 (d, J = 12.9 Hz, 1H), 1.51 (d, J = 12.8 Hz, 1H), | | 459.38 | 0.239043 | >20 |
| | | | 1.44-1.34 (m, 2H), | | | | |
| | | | 1.28 (s, 3H), 1.26- | | | | |
| | | | 1.17 (m, 2H), 0.97 | | | | |
| | | | (d, J = 7.4 Hz, 3H), | | | | |
| | | | 0.91 (d, J = 6.3 Hz, | | | | |
| | | | 3H), 0.86 (s, 1H). | | | | |
| 28 | 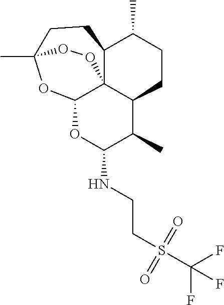 [SMILES: C[C@H]1[C@H](NCCS(=O)(=O)C(F)(F)F)O[C@@H]2O[C@@]3(C)CC[C@H]4[C@H](C)CC[C@@H]1[C@@]24OO3] | (3R,5aS,6R,8aS, 9R,10R,12R, 12aR)-3,6,9- trimethyl-N-(2- ((trifluoromethyl) sulfonyl)ethyl) decahydro-12H- 3,12-epoxy[1,2] dioxepino[4,3-i] isochromen-10- amine | | 444.16 | | 0.2559 | 0.4464 |
| 29 | 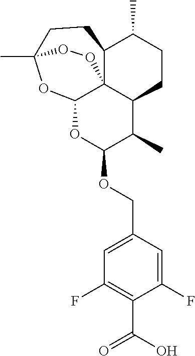 [SMILES: C[C@H]1[C@@H](OCc2cc(F)c(C(=O)O)c(F)c2)O[C@@H]2O[C@@]3(C)CC[C@H]4[C@H](C)CC[C@@H]1[C@@]24OO3] | 2,6-difluoro-4- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, DMSO) δ 13.87 (s, 1H), 7.14 (d, J = 9.6 Hz, 2H), 5.42 (s, 1H), 4.86-4.75 (m, 2H), 4.50 (d, J = 13.7 Hz, 1H), 2.25- 2.13 (m, 1H), 2.05- 1.89 (m, 2H), 1.80 (s, 2H), 1.72 (d, J = 9.3 Hz, 2H), 1.57 (d, J = 13.5 Hz, 1H), 1.46-1.34 (m, 3H), 1.30 (s, 3H), 1.25- 1.07 (m, 2H), 0.94- 0.83 (m, 7H). | 472.47 (M + 18) | | 0.28261 | >20 |
| 30 | 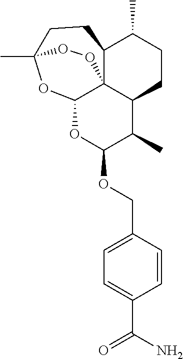 [SMILES: C[C@H]1[C@@H](OCc2ccc(C(N)=O)cc2)O[C@@H]2O[C@@]3(C)CC[C@H]4[C@H](C)CC[C@@H]1[C@@]24OO3] | 4-((((3R,5aS, 6R,8aS,9R,10S, 12R,12aR)-3,6,9- trimethyldeca- hydro-12H-3,12- epoxy[1,2] dioxepino[4,3-i] isochromen-10- yl)oxy)methyl) benzamide | | 418.14 | | 0.2998 | 3.52 |
| 31 | 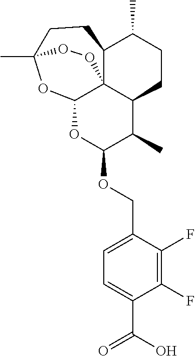 [SMILES: C[C@H]1[C@@H](OCc2ccc(C(=O)O)c(F)c2F)O[C@@H]2O[C@@]3(C)CC[C@H]4[C@H](C)CC[C@@H]1[C@@]24OO3] | 2,3-difluoro-4- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, DMSO) δ 7.69 (t, J = 7.3 Hz, 1H), 7.34 (t, J = 7.3 Hz, 1H), 5.41 (s, 1H), 4.92-4.83 (m, 2H), 4.57 (d, J = 13.1 Hz, 1H), 2.22-2.15 (, 1H), 2.01 (d, J = 14.1 Hz, 1H), 1.91 (s, 1H), 1.86-1.77 (m, 1H), 1.73-1.65 (m, 2H), 1.55 (d, J = 13.6 Hz, 1H), 1.45-1.33 (m, 2H), 1.31 (s, 3H), 1.25- 1.10 (m, 2H), 0.93- 0.81 (m, 7H). | 472.38 (M + 18) | | 0.324137 | >20 |
| 32 | 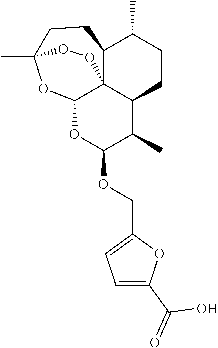 [SMILES: C[C@H]1[C@@H](OCc2ccc(C(=O)O)o2)O[C@@H]2O[C@@]3(C)CC[C@H]4[C@H](C)CC[C@@H]1[C@@]24OO3] | 5-((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) furan-2- carboxylic acid | ¹H NMR (400 MHz, CDCl₃) δ 7.25 (d, J = 3.4 Hz, 1H), 6.44 (d, J = 3.5 Hz, 1H), 5.46 (s, 1H), 4.90 (d, J = 3.5 Hz, 1H), 4.81 (d, J = 13.6 Hz, 1H), 4.61 (d, J = 13.6 Hz, 1H), 2.72-2.60 (m, 1H), 2.37 (ddd, J = 14.6, 13.4, 4.0 Hz, 1H), 2.09-1.98 (m, 1H), 1.88 (ddt, J = 13.6, 6.8, 3.5 Hz, 1H), 1.76 (ddd, J = 12.1, 9.1, 3.4 Hz, 2H), 1.62 (dq, J = 13.2, 3.4 Hz, 1H), 1.56- 1.46 (m, 2H), 1.44 (s, 3H), 1.35 (dddq, J = 12.4, 8.9, 6.2, | 426.11 (M + 18) | | 0.3332 | >20 |
| | | | 3.2 Hz, 1H), 1.28- | | | | |
| | | | 1.22 (m, 1H), 0.98- | | | | |
| | | | 0.86 (m, 7H). | | | | |
| 33 | 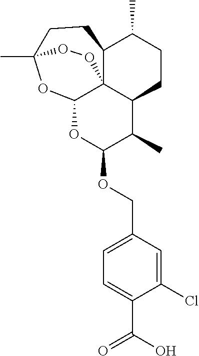 [SMILES: C[C@H]1[C@@H](OCc2ccc(C(=O)O)c(Cl)c2)O[C@@H]2O[C@@]3(C)CC[C@H]4[C@H](C)CC[C@@H]1[C@@]24OO3] | 2-chloro-4- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, CDCl₃) δ 7.99 (d, J = 7.8 Hz, 1H), 7.44 (s, 1H), 7.28 (d, J = 8.2 Hz, 1H), 5.44 (s, 1H), 4.93 (d, J = 9.2 Hz, 1H), 4.91 (s, 1H), 4.56 (d, J = 13.5 Hz, 1H), 2.71 (s, 1H), 2.45-2.32 (m, 1H), 2.05 (d, J = 14.5 Hz, 1H), 1.97-1.85 (m, 1H), 1.83 (m, 2H), 1.66 (d, J = 13.3 Hz, 1H), 1.54-1.47 (m, 2H), 1.37-1.27 (m, 2H), 1.25 (s, 3H), 0.99-0.82 (m, 7H). | 470.2 (M + 18) | | 0.3428 | >20 |
| 34 | 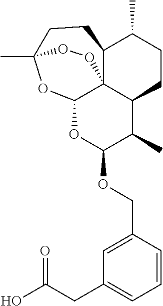 [SMILES: C[C@H]1[C@@H](OCc2cccc(CC(=O)O)c2)O[C@@H]2O[C@@]3(C)CC[C@H]4[C@H](C)CC[C@@H]1[C@@]24OO3] | 2-(3- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) phenyl)acetic acid | ¹H NMR (400 MHz, DMSO) δ 12.33 (s, 1H), 7.32-7.28 (d, J = 7.6 Hz, 1H), 7.25-7.13 (m, 3H), 5.40 (s, 1H), 4.80 (d, J = 3.5 Hz, 1H), 4.75 (d, J = 12.4 Hz, 1H), 4.42 (d, J = 12.4 Hz, 1H), 3.56 (s, 2H), 2.50 (s, 1H), 2.17 (dd, J = 13.8, 4.0 Hz, 1H), 2.00 (dt, J = 14.1, 4.1 Hz, 1H), 1.86- 1.62 (m, 2H), 1.55 (dd, J = 13.2, 3.4 Hz, 1H), 1.50-1.34 (m, 2H), 1.32 (s, | 455.51 | | 0.480461 | >20 |
| | | | 3H), 1.21-1.11 (M, | | | | |
| | | | 1H), 1.07 (d, J = 7.2 | | | | |
| | | | Hz, 1H), 0.97-0.79 | | | | |
| | | | (d, J = 6.7 Hz, 6H). | | | | |
| 35 | 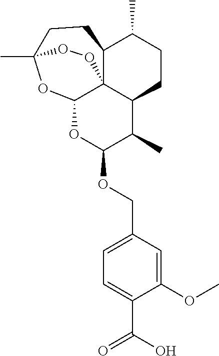 | 2-methoxy-4- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, CDCl₃) δ 8.16 (d, J = 8.0 Hz, 1H), 7.08 (m, 2H), 5.42 (s, 1H), 4.94 (d, J = 10.1 Hz, 1H), 4.92 (s, 1H), 4.62 (d, J = 13.6 Hz, 1H), 4.07 (s, 3H), 2.76-2.66 (m, 1H), 2.42-2.34 (m, 1H), 2.05 (d, J = 14.1 Hz, 1H), 1.96- 1.79 (m, 3H), 1.75- 1.56 (m, 2H), 1.52 (q, J = 7.8 Hz, 1H), 1.35-1.18 (m, 5H), 1.04-0.82 (m, 7H). | | 471.12 | 0.4814 | >20 |
| 36 | 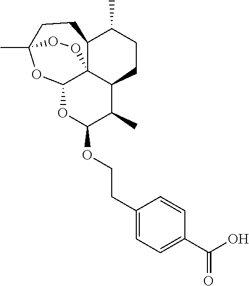 [SMILES: C[C@H]1[C@@H](OCCc2ccc(C(=O)O)cc2)O[C@@H]2O[C@@]3(C)CC[C@H]4[C@H](C)CC[C@@H]1[C@@]24OO3] | 4-(2- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)ethyl) benzoic acid | ¹H NMR (400 MHz, CDCl₃) δ 8.06-7.99 (m, 2H), 7.34 (d, J = 7.6 Hz, 2H), 4.95 (s, 1H), 4.77 (d, J = 3.3 Hz, 1H), 4.15 (q, J = 7.9 Hz, 1H), 3.65 (p, J = 5.0 Hz, 1H), 2.97 (d, J = 7.0 Hz, 2H), 2.55 (s, 1H), 2.40-2.26 (m, 1H), 1.99 (d, J = 14.6 Hz, 1H), 1.84- 1.76 (m, 1H), 1.57 (d, J = 11.7 Hz, 1H), 1.48-1.28 (m, 7H), 1.18-1.06 (m, 1H), 1.01-0.65 (m, 7H). | 433.24 | | 0.5261 | >20 |
| 37 | 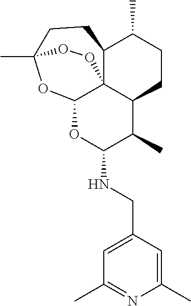 | (3R,5aS,6R,8aS, 9R,10R,12R, 12aR)-N-((2,6- dimethylpyridin- 4-yl)methyl)- 3,6,9- trimethyldeca- hydro-12H-3,12- epoxy[1,2] dioxepino[4,3-i] isochromen-10- amine | ¹H NMR (400 MHz, CDCl₃) δ 7.03 (s, 1H), 6.84 (s, 1H), 5.30 (s, 1H), 4.26-4.16 (m, 2H), 3.99 (d, J = 14.6 Hz, 1H), 2.50 (s, 3H), 2.44-2.37 (m, 0H), 2.37-2.31 (m, 1H), 2.29 (s, 3H), 2.05-1.94 (m, 1H), 1.92-1.81 (m, 1H), 1.78-1.62 (m, 2H), 1.58-1.51 (m, 1H), 1.50-1.41 (m, 1H), 1.40 (s, 3H), 1.38- 1.31 (m, 1H), 1.30- 1.19 (m, 2H), 1.06- 0.96 (m, 1H), 0.95- | 403.14 | | 0.5547 | 0.5904 |
| | | | 0.92 (m, 6H). | | | | |
| 38 | 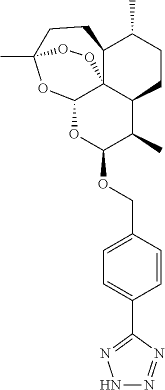 [SMILES: C[C@H]1[C@@H](OCc2ccc(-c3nn[nH]n3)cc2)O[C@@H]2O[C@@]3(C)CC[C@H]4[C@H](C)CC[C@@H]1[C@@]24OO3] | 5-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) phenyl)-2H- tetrazole | | 443.13 | | 0.6164 | 13.06 |
| 39 | 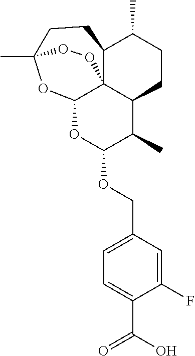 [SMILES: C[C@H]1[C@H](OCc2ccc(C(=O)O)c(F)c2)O[C@@H]2O[C@@]3(C)CC[C@H]4[C@H](C)CC[C@@H]1[C@@]24OO3] | 2-fluoro-4- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, CDCl₃) δ 7.98 (t, J = 7.7 Hz, 1H), 7.21 (dd, J = 9.5, 6.2 Hz, 2H), 5.36 (s, 1H), 5.03 (d, J = 13.8 Hz, 1H), 4.67 (d, J = 13.8 Hz, 1H), 4.54 (d, J = 9.2 Hz, 1H), 2.54 (d, J = 7.1 Hz, 1H), 2.44-2.34 (m, 1H), 2.04 (d, J = 14.5 Hz, 1H), 1.88 (s, 1H), 1.76 (d, J = 13.4 Hz, 1H), 1.68 (d, J = 13.9 Hz, 1H), 161-1.57 (m, 1H), 1.51-1.48 (m, 1H), 1.37-1.16 (m, 7H), 1.08-0.77 (m, 7H). | | 459.13 | 0.6313 | >20 |
| 40 | 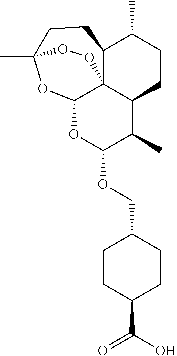 [SMILES: C[C@H]1[C@H](OC[C@H]2CC[C@H](C(=O)O)CC2)O[C@@H]2O[C@@]3(C)CC[C@H]4[C@H](C)CC[C@@H]1[C@@]24OO3] | (1R,4r)-4- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) cyclohexane-1- carboxylic acid | ¹H NMR (500 MHz, CDCl₃) δ 5.32 (s, 1H), 4.39 (d, J = 9.2 Hz, 1H), 3.83 (dd, J = 9.6, 5.8 Hz, 1H), 3.18 (dd, J = 9.6, 7.2 Hz, 1H), 2.45- 2.39 (m, 1H), 2.38- 2.33 (m, 1H), 2.26 (tt, J = 12.2, 3.5 Hz, 1H), 2.07- 1.99 (m, 3H), 1.98- 1.92 (m, 1H), 1.91- 1.79 (m, 3H), 1.79- 1.72 (m, 1H), 1.68 (dd, J = 13.3, 3.3 Hz, 1H), 1.62 (q, J = 5.3 Hz, 0H), 1.54 (dt, J = 13.4, 4.4 Hz, 1H), 1.51- 1.44 (m, 1H), 1.43 (s, 3H), 1.37-1.16 | 442.12 (M + 18) | | 0.6800 | >20 |
| | | | (m, 3H), 1.04-0.97 | | | | |
| | | | (m, 3H), 0.95 (d, J = | | | | |
| | | | 6.1 Hz, 3H), 0.88 (d, | | | | |
| | | | J = 7.1 Hz, 3H). | | | | |
| 41 | 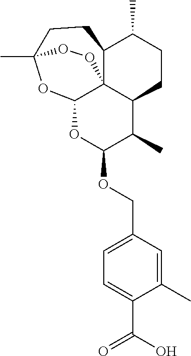 | 2-methyl-4- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, DMSO) δ 12.74 (s, 1H), 7.82 (dd, J = 8.4, 2.7 Hz, 1H), 7.23 (d, J = 5.9 Hz, 2H), 5.40 (d, J = 2.6 Hz, 1H), 4.81 (s, 1H), 4.77 (dd, J = 13.3, 2.8 Hz, 1H), 4.46 (dd, J = 13.1, 2.5 Hz, 1H), 2.48 (s, 3H), 2.19 (t, J = 14.0 Hz, 1H), 2.00 (d, J = 14.5 Hz, 1H), 1.84-1.65 (m, 3H), 1.56 (d, J = 13.0 Hz, 1H), 1.45-1.32 (m, 3H), 1.16 (d, J = 8.6 Hz, 1H), 0.89 (dd, J = 7.2, 4.0 Hz, 7H). | 450.16 (M + 18) | | 0.7353 | >20 |
| 42 | 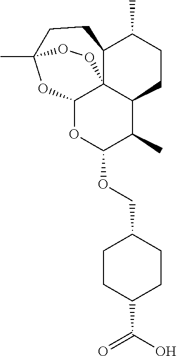 | (1S,4s)-4- ((((3R,5aS,6R, 8aS,9R,10R,12R, 12aR)-3,6,9- trimethyldeca- hydro-12H-3,12- epoxy[1,2] dioxepino[4,3-i] isochromen-10- yl)oxy)methyl) cyclohexane-1- carboxylic acid | ¹H NMR (400 MHz, CDCl₃) δ 5.32 (s, 1H), 4.39 (d, J = 9.2 Hz, 1H), 3.88 (dd, J = 9.6, 5.6 Hz, 1H), 3.23 (t, J = 8.9 Hz, 1H), 2.67-2.57 (m, 2H), 2.37 (td, J = 13.9, 4.2 Hz, 2H), 2.06- 1.94 (m, 4H), 1.93- 1.83 (m, 1H), 1.81- 1.65 (m, 5H), 1.64- 1.48 (m, 5H), 1.41- 1.21 (m, 5H), 1.02 (d, J = 13.4 Hz, 1H), 0.95 (d, J = 6.0 Hz, 4H), 0.88 (d, J = 7.1 Hz, 3H). | | 447.1 | 0.7820 | >20 |
| 43 | 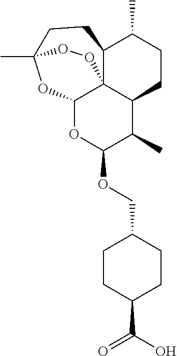 [SMILES: C[C@H]1[C@@H](OC[C@H]2CC[C@H](C(=O)O)CC2)O[C@@H]2O[C@@]3(C)CC[C@H]4[C@H](C)CC[C@@H]1[C@@]24OO3] | (1S,4r)-4- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxylic acid | ¹H NMR (500 MHz, CDCl₃) δ 5.36 (s, 1H), 4.75 (dd, J = 3.6, 1.0 Hz, 1H), 3.69 (dd, J = 9.5, 6.2 Hz, 1H), 3.17 (dd, J = 9.5, 6.1 Hz, 1H), 2.62 (ddd, J = 7.1, 4.7, 3.4 Hz, 1H), 2.40-2.32 (m, 1H), 2.28-2.22 (m, 1H), 2.07-1.99 (m, 3H), 1.92-1.78 (m, 4H), 1.78-1.72 (m, 2H), 1.64 (dq, J = 10.0, 3.4 Hz, 1H), 1.57 (ddq, J = 11.8, 5.8, 2.9 Hz, 1H), 1.50-1.44 (m, 3H), 1.43 (s, 3H), 1.30 (dtd, J = 9.3, 6.2, 3.3 Hz, | | | 0.8916 | >20 |
| | | | 1H), 1.27-1.16 (m, | | | | |
| | | | 2H), 1.04 (dt, J = | | | | |
| | | | 13.1, 3.2 Hz, 3H), | | | | |
| | | | 0.95 (d, J = 6.3 Hz, | | | | |
| | | | 3H), 0.90 (d, J = 7.3 | | | | |
| | | | Hz, 3H). | | | | |
| 44 | 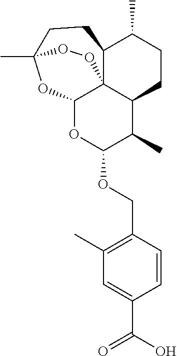 [SMILES: Cc1cc(C(=O)O)ccc1CO[C@@H]1O[C@@H]2O[C@@]3(C)CC[C@H]4[C@H](C)CC[C@@H]([C@H]1C)[C@@]24OO3] | 3-methyl-4- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, DMSO) δ 12.77 (s, 1H), 7.77- 7.75 (m, 2H), 7.48 (d, J = 8.2 Hz, 1H), 5.47 (s, 1H), 4.86 (d, J = 13.1 Hz, 1H), 4.66-4.53 (m, 2H), 2.31 (s, 3H), 2.27-2.13 (m, 2H), 2.01-1.98 (m, 1H), 1.85-1.74 (m, 1H), 1.59 (tt, J = 8.0, 3.5 Hz, 2H), 1.47 (dt, J = 13.7, 4.2 Hz, 1H), 1.40-1.33 (m, 3H), 1.30 (s, 3H), 1.24-1.10 (m, 1H), 1.00-0.77 (m, 7H). | | | 1.0133 | >20 |
| 45 | 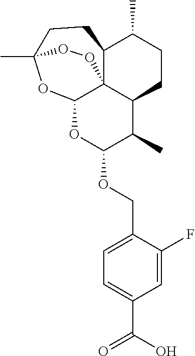 [SMILES: C[C@H]1[C@H](OCc2ccc(C(=O)O)cc2F)O[C@@H]2O[C@@]3(C)CC[C@H]4[C@H](C)CC[C@@H]1[C@@]24OO3] | 3-fluoro-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzoic acid | | | | 0.6625 | >20 |
| 46 | 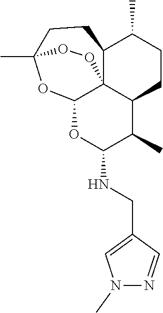 | (3R,5aS,6R,8aS, 9R,10R,12R, 12aR)-3,6,9- trimethyl-N-((1- methyl-1H- pyrazol-4-yl) methyl)decahydro- 12H-3,12- epoxy[1,2] dioxepino[4,3-i] isochromen-10- amine | | | | 1.4559 | 4.378 |
| 47 | 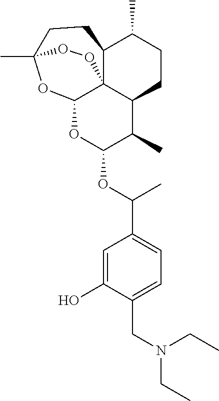 [SMILES: CCN(CC)Cc1ccc(C(C)O[C@@H]2O[C@@H]3O[C@@]4(C)CC[C@H]5[C@H](C)CC[C@@H]([C@H]2C)[C@@]35OO4)cc1O] | 2- ((diethylamino) methyl)-5-(1- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)ethyl) phenol | | 490.3 | | 1.7096 | 4.052 |
| 48 | 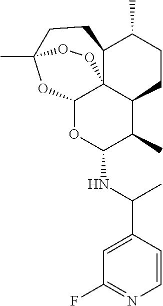 [SMILES: CC(N[C@@H]1O[C@@H]2O[C@@]3(C)CC[C@H]4[C@H](C)CC[C@@H]([C@H]1C)[C@@]24OO3)c1ccnc(F)c1] | (3R,5aS,6R,8aS, 9R,10R,12R, 12aR)-N-((2- fluoropyridin-4- yl)methyl)-3,6,9- trimethyldeca- hydro-12H-3,12- epoxy[1,2] dioxepino[4,3-i] isochromen-10- amine | ¹H NMR (400 MHz, CDCl₃) δ 8.12 (d, J = 5.1 Hz, 1H), 7.20 (d, J = 5.0, 1H), 7.02 (s, 1H), 5.28 (s, 1H), 4.16 (dd, J = 15.9, 0.9 Hz, 1H), 4.07 (d, J = 3.4 Hz, 1H), 4.03 (d, J = 9.5 Hz, 1H), 2.43-2.31 (m, 2H), 2.08-1.98 (m, 1H), 1.94-1.83 (m, 1H), 1.70 (tdd, J = 16.4, 7.1, 3.6 Hz, 2H), 1.59-1.51 (m, 1H), 1.51-1.45 (m, 1H), 1.44 (s, 3H), 1.36-1.16 | 375.14 | | 1.8945 | 3.357 |
| | | | (m, 3H), 1.07-0.98 | | | | |
| | | | (m, 1H), 0.97-0.94 | | | | |
| | | | (m, 6H). | | | | |
| 49 | 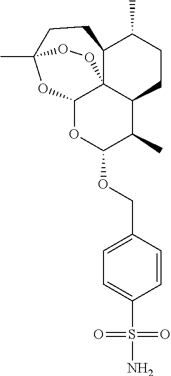 [SMILES: C[C@H]1[C@H](OCc2ccc(S(N)(=O)=O)cc2)O[C@@H]2O[C@@]3(C)CC[C@H]4[C@H](C)CC[C@@H]1[C@@]24OO3] | 4- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) benzene- sulfonamide | ¹H NMR (400 MHz, CDCl₃) δ 7.89 (d, J = 8.2 Hz, 2H), 7.51 (d, J = 8.1 Hz, 2H), 5.35 (s, 1H), 5.05 (d, J = 13.3 Hz, 1H), 4.80 (s, 2H), 4.68 (d, J = 13.4 Hz, 1H), 4.53 (d, J = 9.2 Hz, 1H), 2.56-2.46 (m, 1H), 2.43-2.35 (m, 1H), 2.04 (dd, J = 14.5, 4.2 Hz, 1H), 1.90 (d, J = 13.5 Hz, 1H), 1.80-1.69 (m, 1H), 1.68-1.54 (m, 2H), 1.50 (s, 1H), 1.45 (s, 3H), 1.36-1.17 (m, 3H), 1.05-0.98 (m, 1H), 0.96-0.93 (m, 6H). | 454.49 | | 2.0996 | ≥20 |
| 50 | 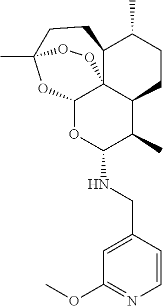 [SMILES: COc1cc(CN[C@@H]2O[C@@H]3O[C@@]4(C)CC[C@H]5[C@H](C)CC[C@@H]([C@H]2C)[C@@]35OO4)ccn1] | (3R,5aS,6R,8aS, 9R,10R,12R, 12aR)-N-((2- methoxypyridin- 4-yl)methyl)- 3,6,9- trimethyldeca- hydro-12H-3,12- epoxy[1,2] dioxepino[4,3-i] isochromen-10- amine | ¹H NMR (400 MHz, CDCl₃) δ 8.12-8.05 (d, J = 5.3 Hz, 1H), 6.96 (d, J = 5.3 Hz, 1H), 6.85 (s, 1H), 5.28 (s, 1H), 4.15-4.07 (m, 2H), 3.98 (d, J = 15.4 Hz, 1H), 3.94 (s, 3H), 2.44- 2.31 (m, 1H), 2.05- 1.99 (m, 1H), 1.89 (ddd, J = 13.7, 6.5, 3.4 Hz, 1H), 1.75- 1.64 (m, 3H), 1.55 (dt, J = 13.7, 4.3 Hz, 1H), 1.50-1.45 (m, 1H), 1.44 (s, 3H), 1.30-1.21 (m, 3H), | 405.15 | | 2.3161 | 4.883 |
| | | | 1.04-1.00 (m, 1H), | | | | |
| | | | 0.97 (d, J = 7.0 Hz, | | | | |
| | | | 3H), 0.95 (d, J = | | | | |
| | | | 6.0 Hz, 3H). | | | | |
| 51 | 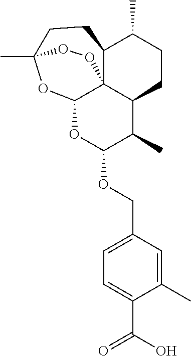 | 2-methyl-4- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, DMSO) δ 12.77 (s, 1H), 7.81 (d, J = 7.8 Hz, 1H), 7.26 (d, J = 8.5 Hz, 2H), 5.47 (s, 1H), 4.82 (d, J = 13.1 Hz, 1H), 4.64-4.48 (m, 2H), 2.50 (s, 3H), 2.19 (t, J = 12.0 Hz, 2H), 2.00 (d, J = 14.3 Hz, 1H), 1.80 (s, 1H), 1.60 (s, 2H), 1.35 (s, 2H), 1.30 (s, 3H), 1.23 (s, 1H), 1.18 (dd, J = 11.0, 6.5 Hz, 2H), 0.89-0.84 (m, 7H). | 450.16 (M + 1) | | 2.4117 | >20 |
| 52 | 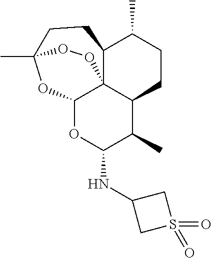 [SMILES: C[C@H]1[C@H](NC2CS(=O)(=O)C2)O[C@@H]2O[C@@]3(C)CC[C@H]4[C@H](C)CC[C@@H]1[C@@]24OO3] | 3- (((3R,5aS,6R,8aS, 9R,10R,12R, 12aR)-3,6,9- trimethyldeca- hydro-12H-3,12- epoxy[1,2] dioxepino[4,3-i] isochromen-10- yl)amino) thietane 1,1- dioxide | ¹H NMR (400 MHz, CDCl₃) δ 5.29 (s, 1H), 4.42- 4.28 (m, 2H), 4.26- 4.05 (m, 3H), 4.04- 3.97 (m, 1H), 2.42- 2.29 (m, 2H), 2.01 (ddd, J = 14.6, 6.5, 3.6 Hz, 1H), 1.93- 1.83 (m, 1H), 1.73 (td, J = 13.9, 3.5 Hz, 3H), 1.56 (dt, J = 13.8, 4.2 Hz, 1H), 1.51-1.40 (m, 1H), 1.38 (s, 3H), 1.34-1.20 (m, 3H), 1.06-0.98 (m, 1H), | | 410.04 | 2.4547 | 3.552 |
| | | | 0.95 (d, J = 6.1 Hz, | | | | |
| | | | 3H), 0.89 (d, J = 7.2 | | | | |
| | | | 3H). | | | | |
| 53 | 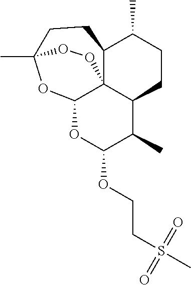 | (3R,5aS,6R,8aS, 9R,10R,12R, 12aR)-3,6,9- trimethyl-10-(2- (methylsulfonyl) ethoxy)deca- hydro-12H- 3,12-epoxy[1,2] dioxepino[4,3-i] isochromene | ¹H NMR (500 MHz, CDCl₃) δ 5.34 (s, 1H), 4.51 (d, J = 9.2 Hz, 1H), 4.27 (ddd, J = 11.4, 5.5, 3.5 Hz, 1H), 4.07 (ddd, J = 11.7, 9.3, 2.9 Hz, 1H), 3.52 (ddd, J = 15.1, 9.3, 3.5 Hz, 1H), 3.17-3.08 (m, 1H), 3.05 (s, 3H), 2.46- 2.32 (m, 2H), 2.07- 1.99 (m, 1H), 1.92- 1.86 (ddt, J = 13.5, 6.7, 3.6 Hz, 1H), 1.76 (dd, J = 13.5, 3.9 Hz, 1H), 1.70 | 408.09 (m + 18) | | 2.7257 | >20 |
| | | | (dd, J = 13.3, 3.3 | | | | |
| | | | Hz, 1H), 1.56-1.53 | | | | |
| | | | (m, 1H), 1.51-1.45 | | | | |
| | | | (m, 1H), 1.45-1.42 | | | | |
| | | | (m, 1H), 1.41 (s, | | | | |
| | | | 3H), 1.31-1.23 (m, | | | | |
| | | | 2H), 1.01 (td, J = | | | | |
| | | | 12.4, 3.4 Hz, 1H), | | | | |
| | | | 0.96 (d, J = 6.1 Hz, | | | | |
| | | | 3H), 0.89 (d, J = 7.1 | | | | |
| | | | Hz, 3H). | | | | |
| 54 | 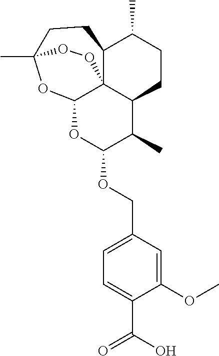 | 2-methoxy-4- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, DMSO) δ 12.52 (s, 1H), 7.63 (dd, J = 6.6, 3.6 Hz, 1H), 7.11 (s, 1H), 6.97 (d, J = 7.8 Hz, 1H), 5.46 (d, J = 2.5 Hz, 1H), 4.83 (d, J = 13.4 Hz, 1H), 4.65-4.57 (m, 2H), 3.81 (d, J = 2.5 Hz, 3H), 2.24-2.14 (m, 2H), 2.00 (d, J = 14.5 Hz, 1H), 1.81 (s, 1H), 1.58 (d, J = 12.1 Hz, 2H), 1.49 (d, J = 13.7 Hz, 1H), 1.40-1.33 (m, 2H), 1.30 (s, 3H), 1.23 (s, 1H), 1.17 (d, J = 5.9 Hz, 1H), 0.99- 0.86 (m, 7H). | 449.15 | | 2.8245 | >20 |
| 55 | 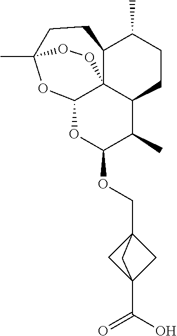 [SMILES: C[C@H]1[C@@H](OCC23CC(C(=O)O)(C2)C3)O[C@@H]2O[C@@]3(C)CC[C@H]4[C@H](C)CC[C@@H]1[C@@]24OO3] | 3- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) bicyclo[1.1.1] pentane-1- carboxylic acid | ¹H NMR (400 MHz, DMSO) δ 12.34 (s, 1H), 5.26 (s, 1H), 4.67 (d, J = 3.4 Hz, 1H), 3.64 (d, J = 11.1 Hz, 1H), 3.38 (d, J = 11.2 Hz, 1H), 2.39 (dt, J = 8.0, 4.3 Hz, 1H), 2.18 (td, J = 14.0, 3.9 Hz, 1H), 1.99 (dq, J = 9.2, 4.1 Hz, 1H), 1.87 (d, J = 3.9 Hz, 6H), 1.83-1.77 (m, 1H), 1.69 (d, J = 4.6 Hz, 1H), 1.58 (dd, J = 12.9, 3.3 Hz, 1H), 1.38 (dt, J = 14.0, 5.4 Hz, 2H), 1.30 (s, 1H), 1.28 (s, 3H), | | 431.39 | 7.40395 | >20 |
| | | | 1.20-1.06 (m, 2H), | | | | |
| | | | 0.95-0.82 (m, 7H). | | | | |
| 56 | 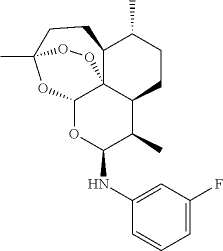 [SMILES: C[C@H]1[C@@H](Nc2cccc(F)c2)O[C@@H]2O[C@@]3(C)CC[C@H]4[C@H](C)CC[C@@H]1[C@@]24OO3] | (3R,5aS,6R,8aS, 9R,10S,12R, 12aR)-N-(3- fluorophenyl)- 3,6,9- trimethyldeca- hydro-12H-3,12- epoxy[1,2] dioxepino[4,3-i] isochromen-10- amine | ¹H NMR (400 MHz, CDCl₃) δ 7.10 (td, J = 8.2, 6.6 Hz, 1H), 6.64 (dt, J = 11.7, 2.3 Hz, 1H), 6.56 (ddd, J = 8.1, 2.2, 0.9 Hz, 1H), 6.52-6.42 (m, 1H), 5.58 (d, J = 9.4 Hz, 1H), 5.47 (s, 1H), 4.21 (s, 1H), 2.34 (ddd, J = 14.6, 13.1, 3.9 Hz, 1H), 2.07 (dt, J = 14.7, 4.0 Hz, 1H), 1.99-1.91 (m, 1H), 1.76-1.69 | 378.38 | | 8.1900 | 10.96 |
| | | | (m, 1H), 1.64-1.60 | | | | |
| | | | (m, 1H), 1.53 (s, | | | | |
| | | | 3H), 1.46-1.44 (m, | | | | |
| | | | 1H), 1.41 (dd, J = | | | | |
| | | | 4.0, 2.4 Hz, 1H), | | | | |
| | | | 1.39-1.34 (m, 2H), | | | | |
| | | | 1.33-1.28 (m, 2H), | | | | |
| | | | 1.17 (d, J = 7.0 Hz, | | | | |
| | | | 3H), 1.09-1.00 (m, | | | | |
| | | | 1H), 0.97 (d, J = 5.6 | | | | |
| | | | Hz, 3H). | | | | |
| 57 | 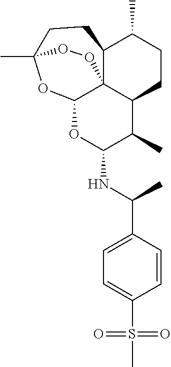 [SMILES: C[C@H]1[C@H](N[C@@H](C)c2ccc(S(C)(=O)=O)cc2)O[C@@H]2O[C@@]3(C)CC[C@H]4[C@H](C)CC[C@@H]1[C@@]24OO3] | (3R,5aS,6R,8aS, 9R,10R,12R, 12aR)-3,6,9- trimethyl-N-((S)- 1-(4-(methyl- sulfonyl)phenyl) ethyl)decahydro- 12H-3,12- epoxy[1,2] dioxepino[4,3-i] isochromen-10- amine | ¹H NMR (400 MHz, CDCl₃) δ 7.89 (d, J = 8.4 Hz, 1H), 7.66 (d, J = 8.0 Hz, 2H), 5.11 (s, 1H), 4.59 (q, J = 6.8 Hz, 1H), 3.70 (d, J = 9.6 Hz, 1H), 3.07 (s, 3H), 2.42- 2.29 (m, 2H), 2.01 (dt, J = 14.7, 4.0 Hz, 1H), 1.86 (d, J = 13.8 Hz, 1H), 1.63-1.56 (m, 3H), 1.50 (dt, J = 12.9, 4.0 Hz, 1H), 1.45 (s, 3H), 1.40 (d, J = 7.0 Hz, 2H), 1.26- 1.15 (m, 2H), 1.02- 0.97 (m, 1H), 0.94 (d, J = 7.3 Hz, 3H), 0.91 (d, J = 5.8 Hz, 3H). | 466.17 | | 8.4933 | 9.893 |
| 58 | 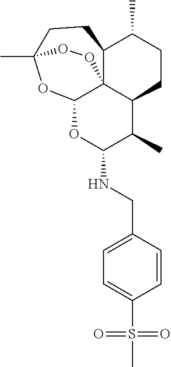 [SMILES: C[C@H]1[C@H](NCc2ccc(S(C)(=O)=O)cc2)O[C@@H]2O[C@@]3(C)CC[C@H]4[C@H](C)CC[C@@H]1[C@@]24OO3] | (3R,5aS,6R,8aS, 9R,10R,12R, 12aR)-3,6,9- trimethyl-N-(4- (methylsulfonyl) benzyl) decahydro-12H- 3,12-epoxy[1,2] dioxepino[4,3-i] isochromen-10- amine | | 452.17 | | 9.1187 | 8.335 |
| 59 | 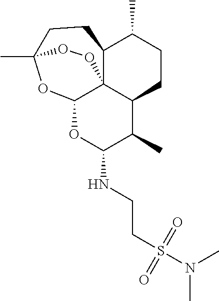 | N,N-dimethyl-2- (((3R,5aS,6R, 8aS,9R,10R,12R, 12aR)-3,6,9- trimethyldeca- hydro-12H-3,12- epoxy[1,2] dioxepino[4,3-i] isochromen-10- yl)amino) ethane-1- sulfonamide | ¹H NMR (400 MHz, CDCl₃) δ 5.28 (d, J = 4.2 Hz, 1H), 4.12 (d, J = 9.7 Hz, 1H), 3.37-3.15 (m, 4H), 2.88 (s, 6H), 2.33 (d, J = 13.4 Hz, 2H), 2.01 (d, J = 15.3 Hz, 1H), 1.86 (s, 1H), 1.69 (d, J = 15.3 Hz, 2H), 1.59 (s, 2H), 1.53-1.44 (m, 1H), 1.38 (d, J = 4.0 Hz, 3H), 1.34-1.20 (m, 2H), 1.12-1.00 (m, 1H), 0.95 (d, J = 5.6 Hz, 3H), 0.87 (d, J = 6.6 Hz, 3H). | 419.15 | | 9.6329 | 10.99 |
| 60 | 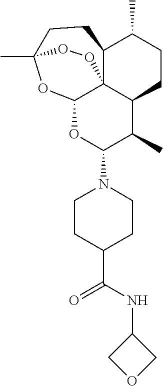 | N-(oxetan-3- yl)-1-((3R,5aS, 6R,8aS,9R,10R, 12R,12aR)-3,6,9- trimethyldeca- hydro-12H-3,12- epoxy[1,2] dioxepino[4,3-i] isochromen-10- yl)piperidine-4- carboxamide | ¹H NMR (400 MHz, DMSO) δ 8.47 (d, J = 6.6 Hz, 1H), 5.30 (s, 1H), 4.76 (s, 1H), 4.68 (t, J = 6.9 Hz, 2H), 4.40 (d, J = 6.6 Hz, 2H), 4.02 (d, J = 10.0 Hz, 1H), 2.92 (d, J = 11.5 Hz, 1H), 2.74 (s, 2H), 2.36-2.30 (m, 1H), 2.23-2.04 (m, 2H), 1.96 (d, J = 14.6 Hz, 2H), 1.76 (s, 1H), 1.63 (d, J = 13.3 Hz, 2H), 1.57 (d, J = 9.1 Hz, 2H), 1.44 (s, 2H), 1.41- 1.30 (m, 2H), 1.26 (s, 3H), 1.23 (s, 1H), 1.13 (s, 1H), 0.98- 0.93 (m, 1H), 0.88 (d, J = 6.3 Hz, 3H), | 451.23 | | 11.1792 | 11.78 |
| | | | 0.71 (d, J = 7.0 Hz, | | | | |
| | | | 3H). | | | | |
| 61 | 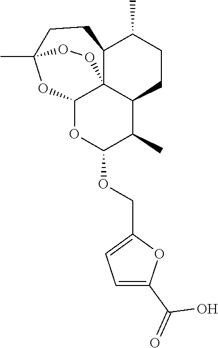 [SMILES: C[C@H]1[C@H](OCc2ccc(C(=O)O)o2)O[C@@H]2O[C@@]3(C)CC[C@H]4[C@H](C)CC[C@@H]1[C@@]24OO3] | 5- ((((3R,5aS,6R, 8aS,9R,10R,12R, 12aR)-3,6,9- trimethyldeca- hydro-12H-3,12- epoxy[1,2] dioxepino[4,3-i] isochromen-10- yl)oxy)methyl) furan-2- carboxylic acid | ¹H NMR (400 MHz, CDCl₃) δ 7.21 (d, J = 3.5 Hz, 1H), 6.47 (d, J = 3.4 Hz, 1H), 5.35 (s, 1H), 4.88 (d, J = 13.6 Hz, 1H), 4.69 (d, 13.6 Hz, 1H), 4.55 (d, J = 9.3 Hz, 1H), 3.33 (s, 1H), 2.50- 2.40 (m, 1H), 2.39- 2.32 (m, 1H), 2.02 (ddd, J = 14.6, 4.9, 3.3 Hz, 1H), 1.91- 1.86 (m, 1H), 1.78- 1.63 (m, 2H), 1.55 (dt, J = 13.9, 4.5 Hz, 1H), 1.50-1.46 (m, 1H), 1.35-1.27 (m, 2H), 1.25 (s, 3H), 1.20 (dd, J = 13.4, | 426.11 (M + 1) | | 13.3549 | >20 |
| | | | 3.1 Hz, 1H), 1.01 (d, | | | | |
| | | | J = 11.7 Hz, 1H), | | | | |
| | | | 0.95 (d, J = 5.9 Hz, | | | | |
| | | | 3H), 0.86 (d, J = 7.1 | | | | |
| | | | Hz, 3H). | | | | |
| 62 | 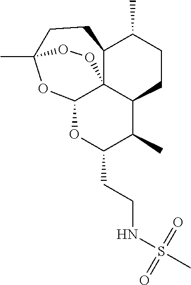 | N-(2- ((3R,5aS,6R,8aS, 9R,10S,12R, 12aR)-3,6,9- trimethyldeca- hydro-12H-3,12- epoxy[1,2] dioxepino[4,3-i] isochromen-10- yl)ethyl) methane- sulfonamide | ¹H NMR (400 MHz, CDCl₃) δ 5.33 (s, 1H), 5.11 (s, 1H), 4.44 (dd, J = 11.3, 6.3 Hz, 1H), 3.37 (s, 1H), 3.31-3.24 (m, 1H), 2.96 (s, 3H), 2.59 (q, J = 7.2 Hz, 1H), 2.31 (ddd, J = 17.3, 13.6, 4.2 Hz, 1H), 2.09-1.99 (m, 1H), 1.97-1.90 (m, 1H), 1.89-1.82 (m, 1H), 1.81-1.76 (m, 1H), 1.73-1.64 (m, 3H), 1.41 (s, 3H), 1.33-1.27 (m, 4H), 0.97 (d, J = 5.6 | | 412.05 | 16.4249 | >20 |
| | | | Hz, 4H), 0.87 (d, J = | | | | |
| | | | 7.6 Hz, 3H). | | | | |
| 63 | 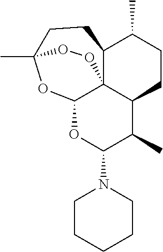 | 1- ((3R,5aS,6R,8aS, 9R,10R,12R, 12aR)-3,6,9- trimethyldeca- hydro-12H-3,12- epoxy[1,2] dioxepino[4,3-i] isochromen-10- yl)piperidine | | 352.09 | | >20 | >20 |
| 64 | 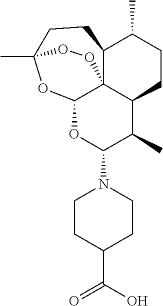 | 1- ((3R,5aS,6R,8aS, 9R,10R,12R, 12aR)-3,6,9- trimethyldeca- hydro-12H-3,12- epoxy[1,2] dioxepino[4,3-i] isochromen-10- yl)piperidine-4- carboxylic acid | ¹H NMR (400 MHz, DMSO) δ 12.09 (s, 1H), 5.30 (s, 1H), 4.03 (d, J = 10.0 Hz, 1H), 2.91 (d, J = 11.9 Hz, 1H), 2.71 (s, 2H), 2.67 (s, 1H), 2.34 (d, J = 11.0 Hz, 2H), 2.18 (d, J = 12.1 Hz, 2H), 1.96 (d, J = 14.7 Hz, 1H), 1.78 (s, 2H), 1.58 (t, J = 11.4 Hz, 2H), 1.44 (s, 2H), 1.33 (d, J = 12.3 Hz, 2H), 1.26 (s, 3H), 1.15-1.08 (m, 1H), 0.99-0.92 (m, 1H), 0.88 (d, J = | 396.10 | | >20 | >20 |
| | | | 6.3 Hz, 3H), 0.71 | | | | |
| | | | (d, J = 7.1 Hz, 3H). | | | | |
| 65 | 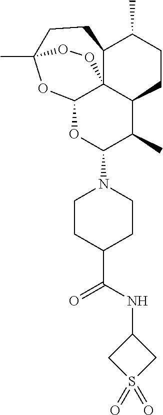 | N-(1,1- dioxidothietan- 3-yl)-1- ((3R,5aS,6R,8aS, 9R,10R,12R, 12aR)-3,6,9- trimethyldeca- hydro-12H-3,12- epoxy[1,2] dioxepino[4,3-i] isochromen-10- yl)piperidine-4- carboxamide | ¹H NMR (400 MHz, DMSO) δ 8.51 (d, J = 5.3 Hz, 1H), 5.30 (s, 1H), 4.54- 4.44 (m, 2H), 4.31 (d, J = 4.9 Hz, 0H), 4.07-3.98 (m, 3H), 2.93 (d, J = 11.8 Hz, 1H), 2.74 (s, 3H), 2.38-2.27 (m, 2H), 2.19-2.07 (m, 2H), 1.96 (d, J = 14.6 Hz, 1H), 1.77 (s, 1H), 1.72-1.60 (m, 2H), 1.57 (d, J = 8.5 Hz, 3H), 1.44 (s, 2H), 1.41- 1.29 (m, 2H), 1.26 (s, 3H), 1.13 (t, J = 8.9 Hz, 1H), 0.99- 0.94 (m, 1H), 0.88 (d, J = 6.3 Hz, 3H), 0.71 (d, J = 7.1 Hz, 3H). | 499.12 | | >20 | >20 |
| 66 | 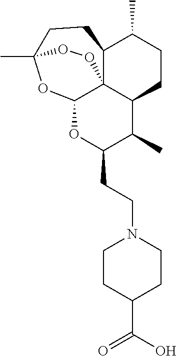 [SMILES: C[C@H]1[C@@H](CCN2CCC(C(=O)O)CC2)O[C@@H]2O[C@@]3(C)CC[C@H]4[C@H](C)CC[C@@H]1[C@@]24OO3] | 1-(2-((3R,5aS,6R, 8aS,9R,10R,12R, 12aR)-3,6,9- trimethyldeca- hydro-12H-3,12- epoxy[1,2] dioxepino[4,3-i] isochromen-10- yl)ethyl) piperidine-4- carboxylic acid | ¹H NMR (400 MHz, DMSO) δ 5.88 (s, 1H), 4.13 (d, J = 7.7 Hz, 1H), 3.98 (t, J = 8.4 Hz, 1H), 3.69 (q, J = 7.7 Hz, 1H), 2.79-2.65 (m, 2H), 2.29-2.09 (m, 4H), 2.04 (s, 3H), 1.90 (q, J = 10.4 Hz, 2H), 1.81-1.69 (m, 5H), 1.49 (s, 4H), 1.37 (q, J = 11.3 Hz, 3H), 1.31-1.14 (m, 2H), 0.94-0.67 (m, 7H). | 424.24 | | >20 | >20 |
| 67 | 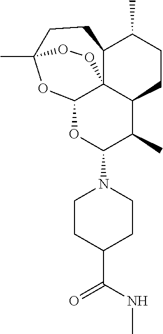 | N-methyl-4- ((3R,5aS,6R,8aS, 9R,10R,12R, 12aR)-3,6,9- trimethyldeca- hydro-12H-3,12- epoxy[1,2] dioxepino[4,3-i] isochromen-10- yl)piperazine-1- carboxamide | ¹H NMR (400 MHz, CDCl₃) δ 5.29 (s, 1H), 4.45 (s, 1H), 4.11 (s, 1H), 3.56- 3.31 (s, 4H), 3.05 (s, 2H), 2.80 (d, J = 4.5 Hz, 3H), 2.75 (s, 1H), 2.64-2.54 (m, 1H), 2.34 (ddd, J = 14.6, 13.3, 4.0 Hz, 1H), 2.05-1.95 (m, 1H), 1.87 (dq, J = 10.0, 3.2 Hz, 1H), 1.74-1.68 (m, 2H), 1.56 (dd, J = 13.5, 4.3 Hz, 1H), 1.51- 1.41 (m, 1H), 1.38 (s, 3H), 1.36-1.28 (m, 2H), 1.27-1.20 (m, 1H), 1.07-0.97 (m, 1H), 0.95 (d, | 410.68 | | >20 | >20 |
| | | | J = 6.2 Hz, 3H), | | | | |
| | | | 0.91-0.82 (m, 3H). | | | | |
| 68 | 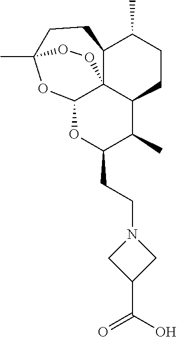 [SMILES: C[C@H]1[C@@H](CCN2CC(C(=O)O)C2)O[C@@H]2O[C@@]3(C)CC[C@H]4[C@H](C)CC[C@@H]1[C@@]24OO3] | 1-(2- ((3R,5aS,6R,8aS, 9R,10R,12R, 12aR)-3,6,9- trimethyldeca- hydro-12H-3,12- epoxy[1,2] dioxepino[4,3-i] isochromen-10- yl)ethyl) azetidine-3- carboxylic acid | | 396.30 | | >20 | >20 |
| 69 | 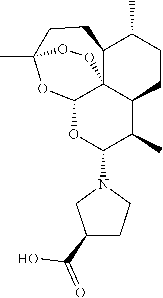 | (R)-1- ((3R,5aS,6R,8aS, 9R,10R,12R, 12aR)-3,6,9- trimethyldeca- hydro-12H-3,12- epoxy[1,2] dioxepino[4,3-i] isochromen-10- yl)pyrrolidine- 3-carboxylic acid | ¹H NMR (400 MHz, CDCl₃) δ 5.37 (s, 1H), 4.44 (d, J = 10.4 Hz, 1H), 3.43- 3.32 (m, 2H), 3.32- 3.24 (m, 1H), 3.18 (dd, J = 8.7, 5.6 Hz, 1H), 3.12-3.05 (m, 1H), 2.52 (ddd, J = 11.0, 7.3, 4.3 Hz, 1H), 2.37 (t, J = 13.9, 4.1 Hz, 1H), 2.30-2.23 (m, 1H), 2.23-2.12 (m, 1H), 2.03 (dt, J = 14.6, 4.3 Hz, 1H), 1.89 (ddd, J = 13.6, 6.6, 3.4 Hz, 1H), 1.74 (ddd, J = 12.7, 7.6, | | 382.10 | >20 | >20 |
| | | | 3.6 Hz, 2H), 1.58 | | | | |
| | | | (dt, J = 13.5, 4.2 | | | | |
| | | | Hz, 1H), 1.54-1.44 | | | | |
| | | | (m, 1H), 1.40 (s, | | | | |
| | | | 3H), 1.37-1.21 (m, | | | | |
| | | | 3H), 1.09-1.00 (m, | | | | |
| | | | 1H), 1.00-0.91 (m, | | | | |
| | | | 6H). | | | | |
| 70 | 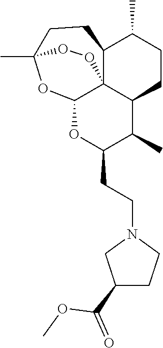 | methyl (R)-1-(2- ((3R,5aS,6R,8aS, 9R,10R,12R, 12aR)-3,6,9- trimethyldeca- hydro-12H-3,12- epoxy[1,2] dioxepino[4,3-i] isochromen-10- yl)ethyl) pyrrolidine-3- carboxylate | | 424.17 | | >20 | >20 |
| 71 | 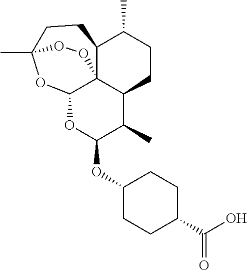 [SMILES: C[C@H]1[C@@H](O[C@H]2CC[C@@H](C(=O)O)CC2)O[C@@H]2O[C@@]3(C)CC[C@H]4[C@H](C)CC[C@@H]1[C@@]24OO3] | (1R,4s)-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclo- hexane-1- carboxylic acid | ¹H NMR (400 MHz, DMSO) δ 12.03 (s, 1H), 5.32 (s, 1H), 4.78 (d, J = 3.5 Hz, 1H), 3.76 (s, 1H), 3.34 (s, 1H), 2.39 (dt, J = 8.1, 4.2 Hz, 1H), 2.24 (dd, J = 14.3, 7.3 Hz, 1H), 2.16 (dd, J = 13.9, 3.9 Hz, 1H), 2.04- 1.94 (m, 1H), 1.80 (td, J = 11.6, 3.6 Hz, 2H), 1.72-1.53 (m, 9H), 1.47-1.31 (m, 3H), 1.28 (s, 3H), 1.16 (dd, J = 11.5, 6.4 Hz, 1H), 0.94- 0.81 (m, 7H). | | 433.45 | | |
| 72 | 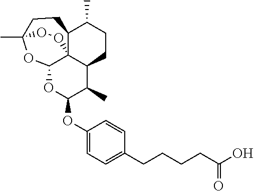 [SMILES: C[C@H]1[C@@H](Oc2ccc(CCCCC(=O)O)cc2)O[C@@H]2O[C@@]3(C)CC[C@H]4[C@H](C)CC[C@@H]1[C@@]24OO3] | 5-(4-(((3R,5aS, 6R,8aS,9R,10S, 12R,12aR)-3,6,9- trimethyldeca- hydro-12H-3,12- epoxy[1,2] dioxepino[4,3-i] isochromen-10- yl)oxy)phenyl) pentanoic acid | ¹H NMR (400 MHz, DMSO) δ 12.00 (s, 1H), 7.11 (d, J = 8.36 Hz, 2H), 6.97 (d, J = 8.24 Hz, 2H), 5.76 (s, 1H), 5.48 (d, J = 3.4 Hz, 2H), 5.42 (s, 1H), 2.63- 2.56 (m, 1H), 2.22 (t, J = 7.0 Hz, 3H), 2.03-1.95 (m, 1H), 1.94-1.87 (m, 1H), 1.86-1.76 (m, 2H), | 461.52 | | 0.11304 | 0.7772 |
| | | | 1.67-1.59 (m, 1H), | | | | |
| | | | 1.59-1.44 (m, 5H), | | | | |
| | | | 1.43-1.33 | | | | |
| | | | (m, 2H), 1.28 (s, | | | | |
| | | | 3H), 1.25-1.13 (m, | | | | |
| | | | 2H), 1.01-0.85 (m, | | | | |
| | | | 7H). | | | | |
| 73 | 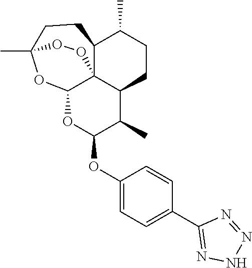 [SMILES: C[C@H]1[C@@H](Oc2ccc(-c3nn[nH]n3)cc2)O[C@@H]2O[C@@]3(C)CC[C@H]4[C@H](C)CC[C@@H]1[C@@]24OO3] | 5-(4- (((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)phenyl)- 2H-tetrazole | | 429.58 | | 0.034612 | 0.0319 |
| 74 | 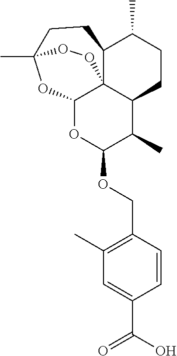 [SMILES: Cc1cc(C(=O)O)ccc1CO[C@H]1O[C@@H]2O[C@@]3(C)CC[C@H]4[C@H](C)CC[C@@H]([C@H]1C)[C@@]24OO3] | 3-methyl-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, DMSO) δ 12.85 (s, 1H), 7.76 (s, 2H), 7.44 (d, J = 7.5 Hz, 1H), 5.41 (s, 1H), 4.95-4.70 (m, 2H), 4.46 (d, J = 13.1 Hz, 1H), 2.31 (s, 3H), 2.24-2.10 (m, 1H), 2.01 (d, J = 14.4 Hz, 1H), 1.86- 1.65 (m, 3H), 1.55 (d, J = 13.0 Hz, 1H), 1.44-1.37 (s, 1H), 1.31 (s, 3H), 1.17 (s, 1H), 0.99- 0.75 (m, 7H). | 433.53 | | 0.8652 | >20 |
| 75 | 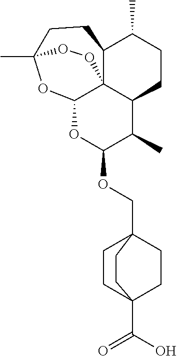 [SMILES: C[C@H]1[C@@H](OCC23CCC(C(=O)O)(CC2)CC3)O[C@@H]2O[C@@]3(C)CC[C@H]4[C@H](C)CC[C@@H]1[C@@]24OO3] | 4- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) bicyclo[2.2.2] octane-1- carboxylic acid | ¹H NMR (400 MHz, DMSO) δ 11.98 (s, 1H), 5.22 (s, 1H), 4.60 (d, J = 3.4 Hz, 1H), 3.37 (d, J = 9.5 Hz, 2H), 2.88 (d, J = 9.2 Hz, 1H), 2.38 (dt, J = 8.0, 4.2 Hz, 1H), 2.17 (td, J = 13.9, 3.8 Hz, 1H), 1.99 (d, J = 14.2 Hz, 1H), 1.84-1.75 (m, 1H), 1.73-1.64 (m, 8H), 1.62-1.54 (m, 1H), 1.32-1.21 (m, 4H), 1.43-1.34 (m, 8H), 1.27 (s, 3H), 1.14 (td, J = 11.3, 6.4 Hz, 1H), 0.96-0.78 (m, 7H). | | | 1.181201 | >20 |
| 76 | 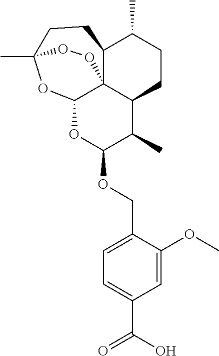 | 3-methoxy-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, DMSO) δ 12.98 (s, 1H), 7.57 (d, J = 7.7 Hz, 1H), 7.48 (s, 1H), 7.41 (d, J = 7.7 Hz, 1H), 5.41 (s, 1H), 4.89-4.73 (m, 2H), 4.42 (d, J = 13.7 Hz, 1H), 3.85 (s, 3H), 2.24-2.13 (m, 1H), 2.00 (d, J = 14.4 Hz, 1H), 1.88- 1.64 (m, 3H), 1.56 (d, J = 13.0 Hz, 1H), 1.45-1.34 (m, 2H), 1.31 (d, J = 4.2 Hz, 3H), 1.27-1.23 (m, 1H), 1.21-1.07 (m, 2H), 1.01-0.80 (m, 7H). | 449.53 | | | |
| 77 | 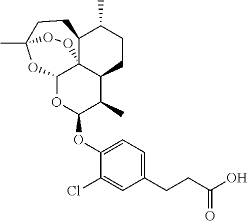 [SMILES: C[C@H]1[C@@H](Oc2ccc(CCC(=O)O)cc2Cl)O[C@@H]2O[C@@]3(C)CC[C@H]4[C@H](C)CC[C@@H]1[C@@]24OO3] | 3-(3-chloro-4- (((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)phenyl) propanoic acid | ¹H NMR (400 MHz, DMSO) δ 12.15 (s, 1H), 7.32 (d, J = 2.0 Hz, 1H), 7.26 (d, J = 8.5 Hz, 1H), 7.21- 7.14 (m, 1H), 5.63 (d, J = 3.5 Hz, 1H), 5.46 (s, 1H), 2.76 (t, J = 7.5 Hz, 2H), 2.61 (s, 1H), 2.25- 2.13 (m, 1H), 2.10- 1.94 (m, 2H), 1.81 (d, J = 13.5 Hz, 2H), 1.63 (d, J = 12.9 Hz, | | 489.58 | | |
| | | | 1H), 1.54 (d, J = | | | | |
| | | | 13.6 Hz, 1H), 1.35 | | | | |
| | | | (dt, J = 13.5, 6.7 | | | | |
| | | | Hz, 2H), 1.28 (s, | | | | |
| | | | 3H), 1.25-1.14 (m, | | | | |
| | | | 2H), 1.00 (d, J = 7.2 | | | | |
| | | | Hz, 3H), 0.91 (d, | | | | |
| | | | J = 6.3 Hz, 3H), | | | | |
| | | | 0.96-0.82 (m, 1H). | | | | |
| 78 | 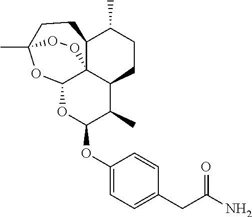 [SMILES: C[C@H]1[C@@H](Oc2ccc(CC(N)=O)cc2)O[C@@H]2O[C@@]3(C)CC[C@H]4[C@H](C)CC[C@@H]1[C@@]24OO3] | 2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetamide | ¹H NMR (400 MHz, DMSO) δ 7.41 (s, 1H), 7.18 (d, J = 8.1 Hz, 2H), 6.99 (d, J = 8.0 Hz, 2H), 6.84 (s, 1H), 5.50 (d, J = 3.4 Hz, 1H), 5.42 (s, 1H), 3.31 (d, J = 7.5 Hz, 2H), 2.64-2.51 (m, 1H), 2.19 (td, J = 14.0, 3.8 Hz, 1H), 1.99 (d, J = 15.7 Hz, 1H), 1.94-1.85 (m, 1H), 1.86-1.75 (m, 2H), | 418.50 | | | |
| | | | 1.63 (d, J = 12.8 Hz, | | | | |
| | | | 1H), 1.57-1.46 (m, | | | | |
| | | | 1H), 1.44-1.32 (m, | | | | |
| | | | 2H), 1.28 (s, 3H), | | | | |
| | | | 1.27-1.14 (m, 1H), | | | | |
| | | | 1.04-0.80 (m, 7H). | | | | |
| 79 | 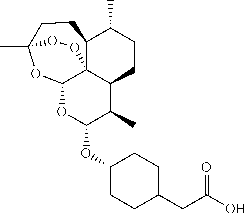 | 2-((1S,4s)-4- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)cyclo- hexyl)acetic acid | ¹H NMR (400 MHz, DMSO) δ 12.00 (s, 1H), 5.41 (s, 1H), 4.52 (d, J = 9.1 Hz, 1H), 3.85 (s, 1H), 2.22-2.06 (m, 4H), 1.97 (d, J = 13.5, Hz, 1H), 1.84-1.67 (m, 5H), 1.63-1.54 (m, 2H), 1.52-1.40 (m, 5H), 1.37-1.29 (m, 3H), 1.27 (s, 3H), 1.24-1.11 (m, 2H), 0.96-0.76 (m, 7H). | | 447.50 | | |
| 80 | 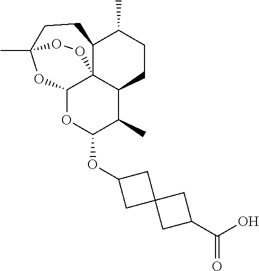 [SMILES: C[C@H]1[C@H](OC2CC3(C2)CC(C(=O)O)C3)O[C@@H]2O[C@@]3(C)CC[C@H]4[C@H](C)CC[C@@H]1[C@@]24OO3] | 6- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)spiro[3.3] heptane-2- carboxylic acid | ¹H NMR (400 MHz, DMSO) δ 12.02 (s, 1H), 5.39 (s, 1H), 4.36 (t, J = 7.7 Hz, 1H), 4.18-4.10 (m, 1H), 3.32 (s, 2H), 2.97-2.89 (m, 1H), 2.37 (dt, J = 11.7, 6.1 Hz, 1H), 2.26- 2.04 (m, 6H), 1.97 (d, J = 13.6 Hz, 1H), 1.93-1.65 (m, 3H), 1.58 (d, J = 11.9 Hz, 2H), 1.44 (d, J = 15.8 Hz, 1H), 1.37-1.30 (m, 2H), 1.27 (s, 3H), 1.19-1.10 (m, 1H), 0.96-0.83 (m, 4H), | | 445.48 | | |
| | | | 0.75 (dd, J = 7.3, | | | | |
| | | | 2.5 Hz, 3H). | | | | |
| 81 | 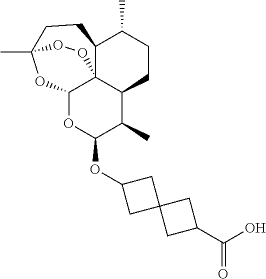 [SMILES: C[C@H]1[C@@H](OC2CC3(C2)CC(C(=O)O)C3)O[C@@H]2O[C@@]3(C)CC[C@H]4[C@H](C)CC[C@@H]1[C@@]24OO3] | 6- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)spiro[3.3] heptane-2- carboxylic acid | ¹H NMR (400 MHz, DMSO) δ 12.02 (s, 1H), 5.27 (d, J = 5.0 Hz, 1H), 4.62 (s, 1H), 4.00 (t, J = 7.1 Hz, 1H), 3.31 (s, 1H), 2.93 (td, J = 8.4, 3.0 Hz, 1H), 2.37 (d, J = 6.1 Hz, 2H), 2.25-2.06 (m, 6H), 2.03-1.86 (m, 2H), 1.85-1.68 (m, 3H), 1.67-1.60 (m, 1H), 1.56 (d, J = 12.9 Hz, 1H), 1.45- 1.29 (m, 3H), 1.27 (s, 3H), 0.89 (d, J = 6.5 Hz, 3H), 0.84- 0.79 (m, 4H). | 423.61 | | | |
| 82 | 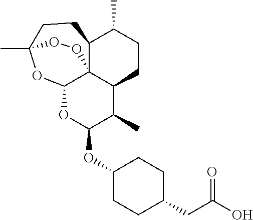 [SMILES: C[C@H]1[C@@H](O[C@H]2CC[C@@H](CC(=O)O)CC2)O[C@@H]2O[C@@]3(C)CC[C@H]4[C@H](C)CC[C@@H]1[C@@]24OO3] | 2-((1R,4s)-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclo- hexyl)acetic acid | ¹H NMR (400 MHz, DMSO) δ 12.01 (s, 1H), 5.32 (s, 1H), 4.76 (d, J = 3.5 Hz, 1H), 3.79 (s, 1H), 2.39 (dt, J = 7.8, 4.3 Hz, 1H), 2.17 (td, J = 13.9, 3.9 Hz, 1H), 2.10- 2.04 (m, 3H), 1.99 (d, J = 14.9 Hz, 1H), 1.88-1.76 (m, 2H), 1.75-1.64 (m, 4H), 1.58 (dd, J = 13.1, 3.4 Hz, 1H), 1.52- | | 447.43 | | |
| | | | 1.46 (m, 3H), 1.44- | | | | |
| | | | 1.29 (m, 5H), 1.27 | | | | |
| | | | (s, 3H), 1.24-1.10 | | | | |
| | | | (m, 3H), 0.95- | | | | |
| | | | 0.76 (m, 7H). | | | | |
| 83 | 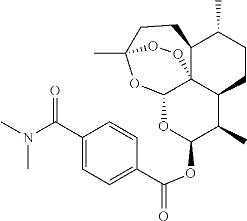 | (3R,5aS,6R,8aS, 9R,10R,12R, 12aR)-3,6,9- trimethyldeca- hydro-12H-3,12- epoxy[1,2] dioxepino[4,3-i] isochromen-10- yl) 4-(dimethyl- carbamoyl) benzoate | ¹H NMR (400 MHz, DMSO) δ 8.02 (d, J = 7.9 Hz, 2H), 7.56 (d, J = 7.9 Hz, 2H), 6.27 (d, J = 3.6 Hz, 1H), 5.62 (s, 1H), 3.00 (s, 3H), 2.88 (s, 3H), 2.72 (s, 1H), 2.21 (t, J = 13.9 Hz, 1H), 2.02 (d, J = 14.7 Hz, 1H), 1.89-1.79 (m, 3H), 1.73-1.65 (m, 1H), 1.61-1.45 (m, | 460.58 | | | |
| | | | 2H), 1.44-1.33 (m, | | | | |
| | | | 1H), 1.31 (s, 3H), | | | | |
| | | | 1.29-1.17 (m, 1H), | | | | |
| | | | 1.02-0.87 (m, 7H). | | | | |
| 84 | 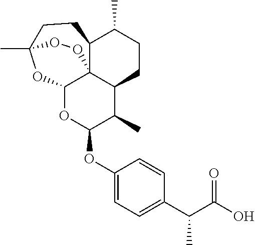 [SMILES: C[C@H]1[C@@H](Oc2ccc([C@@H](C)C(=O)O)cc2)O[C@@H]2O[C@@]3(C)CC[C@H]4[C@H](C)CC[C@@H]1[C@@]24OO3] | (R)-2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) propanoic acid | ¹H NMR (400 MHz, DMSO) δ 12.26 (s, 1H), 7.23 (d, J = 8.1 Hz, 2H), 7.02 (d, J = 8.1 Hz, 2H), 5.53 (d, J = 3.3 Hz, 1H), 5.42 (s, 1H), 3.63 (d, J = 7.2 Hz, 1H), 2.26-2.12 (m, 2H), 2.01 (d, J = 14.6 Hz, 1H), 1.92 (d, J = 13.7 Hz, 1H), 1.88- 1.77 (d, J = 13.5 Hz, 2H), 1.64 (d, J = 12.9 Hz, 1H), 1.53 (d, J = 13.0 Hz, 1H), 1.46-1.36 (m, 1H), | | 455.37 | | |
| | | | 1.34 (d, J = 7.2 Hz, | | | | |
| | | | 3H), 1.29 (s, 3H), | | | | |
| | | | 1.26-1.11 (m, 2H), | | | | |
| | | | 1.02-0.84 (m, 7H). | | | | |
| 85 | 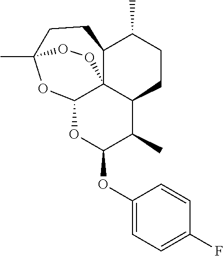 [SMILES: C[C@H]1[C@@H](Oc2ccc(F)cc2)O[C@@H]2O[C@@]3(C)CC[C@H]4[C@H](C)CC[C@@H]1[C@@]24OO3] | (3R,5aS,6R,8aS, 9R,10S,12R, 12aR)-10-(4- fluorophenoxy)- 3,6,9- trimethyldeca- hydro-12H-3,12- epoxy[1,2] dioxepino[4,3-i] isochromene | ¹H NMR (400 MHz, DMSO) δ 7.19-7.05 (m, 4H), 5.47 (d, J = 3.4 Hz, 1H), 5.45 (s, 1H), 2.57 (s, 1H), 2.24-2.13 (m, 1H), 2.00 (d, J = 14.6 Hz, 1H), 1.94- 1.74 (m, 3H), 1.62 (d, J = 12.9 Hz, 1H), 1.51 (d, J = 12.8 Hz, 1H), 1.46-1.33 (m, 2H), 1.28 (s, 3H), 1.20 (td, J = 11.2, 6.4 Hz, 1H), 1.03- 0.74 (m, 7H). | | 401.16 | | |
| 86 | 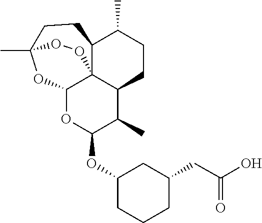 [SMILES: C[C@H]1[C@@H](O[C@H]2CCC[C@@H](CC(=O)O)C2)O[C@@H]2O[C@@]3(C)CC[C@H]4[C@H](C)CC[C@@H]1[C@@]24OO3] | 2-((1R,3S)-3- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclo- hexyl)acetic acid | ¹H NMR (400 MHz, DMSO) δ 12.04 (s, 1H), 5.33 (d, J = 3.9 Hz, 1H), 4.81 (dd, J = 8.0, 3.4 Hz, 1H), 3.49 (t, J = 10.8 Hz, 0H), 2.37 (s, 1H), 2.23-2.08 (m, 3H), 2.03-1.88 (m, 3H), 1.85-1.49 (m, 8H), 1.44-1.32 (m, 3H), 1.27- 1.21 (m, 4H), 1.19- 1.07 (m, 1H), 0.98- 0.75 (m, 8H). | | 447.44 | | |
| 87 | 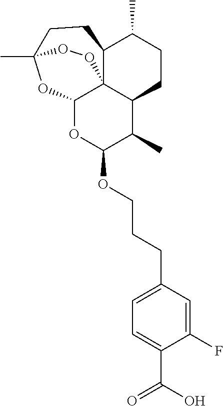 [SMILES: C[C@H]1[C@@H](OCCCc2ccc(C(=O)O)c(F)c2)O[C@@H]2O[C@@]3(C)CC[C@H]4[C@H](C)CC[C@@H]1[C@@]24OO3] | 2-fluoro-4-(3- (((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)propyl) benzoic acid | | | | | |
| 88 | 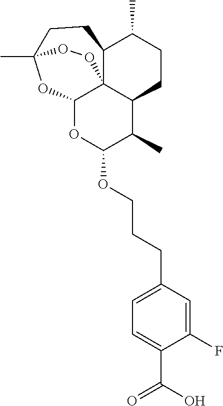 [SMILES: C[C@H]1[C@H](OCCCc2ccc(C(=O)O)c(F)c2)O[C@@H]2O[C@@]3(C)CC[C@H]4[C@H](C)CC[C@@H]1[C@@]24OO3] | 2-fluoro-4-(3- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)propyl) benzoic acid | | | | | |
| 89 | 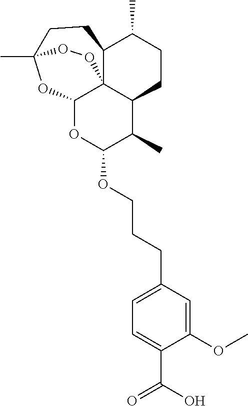 | 2-methoxy-4-(3- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)propyl) benzoic acid | | | | | |
| 90 | 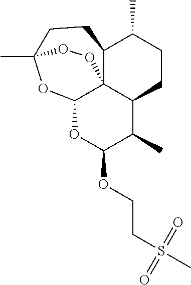 | (3R,5aS,6R,8aS, 9R,10S,12R, 12aR)-3,6,9- trimethyl-10-(2- (methylsulfonyl) ethoxy)deca- hydro-12H-3,12- epoxy[1,2] dioxepino[4,3-i] isochromene | ¹H NMR (500 MHz, CDCl₃) δ 5.41 (s, 1H), 4.84 (dt, J = 3.4, 0.8 Hz, 1H), 4.29 (ddd, J = 11.4, 6.5, 4.2 Hz, 1H), 3.94-3.86 (m, 1H), 3.27 (dddd, J = 8.4, 6.4, 4.1, 0.8 Hz, 2H), 2.96 (s, 3H), 2.69 (ddt, J = 8.1, 6.9, 3.3 Hz, 1H), 2.37 (ddd, J = 14.6, 13.5, 4.0 Hz, 1H), 2.08-2.00 (m, 1H), 1.89 (dddd, J = 13.7, 6.8, 4.0, 3.0 Hz, 1H), 1.77-1.68 (m, | | 413.32 | >20 | >20 |
| | | | 2H), 1.64 (ddq, J = | | | | |
| | | | 13.3, 6.8, 3.4 Hz, | | | | |
| | | | 1H), 1.54-1.44 (m, | | | | |
| | | | 2H), 1.34 (dtdd, | | | | |
| | | | J = 12.2, 8.8, 6.1, | | | | |
| | | | 3.0 Hz, 1H), 1.43 (s, | | | | |
| | | | 3H), 1.29-1.22 (m, | | | | |
| | | | 1H), 0.96 (d, J = 6.3 | | | | |
| | | | Hz, 3H), 0.92 (d, | | | | |
| | | | J = 7.5 Hz, 3H). | | | | |
| 91 | 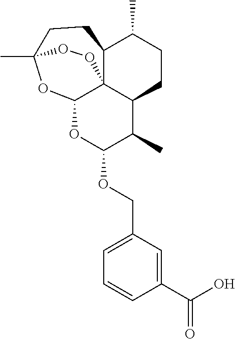 [SMILES: C[C@H]1[C@H](OCc2cccc(C(=O)O)c2)O[C@@H]2O[C@@]3(C)CC[C@H]4[C@H](C)CC[C@@H]1[C@@]24OO3] | 3- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, CDCl₃) δ 8.10 (s, 1H), 8.02 (dt, J = 7.8, 1.4 Hz, 1H), 7.64 (dt, J = 7.9, 1.3 Hz, 1H), 7.45 (t, J = 7.7 Hz, 1H), 5.36 (s, 1H), 5.04 (d, J = 12.7 Hz, 1H), 4.69 (d, J = 12.7 Hz, 1H), 4.54 (d, J = 9.2 Hz, 1H), 2.53 (ddd, J = 9.2, 7.0, 4.6 Hz, 1H), 2.46-2.34 (m, 1H), 2.09-1.99 (m, 1H), 1.94-1.82 (m, 1H), 1.74 (dt, J = 13.1, 3.8 Hz, 1H), 1.67 (dd, J = 13.1, 3.2 Hz, 1H), 1.58 (dt, J = 13.6, 4.5 Hz, | | 441.12 | 17.4641 | >20 |
| | | | 1H), 1.52-1.48 (m, | | | | |
| | | | 1H), 1.47 (s, 3H), | | | | |
| | | | 1.38-1.16 (m, 3H), | | | | |
| | | | 1.05-0.97 (m, 1H), | | | | |
| | | | 0.96-0.93 (m, 6H). | | | | |
| 92 | 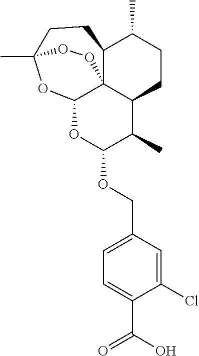 [SMILES: C[C@H]1[C@H](OCc2ccc(C(=O)O)c(Cl)c2)O[C@@H]2O[C@@]3(C)CC[C@H]4[C@H](C)CC[C@@H]1[C@@]24OO3] | 2-chloro-4- ((((3R,5aS,6R, 8aS,9R,10R,12R, 12aR)-3,6,9- trimethyldeca- hydro-12H-3,12- epoxy[1,2] dioxepino[4,3-i] isochromen-10- yl)oxy)methyl) benzoic acid | | | 475.01 | >20 | >20 |
| 93 | 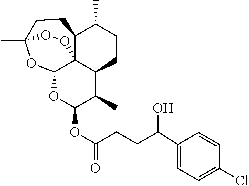 [SMILES: C[C@H]1[C@@H](OC(=O)CCC(O)c2ccc(Cl)cc2)O[C@@H]2O[C@@]3(C)CC[C@H]4[C@H](C)CC[C@@H]1[C@@]24OO3] | (3R,5aS,6R,8aS, 9R,10R,12R, 12aR)-3,6,9- trimethyldeca- hydro-12H-3,12- epoxy[1,2] dioxepino[4,3-i] isochromen-10- yl 4-(4- chlorophenyl)-4- hydroxybutanoate | ¹H NMR (400 MHz, DMSO) δ 7.46 (d, J = 7.8 Hz, 2H), 7.40 (d, J = 8.2 Hz, 2H), 5.57-5.48 (m, 1H), 5.41 (d, J = 4.1 Hz, 1H), 4.94 (d, J = 3.6 Hz, 1H), 2.73-2.66 (m, 2H), 2.64-2.57 (m, 2H), 2.38-2.25 (m, 2H), 2.21-2.10 (m, 1H), 2.06 (dt, J = 10.5, | | | 0.0786 | 0.08769 |
| | | | 5.4 Hz, 1H), 1.97 (d, | | | | |
| | | | J =15.0 Hz, 1H), | | | | |
| | | | 1.88-1.71 (m, | | | | |
| | | | 2H), 1.64 | | | | |
| | | | (dd, J = 13.9, 3.8 | | | | |
| | | | Hz, 1H), 1.58-1.51 | | | | |
| | | | (m, 1H), 1.33 (dt, | | | | |
| | | | J = 9.8, 5.0 Hz, 3H), | | | | |
| | | | 1.25 (s, 3H), 1.12 | | | | |
| | | | (dd, J = 11.4, 6.6 | | | | |
| | | | Hz, 1H), 0.92-0.73 | | | | |
| | | | (m, 7H). | | | | |
| 94 | 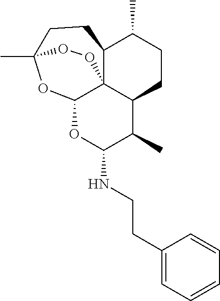 [SMILES: C[C@H]1[C@H](NCCc2ccccc2)O[C@@H]2O[C@@]3(C)CC[C@H]4[C@H](C)CC[C@@H]1[C@@]24OO3] | (3R,5aS,6R,8aS, 9R,10R,12R, 12aR)-3,6,9- trimethyl-N- phenethyldeca- hydro-12H-3,12- epoxy[1,2] dioxepino[4,3-i] isochromen-10- amine | ¹H NMR (500 MHz, CDCl₃) δ 7.34-7.19 (m, 4H), 7.18-7.13 (m, 1H), 5.34 (s, 1H), 4.41 (d, J = 10.3 Hz, 1H), 3.36 (ddd, J = 14.6, 10.4, 6.3 Hz, 1H), 3.17 (ddd, J = 14.9, 9.8, 5.4 Hz, 1H), 2.94 (ddd, J = 14.9, 10.2, 5.2 Hz, 1H), 2.75 (ddd, J = 13.6, 9.8, 6.3 Hz, 1H), 2.49 (ddd, J = 11.0, 7.2, 4.5 Hz, 1H), 2.33 (td, J = 13.9, 3.9 Hz, 1H), 2.03-1.95 (m, 1H), 1.89-1.80 | 388.38 | | 0.2568 | 0.315 |
| | | | (m, 1H), 1.76-1.66 | | | | |
| | | | (ddd, J = 13.4, 6.6, | | | | |
| | | | 3.3 Hz, 4H), 1.53 | | | | |
| | | | (dq, J = 12.2, 4.1 | | | | |
| | | | Hz, 1H), 1.49-1.43 | | | | |
| | | | (m, 1H), 1.39 (s, | | | | |
| | | | 3H), 1.37-1.29 (m, | | | | |
| | | | 2H), 1.29-1.15 (m, | | | | |
| | | | 1H), 0.99 (dd, J = | | | | |
| | | | 12.5, 3.6 Hz, 1H), | | | | |
| | | | 0.94 (d, J = 6.3 Hz, | | | | |
| | | | 3H), 0.79 (d, J = 7.1 | | | | |
| | | | Hz, 3H). | | | | |
| 95 | 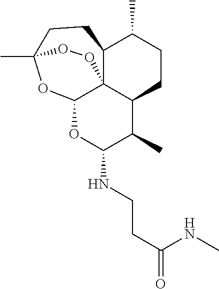 | N-methyl-3- (((3R,5aS,6R,8aS, 9R,10R,12R, 12aR)-3,6,9- trimethyldeca- hydro-12H-3,12- epoxy[1,2] dioxepino[4,3-i] isochromen-10- yl)amino) propanamide | | 369.23 | | 0.3481 | 0.352 |
| 96 | 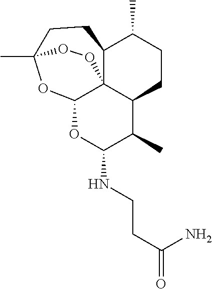 [SMILES: C[C@H]1[C@H](NCCC(N)=O)O[C@@H]2O[C@@]3(C)CC[C@H]4[C@H](C)CC[C@@H]1[C@@]24OO3] | 3- (((3R,5aS,6R,8aS, 9R,10R,12R, 12aR)-3,6,9- trimethyldeca- hydro-12H-3,12- epoxy[1,2] dioxepino[4,3-i] isochromen-10- yl)amino) propanamide | | 355.04 | | 1.6963 | 1.979 |
| 97 | 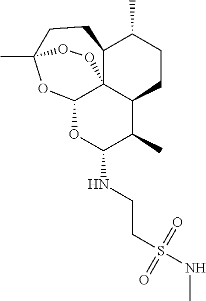 | N-methyl-2- (((3R,5aS,6R,8aS, 9R,10R,12R, 12aR)-3,6,9- trimethyldeca- hydro-12H-3,12- epoxy[1,2] dioxepino[4,3-i] isochromen-10- yl)amino)ethane- 1-sulfonamide | ¹H NMR (400 MHz, CDCl₃) δ 5.30 (s, 1H), 5.04 (d, J = 5.5 Hz, 1H), 4.12 (d, J = 9.8 Hz, 1H), 3.49 (s, 1H), 3.31- 3.17 (m, 1H), 2.85- 2.71 (m, 3H), 2.34 (tdd, J = 13.3, 10.5, 4.2 Hz, 2H), 2.07- 1.97 (m, 1H), 1.94- 1.83 (m, 1H), 1.76- 1.71 (m, 1H), 1.41 (s, 3H), 1.37-1.21 (m, 4H), 1.11-1.03 (m, 1H), 0.96 (d, J = 6.0 Hz, 3H), 0.87 (d, J = 7.2 Hz, 3H). | 405.47 | | 2.3440 | 2.081 |
| 98 | 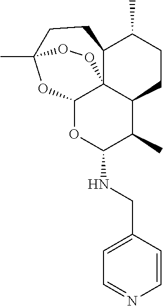 [SMILES: C[C@H]1[C@H](NCc2ccncc2)O[C@@H]2O[C@@]3(C)CC[C@H]4[C@H](C)CC[C@@H]1[C@@]24OO3] | (3R,5aS,6R,8aS, 9R,10R,12R, 12aR)-3,6,9- trimethyl-N- (pyridin-4- ylmethyl)deca- hydro-12H-3,12- epoxy[1,2] dioxepino[4,3-i] isochromen- 10-amine | ¹H NMR (400 MHz, CDCl₃) δ 8.56 (s, 2H), 7.47 (s, 2H), 5.28 (s, 1H), 4.18 (d, J = 16.0 Hz, 1H), 4.06 (d, J = 6.44 Hz 1H), 4.03 (s, 1H), 2.42-2.31 (m, 2H), 2.10-2.03 (m, 1H), 2.03-1.97 (m, 1H), 1.89 (ddd, J = 13.8, 6.5, 3.5 Hz, 1H), 1.77-1.65 (m, 2H), 1.55 (dt, J = 13.9, 4.2 Hz, 1H), 1.48 (dd, J = 11.5, 5.0 Hz, 1H), 1.44 (s, 3H), 1.38- 1.15 (m, 2H), 1.07- | 375.07 | | >20 | >20 |
| | | | 0.99 (m, 1H), 0.97- | | | | |
| | | | 0.94 (m, 6H). | | | | |
| 99 | 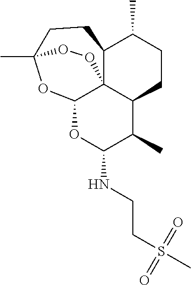 | (3R,5aS,6R,8aS, 9R,10R,12R, 12aR)-3,6,9- trimethyl-N-(2- (methylsulfonyl) ethyl)decahydro- 12H-3,12-epoxy [1,2]dioxepino [4,3-i]isochromen- 10-amine | ¹H NMR (400 MHz, CDCl₃) δ 5.29 (s, 1H), 4.12 (d, J = 9.9 Hz, 1H), 3.54-3.41 (m, 1H), 3.34-3.17 (m, 3H), 2.41-2.25 (m, 2H), 2.06-2.01 (m, 1H), 1.99 (dd, J = 5.0, 3.0 Hz, 1H), 1.93-1.82 (m, 1H), 1.79-1.65 (m, 2H), 1.53 (dt, J = 13.6, 4.3 Hz, 1H), 1.47 (ddd, J = 13.4, 4.8, 2.1 Hz, 1H), 1.39 (s, 3H), 1.36- 1.31 (m, 1H), 1.29 (d, J = 4.6 Hz, 1H), | 390.40 | | 0.6595 | 0.7433 |
| | | | 1.27-1.22 (m, 1H), | | | | |
| | | | 1.07-0.98 (m, 1H), | | | | |
| | | | 0.95 (d, J = 6.2 Hz, | | | | |
| | | | 3H), 0.87 (d, J = 7.2 | | | | |
| | | | Hz, 3H). | | | | |
| 100 | 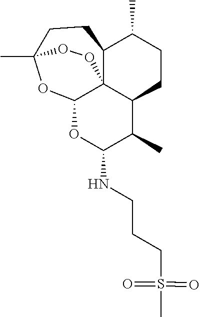 [SMILES: C[C@H]1[C@H](NCCCS(C)(=O)=O)O[C@@H]2O[C@@]3(C)CC[C@H]4[C@H](C)CC[C@@H]1[C@@]24OO3] | (3R,5aS,6R,8aS, 9R,10R,12R, 12aR)-3,6,9- trimethyl-N-(3- (methylsulfonyl) propyl)deca- hydro-12H-3,12- epoxy[1,2] dioxepino[4,3-i] isochromen-10- amine | | 404.29 | | 0.4984 | 0.5354 |
| 101 | 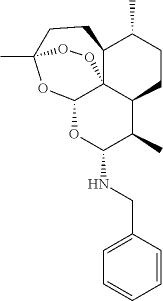 [SMILES: C[C@H]1[C@H](NCc2ccccc2)O[C@@H]2O[C@@]3(C)CC[C@H]4[C@H](C)CC[C@@H]1[C@@]24OO3] | (3R,5aS,6R,8aS, 9R,10R,12R, 12aR)-N-benzyl- 3,6,9- trimethyldeca- hydro-12H-3,12- epoxy[1,2] dioxepino[4,3-i] isochromen-10- amine | ¹H NMR (500 MHz, CDCl₃) δ 7.43-7.37 (m, 2H), 7.30 (t, J = 7.5 Hz, 2H), 7.25-7.20 (m, 1H), 5.29 (s, 1H), 4.18- 4.09 (m, 3H), 3.96 (d, J = 13.9 Hz, 1H), 2.42-2.33 (m, 2H), 2.01 (dd, J = 4.9, 3.0 Hz, 1H), 1.93- 1.83 (m, 1H), 1.76- 1.63 (m, 2H), 1.58- 1.47 (m, 2H), 1.38- 1.32 (m, 1H), 1.30 (dt, J = 5.6, 2.8 Hz, 1H), 1.28- 1.26-1.21 (m, 3H), 1.05-0.97 (m, | | | 0.8346 | 1.315 |
| | | | 1H), 0.95 (d, J = 1.8 | | | | |
| | | | Hz, 3H), 0.94 (d, J = | | | | |
| | | | 2.7 Hz, 3H). | | | | |
| 102 | 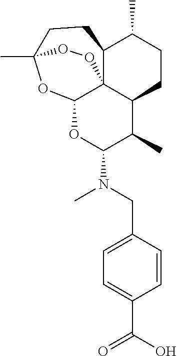 [SMILES: C[C@H]1[C@H](N(C)Cc2ccc(C(=O)O)cc2)O[C@@H]2O[C@@]3(C)CC[C@H]4[C@H](C)CC[C@@H]1[C@@]24OO3] | 4- ((methyl((3R, 5aS,6R,8aS,9R, 10R,12R,12aR)- 3,6,9- trimethyldeca- hydro-12H-3,12- epoxy[1,2] dioxepino[4,3-i] isochromen-10- yl)amino)methyl) benzoic acid | | 432.14 | | >20 | >20 |
| 103 | 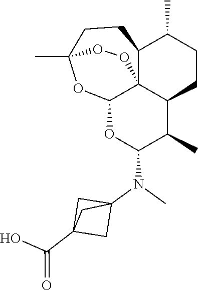 [SMILES: C[C@H]1[C@H](N(C)C23CC(C(=O)O)(C2)C3)O[C@@H]2O[C@@]3(C)CC[C@H]4[C@H](C)CC[C@@H]1[C@@]24OO3] | 3- (methyl((3R,5aS, 6R,8aS,9R,10R, 12R,12aR)-3,6,9- trimethyldeca- hydro-12H-3,12- epoxy[1,2] dioxepino[4,3-i] isochromen-10- yl)amino)bicyclo [1.1.1]pentane-1- carboxylic acid | | 408.34 | | >20 | >20 |
| 104 | 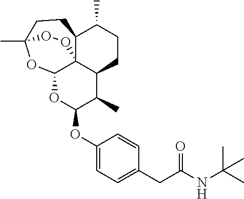 [SMILES: C[C@H]1[C@@H](Oc2ccc(CC(=O)NC(C)(C)C)cc2)O[C@@H]2O[C@@]3(C)CC[C@H]4[C@H](C)CC[C@@H]1[C@@]24OO3] | N-(tert-butyl)- 2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetamide | ¹H NMR (400 MHz, DMSO) δ 7.63 (s, 1H), 7.16 (d, J = 8.3 Hz, 2H), 6.97 (d, J = 8.4 Hz, 2H), 5.49 (d, J = 3.4 Hz, 1H), 5.42 (s, 1H), 3.27 (s, 2H), 2.58 (dt, J = 7.9, 4.2 Hz, 1H), 2.19 (td, J = 13.9, 3.8 Hz, 1H), 1.99 (dt, J = 14.5, 4.0 Hz, 1H), 1.90 (td, J = 13.6, 3.3 Hz, | | | 0.0523 | 0.16 |
| | | | 1H), 1.83-1.79 (m, | | | | |
| | | | 2H), 1.63 (dd, J = | | | | |
| | | | 13.2, 3.3 Hz, 1H), | | | | |
| | | | 1.51 (dt, J = 13.2, | | | | |
| | | | 4.7 Hz, 1H), 1.43- | | | | |
| | | | 1.34 (m, 1H), 1.28 | | | | |
| | | | (s, 3H), 1.23 (s, 9H), | | | | |
| | | | 1.18 (dd, J = 11.4, | | | | |
| | | | 6.6 Hz, 1H), 1.01- | | | | |
| | | | 0.86 (m, 7H). | | | | |
| 105 | 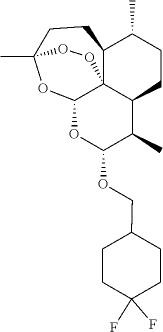 | (3R,5aS,6R,8aS, 9R,10R,12R, 12aR)-10-((4,4- difluorocyclo- hexyl)methoxy)- 3,6,9- trimethyldeca- hydro-12H-3,12- epoxy[1,2] dioxepino[4,3-i] isochromene | ¹H NMR (400 MHz, DMSO) δ 5.41 (s, 1H), 4.45 (d, J = 9.2 Hz, 1H), 3.66 (dd, J = 9.7, 5.9 Hz, 1H), 3.24 (dd, J = 9.8, 6.9 Hz, 1H), 2.24-2.06 (m, 2H), 2.03-1.95 (m, 3H), 1.88-1.67 (m, 6H), 1.64-1.56 (m, 2H), 1.44 (d, J = 4.7 Hz, 1H), 1.41-1.31 (m, 3H), 1.28 (s, 3H), 1.26- 1.09 (m, 3H), 0.95- 0.92 (m, 1H), 0.89 (d, J = 6.3 Hz, 3H), 0.80 (d, J = 7.1 Hz, 3H). | | | 0.0484 | 0.20 |
| 106 | 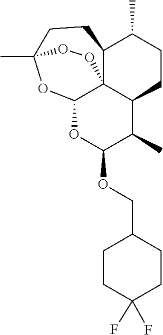 [SMILES: C[C@H]1[C@@H](OCC2CCC(F)(F)CC2)O[C@@H]2O[C@@]3(C)CC[C@H]4[C@H](C)CC[C@@H]1[C@@]24OO3] | (3R,5aS,6R,8aS, 9R,10S,12R, 12aR)-10-((4,4- difluorocyclo- hexyl)methoxy)- 3,6,9- trimethyldeca- hydro-12H-3,12- epoxy[1,2] dioxepino[4,3-i] isochromene | ¹H NMR (400 MHz, DMSO) δ 5.41 (s, 1H), 4.45 (d, J = 9.2 Hz, 1H), 3.66 (dd, J = 9.7, 5.9 Hz, 1H), 3.24 (dd, J = 9.8, 6.9 Hz, 1H), 2.24- 2.06 (m, 2H), 2.03- 1.95 (m, 3H), 1.88- 1.67 (m, 6H), 1.64- 1.56 (m, 2H), 1.44 (d, J = 4.7 Hz, 1H), 1.41-1.31 (m, 3H), 1.28 (s, 3H), 1.26- 1.09 (m, 3H), 0.95- 0.92 (m, 1H), 0.89 (d, J = 6.3 Hz, 3H), 0.80 (d, J = 7.1 Hz, 3H). | | | 0.0204 | 0.10 |
| 107 | 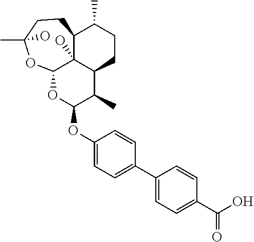 [SMILES: C[C@H]1[C@@H](Oc2ccc(-c3ccc(C(=O)O)cc3)cc2)O[C@@H]2O[C@@]3(C)CC[C@H]4[C@H](C)CC[C@@H]1[C@@]24OO3] | 4′- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)-[1,1′- biphenyl]-4- carboxylic acid | ¹H NMR (400 MHz, DMSO) δ 12.93 (s, 1H), 8.00 (d, J = 8.2 Hz, 2H), 7.76 (d, J = 8.2 Hz, 2H), 7.71 (d, J = 8.7 Hz, 2H), 7.19 (d, J = 8.8 Hz, 2H), 5.64 (d, J = 3.4 Hz, 1H), 5.44 (s, 1H), 2.61 (dq, J = 11.4, 5.8 Hz, 1H), 2.20 (td, J = 14.0, 4.0 Hz, 1H), 2.01 (d, J = 4.1 Hz, 1H), 1.99 (d, | | | 0.0235 | 0.04 |
| | | | J = 3.4 Hz, 1jH), | | | | |
| | | | 1.94 (dd, J = 13.6, | | | | |
| | | | 3.1 Hz, 1H), 1.89- | | | | |
| | | | 1.79 (m, 2H), 1.69- | | | | |
| | | | 1.60 (m, 1H), 1.54 | | | | |
| | | | (dt, J = 13.1, 4.7 | | | | |
| | | | Hz, 1H), 1.46-1.34 | | | | |
| | | | (m, 2H), 1.29 (s, | | | | |
| | | | 3H), 1.27-1.15 (m, | | | | |
| | | | 2H), 0.99 (d, J = 7.3 | | | | |
| | | | Hz, 3H), 0.91 (d, | | | | |
| | | | J = 6.3 Hz, 3H). | | | | |
| 108 | 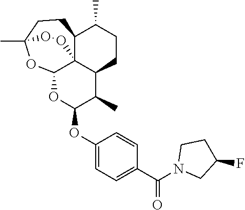 [SMILES: C[C@H]1[C@@H](Oc2ccc(C(=O)N3CC[C@@H](F)C3)cc2)O[C@@H]2O[C@@]3(C)CC[C@H]4[C@H](C)CC[C@@H]1[C@@]24OO3] | ((R)-3- fluoropyrrolidin- 1-yl)(4-(((3R,5aS, 6R,8aS,9R,10S, 12R,12aR)- 3,6,9- trimethyldeca- hydro-12H-3,12- epoxy[1,2] dioxepino[4,3-i] isochromen-10- yl)oxy)phenyl) methanone | | | | | |
| 109 | 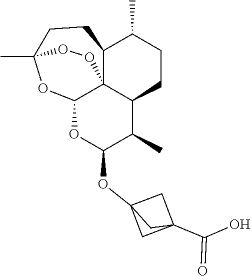 [SMILES: C[C@H]1[C@@H](OC23CC(C(=O)O)(C2)C3)O[C@@H]2O[C@@]3(C)CC[C@H]4[C@H](C)CC[C@@H]1[C@@]24OO3] | 3-(((3R,5aS,6R, 8aS,9R,10S,12R, 12aR)- 3,6,9- trimethyldeca- hydro-12H-3,12- epoxy[1,2] dioxepino[4,3-i] isochromen-10- yl)oxy)bicyclo [1.1.1]pentane-1- carboxylic acid | | | | | |
| 110 | 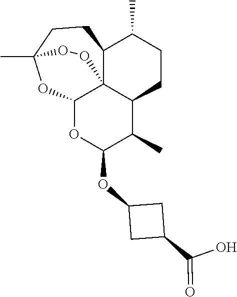 [SMILES: C[C@H]1[C@@H](O[C@H]2C[C@@H](C(=O)O)C2)O[C@@H]2O[C@@]3(C)CC[C@H]4[C@H](C)CC[C@@H]1[C@@]24OO3] | (1R,3s)-3- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclo- butane-1- carboxylic acid | | | | | |
| 111 | 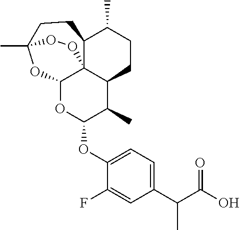 [SMILES: CC(C(=O)O)c1ccc(O[C@@H]2O[C@@H]3O[C@@]4(C)CC[C@H]5[C@H](C)CC[C@@H]([C@H]2C)[C@@]35OO4)c(F)c1] | 2-(3-fluoro-4- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)phenyl) propanoic acid | | | | | |
| 112 | 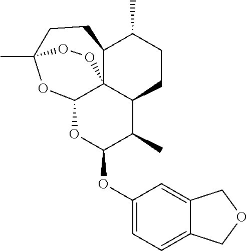 [SMILES: C[C@H]1[C@@H](Oc2ccc3c(c2)COC3)O[C@@H]2O[C@@]3(C)CC[C@H]4[C@H](C)CC[C@@H]1[C@@]24OO3] | (3R,5aS,6R,8aS, 9R,10S,12R, 12aR)-10-((1,3- dihydroisobenzo- furan-5-yl)oxy)- 3,6,9- trimethyldeca- hydro-12H-3,12- epoxy[1,2] dioxepino[4,3-i] isochromene | | | | | |
| 113 | 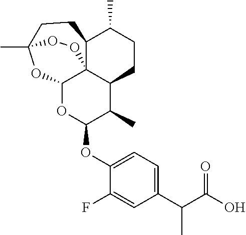 [SMILES: CC(C(=O)O)c1ccc(O[C@H]2O[C@@H]3O[C@@]4(C)CC[C@H]5[C@H](C)CC[C@@H]([C@H]2C)[C@@]35OO4)c(F)c1] | 2-(3-fluoro-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) propanoic acid | | | | | |
| 114 | 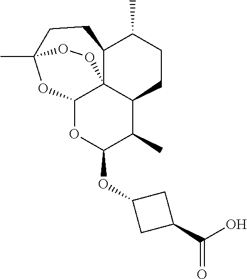 [SMILES: C[C@H]1[C@@H](O[C@H]2C[C@H](C(=O)O)C2)O[C@@H]2O[C@@]3(C)CC[C@H]4[C@H](C)CC[C@@H]1[C@@]24OO3] | (1S,3r)-3- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclo- butane-1- carboxylic acid | | | | | |
| 115 | 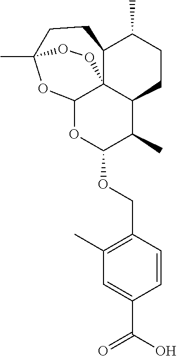 [SMILES: Cc1cc(C(=O)O)ccc1CO[C@@H]1OC2O[C@@]3(C)CC[C@H]4[C@H](C)CC[C@@H]([C@H]1C)[C@@]24OO3] | 3-methyl-4- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) benzoic acid | | | | | |
| 116 | 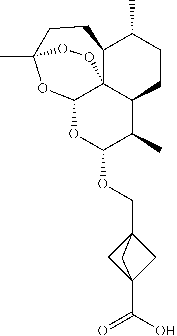 [SMILES: C[C@H]1[C@H](OCC23CC(C(=O)O)(C2)C3)O[C@@H]2O[C@@]3(C)CC[C@H]4[C@H](C)CC[C@@H]1[C@@]24OO3] | 3- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) bicyclo[1.1.1] pentane-1- carboxylic acid | | | | | |
| 117 | 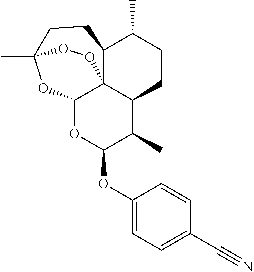 [SMILES: C[C@H]1[C@@H](Oc2ccc(C#N)cc2)O[C@@H]2O[C@@]3(C)CC[C@H]4[C@H](C)CC[C@@H]1[C@@]24OO3] | 4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) benzonitrile | | | | | |
| 118 | 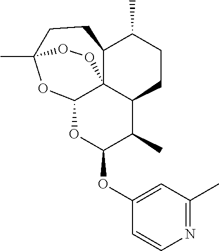 | 2-methyl-4- (((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)pyridine | | | | | |
| 119 | 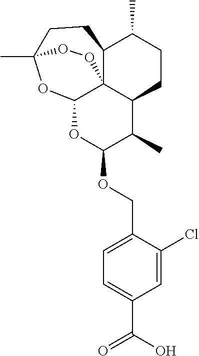 [SMILES: C[C@H]1[C@@H](OCc2ccc(C(=O)O)cc2Cl)O[C@@H]2O[C@@]3(C)CC[C@H]4[C@H](C)CC[C@@H]1[C@@]24OO3] | 3-chloro-4- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) benzoic acid | | | | 0.1103 | >20 |
| 120 | 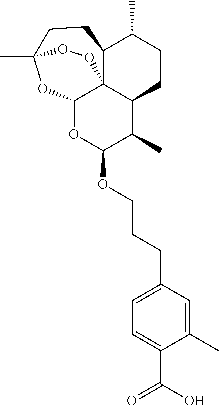 | 2-methyl-4-(3- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)propyl) benzoic acid | | | | | |
| 121 | 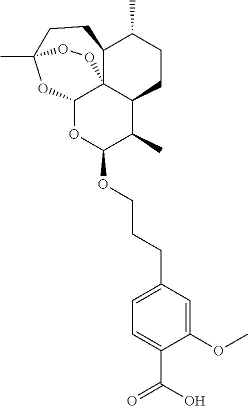 | 2-methoxy-4- (3- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)propyl) benzoic acid | | | | | |
| 122 | 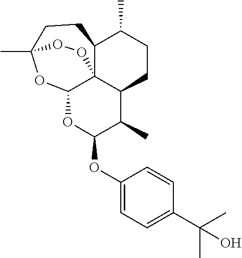 [SMILES: C[C@H]1[C@@H](Oc2ccc(C(C)(C)O)cc2)O[C@@H]2O[C@@]3(C)CC[C@H]4[C@H](C)CC[C@@H]1[C@@]24OO3] | 2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) propan-2-ol | | | | | |
| 123 | 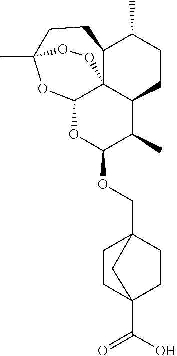 [SMILES: C[C@H]1[C@@H](OCC23CCC(C(=O)O)(CC2)C3)O[C@@H]2O[C@@]3(C)CC[C@H]4[C@H](C)CC[C@@H]1[C@@]24OO3] | 4- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) bicyclo[2.2.1] heptane-1- carboxylic acid | | | | | |
| 124 | 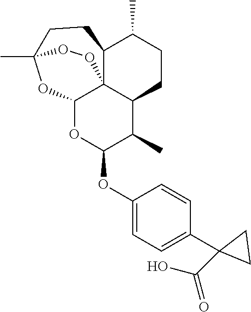 [SMILES: C[C@H]1[C@@H](Oc2ccc(C3(C(=O)O)CC3)cc2)O[C@@H]2O[C@@]3(C)CC[C@H]4[C@H](C)CC[C@@H]1[C@@]24OO3] | 1-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) cyclopropane-1- carboxylic acid | ¹H NMR (400 MHz, DMSO) δ 12.24 (s, 1H), 7.24 (d, J = 8.6 Hz, 2H), 6.98 (d, J = 8.6 Hz, 2H), 5.53 (d, J = 3.4 Hz, 1H), 5.41 (s, 1H), 2.64-2.54 (m, 1H), 2.19 (td, J = 13.9, 3.8 Hz, 1H), 2.04- 1.96 (m, 1H), 1.91 (dd, J = 13.6, 3.4 Hz, 1H), 1.83 (ddt, J = 14.0, 9.4, 4.2 Hz, 2H), 1.63 (dd, J = 13.2, 3.4 Hz, 1H), 1.52 (dt, J = 13.2, 4.7 Hz, 1H), 1.43-1.40 (m, 3H), | | 467.49 | 0.0288 | 2.45 |
| | | | 1.39-1.32 (m, 1H), | | | | |
| | | | 1.28 (s, 3H), 1.20 | | | | |
| | | | (td, J = 11.3, 6.5 | | | | |
| | | | Hz, 1H), 1.08 (q, | | | | |
| | | | J = 3.6 Hz, 2H), | | | | |
| | | | 1.00-0.87 (m, 7H). | | | | |
| 125 | 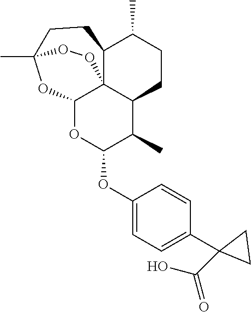 [SMILES: C[C@H]1[C@H](Oc2ccc(C3(C(=O)O)CC3)cc2)O[C@@H]2O[C@@]3(C)CC[C@H]4[C@H](C)CC[C@@H]1[C@@]24OO3] | 1-(4- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)phenyl) cyclopropane-1- carboxylic acid | | 467.53 | | | |
| 126 | 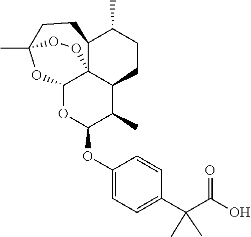 [SMILES: C[C@H]1[C@@H](Oc2ccc(C(C)(C)C(=O)O)cc2)O[C@@H]2O[C@@]3(C)CC[C@H]4[C@H](C)CC[C@@H]1[C@@]24OO3] | 2-methyl-2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) propanoic acid | ¹H NMR (400 MHz, DMSO) δ 12.27 (s, 1H), 7.27 (d, J = 8.8 Hz, 2H), 7.02 (d, J = 8.8 Hz, 2H), 5.53 (d, J = 3.4 Hz, 1H), 5.41 (s, 1H), 2.64-2.55 (m, 1H), 2.19 (td, J = 13.9, 3.8 Hz, 1H), 2.04- 1.95 (m, 1H), 1.91 (dd, J = 13.5, 3.3 Hz, 1H), 1.86-1.78 (m, 2H), 1.64 (dd, J = 13.1, 3.7 Hz, 1H), 1.52 (dt, J = | | 469.44 | 0.0156 | 5.50 |
| | | | 13.0, 4.7 Hz, 1H), | | | | |
| | | | 1.45 (s, 6H), 1.41 (d, | | | | |
| | | | J = 3.7 Hz, 1H), | | | | |
| | | | 1.36 (dd, J = 12.9, | | | | |
| | | | 4.1 Hz, 1H), 1.28 (s, | | | | |
| | | | 3H), 1.20 (td, J = | | | | |
| | | | 11.4, 6.5 Hz, 1H), | | | | |
| | | | 1.12 (d, J = 7.1 Hz, | | | | |
| | | | 1H), 0.96 (d, J = 7.4 | | | | |
| | | | Hz, 3H), 0.91 | | | | |
| | | | (d, J = 6.3 Hz, 3H). | | | | |
| 127 | 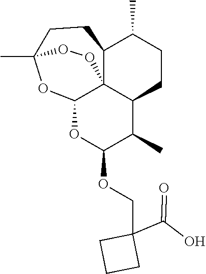 [SMILES: C[C@H]1[C@@H](OCC2(C(=O)O)CCC2)O[C@@H]2O[C@@]3(C)CC[C@H]4[C@H](C)CC[C@@H]1[C@@]24OO3] | 1- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) cyclobutane-1- carboxylic acid | ¹H NMR (400 MHz, DMSO) δ 12.26 (s, 1H), 5.31 (s, 1H), 4.70 (d, J = 3.4 Hz, 1H), 3.97 (d, J = 9.1 Hz, 1H), 3.48 (d, J = 9.1 Hz, 1H), 2.44-2.36 (m, 1H), 2.31 (dd, J = 9.1, 4.3 Hz, 1H), 2.28-2.23 (m, 1H), 2.18 (td, J = 13.9, 3.9 Hz, 1H), 2.05-1.92 (m, 1H), 1.94-1.86 (m, 3H), 1.89-1.75 (m, 2H), 1.70-1.62 (m, 1H), 1.61-1.46 (m, | | 419.40 | 8.8766 | >20 |
| | | | 2H), 1.38 (d, J = 4.3 | | | | |
| | | | Hz, 1H), 1.34 (d, | | | | |
| | | | J = 4.5 Hz, 1H), | | | | |
| | | | 1.31 (s, 1H), 1.29 | | | | |
| | | | (s, 3H), 1.18-1.07 | | | | |
| | | | (m, 1H), 0.89 (d, | | | | |
| | | | J = 6.3 Hz, 3H), | | | | |
| | | | 0.87-0.86 (m, 1H), | | | | |
| | | | 0.82 (d, J = 7.4 Hz, | | | | |
| | | | 3H). | | | | |
| 128 | 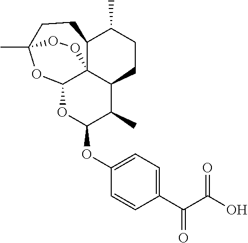 [SMILES: C[C@H]1[C@@H](Oc2ccc(C(=O)C(=O)O)cc2)O[C@@H]2O[C@@]3(C)CC[C@H]4[C@H](C)CC[C@@H]1[C@@]24OO3] | 2-oxo-2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 7.78 (d, J = 8.9 Hz, 2H), 7.11 (d, J = 8.8 Hz, 2H), 5.68 (d, J = 3.4 Hz, 1H), 5.41 (s, 1H), 2.62 (dt, J = 7.8, 4.1 Hz, 1H), 2.26-2.11 (m, 1H), 2.00 (d, J = 14.7 Hz, 1H), 1.94- 1.88 (m, 1H), 1.86- 1.76 (m, 2H), 1.65 (dd, J = 13.1, 3.4 Hz, 1H), 1.56-1.51 (m, 1H), 1.47-1.33 | | | 1.2205 | 20.00 |
| | | | (m, 2H), 1.30 (s, | | | | |
| | | | 3H), 1.20 (td, J = | | | | |
| | | | 11.3, 6.4 Hz, 1H), | | | | |
| | | | 1.00-0.87 (m, 7H). | | | | |
| 129 | 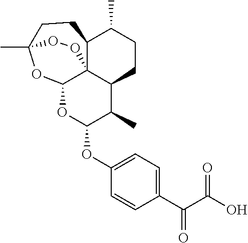 [SMILES: C[C@H]1[C@H](Oc2ccc(C(=O)C(=O)O)cc2)O[C@@H]2O[C@@]3(C)CC[C@H]4[C@H](C)CC[C@@H]1[C@@]24OO3] | 2-oxo-2-(4- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 7.76 (d, J = 8.5 Hz, 2H), 7.14 (d, J = 8.5 Hz, 2H), 5.74 (s, 1H), 5.33 (d, J = 9.2 Hz, 1H), 2.42 (t, J = 7.2 Hz, 1H), 2.19 (td, J = 13.9, 3.8 Hz, 1H), 1.99 (dd, J = 14.5, 4.3 Hz, 1H), 1.88-1.78 (m, 1H), 1.67-1.55 (m, 4H), 1.43-1.27 (m, 2H), 1.25 (s, 3H), 1.23- 1.15 (m, 1H), 1.03- 0.94 (m, 1H), 0.92- | | | 3.3756 | 10.50 |
| | | | 0.88 (m, 6H). | | | | |
| 130 | 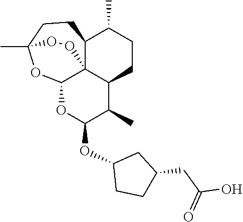 [SMILES: C[C@H]1[C@@H](O[C@H]2CC[C@@H](CC(=O)O)C2)O[C@@H]2O[C@@]3(C)CC[C@H]4[C@H](C)CC[C@@H]1[C@@]24OO3] | 2-((1R,3S)-3- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclo- pentyl)acetic acid | ¹H NMR (400 MHz, DMSO) δ 11.99 (s, 1H), 5.29 (d, J = 3.5 Hz, 1H), 4.69 (d, J = 3.5 Hz, 1H), 4.13 (p, J = 5.0 Hz, 1H), 2.37 (dt, J = 7.9, 4.1 Hz, 1H), 2.28-2.24 (m, 2H), 2.17 (ddd, J = 13.6, 11.3, 3.8 Hz, 2H), 2.11-2.03 (m, 1H), 2.03-1.94 (m, 2H), 1.84-1.66 (m, 4H), 1.66-1.52 (m, 3H), 1.35 (dq, J = 14.0, 4.6 Hz, | | | 0.2408 | >20 |
| | | | 3H), 1.27 (s, 3H), | | | | |
| | | | 1.25-1.18 (m, 1H), | | | | |
| | | | 1.13 (td, J = 11.4, | | | | |
| | | | 6.6 Hz, 1H), 0.89 | | | | |
| | | | (d, J = 6.4 Hz, 3H), | | | | |
| | | | 0.85 (d, J = 10.8 | | | | |
| | | | Hz, 1H), 0.81 (d, | | | | |
| | | | J = 7.3 Hz, 3H). | | | | |
| 131 | 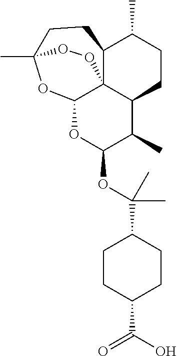 [SMILES: C[C@H]1[C@@H](OC(C)(C)[C@H]2CC[C@@H](C(=O)O)CC2)O[C@@H]2O[C@@]3(C)CC[C@H]4[C@H](C)CC[C@@H]1[C@@]24OO3] | (1R,4s)-4-(2- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)propan- 2-yl)cyclohexane- 1-carboxylic acid | ¹H NMR (400 MHz, DMSO) δ 11.96 (s, 1H), 5.33 (s, 1H), 4.98 (d, J = 3.6 Hz, 1H), 2.37 (dt, J = 8.0, 4.2 Hz, 1H), 2.16 (td, J = 14.0, 3.9 Hz, 1H), 2.08 (d, J = 13.0 Hz, 2H), 1.98 (d, J = 14.6 Hz, 1H), 1.82-1.72 (m, 2H), 1.69-1.55 (m, 4H), 1.42-1.29 (m, 5H), 1.26 (s, 3H), 1.23-1.18 (m, 4H), 1.15 (s, 3H), 1.13-1.08 (m, 1H), 1.06 (s, 3H), 0.91 (d, J = 6.3 Hz, 3H), 0.87-0.81 (m, 1H), 0.76 (d, J = 7.2 Hz, 3H). | | | 0.1588 | 16.49 |
| 132 | 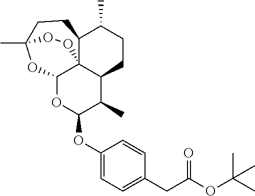 [SMILES: C[C@H]1[C@@H](Oc2ccc(CC(=O)OC(C)(C)C)cc2)O[C@@H]2O[C@@]3(C)CC[C@H]4[C@H](C)CC[C@@H]1[C@@]24OO3] | tert-butyl 2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetate | ¹H NMR (400 MHz, DMSO) δ 7.17 (d, J = 8.6 Hz, 2H), 7.00 (d, J = 8.6 Hz, 2H), 5.51 (d, J = 3.4 Hz, 1H), 5.42 (s, 1H), 3.47 (s, 2H), 2.64-2.52 (m, 1H), 2.19 (td, J = 13.9, 3.9 Hz, 1H), 2.04- 1.95 (m, 1H), 1.91 (dd, J = 13.6, 3.4 Hz, 1H), 1.82 (dtt, | | 497.51 | 0.0124 | 0.01 |
| | | | J = 10.1, 6.7, 3.3 | | | | |
| | | | Hz, 2H), 1.67-1.57 | | | | |
| | | | (m, 1H), 1.51 (dt, | | | | |
| | | | J = 15.3, 6.0 Hz, | | | | |
| | | | 1H), 1.45-1.33 (m, | | | | |
| | | | 11H), 1.28 (s, 2H), | | | | |
| | | | 1.26-1.14 (m, 1H), | | | | |
| | | | 0.97-0.83 (m, 7H). | | | | |
| 133 | 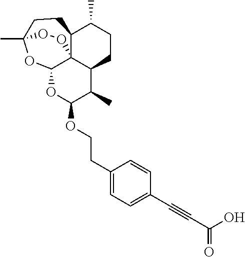 [SMILES: C[C@H]1[C@@H](OCCc2ccc(C#CC(=O)O)cc2)O[C@@H]2O[C@@]3(C)CC[C@H]4[C@H](C)CC[C@@H]1[C@@]24OO3] | 3-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) propiolic acid | ¹H NMR (400 MHz, DMSO) δ 7.61 (d, J = 8.8 Hz, 2H), 7.15 (d, J = 8.8 Hz, 2H), 5.68 (d, J = 3.4 Hz, 1H), 5.41 (s, 1H), 2.66-2.57 (m, 1H), 2.19 (td, J = 13.9, 3.9 Hz, 1H), 2.04-1.96 (m, 1H), 1.85 (q, J = 14.5 Hz, 3H), 1.63 (dd, J = 13.0, 3.4 Hz, 1H), 1.58-1.50 (m, 1H), 1.48-1.38 (m, 1H), 1.35 (dd, J = 13.0, 4.3 Hz, 1H), | | | 0.0249 | 1.77 |
| | | | 1.29 (s, 3H), 1.20 | | | | |
| | | | (td, J = 11.2, 6.3 | | | | |
| | | | Hz, 1H), 0.96 (d, | | | | |
| | | | J = 7.3 Hz, 3H), | | | | |
| | | | 0.90 (d, | | | | |
| | | | J = 6.3 Hz, 3H). | | | | |
| 134 | 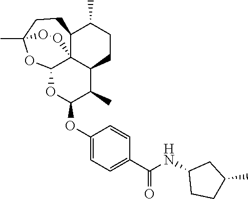 | (1R,3S)-3-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) benzamido)cyclo- pentane-1- carboxylic acid | ¹H NMR (400 MHz, DMSO) δ 12.12 (s, 1H), 8.24 (d, J = 7.4 Hz, 1H), 7.82 (d, J = 8.8 Hz, 2H), 7.10 (d, J = 8.8 Hz, 2H), 5.66 (d, J = 3.4 Hz, 1H), 5.39 (s, 1H), 4.24 (q, J = 7.6 Hz, 1H), 2.75 (p, J = 8.4 Hz, 1H), 2.68-2.55 (m, 1H), 2.30-2.09 (m, 2H), 2.07-1.95 (m, 1H), | | | 0.0244 | 8.00 |
| | | | 1.93-1.68 (m, 7H), | | | | |
| | | | 1.69-1.48 (m, 3H), | | | | |
| | | | 1.47-1.32 (m, 2H), | | | | |
| | | | 1.28 (s, 3H), 1.26- | | | | |
| | | | 1.12 (m, 2H), 0.96 | | | | |
| | | | (d, J = 7.3 Hz, 3H), | | | | |
| | | | 0.90 (d, J = 6.3 Hz, | | | | |
| | | | 3H). | | | | |
| 135 | 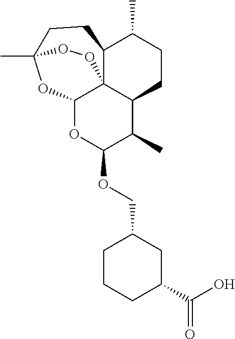 [SMILES: C[C@H]1[C@@H](OC[C@H]2CCC[C@@H](C(=O)O)C2)O[C@@H]2O[C@@]3(C)CC[C@H]4[C@H](C)CC[C@@H]1[C@@]24OO3] | (1R,3S)-3- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxylic acid | ¹H NMR (400 MHz, DMSO) δ 12.01 (s, 1H), 5.26 (s, 1H), 4.64 (d, J = 3.5 Hz, 1H), 3.52 (dd, J = 9.5, 6.2 Hz, 1H), 3.13 (dt, J = 9.8, 6.6 Hz, 1H), 2.39 (dq, J = 7.5, 3.8 Hz, 1H), 2.18 (qd, J = 11.2, 4.4 Hz, 2H), 2.04-1.94 (m, 1H), 1.90 (s, 1H), 1.86 (d, J = 13.5 Hz, 1H), 1.80-1.62 (m, 5H), 1.61-1.52 (m, 2H), 1.37 (dq, J = 12.9, 4.2 Hz, 2H), 1.31- 1.24 (m, 5H), 1.22- 1.08 (m, 3H), 0.99 (qd, J = 12.3, 3.2 | | | 0.2251 | >20 |
| | | | Hz, 1H), 0.89 (d, J = | | | | |
| | | | 6.2 Hz, 3H), 0.84 (d, | | | | |
| | | | J = 7.4 Hz, 3H). | | | | |
| 136 | 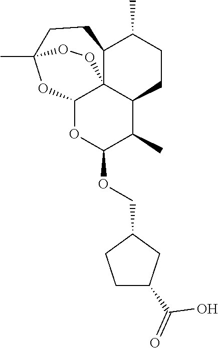 [SMILES: C[C@H]1[C@@H](OC[C@H]2CC[C@@H](C(=O)O)C2)O[C@@H]2O[C@@]3(C)CC[C@H]4[C@H](C)CC[C@@H]1[C@@]24OO3] | (1R,3S)-3- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) cyclopentane-1- carboxylic acid | ¹H NMR (400 MHz, DMSO) δ 12.00 (s, 1H), 5.30 (d, J = 4.6 Hz, 1H), 4.67 (d, J = 3.5 Hz, 1H), 3.59 (ddd, J = 9.4, 6.8, 4.6 Hz, 1H), 3.23 (ddd, J = 9.1, 6.2, 2.5 Hz, 1H), 2.72-2.63 (m, 2H), 2.43-2.33 (m, 1H), 2.24-2.10 (m, 2H), 2.04-1.89 (m, 2H), 1.85-1.61 (m, 6H), 1.56 (dd, J = 13.0, 3.3 Hz, 1H), 1.48- 1.31 (m, 5H), 1.29 (s, 3H), 1.14 (td, J = 11.4, 6.4 Hz, 1H), 0.90 (d, J = 6.3 Hz, 3H), 0.84 (d, J = 7.3 Hz, 3H). | | | 0.1972 | >20 |
| 137 | 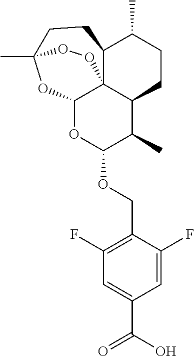 [SMILES: C[C@H]1[C@H](OCc2c(F)cc(C(=O)O)cc2F)O[C@@H]2O[C@@]3(C)CC[C@H]4[C@H](C)CC[C@@H]1[C@@]24OO3] | 3,5-difluoro-4- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, DMSO) δ 13.67 (s, 1H), 7.59 (d, J = 7.5 Hz, 1H), 5.46 (s, 1H), 4.87 (d, J = 11.4 Hz, 1H), 4.63 (d, J = 11.5 Hz, 1H), 4.58 (d, J = 9.2 Hz, 1H), 2.20 (dd, J = 13.9, 3.8 Hz, 1H), 2.17- 2.09 (m, 1H), 2.00 (d, J = 14.7 Hz, 1H), 1.86-1.77 (m, 1H), 1.60 (s, 1H), 1.57 (d, J = 3.3 Hz, 1H), 1.52-1.42 (m, 2H), 1.42-1.32 (m, 2H), 1.30 (s, 3H), 1.24 (s, 1H), 1.19-1.12 (m, 1H), 0.89 (d, J = 6.3 Hz, 3H), 0.71 (d, J = 7.1 Hz, 3H). | | | 0.0868 | >20 |
| 138 | 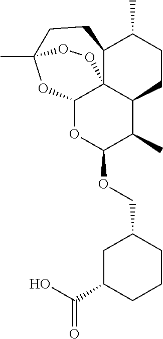 [SMILES: C[C@H]1[C@@H](OC[C@@H]2CCC[C@H](C(=O)O)C2)O[C@@H]2O[C@@]3(C)CC[C@H]4[C@H](C)CC[C@@H]1[C@@]24OO3] | (1S,3R)-3- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxylic acid | ¹H NMR (400 MHz, DMSO) δ 12.02 (s, 1H), 5.26 (s, 1H), 4.64 (d, J = 3.4 Hz, 1H), 3.56-3.47 (m, 1H), 3.12 (ddd, J = 9.4, 7.3, 5.8 Hz, 1H), 2.44-2.34 (m, 1H), 2.24-2.11 (m, 2H), 2.03-1.94 (m, 1H), 1.93-1.81 (m, 2H), 1.82-1.76 (m, 1H), 1.76-1.62 (m, 4H), 1.55 (d, J = 12.8 Hz, 2H), 1.37 (dq, J = 12.9, 4.4 Hz, 2H), 1.31-1.23 (m, 5H), 1.17-1.09 (m, 2H), 1.00 (td, J = 12.4, 3.4 Hz, 1H), 0.91-0.79 (m, 8H). | | | 0.1201 | >20 |
| 139 | 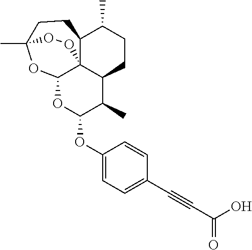 [SMILES: C[C@H]1[C@H](Oc2ccc(C#CC(=O)O)cc2)O[C@@H]2O[C@@]3(C)CC[C@H]4[C@H](C)CC[C@@H]1[C@@]24OO3] | 3-(4- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)phenyl) propiolic acid | ¹H NMR (400 MHz, DMSO) δ 7.57 (d, J = 8.7 Hz, 2H), 7.17 (d, J = 8.8 Hz, 2H), 5.74 (s, 1H), 5.32 (d, J = 9.2 Hz, 1H), 2.41 (q, J = 5.0 Hz, 1H), 2.19 (td, J = 13.9, 3.8 Hz, 1H), 2.05-1.96 (m, 1H), 1.86-1.81 (m, 1H), 1.69-1.53 (m, 4H), 1.35 (dq, J = 13.3, 6.8 Hz, 2H), 1.25 (s, 3H), 1.23- 1.14 (m, 1H), 0.98 (d, J = 11.2 Hz, | | | 0.0309 | 3.00 |
| | | | 1H), 0.92 (d, J = 6.3 | | | | |
| | | | Hz, 3H), 0.89 (d, J = | | | | |
| | | | 7.1 Hz, 3H). | | | | |
| 140 | 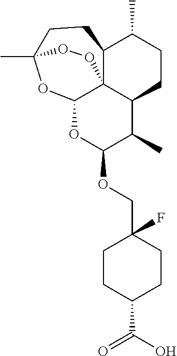 [SMILES: C[C@H]1[C@@H](OC[C@]2(F)CC[C@H](C(=O)O)CC2)O[C@@H]2O[C@@]3(C)CC[C@H]4[C@H](C)CC[C@@H]1[C@@]24OO3] | (1S,4r)-4- fluoro-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxylic acid | ¹H NMR (400 MHz, DMSO) δ 12.19 (s, 1H), 5.29 (s, 1H), 4.71 (d, J = 3.4 Hz, 1H), 3.74 (dd, J = 22.4, 11.2 Hz, 1H), 3.51-3.38 (m, 1H), 2.48-2.44 (s, 1H), 2.41 (dt, J = 7.9, 4.1 Hz, 1H), 2.18 (td, J = 13.9, 3.9 Hz, 1H), 2.04-1.96 (m, 1H), 1.83-1.73 (m, 5H), 1.72-1.53 (m, 7H), 1.39 (dt, J = 13.9, 6.7 Hz, 2H), 1.29 (s, 3H), 1.25 (d, J = 9.6 Hz, 1H), 1.20-1.07 (m, 1H), 0.92-0.82 (m, 7H). | | | 0.3142 | 18.97 |
| 141 | 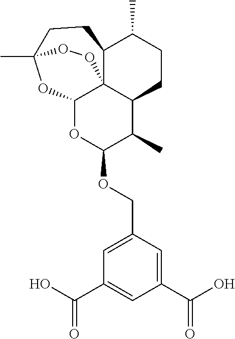 [SMILES: C[C@H]1[C@@H](OCc2cc(C(=O)O)cc(C(=O)O)c2)O[C@@H]2O[C@@]3(C)CC[C@H]4[C@H](C)CC[C@@H]1[C@@]24OO3] | 5- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) isophthalic acid | ¹H NMR (400 MHz, DMSO) δ 13.25 (s, 2H), 8.38 (d, J = 1.7 Hz, 1H), 8.13 (d, J = 1.6 Hz, 2H), 5.40 (s, 1H), 4.92-4.82 (m, 2H), 4.60 (d, J = 13.1 Hz, 1H), 2.46 (dd, J = 7.7, 4.0 Hz, 1H), 2.18 (td, J = 13.9, 3.8 Hz, 1H), 2.06-1.94 (m, 1H), 1.85-1.74 (m, 2H), 1.69 (dd, J = 13.9, 4.0 Hz, 1H), 1.59- 1.49 (m, 1H), 1.41 (dt, J = 13.3, 4.7 Hz, 1H), 1.35 (dd, J = 12.0, 5.1 Hz, 2H), 1.29 (s, 3H), 1.14 (td, J = 11.5, 6.7 Hz, | | | >20 | >20 |
| | | | 1H), 0.91-0.82 | | | | |
| | | | (m, 7H). | | | | |
| 142 | 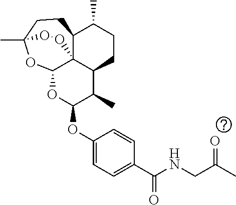 [SMILES: CC(=O)CNC(=O)c1ccc(O[C@H]2O[C@@H]3O[C@@]4(C)CC[C@H]5[C@H](C)CC[C@@H]([C@H]2C)[C@@]35OO4)cc1] | (4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzoyl) glycine | ¹H NMR (400 MHz, DMSO) δ 8.71 (t, J = 5.9 Hz, 1H), 7.85 (d, J = 8.3 Hz, 2H), 7.14 (d, J = 8.4 Hz, 2H), 5.67 (d, J = 3.4 Hz, 1H), 5.41 (s, 1H), 3.90 (d, J = 5.5 Hz, 2H), 2.61 (s, 1H), 2.26-2.13 (m, 1H), 2.00 (d, J = 14.7 Hz, 1H), 1.91 (t, J = 6.4 Hz, 1H), 1.88-1.77 (m, 2H), | | | 1.8212 | >20 |
| | | | 1.64 (d, J = 12.7 Hz, | | | | |
| | | | 1H), 1.59-1.49 (m, | | | | |
| | | | 1H), 1.47-1.32 (m, | | | | |
| | | | 2H), 1.29 (s, 3H), | | | | |
| | | | 1.26-1.16 (m, 2H), | | | | |
| | | | 0.97 (d, J = 7.4 Hz, | | | | |
| | | | 3H), 0.91 (d, J = 6.5 | | | | |
| | | | Hz, 3H). | | | | |
| 143 | 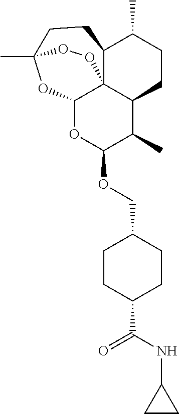 [SMILES: C[C@H]1[C@@H](OC[C@H]2CC[C@@H](C(=O)NC3CC3)CC2)O[C@@H]2O[C@@]3(C)CC[C@H]4[C@H](C)CC[C@@H]1[C@@]24OO3] | (1R,4s)-N- cyclopropyl-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxamide | ¹H NMR (400 MHz, DMSO) δ 7.69 (d, J = 4.2 Hz, 1H), 5.26 (s, 1H), 4.65 (d, J = 3.5 Hz, 1H), 3.66-3.59 (m, 1H), 3.22-3.15 (m, 1H), 2.60-2.54 (m, 2), 2.43-2.31 (m, 1H), 2.21-2.13 (m, 2H), 1.99 (d, J = 14.2 Hz, 1H), 1.84-1.76 (m, 1H), 1.72 (d, J = 10.4 Hz, 2H), 1.68- 1.53 (m, 4H), 1.52- 1.32 (m, 8H), 1.28 (s, 3H), 1.19-1.04 (m, 2H), 0.89 (d, J = 6.2 Hz, 3H), 0.83 (d, J = 7.4 Hz, 3H), 0.56 (td, J = 6.9, 4.7 Hz, 2H), 0.40-0.28 (m, 2H). | 464.62 | | 0.0726 | 1.00 |
| 144 | 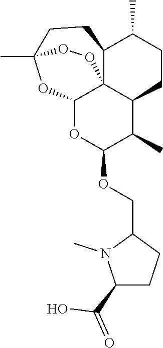 | (2S,5R)-1- methyl-5- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) pyrrolidine-2- carboxylic acid | | | | 3.3529 | 14.44 |
| 145 | 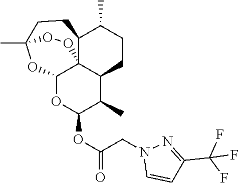 [SMILES: C[C@H]1[C@@H](OC(=O)Cn2ccc(C(F)(F)F)n2)O[C@@H]2O[C@@]3(C)CC[C@H]4[C@H](C)CC[C@@H]1[C@@]24OO3] | (3R,5aS,6R,8aS, 9R,10R,12R, 12aR)-3,6,9- trimethyldeca- hydro-12H-3,12- epoxy[1,2] dioxepino[4,3-i] isochromen-10-yl 2-(3-(trifluoro- methyl)-1H- pyrazol-1- yl)acetate | ¹H NMR (400 MHz, DMSO) δ 8.09-8.03 (m, 1H), 6.79 (d, J = 2.4 Hz, 1H), 6.02 (d, J = 3.4 Hz, 1H), 5.45-5.16 (m, 3H), 2.17 (td, J = 13.9, 3.9 Hz, 1H), 2.03- 1.97 (m, 1H), 1.87- 1.77 (m, 1H), 1.40 (q, J = 12.4 Hz, 3H), 1.31-1.24 (m, 4H), 1.23-1.09 (m, 1H), | | | 0.0732 | 0.11 |
| | | | 1.05-0.91 (m, 1H), | | | | |
| | | | 0.89 (d, J = 5.8 Hz, | | | | |
| | | | 3H), 0.86-0.80 (m, | | | | |
| | | | 1H), 0.74 (d, J = 7.3 | | | | |
| | | | Hz, 3H). | | | | |
| 146 | 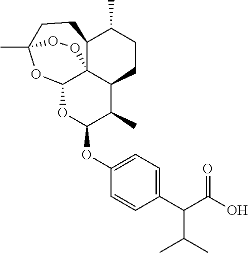 [SMILES: CC(C)C(C(=O)O)c1ccc(O[C@H]2O[C@@H]3O[C@@]4(C)CC[C@H]5[C@H](C)CC[C@@H]([C@H]2C)[C@@]35OO4)cc1] | 3-methyl-2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) butanoic acid | | | | 0.0596 | 0.80 |
| 147 | 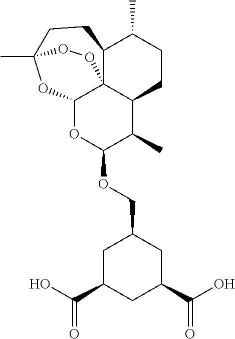 [SMILES: C[C@H]1[C@@H](OC[C@H]2C[C@@H](C(=O)O)C[C@@H](C(=O)O)C2)O[C@@H]2O[C@@]3(C)CC[C@H]4[C@H](C)CC[C@@H]1[C@@]24OO3] | (1R,3S,5r)-5- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1,3- dicarboxylic acid | | | | >20 | >20 |
| 148 | 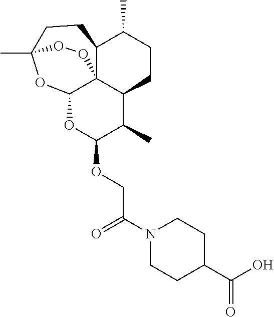 [SMILES: C[C@H]1[C@@H](OCC(=O)N2CCC(C(=O)O)CC2)O[C@@H]2O[C@@]3(C)CC[C@H]4[C@H](C)CC[C@@H]1[C@@]24OO3] | 1-(2- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)acetyl) piperidine-4- carboxylic acid | | | | 6.8741 | >20 |
| 149 | 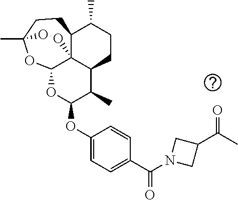 [SMILES: CC(=O)C1CN(C(=O)c2ccc(O[C@H]3O[C@@H]4O[C@@]5(C)CC[C@H]6[C@H](C)CC[C@@H]([C@H]3C)[C@@]46OO5)cc2)C1] | 1-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzoyl) azetidine-3- carboxylic acid | ¹H NMR (400 MHz, DMSO) δ 12.73 (s, 1H), 7.62 (d, J = 8.4 Hz, 2H), 7.11 (d, J = 8.4 Hz, 2H), 5.65 (d, J = 3.4 Hz, 1H), 5.41 (s, 1H), 4.47 (s, 1H), 4.36 (s, 1H), 4.20 (s, 1H), 4.05 (s, 1H), 3.57 (s, 1H), 3.49-3.42 (s, 1H), 2.61 (s, 1H), 2.19 (t, J = 13.7 Hz, 1H), 2.00 (d, J = 14.6 Hz, 1H), 1.89 (d, J = 11.5 Hz, 1H), 1.84- | | | 1.2402 | >20 |
| | | | 1.77 (m, 2H), 1.63 | | | | |
| | | | (d, J = 12.8 Hz, 1H), | | | | |
| | | | 1.54 (d, J = 12.7 Hz, | | | | |
| | | | 1H), 1.47-1.32 (m, | | | | |
| | | | 2H), 1.29 (s, 3H), | | | | |
| | | | 1.26-1.14 (m, 1H), | | | | |
| | | | 0.96 (d, J = 7.3 Hz, | | | | |
| | | | 3H), 0.90 (d, J = 6.3 | | | | |
| | | | 3H). | | | | |
| 150 | 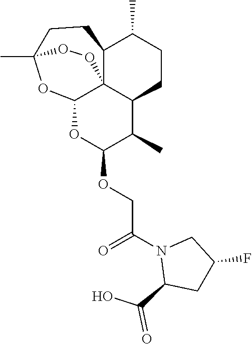 [SMILES: C[C@H]1[C@@H](OCC(=O)N2C[C@H](F)C[C@H]2C(=O)O)O[C@@H]2O[C@@]3(C)CC[C@H]4[C@H](C)CC[C@@H]1[C@@]24OO3] | (2S,4R)-4- fluoro-1-(2- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)acetyl) pyrrolidine-2- carboxylic acid | | | | >20 | >20 |
| 151 | 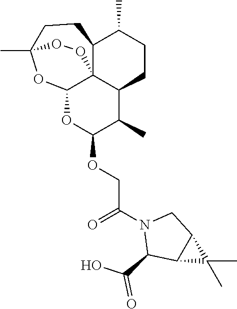 | (1R,2S,5S)-6,6- dimethyl-3-(2- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)acetyl)-3- azabicyclo[3.1.0] hexane-2- carboxylic acid | | | | 1.5734 | >20 |
| 152 | 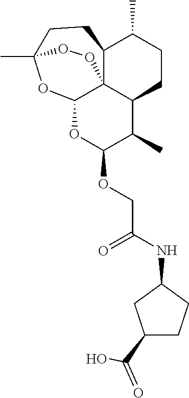 [SMILES: C[C@H]1[C@@H](OCC(=O)N[C@H]2CC[C@@H](C(=O)O)C2)O[C@@H]2O[C@@]3(C)CC[C@H]4[C@H](C)CC[C@@H]1[C@@]24OO3] | (1R,3S)-3-(2- (((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)acetamido) cyclopentane- 1-carboxylic acid | | | | 8.4784 | >20 |
| 153 | 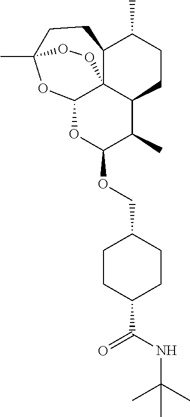 [SMILES: C[C@H]1[C@@H](OC[C@H]2CC[C@@H](C(=O)NC(C)(C)C)CC2)O[C@@H]2O[C@@]3(C)CC[C@H]4[C@H](C)CC[C@@H]1[C@@]24OO3] | (1R,4s)-N-(tert- butyl)-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxamide | ¹H NMR (400 MHz, DMSO) δ 7.17 (s, 1H), 5.27 (s, 1H), 4.66 (d, J = 3.4 Hz, 1H), 3.65 (dd, J = 9.4, 7.5 Hz, 1H), 3.20 (dd, J = 9.4, 6.7 Hz, 1H), 2.46-2.33 (m, 1H), 2.18 (td, J = 13.9, 4.1 Hz, 2H), 1.99 (dt, J = 14.4, 4.1 Hz, 1H), 1.80 (dq, J = 9.9, 3.3 Hz, 1H), 1.77-1.70 (m, 2H), 1.67 (dt, J = 8.2, 4.0 Hz, 1H), 1.63-1.50 (m, 5H), 1.40 (qd, J = 8.5, 3.6 Hz, 6H), 1.29 (s, 3H), 1.26 (s, 1H), 1.22 (s, 9H), 1.14 (td, J = 11.3, 6.5 Hz, 1H), 0.93-0.82 (m, 7H). | | 502.4 | 0.0973 | 0.54 |
| 154 | 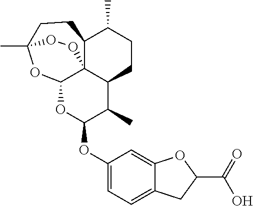 [SMILES: C[C@H]1[C@@H](Oc2ccc3c(c2)OC(C(=O)O)C3)O[C@@H]2O[C@@]3(C)CC[C@H]4[C@H](C)CC[C@@H]1[C@@]24OO3] | 6- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)-2,3- dihydrobenzo- furan-2- carboxylic acid | ¹H NMR (400 MHz, DMSO) δ 7.10 (dd, J = 8.2, 2.4 Hz, 1H), 6.63-6.41 (m, 2H), 5.48 (dd, J = 6.3, 3.5 Hz, 1H), 5.42 (s, 1H), 5.23 (dd, J = 10.6, 5.9 Hz, 1H), 3.47 (dd, J = 15.8, 10.7 Hz, 1H), 3.16 (dd, J = 15.7, 5.9 Hz, 1H), 2.57 (dt, J = 7.8, 4.2 Hz, 1H), | | | 0.0708 | 9.00 |
| | | | 2.19 (td, J = 13.9, | | | | |
| | | | 4.0 Hz, 1H), 2.05- | | | | |
| | | | 1.95 (m, 1H), 1.92- | | | | |
| | | | 1.77 (m, 3H), 1.63 | | | | |
| | | | (dd, J = 13.1, 3.6 | | | | |
| | | | Hz, 1H), 1.51 (dt, | | | | |
| | | | J = 12.8, 4.7 Hz, | | | | |
| | | | 1H), 1.44-1.34 (m, | | | | |
| | | | 3H), 1.29 (d, J = 4.7 | | | | |
| | | | Hz, 3H), 1.20 (td, | | | | |
| | | | J = 11.4, 6.5 Hz, | | | | |
| | | | 1H), 1.11 (d, J = 7.0 | | | | |
| | | | Hz, 1H), 0.96 (d, J = | | | | |
| | | | 7.3 Hz, 3H), 0.91 (d, | | | | |
| | | | J = 6.3 Hz, 3H). | | | | |
| 155 | 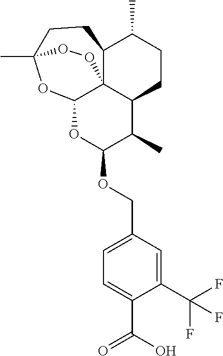 [SMILES: C[C@H]1[C@@H](OCc2ccc(C(=O)O)c(C(F)(F)F)c2)O[C@@H]2O[C@@]3(C)CC[C@H]4[C@H](C)CC[C@@H]1[C@@]24OO3] | 2- (trifluoromethyl)- 4- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) benzoic acid | | | | 0.1753 | >20 |
| 156 | 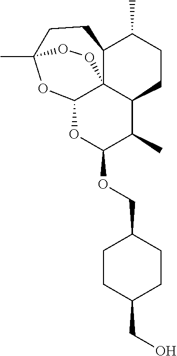 [SMILES: C[C@H]1[C@@H](OC[C@H]2CC[C@@H](CO)CC2)O[C@@H]2O[C@@]3(C)CC[C@H]4[C@H](C)CC[C@@H]1[C@@]24OO3] | ((1R,4s)-4- ((((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)methyl) cyclohexyl) methanol | ¹H NMR (400 MHz, DMSO) δ 5.26 (s, 1H), 4.65 (d, J = 3.4 Hz, 1H), 4.36 (t, J = 5.3 Hz, 1H), 3.60 (dd, J = 9.4, 7.2 Hz, 1H), 3.29 (dd, J = 6.9, 5.3 Hz, 2H), 3.18 (dd, J = 9.4, 6.6 Hz, 1H), 2.39 (dt, J = 7.8, 4.1 Hz, 1H), 2.18 (td, J = 14.1, 3.9 Hz, 1H), 2.04- 1.95 (m, 1H), 1.85- 1.77 (m, 1H), 1.77- 1.62 (m, 3H), 1.59- 1.49 (m, 2H), 1.47- 1.31 (m, 10H), 1.28 (s, 3H), 1.25 (d, J = 8.8 Hz, 1H), 1.14 (td, J = 11.4, 6.4 Hz, 1H), 0.94-0.83 (m, 7H). | | 433.4 | 0.0766 | 0.27 |
| 157 | 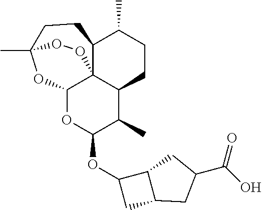 | (1R,5R)-6- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)bicyclo [3.2.0]heptane-3- carboxylic acid | | | | 0.7969 | >20 |
| 158 | 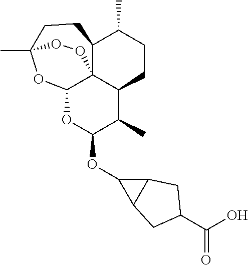 [SMILES: C[C@H]1[C@@H](OC2C3CC(C(=O)O)CC32)O[C@@H]2O[C@@]3(C)CC[C@H]4[C@H](C)CC[C@@H]1[C@@]24OO3] | 6- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)bicyclo [3.1.0]hexane-3- carboxylic acid | ¹H NMR (400 MHz, DMSO) δ 11.88 (s, 1H), 5.26 (s, 1H), 4.65 (d, J = 3.5 Hz, 1H), 4.21 (t, J = 6.4 Hz, 1H), 2.37 (dt, J = 7.6, 4.0 Hz, 1H), 2.24-2.09 (m, 2H), 2.04-1.91 (m, 3H), 1.86 (d, J = 14.4 Hz, 1H), 1.84-1.77 (m, 2H), 1.73 (d, J = 3.0 Hz, 2H), 1.70-1.62 (m, 2H), 1.61-1.54 (m, 2H), 1.43-1.31 (m, 3H), 1.28 (s, 3H), 1.15 (dt, J = 11.3, 5.7 Hz, 1H), | | | 4.2473 | >20 |
| | | | 0.90 (d, J = 6.3 Hz, | | | | |
| | | | 3H), 0.88-0.84 (m, | | | | |
| | | | 1H), 0.81 (d, J = 7.3 | | | | |
| | | | Hz, 4H). | | | | |
| 159 | 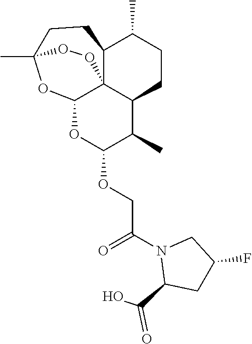 [SMILES: C[C@H]1[C@H](OCC(=O)N2C[C@H](F)C[C@H]2C(=O)O)O[C@@H]2O[C@@]3(C)CC[C@H]4[C@H](C)CC[C@@H]1[C@@]24OO3] | (2S,4R)-4- fluoro-1-(2- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)acetyl) pyrrolidine-2- carboxylic acid | | | | >20 | >20 |
| 160 | 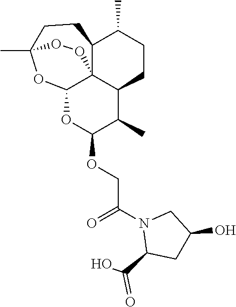 [SMILES: C[C@H]1[C@@H](OCC(=O)N2C[C@@H](O)C[C@H]2C(=O)O)O[C@@H]2O[C@@]3(C)CC[C@H]4[C@H](C)CC[C@@H]1[C@@]24OO3] | (2S,4S)-4- hydroxy-1-(2- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)acetyl) pyrrolidine-2- carboxylic acid | | | | >20 | >20 |
| 161 | 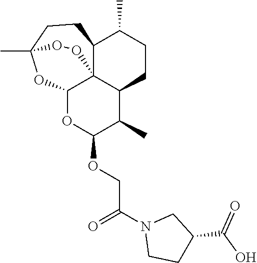 [SMILES: C[C@H]1[C@@H](OCC(=O)N2CC[C@@H](C(=O)O)C2)O[C@@H]2O[C@@]3(C)CC[C@H]4[C@H](C)CC[C@@H]1[C@@]24OO3] | (R)-1-(2- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)acetyl) pyrrolidine-3- carboxylic acid | | | | >20 | >20 |
| 162 | 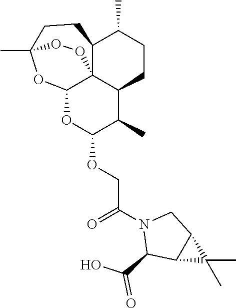 | (1R,2S,5S)-6,6- dimethyl-3-(2- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)acetyl)-3- azabicyclo[3.1.0] hexane-2- carboxylic acid | | | | 5.7516 | >20 |
| 163 | 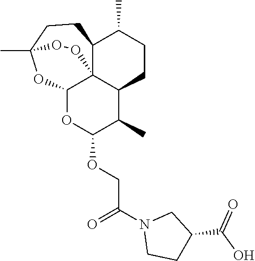 [SMILES: C[C@H]1[C@H](OCC(=O)N2CC[C@@H](C(=O)O)C2)O[C@@H]2O[C@@]3(C)CC[C@H]4[C@H](C)CC[C@@H]1[C@@]24OO3] | (R)-1-(2- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)acetyl) pyrrolidine-3- carboxylic acid | ¹H NMR (400 MHz, DMSO) δ 12.54 (s, 1H), 5.41 (d, J = 6.5 Hz, 1H), 4.57 (dd, J = 9.2, 4.2 Hz, 1H), 4.31-4.18 (m, 1H), 4.16 (d, J = 14.5 Hz, 1H), 3.73-3.42 (m, 3H), 3.05 (dp, J = 41.2, 7.4 Hz, 1H), 2.26-1.88 (m, 5H), 1.79 (dd, J = 7.8, 4.3 Hz, 1H), 1.63-1.54 (m, 2H), 1.49-1.41 (m, 1H), 1.39-1.30 (m, 3H), 1.28 (s, 3H), 1.22 (d, J = 4.4 Hz, 1H), 1.15 (td, | | | >20 | >20 |
| | | | J = 11.1, 6.4 Hz, | | | | |
| | | | 1H), 0.94 (t, J = | | | | |
| | | | 11.0 Hz, 1H), 0.88 | | | | |
| | | | (d, J = 6.3 Hz, 3H), | | | | |
| | | | 0.85 (d, J = 7.1 Hz, | | | | |
| | | | 3H). | | | | |
| 164 | 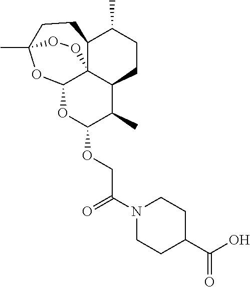 [SMILES: C[C@H]1[C@H](OCC(=O)N2CCC(C(=O)O)CC2)O[C@@H]2O[C@@]3(C)CC[C@H]4[C@H](C)CC[C@@H]1[C@@]24OO3] | 1-(2- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)acetyl) piperidine-4- carboxylic acid | | | | >20 | >20 |
| 165 | 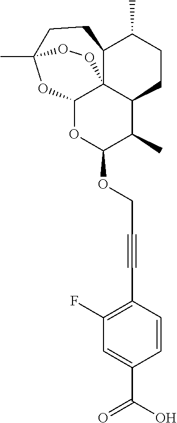 [SMILES: C[C@H]1[C@@H](OCC#Cc2ccc(C(=O)O)cc2F)O[C@@H]2O[C@@]3(C)CC[C@H]4[C@H](C)CC[C@@H]1[C@@]24OO3] | 3-fluoro-4-(3- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)prop-1- yn-1-yl) benzoic acid | ¹H NMR (400 MHz, DMSO) δ 13.47 (s, 1H), 8.00-7.40 (m, 3H), 5.44 (s, 1H), 4.95 (d, J = 3.5 Hz, 1H), 4.58 (d, J = 5.5 Hz, 2H), 2.20 (td, J = 13.9, 3.9 Hz, 1H), 2.01 (ddd, J = 14.5, 4.9, 2.9 Hz, 1H), 1.82 (ddt, J = 13.2, 6.4, 2.8 Hz, 1H), 1.76-1.66 (m, 2H), 1.58 (dt, J = 13.1, 3.4 Hz, 1H), 1.46-1.32 (m, 3H), 1.30 (s, 3H), 1.23- 1.07 (m, 1H), 0.91- 0.87 (m, 6H). | | | 0.0795 | 0.30 |
| 166 | 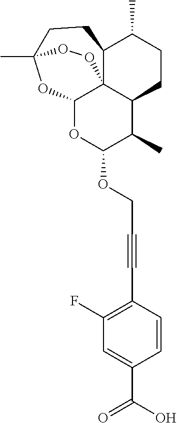 [SMILES: C[C@H]1[C@H](OCC#Cc2ccc(C(=O)O)cc2F)O[C@@H]2O[C@@]3(C)CC[C@H]4[C@H](C)CC[C@@H]1[C@@]24OO3] | 3-fluoro-4-(3- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)prop-1-yn- 1-yl)benzoic acid | | | | 0.0669 | 1.80 |
| 167 | 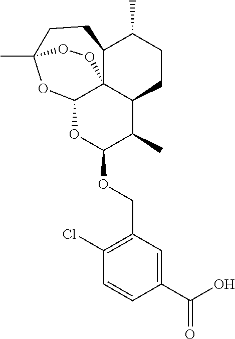 [SMILES: C[C@H]1[C@@H](OCc2cc(C(=O)O)ccc2Cl)O[C@@H]2O[C@@]3(C)CC[C@H]4[C@H](C)CC[C@@H]1[C@@]24OO3] | 4-chloro-3- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzoic acid | | | | 0.1320 | 10.50 |
| 168 | 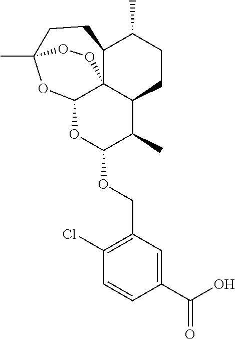 [SMILES: C[C@H]1[C@H](OCc2cc(C(=O)O)ccc2Cl)O[C@@H]2O[C@@]3(C)CC[C@H]4[C@H](C)CC[C@@H]1[C@@]24OO3] | 4-chloro-3- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) benzoic acid | | | | 0.0453 | 6.50 |
| 169 | 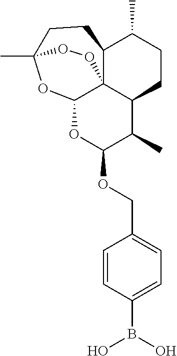 [SMILES: C[C@H]1[C@@H](OCc2ccc(B(O)O)cc2)O[C@@H]2O[C@@]3(C)CC[C@H]4[C@H](C)CC[C@@H]1[C@@]24OO3] | (4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) phenyl)boronic acid | ¹H NMR (400 MHz, DMSO) δ 8.07 (s, 2H), 7.76 (d, J = 7.7 Hz, 2H), 7.28 (d, J = 7.7 Hz, 2H), 5.40 (s, 1H), 4.84-4.73 (m, 2H), 2.47-2.39 (m, 1H), 2.25-2.13 (m, 1H), 2.01 (d, J = 14.3 Hz, 1H), 1.87- 1.74 (m, 2H), 1.70 (dd, J = 11.3, 7.3 Hz, 1H), 1.58-1.51 (m, 1H), 1.46-1.35 (m, 1H), 1.34 (s, 1H), 1.31 (s, 3H), 1.17 (dd, J = 11.5, 6.5 Hz, 1H), 0.92- 0.82 (m, 7H). | | | 0.1285 | 0.27 |
| 170 | 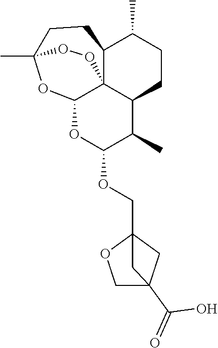 [SMILES: C[C@H]1[C@H](OCC23CC(C(=O)O)(CO2)C3)O[C@@H]2O[C@@]3(C)CC[C@H]4[C@H](C)CC[C@@H]1[C@@]24OO3] | 1- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl)-2- oxabicyclo[2.1.1] hexane-4- carboxylic acid | ¹H NMR (400 MHz, DMSO) δ 12.68 (s, 1H), 5.41 (s, 1H), 4.52 (d, J = 9.2 Hz, 1H), 3.88 (d, J = 11.7 Hz, 1H), 3.76 (s, 2H), 3.66 (d, J = 11.7 Hz, 1H), 2.22- 2.08 (m, 2H), 2.00 (dt, J = 17.6, 8.9 Hz, 3H), 1.85-1.75 (m, 1H), 1.70 (t, J = 4.4 Hz, 2H), 1.62- 1.56 (m, 2H), 1.50- 1.38 (m, 1H), 1.47- 1.39 (m, 1H), 1.37- 1.29 (m, 2H), 1.28 (s, 3H), 1.26-1.17 (m, 1H), 1.17-1.09 (m, 1H), 0.93 (d, J = 11.1 Hz, 1H), 0.88 (d, J = 6.3 Hz, | | | | |
| | | | 3H), 0.80 (d, J = | | | | |
| | | | 7.1 Hz, 3H). | | | | |
| 171 | 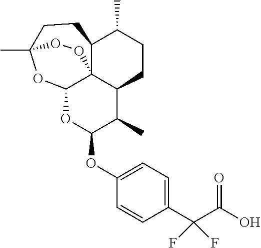 [SMILES: C[C@H]1[C@@H](Oc2ccc(C(F)(F)C(=O)O)cc2)O[C@@H]2O[C@@]3(C)CC[C@H]4[C@H](C)CC[C@@H]1[C@@]24OO3] | 2,2-difluoro-2- (4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 12.77 (s, 1H), 7.43 (d, J = 8.4 Hz, 2H), 7.05 (d, J = 8.6 Hz, 2H), 5.57 (d, J = 3.4 Hz, 1H), 5.41 (s, 1H), 2.61- 2.56 (m, 1H), 2.19 (t, J = 13.5 Hz, 1H), 1.99 (d, J = 15.6 Hz, 1H), 1.91 (d, J = 13.0 Hz, 1H), 1.86- 1.77 (m, 2H), 1.64 (d, J = 13.0 Hz, 1H), 1.52 (d, J = 12.9 Hz, | | | 7.0715 | >20 |
| | | | 1H), 1.44-1.33 (m, | | | | |
| | | | 2H), 1.28 (s, 3H), | | | | |
| | | | 1.25-1.14 (m, 1H), | | | | |
| | | | 0.97-0.89 (m, 7H). | | | | |
| 172 | 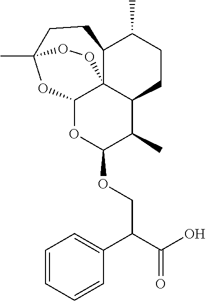 [SMILES: C[C@H]1[C@@H](OCC(C(=O)O)c2ccccc2)O[C@@H]2O[C@@]3(C)CC[C@H]4[C@H](C)CC[C@@H]1[C@@]24OO3] | 2-phenyl-3- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) propanic acid | ¹H NMR (400 MHz, DMSO) δ 12.55 (s, 1H), 7.75-6.96 (m, 5H), 4.95 (s, 1H), 4.69 (d, J = 3.4 Hz, 1H), 3.97 (dd, J = 9.0, 7.3 Hz, 1H), 3.83 (t, J = 6.9 Hz, 1H), 3.74 (dd, J = 8.9, 6.9 Hz, 1H), 2.32 (dt, J = 7.8, 4.1 Hz, 1H), 2.13 (td, J = 14.0, 4.0 Hz, 1H), 1.95 (dt, J = 14.4, 3.9 Hz, 1H), 1.86-1.68 (m, 1H), 1.55-1.32 (m, 3H), 1.28 (d, J = 3.5 Hz, | | | 0.2732 | >20 |
| | | | 1H), 1.26 (s, 3H), | | | | |
| | | | 1.23-1.18 (m, 1H), | | | | |
| | | | 1.15-0.93 (m, 2H), | | | | |
| | | | 0.89 (d, J = 6.2 Hz, | | | | |
| | | | 1H), 0.83 (d, J = 5.8 | | | | |
| | | | Hz, 3H), 0.77 (d, J = | | | | |
| | | | 7.3 Hz, 3H). | | | | |
| 173 | 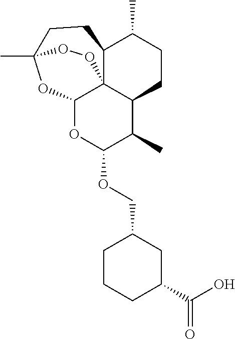 [SMILES: C[C@H]1[C@H](OC[C@H]2CCC[C@@H](C(=O)O)C2)O[C@@H]2O[C@@]3(C)CC[C@H]4[C@H](C)CC[C@@H]1[C@@]24OO3] | (1R,3S)-3- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) cyclohexane-1- carboxylic acid | ¹H NMR (400 MHz, DMSO) δ 12.01 (s, 1H), 5.26 (s, 1H), 4.64 (d, J = 3.5 Hz, 1H), 3.52 (dd, J = 9.5, 6.2 Hz, 1H), 3.13 (dt, J = 9.8, 6.6 Hz, 1H), 2.39 (dq, J = 7.5, 3.8 Hz, 1H), 2.18 (qd, J = 11.2, 4.4 Hz, 2H), 2.06- 1.94 (m, 1H), 1.90 (s, 1H), 1.86 (d, J = 13.5 Hz, 1H), 1.80- 1.72 (m, 1H), 1.69- 1.62 (m, 2H), 1.62- 1.52 (m, 2H), 1.36 (ddt, J = 15.6, 11.5, 6.7 Hz, 2H), 1.32- 1.22 (m, 5H), 1.19- 1.09 (m, 2H), 0.99 | | | 0.1699 | >20 |
| | | | (qd, J = 12.3, 3.2 | | | | |
| | | | Hz, 1H), 0.92-0.78 | | | | |
| | | | (m, 8H). | | | | |
| 174 | 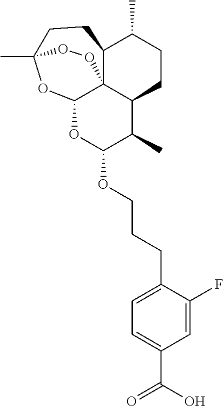 [SMILES: C[C@H]1[C@H](OCCCc2ccc(C(=O)O)cc2F)O[C@@H]2O[C@@]3(C)CC[C@H]4[C@H](C)CC[C@@H]1[C@@]24OO3] | 3-fluoro-4-(3- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)propyl) benzoic acid | | | | 0.1112 | 7.00 |
| 175 | 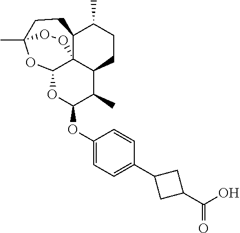 [SMILES: C[C@H]1[C@@H](Oc2ccc(C3CC(C(=O)O)C3)cc2)O[C@@H]2O[C@@]3(C)CC[C@H]4[C@H](C)CC[C@@H]1[C@@]24OO3] | 3-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) cyclobutane-1- carboxylic acid | | | | 0.0581 | 1.50 |
| 176 | 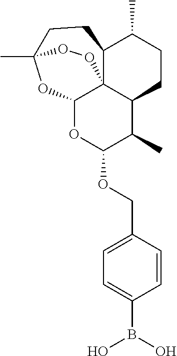 [SMILES: C[C@H]1[C@H](OCc2ccc(B(O)O)cc2)O[C@@H]2O[C@@]3(C)CC[C@H]4[C@H](C)CC[C@@H]1[C@@]24OO3] | (4- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) phenyl)boronic acid | | | | 0.2056 | 0.40 |
| 177 | 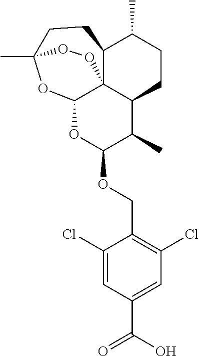 [SMILES: C[C@H]1[C@@H](OCc2c(Cl)cc(C(=O)O)cc2Cl)O[C@@H]2O[C@@]3(C)CC[C@H]4[C@H](C)CC[C@@H]1[C@@]24OO3] | 3,5-dichloro-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzoic acid | | | | 0.0445 | 0.66 |
| 178 | 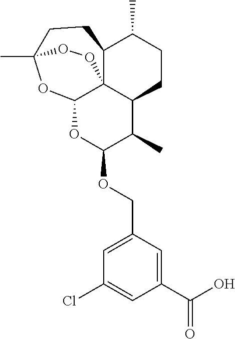 [SMILES: C[C@H]1[C@@H](OCc2cc(Cl)cc(C(=O)O)c2)O[C@@H]2O[C@@]3(C)CC[C@H]4[C@H](C)CC[C@@H]1[C@@]24OO3] | 3-chloro-5- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, DMSO) δ 13.15 (s, 1H), 7.88 (s, 1H), 7.80 (s, 1H), 7.64 (d, J = 1.9 Hz, 1H), 5.40 (s, 1H), 4.84 (s, 1H), 4.82 (d, J = 9.7 Hz, 1H), 4.54 (d, J = 13.2 Hz, 1H), 2.46 (dd, J = 7.9, 4.1 Hz, 1H), 2.18 (td, J = 13.9, 3.9 Hz, 1H), 2.00 (dt, J = 14.0, 4.0 Hz, 1H), 1.78 (td, J = 12.2, 5.1 Hz, 2H), 1.73-1.66 (m, 1H), 1.55 (dd, J = 13.2, 3.3 Hz, 1H), 1.45-1.33 (m, 3H), 1.31 (s, 1H), 1.30 (s, 3H), 1.16 (dp, J = | | | 0.2427 | 4.00 |
| | | | 11.4, 5.6 Hz, 1H), | | | | |
| | | | 0.95-0.82 (m, 7H). | | | | |
| 179 | 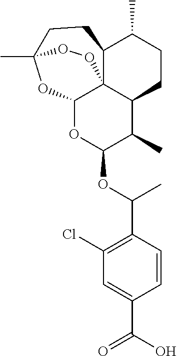 [SMILES: CC(O[C@H]1O[C@@H]2O[C@@]3(C)CC[C@H]4[C@H](C)CC[C@@H]([C@H]1C)[C@@]24OO3)c1ccc(C(=O)O)cc1Cl] | 3-chloro-4-(1- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)ethyl) benzoic acid | ¹H NMR (400 MHz, DMSO) δ 13.31 (s, 1H), 7.97-7.91 (m, 1H), 7.90 (s, 1H), 7.59 (d, J = 8.0 Hz, 1H), 5.49 (s, 1H), 5.22 (q, J = 6.4 Hz, 1H), 4.51 (d, J = 3.4 Hz, 1H), 2.43-2.31 (m, 1H), 2.19 (t, J = 13.0 Hz, 1H), 2.02 (d, J = 14.6 Hz, 1H), 1.96-1.73 (m, 3H), 1.62 (d, J = 12.7 Hz, 1H), 1.49-1.36 (m, 6H), 1.30 (s, 3H), 1.27-1.12 (m, 2H), 0.92 (d, J = 6.2 Hz, 3H), 0.87 (d, J = 7.3 Hz, 3H). | | | 0.0658 | 1.50 |
| 180 | 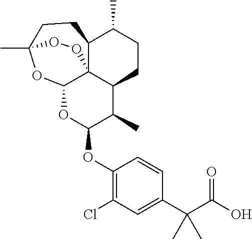 [SMILES: C[C@H]1[C@@H](Oc2ccc(C(C)(C)C(=O)O)cc2Cl)O[C@@H]2O[C@@]3(C)CC[C@H]4[C@H](C)CC[C@@H]1[C@@]24OO3] | 2-(3-chloro-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl)-2- methylpropanoic acid | ¹H NMR (400 MHz, MeOD) δ 12.45 (s, 1H), 7.44-7.19 (m, 3H), 5.67 (d, J = 3.5 Hz, 1H), 5.45 (s, 1H), 2.62 (dt, J = 8.0, 4.2 Hz, 1H), 2.19 (td, J = 13.8, 3.8 Hz, 1H), 2.13- 1.93 (m, 2H), 1.86- 1.77 (m, 2H), 1.63 (dd, J = 13.3, 3.4 Hz, 1H), 1.54 (dt, J = 13.5, 4.6 Hz, 1H), 1.45 (s, 6H), 1.36 (dd, J = 12.7, 5.3 Hz, 2H), 1.28 (s, | | | 0.0402 | 0.16 |
| | | | 3H), 1.21 (dq, J = | | | | |
| | | | 11.1, 6.0 Hz, 1H), | | | | |
| | | | 1.00 (d, J = 7.2 Hz, | | | | |
| | | | 3H), 0.97-0.93 (s, | | | | |
| | | | 1H), 0.91 (d, J = 6.2 | | | | |
| | | | Hz, 3H). | | | | |
| 181 | 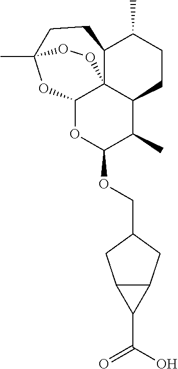 [SMILES: C[C@H]1[C@@H](OCC2CC3C(C2)C3C(=O)O)O[C@@H]2O[C@@]3(C)CC[C@H]4[C@H](C)CC[C@@H]1[C@@]24OO3] | 3- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) bicyclo[3.1.0] hexane-6- carboxylic acid | | | | 0.1397 | >20 |
| 182 | 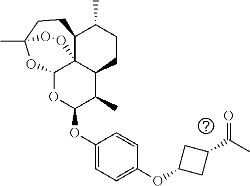 [SMILES: CC(=O)[C@H]1C[C@@H](Oc2ccc(O[C@H]3O[C@@H]4O[C@@]5(C)CC[C@H]6[C@H](C)CC[C@@H]([C@H]3C)[C@@]46OO5)cc2)C1] | (1R,3s)-3-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenoxy) cyclobutane-1- carboxylic acid | ¹H NMR (400 MHz, DMSO) δ 12.28 (s, 1H), 6.98 (d, J = 9.0 Hz, 2H), 6.80 (d, J = 9.0 Hz, 2H), 5.45 (s, 1H), 5.38 (d, J = 3.5 Hz, 1H), 4.56 (t, J = 7.0 Hz, 1H), 2.71 (dq, J = 25.0, 8.9 Hz, 3H), 2.56 (dt, J = 7.7, 4.1 Hz, 1H), 2.28-2.06 (m, 3H), 2.00 (d, J = 14.0 | | | 0.0241 | 1.00 |
| | | | Hz, 1H), 1.93-1.75 | | | | |
| | | | (m, 3H), 1.63 | | | | |
| | | | (d, J = 12.9 Hz, | | | | |
| | | | 1H), 1.54-1.46 (m, | | | | |
| | | | 1H), 1.46-1.33 (m, | | | | |
| | | | 2H), 1.28 (s, 2H), | | | | |
| | | | 1.24 (s, 1H), 1.23- | | | | |
| | | | 1.15 (m, 1H), 0.97 | | | | |
| | | | (d, J = 7.3 Hz, 3H), | | | | |
| | | | 0.91 (d, J = 6.3 Hz, | | | | |
| | | | 3H). | | | | |
| 183 | 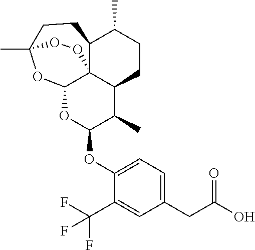 [SMILES: C[C@H]1[C@@H](Oc2ccc(CC(=O)O)cc2C(F)(F)F)O[C@@H]2O[C@@]3(C)CC[C@H]4[C@H](C)CC[C@@H]1[C@@]24OO3] | 2-(3- (trifluoromethyl)- 4-(((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 12.71 (s, 1H), 7.51 (d, J = 7.6 Hz, 2H), 7.39 (d, J = 8.9 Hz, 1H), 5.74 (d, J = 3.6 Hz, 1H), 5.38 (s, 1H), 3.56 (s, 2H), 2.65 (s, 1H), 2.25-2.15 (m, 1H), 2.01 (d, J = 14.6 Hz, 1H), 1.89-1.72 (m, 3H), 1.68-1.49 (m, 1H), 1.39-1.28 (m, 4H), 1.27-1.18 (m, 3H), 0.95 (d, J = 7.3 Hz, 3H), 0.90 (d, J = 6.1 Hz, 3H). | | | 0.0471 | 0.90 |
| 184 | 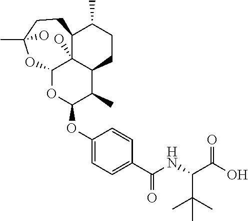 [SMILES: C[C@H]1[C@@H](Oc2ccc(C(=O)N[C@H](C(=O)O)C(C)(C)C)cc2)O[C@@H]2O[C@@]3(C)CC[C@H]4[C@H](C)CC[C@@H]1[C@@]24OO3] | (S)-3,3- dimethyl-2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) benzamido) butanoic acid | ¹H NMR (400 MHz, DMSO) δ 12.63 (s, 1H), 7.99 (d, J = 8.9 Hz, 1H), 7.87 (d, J = 8.8 Hz, 2H), 7.12 (d, J = 8.8 Hz, 2H), 5.68 (d, J = 3.4 Hz, 1H), 5.39 (s, 1H), 4.36 (d, J = 8.8 Hz, 1H), 2.62 (dt, J = 7.9, 4.2 Hz, 1H), 2.19 (td, J = 13.9, 3.9 Hz, 1H), 2.04- 1.96 (m, 1H), 1.94- 1.77 (m, 3H), 1.65 (dd, J = 13.2, 3.4 | | | 0.0170 | 0.80 |
| | | | Hz, 1H), 1.59-1.49 | | | | |
| | | | (m, 1H), 1.46-1.32 | | | | |
| | | | (m, 2H), 1.28 (s, | | | | |
| | | | 3H), 1.21 (td, J = | | | | |
| | | | 11.4, 6.3 Hz, 1H), | | | | |
| | | | 1.03 (s, 9H), 0.97 | | | | |
| | | | (d, J = 7.3 Hz, 3H), | | | | |
| | | | 0.94-0.83 (m, 4H). | | | | |
| 185 | 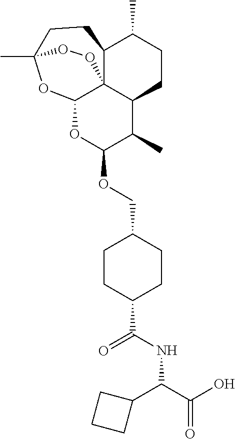 | (S)-2- cyclobutyl-2- ((1R,4R)-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxamido) acetic acid | ¹H NMR (400 MHz, DMSO) δ 12.37 (s, 1H), 7.85 (d, J = 8.1 Hz, 1H), 5.26 (s, 1H), 4.65 (d, J = 3.5 Hz, 1H), 4.16 (t, J = 8.4 Hz, 1H), 3.66- 3.57 (m, 1H), 3.19 (dd, J = 9.4, 6.7 Hz, 1H), 2.59 (q, J = 8.5 Hz, 1H), 2.43-2.30 (m, 2H), 2.17 (td, J = 13.9, 3.9 Hz, 1H), 1.99 (d, J = 14.3 Hz, 1H), 1.94- 1.85 (m, 2H), 1.84- 1.61 (m, 9H), 1.59- 1.50 (m, 2H), 1.50- 1.31 (m, 7H), 1.31- 1.21 (m, 5H), 1.18- 1.05 (m, 1H), 0.92- 0.78 (m, 7H). | | | 0.0502 | >20 |
| 186 | 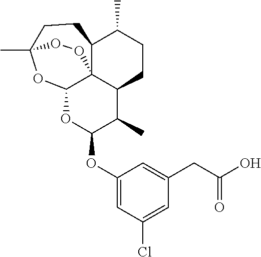 [SMILES: C[C@H]1[C@@H](Oc2cc(Cl)cc(CC(=O)O)c2)O[C@@H]2O[C@@]3(C)CC[C@H]4[C@H](C)CC[C@@H]1[C@@]24OO3] | 2-(3-chloro-5- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetic acid | | | 475.2 | 0.0624 | 4.00 |
| 187 | 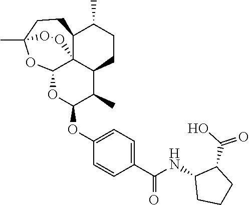 [SMILES: C[C@H]1[C@@H](Oc2ccc(C(=O)N[C@H]3CCC[C@H]3C(=O)O)cc2)O[C@@H]2O[C@@]3(C)CC[C@H]4[C@H](C)CC[C@@H]1[C@@]24OO3] | (1R,2S)-2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzamido) cyclopentane-1- carboxylic acid | ¹H NMR (400 MHz, DMSO) δ 11.98 (s, 1H), 8.06 (d, J = 8.2 Hz, 1H), 7.78 (d, J = 6.6 Hz, 2H), 7.10 (d, J = 8.8 Hz, 2H), 5.67 (d, J = 2.8 Hz, 1H), 5.39 (s, 1H), 4.54 (p, J = 7.5 Hz, 1H), 2.93 (q, J = 7.5 Hz, 1H), 2.61 (dt, J = 7.8, 4.2 Hz, 1H), 2.25-2.13 (m, 1H), | | | 0.0333 | 2.40 |
| | | | 2.04-1.72 (m, 9H), | | | | |
| | | | 1.64 (d, J = 12.8 Hz, | | | | |
| | | | 1H), 1.53 (td, | | | | |
| | | | J = 9.5, 6.1 Hz, | | | | |
| | | | 2H), 1.45-1.32 | | | | |
| | | | (m, 2H), 1.28 (s, | | | | |
| | | | 3H), 1.25-1.15 (m, | | | | |
| | | | 1H), 0.97 (d, J = | | | | |
| | | | 7.3 Hz, 3H), 0.91 | | | | |
| | | | (d, J = 6.3 Hz, 3H). | | | | |
| 188 | 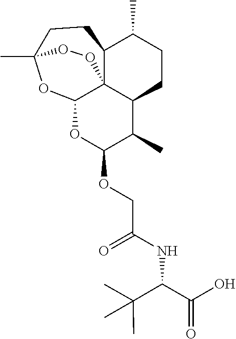 [SMILES: C[C@H]1[C@@H](OCC(=O)N[C@H](C(=O)O)C(C)(C)C)O[C@@H]2O[C@@]3(C)CC[C@H]4[C@H](C)CC[C@@H]1[C@@]24OO3] | (S)-3,3- dimethyl-2-(2- (((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)acetamido) butanoic acid | ¹H NMR (400 MHz, DMSO) δ 12.87 (s, 1H), 7.41 (d, J = 9.3 Hz, 1H), 5.47 (s, 1H), 4.78 (d, J = 3.5 Hz, 1H), 4.18-4.09 (m, 2H), 4.01 (d, J = 14.7 Hz, 1H), 2.45 (dt, J = 7.7, 4.0 Hz, 1H), 2.27- 2.11 (m, 1H), 2.03- 1.94 (m, 1H), 1.84- 1.76 (m, 4H), 1.76- 1.67 (m, 1H), 1.55 (dd, J = 13.0, 3.4 Hz, 1H), 1.45-1.33 (m, 3H), 1.28 (s, 3H), 1.24-1.08 (m, 2H), 0.93 (s, 9H), 0.92-0.87 (m, 6H). | | | 5.0602 | >20 |
| 189 | 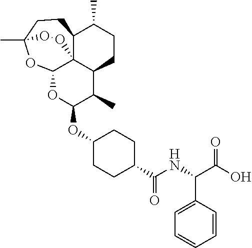 [SMILES: C[C@H]1[C@@H](O[C@H]2CC[C@@H](C(=O)N[C@H](C(=O)O)c3ccccc3)CC2)O[C@@H]2O[C@@]3(C)CC[C@H]4[C@H](C)CC[C@@H]1[C@@]24OO3] | (S)-2-phenyl-2- ((1R,4R)-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclo- hexane-1- carboxamido) acetic acid | | | | 1.8921 | 15.23 |
| 190 | 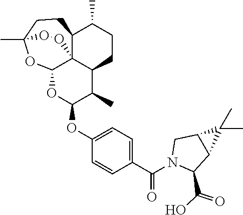 | (1R,2S,5S)-6,6- dimethyl-3-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzoyl)-3- azabicyclo[3.1.0] hexane-2- carboxylic acid | | | | 0.0803 | 0.33 |
| 191 | 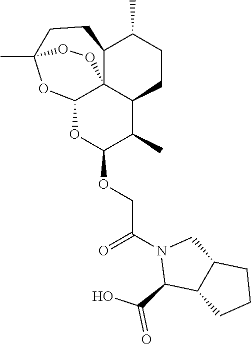 [SMILES: C[C@H]1[C@@H](OCC(=O)N2C[C@@H]3CCC[C@@H]3[C@H]2C(=O)O)O[C@@H]2O[C@@]3(C)CC[C@H]4[C@H](C)CC[C@@H]1[C@@]24OO3] | (1S,3aR,6aS)-2- (2- (((3R,5aS,6R, 8aS,9R,10S,12R, 12aR)-3,6,9- trimethyldeca- hydro-12H-3,12- epoxy[1,2] dioxepino[4,3-i] isochromen-10- yl)oxy)acetyl) octahydrocyclo- penta[c]pyrrole- 1-carboxlic acid | | | | 9.7323 | >20 |
| 192 | 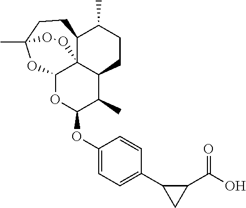 [SMILES: C[C@H]1[C@@H](Oc2ccc(C3CC3C(=O)O)cc2)O[C@@H]2O[C@@]3(C)CC[C@H]4[C@H](C)CC[C@@H]1[C@@]24OO3] | 2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) cyclopropane-1- carboxylic acid | ¹H NMR (400 MHz, DMSO) δ 12.11 (s, 1H), 7.11 (dd, J = 8.4, 5.9 Hz, 2H), 6.98 (dd, J = 8.7, 2.6 Hz, 2H), 5.48 (d, J = 3.4 Hz, 1H), 5.41 (d, J = 10.1 Hz, 1H), 2.57 (s, 1H), 2.23-2.13 (m, 2H), 1.99 (d, J = 14.8 Hz, 1H), 1.91 (d, J = 12.5 Hz, 0H), 1.86- 1.69 (m, 4H), 1.63 | | | 0.0672 | 3.50 |
| | | | (d, J = 12.8 Hz, 1H), | | | | |
| | | | 1.55-1.47 (m, 1H), | | | | |
| | | | 1.44-1.33 (m, 2H), | | | | |
| | | | 1.28 (s, 3H), 1.24- | | | | |
| | | | 1.14 (m, 2H), 0.99- | | | | |
| | | | 0.87 (m, 7H). | | | | |
| 193 | 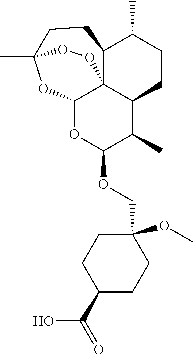 | (1R,4s)-4- methoxy-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxylic acid | | | | 7.0345 | >20 |
| 194 | 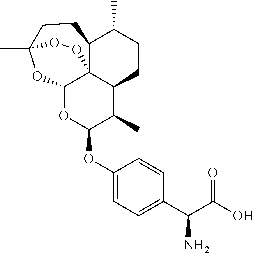 [SMILES: C[C@H]1[C@@H](Oc2ccc([C@H](N)C(=O)O)cc2)O[C@@H]2O[C@@]3(C)CC[C@H]4[C@H](C)CC[C@@H]1[C@@]24OO3] | (S)-2-amino-2- (4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetic acid | | | | 17.7137 | >20 |
| 195 | 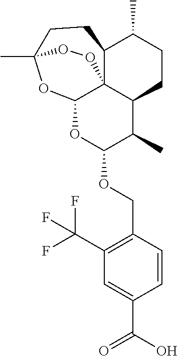 [SMILES: C[C@H]1[C@H](OCc2ccc(C(=O)O)cc2C(F)(F)F)O[C@@H]2O[C@@]3(C)CC[C@H]4[C@H](C)CC[C@@H]1[C@@]24OO3] | 3- (trifluoromethyl)- 4- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) benzoic acid | | | | 0.1558 | 10.00 |
| 196 | 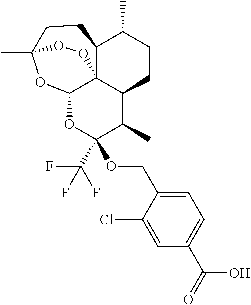 [SMILES: C[C@@H]1CC[C@H]2[C@@H](C)[C@](OCc3ccc(C(=O)O)cc3Cl)(C(F)(F)F)O[C@@H]3O[C@@]4(C)CC[C@@H]1[C@]32OO4] | 3-chloro-4- ((((3R,5aS,6R,8aS, 9R,10R,12R, 12aR-3,6,9- trimethyl-10- (trifluoromethyl) decahydro-12H- 3,12-epoxy[1,2] dioxepino[4,3-i] isochromen-10- yl)oxy)methyl) benzoic acid | ¹H NMR (400 MHz, DMSO) δ 12.71 (s, 1H), 7.87 (s, 1H), 7.85 (d, J = 8.7 Hz, 1H), 7.53 (d, J = 7.9 Hz, 1H), 5.64 (s, 1H), 4.93 (d, J = 12.9 Hz, 1H), 4.72 (d, J = 12.8 Hz, 1H), 2.74 (d, J = 7.8 Hz, 1H), 2.22 (td, J = 13.9, 3.9 Hz, 1H), 2.09-2.01 (m, 1H), 1.83 (d, J = 12.1 Hz, 1H), 1.69 (d, J = 8.6 Hz, 1H), 1.60-1.46 (m, 3H), 1.43-1.31 (m, 5H), 1.30-1.15 (m, 2H), 1.01 (d, J = 7.2 Hz, 3H), 0.92- 0.80 (m, 4H). | | | 0.0103 | 0.03 |
| 197 | 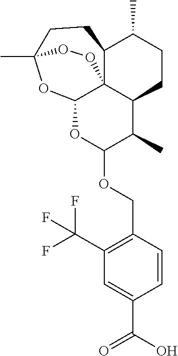 | 3- (trifluoromethyl)- 4- ((((3R,5aS,6R,8aS, 9R,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, DMSO) δ 13.50 (s, 1H), 8.25 (d, J = 8.5 Hz, 1H), 8.19 (s, 1H), 7.83 (d, J = 8.1 Hz, 1H), 5.43 (s, 1H), 4.99 (d, J = 13.7 Hz, 1H), 4.91 (d, J = 3.6 Hz, 2H), 4.64 (d, J = 13.7 Hz, 1H), 2.19 (td, J = 14.0, 3.9 Hz, 1H), 2.01 (d, J = 13.9 Hz, 1H), 1.88-1.77 (m, 1H), 1.77-1.65 (m, 2H), 1.61-1.48 (m, 1H), 1.47-1.34 (m, 2H), 1.31 (s, 6H), 1.30-1.06 (m, 2H), 0.91 (d, J = 7.4 Hz, 3H), 0.88 (d, J = 6.3 Hz, 3H). | | | 0.0660 | 5.38 |
| 198 | 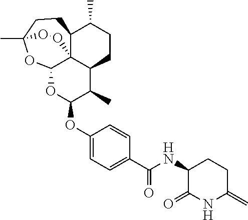 | N-((S)-2,6- dioxopiperidin- 3-yl)-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzamide | ¹H NMR (400 MHz, DMSO) δ 10.85 (s, 1H), 8.64 (d, J = 8.4 Hz, 1H), 7.86 (d, J = 8.7 Hz, 2H), 7.20-7.12 (m, 2H), 5.68 (d, J = 3.4 Hz, 1H), 5.41 (s, 1H), 4.77 (ddd, J = 13.0, 8.3, 5.3 Hz, 1H), 2.80 (ddd, J = 18.0, 13.3, 5.5 Hz, 1H), 2.62 (t, J = 7.7 Hz, 1H), 2.56 (d, J = 3.9 Hz, 1H), 2.25-2.05 (m, 2H), 2.05-1.92 | 515.2 | | 0.0479 | 4.00 |
| | | | (m, 2H), 1.90-1.75 | | | | |
| | | | (m, 3H), 1.64 (d, J = | | | | |
| | | | 12.9 Hz, 1H), 1.59- | | | | |
| | | | 1.50 (m, 1H), 1.47- | | | | |
| | | | 1.32 (m, 2H), 1.29 | | | | |
| | | | (s, 3H), 1.27-1.14 | | | | |
| | | | (m, 1H), 0.97 (d, J = | | | | |
| | | | 7.3 Hz, 3H), 0.91 | | | | |
| | | | (d, J = 6.3 Hz, 3H). | | | | |
| 199 | 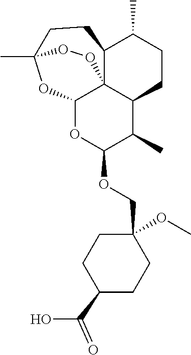 | (1S,4r)-4- methoxy-4- ((((3R,5aS,6R,8aS, 9R,10S,12R, 12aR)-3,6,9- trimethyldeca- hydro-12H-3,12- epoxy[1,2] dioxepino[4,3-i] isochromen-10-yl) oxy)methyl) cyclohexane-1- carboxylic acid | | | | 6.4834 | >20 |
| 200 | 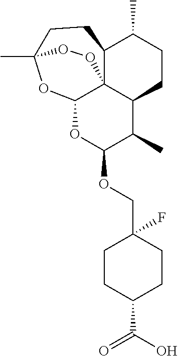 [SMILES: C[C@H]1[C@@H](OC[C@]2(F)CC[C@@H](C(=O)O)CC2)O[C@@H]2O[C@@]3(C)CC[C@H]4[C@H](C)CC[C@@H]1[C@@]24OO3] | (1R,4s)-4- fluoro-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxylic acid | ¹H NMR (400 MHz, DMSO) δ 12.12 (s, 1H), 5.31 (s, 1H), 4.71 (d, J = 3.4 Hz, 1H), 3.71 (dd, J = 20.7, 10.9 Hz, 1H), 2.43 (dt, J = 8.0, 4.2 Hz, 1H), 2.20 (td, J = 13.7, 3.8 Hz, 2H), 2.01 (d, J = 15.1 Hz, 1H), 1.87 (d, J = 14.6 Hz, 2H), 1.82-1.74 (m, 3H), 1.75-1.67 (m, 2H), 1.63-1.50 (m, 4H), 1.50-1.34 (m, 3H), 1.34-1.23 (m, 6H), 1.17 (td, J = 11.4, 6.4 Hz, 1H), 0.90 (m, 7H). | | | 4.4662 | 15.35 |
| 201 | 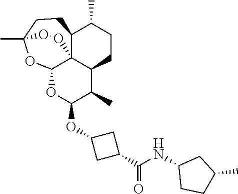 | (1R,3S)-3- ((1R,3R)-3- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclo- butane-1- carboxamido) cyclopentane-1- | ¹H NMR (400 MHz, DMSO) δ 12.10 (s, 1H), 7.83 (d, J = 7.4 Hz, 1H), 5.29 (s, 1H), 4.67 (s, 1H), 3.99 (d, J = 8.3 Hz, 2H), 2.68 (s, 1H), 2.43 (d, J = 8.5 Hz, 1H), 2.41-2.23 (m, 3H), 2.22-2.05 (m, 1H), 2.04-1.95 (m, 2H), 1.89 (d, J = 10.3 Hz, 1H), 1.85- 1.70 (m, 4H), 1.70- | | | 6.7254 | >20 |
| | | carboxylic acid | 1.46 (m, 3H), 1.45- | | | | |
| | | | 1.31 (m, 3H), 1.27 | | | | |
| | | | (s, 3H), 1.24 (s, 1H), | | | | |
| | | | 1.19-1.08 (d, J = | | | | |
| | | | 10.8 Hz, 1H), 0.97- | | | | |
| | | | 0.76 (m, 7H). | | | | |
| 202 | 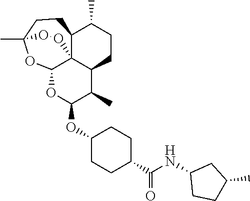 | (1R,3S)-3- ((1R,4R)-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclo- hexane-1- carboxamido) cyclopentane- | ¹H NMR (400 MHz, DMSO) δ 12.08 (s, 1H), 7.70 (d, J = 7.2 Hz, 1H), 5.32 (s, 1H), 4.76 (d, J = 3.5 Hz, 1H), 3.96 (q, J = 7.3 Hz, 1H), 3.79 (s, 1H), 2.72- 2.63 (m, 2H), 2.39 (dt, J = 7.9, 4.2 Hz, 1H), 2.24-2.13 (m, 1H), 2.13-2.03 (m, 2H), 1.99 (d, J = 13.9 Hz, 1H), 1.90- | | | 4.0082 | 20.00 |
| | | carboxylic acid | 1.69 (m, 7H), 1.68- | | | | |
| | | | 1.51 (m, 4H), 1.51- | | | | |
| | | | 1.33 (m, 6H), 1.32- | | | | |
| | | | 1.21 (m, 5H), 1.22- | | | | |
| | | | 1.10 (m, 1H), 0.90 | | | | |
| | | | (d, J = 6.2 Hz, 3H), | | | | |
| | | | 0.85 (d, J = 7.3 Hz, | | | | |
| | | | 3H). | | | | |
| 203 | 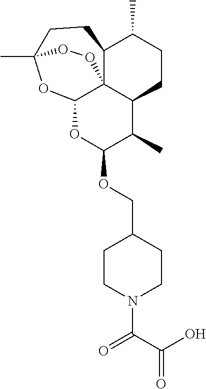 [SMILES: C[C@H]1[C@@H](OCC2CCN(C(=O)C(=O)O)CC2)O[C@@H]2O[C@@]3(C)CC[C@H]4[C@H](C)CC[C@@H]1[C@@]24OO3] | 2-oxo-2-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) piperidin-1- yl)acetic acid | | | | 17.4859 | >20 |
| 204 | 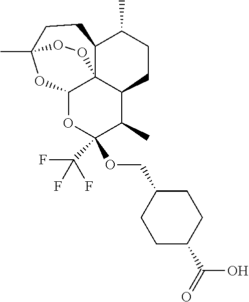 [SMILES: C[C@@H]1CC[C@H]2[C@@H](C)[C@](OC[C@H]3CC[C@@H](C(=O)O)CC3)(C(F)(F)F)O[C@@H]3O[C@@]4(C)CC[C@@H]1[C@]32OO4] | (1S,4s)-4- ((((3R,5aS,6R,8aS, 9R,10R,12R, 12aR)-3,6,9- trimethyl-10- (trifluoromethyl) decahydro-12H- 3,12-epoxy[1,2] dioxepino[4,3-i] isochromen-10- yl)oxy)methyl) cyclohexane-1- carboxylic acid | ¹H NMR (400 MHz, DMSO) δ 12.08 (s, 1H), 5.42 (s, 1H), 3.59 (dd, J = 9.3, 5.7 Hz, 1H), 2.68-2.60 (m, 1H), 2.48-2.44 (m, 1H), 2.20 (td, J = 13.9, 3.9 Hz, 1H), 2.03 (d, J = 15.1 Hz, 1H), 1.91- 1.78 (m, 3H), 1.74- 1.56 (m, 5H), 1.55- 1.34 (m, 7H), 1.30 (s, 3H), 1.28-1.13 (m, 4H), 0.99-0.81 (m, 7H). | | | | |
| 205 | 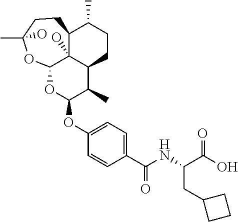 [SMILES: C[C@H]1[C@@H](Oc2ccc(C(=O)N[C@@H](CC3CCC3)C(=O)O)cc2)O[C@@H]2O[C@@]3(C)CC[C@H]4[C@H](C)CC[C@@H]1[C@@]24OO3] | (S)-3- cyclobutyl-2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) benzamido) propanoic acid | | | | 0.0351 | 0.11 |
| 206 | 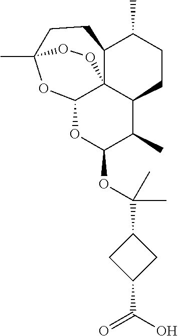 [SMILES: C[C@H]1[C@@H](OC(C)(C)[C@H]2C[C@@H](C(=O)O)C2)O[C@@H]2O[C@@]3(C)CC[C@H]4[C@H](C)CC[C@@H]1[C@@]24OO3] | (1R,3s)-3-(2- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)propan-2- yl)cyclobutane- 1-carboxylic acid | | | | 3.8318 | >20 |
| 207 | 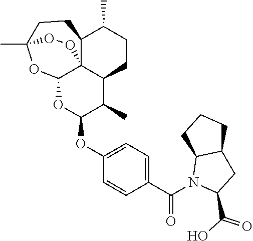 [SMILES: C[C@H]1[C@@H](Oc2ccc(C(=O)N3[C@H](C(=O)O)C[C@@H]4CCC[C@@H]43)cc2)O[C@@H]2O[C@@]3(C)CC[C@H]4[C@H](C)CC[C@@H]1[C@@]24OO3] | (2S,3aS,6aS)-1- (4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzoyl) octahydrocyclo penta[b]pyrrole- 2-carboxylic acid | | | | 0.1499 | 0.68 |
| 208 | 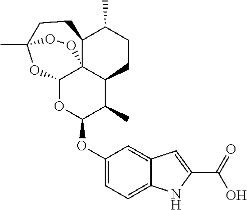 [SMILES: C[C@H]1[C@@H](Oc2ccc3[nH]c(C(=O)O)cc3c2)O[C@@H]2O[C@@]3(C)CC[C@H]4[C@H](C)CC[C@@H]1[C@@]24OO3] | 5- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)-1H- indole-2- carboxylic acid | ¹H NMR (400 MHz, DMSO) δ 12.92 (s, 1H), 11.67 (s, 1H), 7.35 (d, J = 8.9 Hz, 1H), 7.31 (d, J = 2.3 Hz, 1H), 7.02 (d, J = 2.8 Hz, 1H), 7.00 (dd, J = 8.9, 2.4 Hz, 1H), 5.52 (s, 1H), 5.44 (d, J = 3.4 Hz, 1H), 2.59 (dt, J = 7.8, 4.3 Hz, 1H), 2.20 (td, J = 14.0, 3.8 Hz, 1H), 2.07- 1.93 (m, 2H), 1.83 | | | 0.0693 | 0.37 |
| | | | (d, J = 13.7 Hz, 2H), | | | | |
| | | | 1.69-1.60 (m, 1H), | | | | |
| | | | 1.57-1.48 (m, 1H), | | | | |
| | | | 1.46-1.38 (m, 1H), | | | | |
| | | | 1.35 (d, J = 14.0 Hz, | | | | |
| | | | 1H), 1.27 (s, 3H), | | | | |
| | | | 1.25-1.19 (m, 1H), | | | | |
| | | | 1.01 (d, J = | | | | |
| | | | 7.3 Hz, 3H), 0.98- | | | | |
| | | | 0.86 (m, 4H). | | | | |
| 209 | 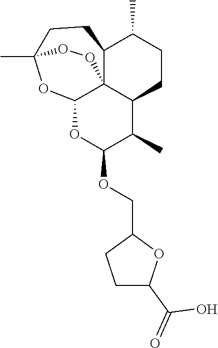 [SMILES: C[C@H]1[C@@H](OCC2CCC(C(=O)O)O2)O[C@@H]2O[C@@]3(C)CC[C@H]4[C@H](C)CC[C@@H]1[C@@]24OO3] | 5- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) tetrahydrofuran- 2-carboxylic acid | | | | 15.8345 | >20 |
| 210 | 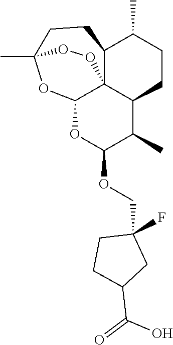 [SMILES: C[C@H]1[C@@H](OC[C@@]2(F)CCC(C(=O)O)C2)O[C@@H]2O[C@@]3(C)CC[C@H]4[C@H](C)CC[C@@H]1[C@@]24OO3] | (3R)-3-fluoro-3- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclopentane-1- carboxylic acid | | | | 2.4985 | 7.58 |
| 211 | 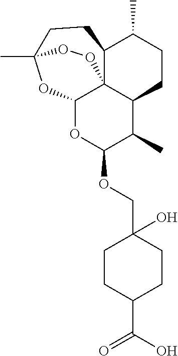 [SMILES: C[C@H]1[C@@H](OCC2(O)CCC(C(=O)O)CC2)O[C@@H]2O[C@@]3(C)CC[C@H]4[C@H](C)CC[C@@H]1[C@@]24OO3] | 4-hydroxy-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxylic acid | | | | 11.4056 | >20 |
| 212 | 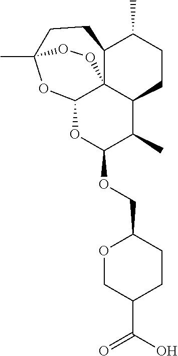 [SMILES: C[C@H]1[C@@H](OC[C@H]2CCC(C(=O)O)CO2)O[C@@H]2O[C@@]3(C)CC[C@H]4[C@H](C)CC[C@@H]1[C@@]24OO3] | (6R)-6- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) tetrahydro-2H- pyran-3- carboxylic acid | | | | 20.5180 | >20 |
| 213 | 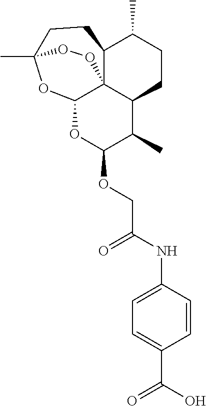 [SMILES: C[C@H]1[C@@H](OCC(=O)Nc2ccc(C(=O)O)cc2)O[C@@H]2O[C@@]3(C)CC[C@H]4[C@H](C)CC[C@@H]1[C@@]24OO3] | 4-(2- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)actamido) benzoic acid | ¹H NMR (400 MHz, DMSO) δ 12.72 (s, 1H), 10.12 (s, 1H), 7.90 (d, J = 8.7 Hz, 2H), 7.69 (d, J = 8.7 Hz, 2H), 5.53 (s, 1H), 4.79 (d, J = 3.5 Hz, 1H), 4.23 (s, 2H), 2.45 (dt, J = 7.9, 4.1 Hz, 1H), 2.23-2.13 (m, 1H), 1.99 (d, J = 15.4 Hz, 1H), 1.91 (d, J = 10.6 Hz, 1H), 1.87- 1.76 (m, 1H), 1.73- 1.65 (m, 1H), 1.59 (d, J = 12.7 Hz, 1H), 1.44-1.31 (m, 3H), 1.27 (s, 3H), 1.17 (dd, J = 11.4, 6.5 Hz, 1H), 0.96-0.86 (m, 7H). | | 484.1 | 0.3252 | >20 |
| 214 | 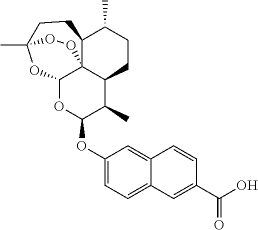 [SMILES: C[C@H]1[C@@H](Oc2ccc3cc(C(=O)O)ccc3c2)O[C@@H]2O[C@@]3(C)CC[C@H]4[C@H](C)CC[C@@H]1[C@@]24OO3] | 6- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)-2- naphthoic acid | ¹H NMR (400 MHz, DMSO) δ 8.53 (d, J = 1.5 Hz, 1H), 8.06 (d, J = 9.0 Hz, 1H), 7.94 (dd, J = 8.6, 1.6 Hz, 1H), 7.89 (d, J = 8.7 Hz, 1H), 7.60 (d, J = 2.5 Hz, 1H), 7.35 (dd, J = 8.9, 2.4 Hz, 1H), 5.81 (d, J = 3.4 Hz, 1H), 5.47 (s, 1H), 2.70-2.62 (m, 1H), 2.20 (td, J = 13.9, 3.9 Hz, 1H), 1.99 (d, | | | 0.0537 | 0.22 |
| | | | J = 14.9 Hz, 2H), | | | | |
| | | | 1.91-1.78 (m, 2H), | | | | |
| | | | 1.67 (d, J = 12.9 Hz, | | | | |
| | | | 1H), 1.62-1.53 (m, | | | | |
| | | | 1H), 1.49-1.39 (m, | | | | |
| | | | 1H), 1.38-1.33 (m, | | | | |
| | | | 1H), 1.29 (s, 3H), | | | | |
| | | | 1.22 (td, J = 11.2, | | | | |
| | | | 6.3 Hz, 1H), 1.01 (d, | | | | |
| | | | J = 7.3 Hz, 3H), | | | | |
| | | | 0.99-0.94 (m, 1H), | | | | |
| | | | 0.91 (d, J = 6.3 Hz, | | | | |
| | | | 3H). | | | | |
| 215 | 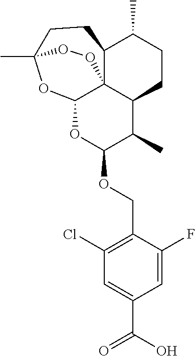 [SMILES: C[C@H]1[C@@H](OCc2c(F)cc(C(=O)O)cc2Cl)O[C@@H]2O[C@@]3(C)CC[C@H]4[C@H](C)CC[C@@H]1[C@@]24OO3] | 3-chloro-5- fluoro-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, DMSO) δ 13.52 (s, 1H), 7.82 (s, 1H), 7.70 (d, J = 9.4 Hz, 1H), 5.40 (s, 1H), 4.91-4.82 (m, 2H), 4.55 (d, J = 10.9 Hz, 1H), 2.43 (dt, J = 8.3, 4.5 Hz, 1H), 2.25-2.12 (m, 1H), 2.01 (d, J = 14.5 Hz, 1H), 1.80 (d, J = 13.4 Hz, 1H), 1.66- 1.46 (m, 3H), 1.42- 1.33 (m, 2H), 1.31 (s, 3H), 1.28-1.11 (m, 3H), 0.88 (d, J = 6.2 Hz, 3H), 0.85-0.80 (m, 1H), 0.78 (d, J = 7.3 Hz, 3H). | | | 0.0665 | 5.51 |
| 216 | 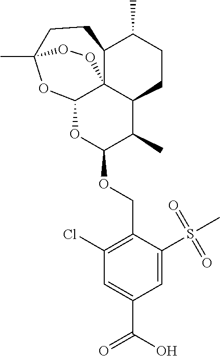 | 3-chloro-5- (methylsulfonyl)- 4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, DMSO) δ 13.82 (s, 1H), 8.44 (d, J = 1.7 Hz, 1H), 8.26 (d, J = 1.8 Hz, 1H), 5.58 (s, 1H), 5.37 (d, J = 10.8 Hz, 1H), 5.07 (d, J = 4.2 Hz, 1H), 4.87 (d, J = 10.7 Hz, 1H), 3.39 (s, 3H), 2.22-2.10 (m, 1H), 2.06-1.96 (m, 1H), 1.82 (s, 1H), 1.62 (t, J = 12.5 Hz, 1H), 1.53-1.39 (m, 4H), 1.35 (s, 3H), 1.26 (dd, J = 12.2, 7.1 Hz, 2H), 1.19 (dd, J = 11.1, 6.2 Hz, 1H), 1.12 (d, J = 7.3 Hz, 3H), 0.98-0.81 (m, 5H). | | | 7.1794 | >20 |
| 217 | 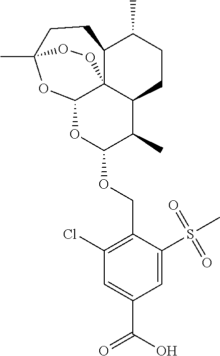 | 3-chloro-5- (methylsulfonyl)- 4- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) benzoic acid | | | | 0.0531 | 3.00 |
| 218 | 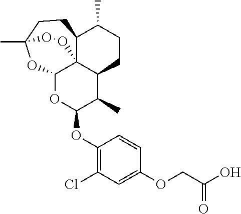 [SMILES: C[C@H]1[C@@H](Oc2ccc(OCC(=O)O)cc2Cl)O[C@@H]2O[C@@]3(C)CC[C@H]4[C@H](C)CC[C@@H]1[C@@]24OO3] | 2-(3-chloro-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenoxy) acetic acid | ¹H NMR (400 MHz, DMSO) δ 13.05 (s, 1H), 7.27 (d, J = 9.1 Hz, 1H), 7.03 (d, J = 3.0 Hz, 1H), 6.91 (dd, J = 9.1, 3.0 Hz, 1H), 5.53 (d, J = 3.5 Hz, 1H), 5.51 (s, 1H), 4.66 (s, 2H), 2.60 (dq, J = 11.4, 5.7 Hz, 1H), 2.19 (td, J = 13.9, 3.9 Hz, 1H), 2.10-1.96 (m, 2H), 1.81 (dq, J = | | | 0.1013 | 0.69 |
| | | | 10.9, 4.1 Hz, 2H), | | | | |
| | | | 1.63 (dd, J = 13.2, | | | | |
| | | | 3.3 Hz, 1H), 1.53 | | | | |
| | | | (dt, J = 13.6, 4.6 | | | | |
| | | | Hz, 1H), 1.37 (qd, | | | | |
| | | | J = 13.0, 4.8 Hz, | | | | |
| | | | 2H), 1.28 (s, 3H), | | | | |
| | | | 1.21 (td, J = 11.4, | | | | |
| | | | 6.4 Hz, 1H), 1.01 | | | | |
| | | | (d, J = 7.3 Hz, 3H), | | | | |
| | | | 0.98-0.93 (s, 1H), | | | | |
| | | | 0.91 (d, J = 6.4 Hz, | | | | |
| | | | 3H). | | | | |
| 219 | 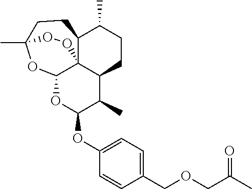 [SMILES: CC(=O)COCc1ccc(O[C@H]2O[C@@H]3O[C@@]4(C)CC[C@H]5[C@H](C)CC[C@@H]([C@H]2C)[C@@]35OO4)cc1] | 2-((4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzyl) oxy)acetic acid | ¹H NMR (400 MHz, DMSO) δ 12.67 (s, 1H), 7.30 (d, J = 8.5 Hz, 2H), 7.06 (d, J = 8.6 Hz, 2H), 5.56 (d, J = 3.4 Hz, 1H), 5.43 (s, 1H), 4.48 (s, 2H), 4.04 (s, 2H), 2.61 (dq, J = 7.8, 4.0 Hz, 1H), 2.21 (td, J = 13.9, 3.9 Hz, 1H), 2.01 (dt, | | | 0.0620 | 13.12 |
| | | | J = 14.3, 3.9 Hz, | | | | |
| | | | 1H), 1.93 (dd, J = | | | | |
| | | | 13.6, 3.4 Hz, 1H), | | | | |
| | | | 1.84 (ddt, J = 14.6, | | | | |
| | | | 9.5, 4.4 Hz, 1H), | | | | |
| | | | 1.65 (dd, J = 13.1, | | | | |
| | | | 3.4 Hz, 1H), 1.54 | | | | |
| | | | (dt, J = 13.3, 4.7 | | | | |
| | | | Hz, 1H), 1.48-1.35 | | | | |
| | | | (m, 2H), 1.30 (s, | | | | |
| | | | 3H), 1.22 (td, J = | | | | |
| | | | 11.2, 6.3 Hz, 1H), | | | | |
| | | | 1.01-0.88 (m, 7H). | | | | |
| 220 | 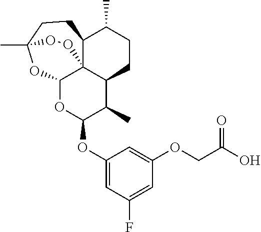 [SMILES: C[C@H]1[C@@H](Oc2cc(F)cc(OCC(=O)O)c2)O[C@@H]2O[C@@]3(C)CC[C@H]4[C@H](C)CC[C@@H]1[C@@]24OO3] | 2-(3-fluoro-5- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenoxy) acetic acid | ¹H NMR (400 MHz, DMSO) δ 12.96 (s, 1H), 6.57 (dt, J = 10.7, 2.2 Hz, 1H), 6.52-6.43 (m, 2H), 5.59 (d, J = 3.4 Hz, 1H), 5.41 (s, 1H), 4.69 (s, 2H), 2.64- 2.52 (m, 1H), 2.19 (td, J = 13.7, 3.8 Hz, 1H), 2.00 (ddd, J = 14.7, 5.3, 3.0 Hz, 1H), 1.81 (td, J = 9.9, 3.9 Hz, 3H), 1.62 (dd, | | | 0.0708 | 1.88 |
| | | | J = 13.1, 3.4 Hz, | | | | |
| | | | 1H), 1.51 (dt, J = | | | | |
| | | | 12.6, 4.9 Hz, 1H), | | | | |
| | | | 1.46-1.32 (m, 2H), | | | | |
| | | | 1.29 (s, 3H), 1.26- | | | | |
| | | | 1.12 (m, 2H), 0.94 | | | | |
| | | | (d, J = 7.3 Hz, 3H), | | | | |
| | | | 0.90 (d, J = 6.3 Hz, | | | | |
| | | | 3H). | | | | |
| 221 | 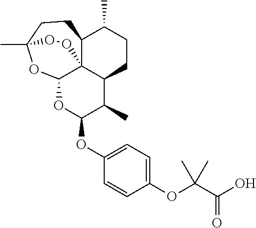 [SMILES: C[C@H]1[C@@H](Oc2ccc(OC(C)(C)C(=O)O)cc2)O[C@@H]2O[C@@]3(C)CC[C@H]4[C@H](C)CC[C@@H]1[C@@]24OO3] | 2-methyl-2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenoxy) propanoic acid | | | | 0.1236 | 9.61 |
| 222 | 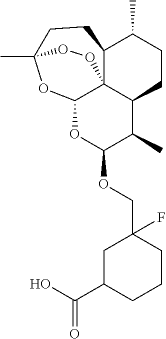 [SMILES: C[C@H]1[C@@H](OCC2(F)CCCC(C(=O)O)C2)O[C@@H]2O[C@@]3(C)CC[C@H]4[C@H](C)CC[C@@H]1[C@@]24OO3] | 3-fluoro-3- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxylic acid | | | | 0.1337 | 19.00 |
| 223 | 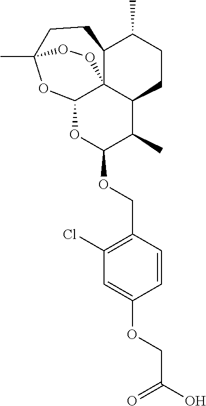 [SMILES: C[C@H]1[C@@H](OCc2ccc(OCC(=O)O)cc2Cl)O[C@@H]2O[C@@]3(C)CC[C@H]4[C@H](C)CC[C@@H]1[C@@]24OO3] | 2-(3-chloro-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) phenoxy)acetic acid | ¹H NMR (400 MHz, DMSO) δ 7.39 (d, J = 8.5 Hz, 1H), 7.04 (d, J = 2.6 Hz, 1H), 6.92 (dd, J = 8.5, 2.6 Hz, 1H), 5.45 (s, 1H), 4.84 (d, J = 3.5 Hz, 1H), 4.77 (d, J = 11.8 Hz, 1H), 4.34 (d, J = 11.8 Hz, 1H), 2.50- 2.41 (m, 1H), 2.19 (td, J = 13.9, 3.9 Hz, 1H), 2.04-1.96 (m, 1H), 1.86-1.77 (m, 1H), 1.73 (dd, J = 13.6, 3.4 Hz, 1H), 1.65 (ddd, J = 13.7, 10.7, 3.6 Hz, 1H), 1.53 (dd, J = 13.0, 3.3 Hz, 1H), 1.39 (dq, J = 13.0, 4.3 Hz, 2H), 1.34-1.26 (m, 4H), 1.23 (s, 1H), 1.20- | | | 0.1110 | 2.84 |
| | | | 1.09 (m, 1H), 0.91- | | | | |
| | | | 0.79 (m, 7H). | | | | |
| 224 | 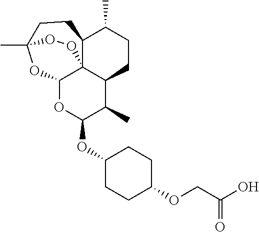 [SMILES: C[C@H]1[C@@H](O[C@H]2CC[C@@H](OCC(=O)O)CC2)O[C@@H]2O[C@@]3(C)CC[C@H]4[C@H](C)CC[C@@H]1[C@@]24OO3] | 2-(((1R,4s)-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclohexyl) oxy)acetic acid | | | | 8.0366 | >20 |
| 225 | 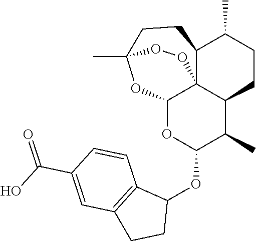 [SMILES: C[C@H]1[C@H](OC2CCc3cc(C(=O)O)ccc32)O[C@@H]2O[C@@]3(C)CC[C@H]4[C@H](C)CC[C@@H]1[C@@]24OO3] | 1- (((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)-2,3- dihydro-1H- indene-5- carboxylic acid | ¹H NMR (400 MHz, DMSO) δ 7.80 (s, 1H), 7.78 (d, J = 8.4 Hz, 1H), 7.65 (d, J = 7.9 Hz, 1H), 5.52 (s, 1H), 5.26 (t, J = 6.7 Hz, 1H), 4.73 (d, J = 9.1 Hz, 1H), 3.03- 2.91 (m, 1H), 2.79 (dt, J = 15.8, 7.8 Hz, 1H), 2.44 (dp, J = 12.2, 3.8 Hz, 1H), 2.27-2.09 (m, 2H), 2.02 (d, J = 15.5 Hz, 1H), 1.92 | | | 0.2034 | 20.00 |
| | | | (dt, J = 13.4, 7.2 Hz, | | | | |
| | | | 1H), 1.87-1.80 (m, | | | | |
| | | | 1H), 1.63 (t, J = | | | | |
| | | | 11.1 Hz, 2H), 1.46 | | | | |
| | | | (d, J = 9.2 Hz, 2H), | | | | |
| | | | 1.43-1.37 (m, 1H), | | | | |
| | | | 1.35 (s, 3H), 1.23 (s, | | | | |
| | | | 1H), 1.21-1.14 (m, | | | | |
| | | | 1H), 0.96 (d, J = | | | | |
| | | | 12.1 Hz, 3H), 0.80 | | | | |
| | | | (d, J = 7.1 Hz, 3H). | | | | |
| 226 | 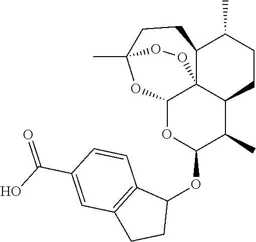 [SMILES: C[C@H]1[C@@H](OC2CCc3cc(C(=O)O)ccc32)O[C@@H]2O[C@@]3(C)CC[C@H]4[C@H](C)CC[C@@H]1[C@@]24OO3] | 1- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)-2,3- dihydro-1H- indene-5- carboxylic acid | ¹H NMR (400 MHz, DMSO) δ 12.88 (s, 1H), 7.81 (d, J = 7.7 Hz, 2H), 7.41 (d, J = 7.9 Hz, 1H), 5.39 (s, 1H), 5.13-5.04 (m, 2H), 3.30-3.23 (m, 1H), 2.96 (ddd, J = 16.0, 8.7, 3.8 Hz, 1H), 2.80 (dt, J = 15.8, 7.8 Hz, 1H), 2.50-2.42 (m, 1H), 2.19 (td, J = 13.9, 3.8 Hz, 1H), 1.98 (td, J = 15.0, 7.9 Hz, | | | 0.0512 | 12.00 |
| | | | 2H), 1.86-1.75 | | | | |
| | | | (m, 1H), 1.70-1.55 | | | | |
| | | | (m, 2H), 1.54-1.46 | | | | |
| | | | (m, 1H), 1.45-1.34 | | | | |
| | | | (m, 2H), 1.34-1.25 | | | | |
| | | | (m, 4H), 1.15 (td, | | | | |
| | | | J = 11.4, 6.5 Hz, | | | | |
| | | | 1H), 0.92-0.77 (m, | | | | |
| | | | 7H). | | | | |
| 227 | 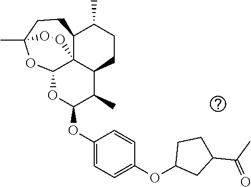 [SMILES: CC(=O)C1CCC(Oc2ccc(O[C@H]3O[C@@H]4O[C@@]5(C)CC[C@H]6[C@H](C)CC[C@@H]([C@H]3C)[C@@]46OO5)cc2)C1] | 3-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenoxy) cyclopentane-1- carboxylic acid | ¹H NMR (400 MHz, DMSO) δ 12.16 (s, 1H), 7.04-6.95 (m, 1H), 6.89-6.80 (m, 1H), 5.45 (s, 1H), 5.37 (d, J = 3.4 Hz, 1H), 4.84-4.77 (m, 1H), 2.89 (p, J = 8.0 Hz, 1H), 2.55 (dt, J = 7.7, 4.2 Hz, 1H), 2.19 (td, J = 14.0, 3.9 Hz, 1H), 2.09- 1.93 (m, 5H), 1.92- | | | 0.0329 | 0.70 |
| | | | 1.67 (m, 5H), 1.66- | | | | |
| | | | 1.58 (m, 1H), 1.50 | | | | |
| | | | (dt, J = 13.3, 4.6 | | | | |
| | | | Hz, 1H), 1.46-1.34 | | | | |
| | | | (m, 2H), 1.27 (s, | | | | |
| | | | 3H), 1.24-1.09 (m, | | | | |
| | | | 1H), 0.96 (d, J = 7.3 | | | | |
| | | | Hz, 3H), 0.91 (d, J = | | | | |
| | | | 6.4 Hz, 3H). | | | | |
| 228 | 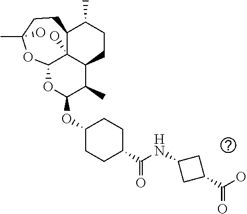 [SMILES: C[C@H]1[C@@H](O[C@H]2CC[C@@H](C(=O)N[C@H]3C[C@@H](C(=O)O)C3)CC2)O[C@@H]2O[C@@]3(C)CC[C@H]4[C@H](C)CC[C@@H]1[C@@]24OO3] | (1S,3s)-3- ((1R,4S)-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclo- hexane-1- carboxamido) cyclobutane-1- | ¹H NMR (400 MHz, DMSO) δ 12.13 (s, 1H), 7.99 (d, J = 7.8 Hz, 1H), 5.32 (s, 1H), 4.76 (d, J = 3.5 Hz, 1H), 4.09 (hept, J = 7.9 Hz, 1H), 3.78 (s, 1H), 2.75- 2.62 (m, 1H), 2.36 (dtt, J = 19.1, 7.9, 3.5 Hz, 3H), 2.18 (td, J = 14.0, 3.9 Hz, 1H), 2.10-1.94 (m, 4H), 1.91-1.64 (m, | | | 9.1998 | >20 |
| | | carboxylic acid | 5H), 1.63-1.52 (m, | | | | |
| | | | 3H), 1.51-1.38 (m, | | | | |
| | | | 4H), 1.38-1.22 (m, | | | | |
| | | | 6H), 1.15 (td, J = | | | | |
| | | | 11.5, 6.7 Hz, 1H), | | | | |
| | | | 0.94-0.79 (m, 7H). | | | | |
| 229 | 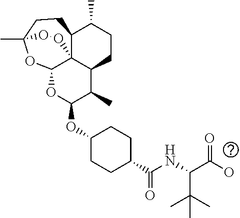 [SMILES: C[C@H]1[C@@H](O[C@H]2CC[C@@H](C(=O)N[C@H](C(=O)O)C(C)(C)C)CC2)O[C@@H]2O[C@@]3(C)CC[C@H]4[C@H](C)CC[C@@H]1[C@@]24OO3] | (S)-3,3- dimethyl-2- ((1R,4R)-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) cyclohexane-1- carboxamido) butanoic acid | | | | 0.2036 | >20 |
| 230 | 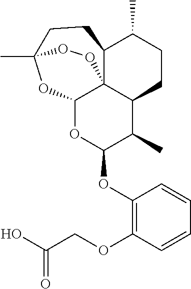 [SMILES: C[C@H]1[C@@H](Oc2ccccc2OCC(=O)O)O[C@@H]2O[C@@]3(C)CC[C@H]4[C@H](C)CC[C@@H]1[C@@]24OO3] | 2-(2- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenoxy) acetic acid | ¹H NMR (400 MHz, DMSO) δ 13.02 (s, 1H), 7.16 (dd, J = 7.8, 1.5 Hz, 1H), 7.06-6.66 (m, 3H), 5.65 (s, 1H), 5.53 (d, J = 3.4 Hz, 1H), 4.61 (s, 2H), 2.57 (dt, J = 7.8, 4.2 Hz, 1H), 2.26-2.08 (m, 2H), 1.98 (d, J = 14.4 Hz, 1H), 1.77 (ddd, J = 18.0, 13.9, 3.6 Hz, 2H), 1.68- 1.60 (m, 1H), 1.56- 1.44 (m, 2H), 1.43- 1.32 (m, 1H), 1.24 (s, 3H), 1.21-1.13 | | | 2.8700 | >20 |
| | | | (m, 1H), 1.02 (d, J = | | | | |
| | | | 7.3 Hz, 3H), 0.97- | | | | |
| | | | 0.82 (m, 4H). | | | | |
| 231 | 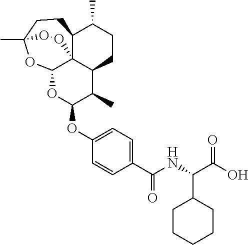 [SMILES: C[C@H]1[C@@H](Oc2ccc(C(=O)N[C@H](C(=O)O)C3CCCCC3)cc2)O[C@@H]2O[C@@]3(C)CC[C@H]4[C@H](C)CC[C@@H]1[C@@]24OO3] | (S)-2- cyclohexyl-2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) benzamido)acetic acid | ¹H NMR (400 MHz, DMSO) δ 8.20 (d, J = 8.0 Hz, 1H), 7.91-7.83 (m, 2H), 7.16-7.08 (m, 2H), 5.67 (d, J = 3.4 Hz, 1H), 5.39 (s, 1H), 4.23 (t, J = 7.5 Hz, 1H), 2.68-2.56 (m, 1H), 2.19 (td, J = 13.9, 3.9 Hz, 1H), 2.05-1.95 (m, 1H), 1.94-1.77 (m, 4H), 1.73-1.51 (m, 7H), 1.47-1.32 (m, 2H), 1.29 (s, 3H), 1.26- 1.03 (m, 6H), 0.97 (d, J = 7.3 Hz, 3H), | | | 0.0170 | 0.08 |
| | | | 0.91 (d, J = 6.3 Hz, | | | | |
| | | | 3H). | | | | |
| 232 | 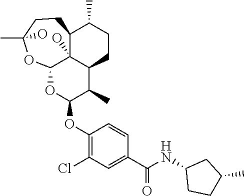 | (1R,3S)-3-(3- chloro-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) benzamido)cyclo- pentane-1- carboxylic acid | | | | 0.0299 | 0.62 |
| 233 | 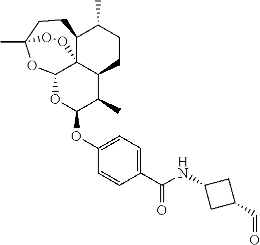 [SMILES: C[C@H]1[C@@H](Oc2ccc(C(=O)N[C@H]3C[C@@H](C=O)C3)cc2)O[C@@H]2O[C@@]3(C)CC[C@H]4[C@H](C)CC[C@@H]1[C@@]24OO3] | (1R,3s)-3-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzamido) cyclobutane-1- carboxylic acid | ¹H NMR (400 MHz, DMSO) δ 12.16 (s, 1H), 8.55 (d, J = 7.7 Hz, 1H), 7.84 (d, J = 8.3 Hz, 2H), 7.11 (d, J = 8.3 Hz, 2H), 5.67 (d, J = 3.4 Hz, 1H), 5.40 (s, 1H), 4.36 (q, J = 8.4 Hz, 1H), 2.76 (p, J = 8.9 Hz, 1H), 2.61 (s, 1H), 2.49-2.37 (m, 2H), 2.29-2.13 (m, 3H), 2.00 (d, J = 14.7 Hz, 1H), 1.90 | | | 0.0936 | 20.00 |
| | | | (d, J = 12.6 Hz, 1H), | | | | |
| | | | 1.83 (d, J = 12.4 | | | | |
| | | | Hz, 2H), 1.64 (d, J = | | | | |
| | | | 12.5 Hz, 1H), 1.53 | | | | |
| | | | (d, J = 12.8 Hz, 1H), | | | | |
| | | | 1.42-1.31 (m, 2H), | | | | |
| | | | 1.29 (s, 3H), 1.25- | | | | |
| | | | 1.11 (m, 2H), 0.96 | | | | |
| | | | (d, J = 7.3 Hz, 3H), | | | | |
| | | | 0.90 (d, J = 6.3 Hz, | | | | |
| | | | 3H). | | | | |
| 234 | 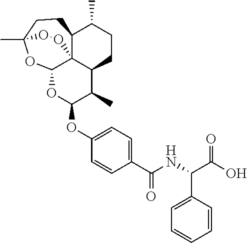 [SMILES: C[C@H]1[C@@H](Oc2ccc(C(=O)N[C@H](C(=O)O)c3ccccc3)cc2)O[C@@H]2O[C@@]3(C)CC[C@H]4[C@H](C)CC[C@@H]1[C@@]24OO3] | (S)-2-phenyl-2- (4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzamido) acetic acid | ¹H NMR (400 MHz, DMSO) δ 12.89 (s, 1H), 8.89 (d, J = 7.6 Hz, 1H), 7.92 (d, J = 8.3 Hz, 2H), 7.49 (d, J = 7.5 Hz, 2H), 7.42-7.31 (m, 3H), 7.12 (d, J = 8.3 Hz, 2H), 5.68 (s, 1H), 5.58 (d, J = 7.4 Hz, 1H), 5.40 (s, 1H), 2.61 (s, 1H), 2.19 (t, J = 13.8 Hz, 1H), 2.00 (d, J = 14.3 Hz, 1H), 1.94-1.75 (m, 3H), 1.64 (d, J = | | | 0.1118 | 1.27 |
| | | | 12.5 Hz, 1H), 1.55 | | | | |
| | | | (s, 0H), 1.42-1.31 | | | | |
| | | | (m, 2H), 1.28 (s, | | | | |
| | | | 3H), 1.23 (s, 3H), | | | | |
| | | | 0.97 (d, J = 7.3 Hz, | | | | |
| | | | 3H), 0.90 (d, J = 6.3 | | | | |
| | | | Hz, 3H). | | | | |
| 235 | 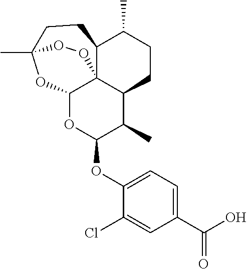 [SMILES: C[C@H]1[C@@H](Oc2ccc(C(=O)O)cc2Cl)O[C@@H]2O[C@@]3(C)CC[C@H]4[C@H](C)CC[C@@H]1[C@@]24OO3] | 3-chloro-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzoic acid | ¹H NMR (400 MHz, DMSO) δ 13.06 (s, 1H), 7.97-7.87 (m, 2H), 7.49-7.42 (m, 1H), 5.81 (d, J = 3.4 Hz, 1H), 5.44 (s, 1H), 2.66 (dt, J = 7.8, 4.3 Hz, 1H), 2.20 (td, J = 13.9, 3.9 Hz, 1H), 2.12- 1.96 (m, 2H), 1.89- 1.77 (m, 2H), 1.64 (dd, J = 13.1, 3.3 Hz, 1H), 1.57 (dt, J = 13.6, 4.6 Hz, 1H), 1.47-1.32 (m, 2H), 1.30 (s, 3H), 1.23 (ddd, J = 13.3, | | | 0.0375 | 15.00 |
| | | | 10.0, 3.6 Hz, 2H), | | | | |
| | | | 1.00 (d, J = 7.3 Hz, | | | | |
| | | | 3H), 0.98-0.92 (m, | | | | |
| | | | 1H), 0.90 (d, J = 6.3 | | | | |
| | | | Hz, 3H). | | | | |
| 236 | 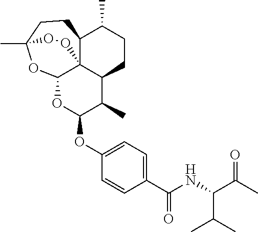 [SMILES: CC(=O)[C@@H](NC(=O)c1ccc(O[C@H]2O[C@@H]3O[C@@]4(C)CC[C@H]5[C@H](C)CC[C@@H]([C@H]2C)[C@@]35OO4)cc1)C(C)C] | (4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzoyl)- L-valine | | | | 0.0474 | 9.10 |
| 237 | 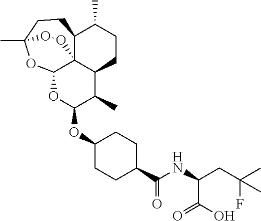 [SMILES: C[C@H]1[C@@H](O[C@H]2CC[C@@H](C(=O)N[C@@H](CC(C)(C)F)C(=O)O)CC2)O[C@@H]2O[C@@]3(C)CC[C@H]4[C@H](C)CC[C@@H]1[C@@]24OO3] | (S)-4,4,4- trifluoro-2- ((1R,4R)-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclo- hexane-1- carboxamido) | ¹H NMR (400 MHz, DMSO) δ 13.01 (s, 1H), 8.24 (d, J = 8.2 Hz, 1H), 5.32 (s, 1H), 4.77 (d, J = 3.5 Hz, 1H), 4.47 (t, J = 8.2 Hz, 1H), 3.79 (s, 1H), 2.81 (d, J = 64 Hz, 1H), 2.78 (ddd, J = 15.0, 11.4, 3.7 Hz, 1H), 2.64 (dt, J = 15.3, 10.5 Hz, 1H), 2.39 (dt, J = 7.9, 4.1 Hz, 1H), | | | 7.2605 | >20 |
| | | butanoic acid | 2.24-2.11 (m, 2H), | | | | |
| | | | 1.99 (d, J = 14.3 | | | | |
| | | | Hz, 1H), 1.91-1.72 | | | | |
| | | | (m, 4H), 1.73-1.43 | | | | |
| | | | (m, 7H), 1.42-1.30 | | | | |
| | | | (m, 3H), 1.27 (s, | | | | |
| | | | 3H), 1.21-1.09 (m, | | | | |
| | | | 1H), 0.94-0.88 (m, | | | | |
| | | | 4H), 0.85 (d, J = 7.3 | | | | |
| | | | Hz, 3H). | | | | |
| 238 | 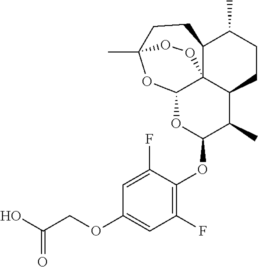 [SMILES: C[C@H]1[C@@H](Oc2c(F)cc(OCC(=O)O)cc2F)O[C@@H]2O[C@@]3(C)CC[C@H]4[C@H](C)CC[C@@H]1[C@@]24OO3] | 2-(3,5-difluoro- 4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenoxy) acetic acid | ¹H NMR (400 MHz, DMSO) δ 13.14 (s, 1H), 6.91-6.80 (m, 2H), 5.62 (s, 1H), 5.21 (d, J = 3.4 Hz, 1H), 4.73 (s, 2H), 2.66-2.57 (m, 1H), 2.22 (td, J = 14.0, 3.9 Hz, 1H), 2.06- 1.98 (m, 1H), 1.91- 1.78 (m, 3H), 1.66 (dd, J = 13.2, 3.4 Hz, 1H), 1.60-1.50 (m, 1H), 1.48-1.29 (m, 2H), 1.28 (s, 3H), 1.26-1.23 (m, 1H), 1.07 (d, J = 7.3 Hz, 3H), 1.03-0.91 | | | 0.0713 | 6.91 |
| | | | (m, 4H). | | | | |
| 239 | 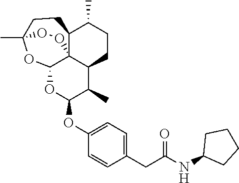 | (1R,3S)-3-(2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetamido)cyclo- pentane-1- carboxylic acid | ¹H NMR (400 MHz, DMSO) δ 12.10 (s, 1H), 8.08 (d, J = 7.3 Hz, 1H), 7.17 (d, J = 8.6 Hz, 2H), 6.98 (d, J = 8.6 Hz, 2H), 5.49 (d, J = 3.4 Hz, 1H), 5.42 (s, 1H), 3.96 (p, J = 7.3 Hz, 1H), 3.31 (d, J = 9.0 Hz, 2H), 2.76-2.64 (m, 1H), 2.58 (dt, J = 7.9, 4.3 | | | 0.0219 | 16.24 |
| | | | Hz, 1H), 2.25-2.07 | | | | |
| | | | (m, 2H), 2.03-1.96 | | | | |
| | | | (m, 1H), 1.94-1.88 | | | | |
| | | | (m, 1H), 1.87-1.74 | | | | |
| | | | (m, 5H), 1.68-1.59 | | | | |
| | | | (m, 1H), 1.58-1.48 | | | | |
| | | | (m, 2H), 1.46-1.33 | | | | |
| | | | (m, 3H), 1.28 (s, | | | | |
| | | | 3H), 1.19 (td, J = | | | | |
| | | | 11.4, 6.5 Hz, 1H), | | | | |
| | | | 1.00-0.85 (m, 7H). | | | | |
| 240 | 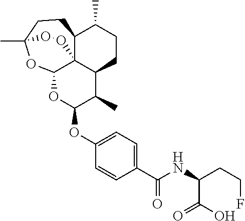 [SMILES: C[C@H]1[C@@H](Oc2ccc(C(=O)N[C@@H](CCF)C(=O)O)cc2)O[C@@H]2O[C@@]3(C)CC[C@H]4[C@H](C)CC[C@@H]1[C@@]24OO3] | (S)-4,4,4- trifluoro-2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzamido) butanoic acid | | | | 1.0196 | >20 |
| 241 | 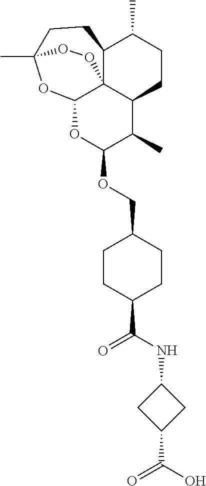 [SMILES: C[C@H]1[C@@H](OC[C@H]2CC[C@@H](C(=O)N[C@H]3C[C@@H](C(=O)O)C3)CC2)O[C@@H]2O[C@@]3(C)CC[C@H]4[C@H](C)CC[C@@H]1[C@@]24OO3] | (1S,3s)-3- ((1R,4S)-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxamido) cyclobutane-1- carboxylic acid | ¹H NMR (400 MHz, DMSO) δ 12.12 (s, 1H), 7.93 (d, J = 7.8 Hz, 1H), 5.26 (s, 1H), 4.65 (d, J = 3.4 Hz, 1H), 4.18-4.03 (m, 1H), 3.62 (dd, J = 9.4, 7.4 Hz, 1H), 3.19 (dd, J = 9.5, 6.8 Hz, 1H), 2.69 (ddd, J = 17.9, 9.9, 7.9 Hz, 1H), 2.36 (dtd, J = 9.1, 7.9, 3.4 Hz, 3H), 2.17 (td, J = 13.6, 3.8 Hz, 2H), 2.08-1.94 (m, 3H), 1.83-1.51 (m, 7H), 1.51-1.33 (m, 8H), 1.29 (s, 3H), 1.25 (d, J = 10.6 Hz, 1H), 1.12 (ddd, J = 24.8, 12.5, 6.9 Hz, 1H), 0.93- 0.76 (m, 7H). | | | 0.2139 | 18.79 |
| 242 | 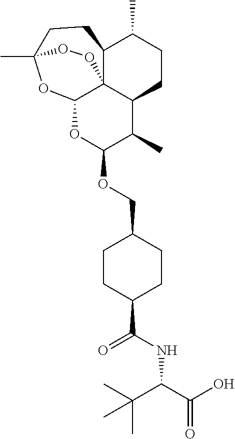 [SMILES: C[C@H]1[C@@H](OC[C@H]2CC[C@@H](C(=O)N[C@H](C(=O)O)C(C)(C)C)CC2)O[C@@H]2O[C@@]3(C)CC[C@H]4[C@H](C)CC[C@@H]1[C@@]24OO3] | (S)-3,3- dimethyl-2- ((1R,4R)-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxamido) butanoic acid | | | | 0.0213 | 1.27 |
| 243 | 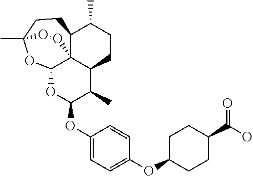 [SMILES: C[C@H]1[C@@H](Oc2ccc(O[C@H]3CC[C@@H](C(=O)O)CC3)cc2)O[C@@H]2O[C@@]3(C)CC[C@H]4[C@H](C)CC[C@@H]1[C@@]24OO3] | (1R,4s)-4-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenoxy) cyclohexane-1- carboxylic acid | | | | 0.0742 | 0.72 |
| 244 | 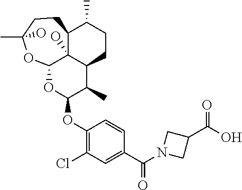 [SMILES: C[C@H]1[C@@H](Oc2ccc(C(=O)N3CC(C(=O)O)C3)cc2Cl)O[C@@H]2O[C@@]3(C)CC[C@H]4[C@H](C)CC[C@@H]1[C@@]24OO3] | 1-(3-chloro-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzoyl) azetidine-3- carboxylic acid | | 522.4 | | 0.1010 | 2.46 |
| 245 | 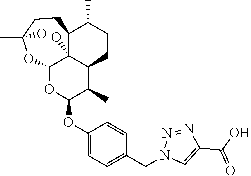 [SMILES: C[C@H]1[C@@H](Oc2ccc(Cn3cc(C(=O)O)nn3)cc2)O[C@@H]2O[C@@]3(C)CC[C@H]4[C@H](C)CC[C@@H]1[C@@]24OO3] | 1-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzyl)- 1H-1,2,3-triazole- 4-carboxylic acid | ¹H NMR (400 MHz, DMSO) δ 13.10 (s, 1H), 8.68 (s, 1H), 7.34 (d, J = 8.6 Hz, 2H), 7.06 (d, J = 8.6 Hz, 2H), 5.55 (d, J = 3.1 Hz, 3H), 5.38 (s, 1H), 2.62- 2.55 (m, 1H), 2.23- 2.12 (m, 1H), 1.99 (d, J = 14.7 Hz, 1H), 1.89 (d, J = 13.6 Hz, 1H), 1.84- | | | 1.3439 | >20 |
| | | | 1.75 (m, 2H), 1.62 | | | | |
| | | | (d, J = 12.9 Hz, 1H), | | | | |
| | | | 1.55-1.47 (m, 1H), | | | | |
| | | | 1.44-1.31 (m, 2H), | | | | |
| | | | 1.27 (s, 3H), 1.23 (s, | | | | |
| | | | 1H), 1.21-1.14 (m, | | | | |
| | | | 1H), 0.95 (d, J = 7.2 | | | | |
| | | | Hz, 3H), 0.89 (d, J = | | | | |
| | | | 6.4 Hz, 3H). | | | | |
| 246 | 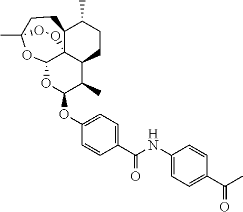 [SMILES: CC(=O)c1ccc(NC(=O)c2ccc(O[C@H]3O[C@@H]4O[C@@]5(C)CC[C@H]6[C@H](C)CC[C@@H]([C@H]3C)[C@@]46OO5)cc2)cc1] | 4-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) benzamido) benzoic acid | | 524.5 | | 0.0158 | 0.04 |
| 247 | 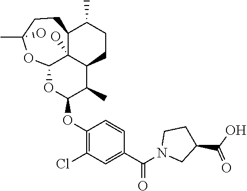 [SMILES: C[C@H]1[C@@H](Oc2ccc(C(=O)N3CC[C@@H](C(=O)O)C3)cc2Cl)O[C@@H]2O[C@@]3(C)CC[C@H]4[C@H](C)CC[C@@H]1[C@@]24OO3] | (R)-1-(3-chloro-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzoyl) pyrrolidine-3- carboxylic acid | ¹H NMR (400 MHz, DMSO) δ 12.54 (s, 1H), 7.60 (s, 1H), 7.53 (d, J = 8.8 Hz, 1H), 7.40 (d, J = 8.8 Hz, 1H), 5.76 (d, J = 3.4 Hz, 1H), 5.46 (s, 1H), 3.72-3.59 (m, 2H), 3.51 (q, J = 7.8 Hz, 2H), 3.19-2.98 (m, 1H), 2.74-2.58 (m, 1H), 2.18 (qd, | | | 0.0561 | 1.42 |
| | | | J = 13.9, 4.8 Hz, | | | | |
| | | | 2H), 2.09-1.96 (m, | | | | |
| | | | 3H), 1.84 (dd, J = | | | | |
| | | | 10.5, 3.9 Hz, 2H), | | | | |
| | | | 1.71-1.61 (m, 1H), | | | | |
| | | | 1.56 (dt, J = 13.5, | | | | |
| | | | 4.6 Hz, 1H), 1.37 (d, | | | | |
| | | | J = 11.5 Hz, 2H), | | | | |
| | | | 1.30 (s, 3H), 1.27- | | | | |
| | | | 1.15 (m, 1H), 1.01 | | | | |
| | | | (d, J = 7.3 Hz, 3H), | | | | |
| | | | 0.99-0.93 (m, 1H), | | | | |
| | | | 0.91 (d, J = 6.3 Hz, | | | | |
| | | | 3H). | | | | |
| 248 | 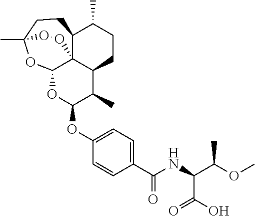 | O-methyl-N-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzoyl)- L-threonine | | | | 0.4117 | >20 |
| 249 | 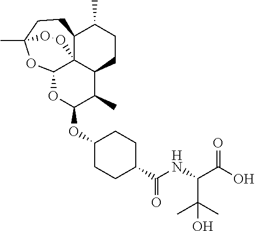 [SMILES: C[C@H]1[C@@H](O[C@H]2CC[C@@H](C(=O)N[C@H](C(=O)O)C(C)(C)O)CC2)O[C@@H]2O[C@@]3(C)CC[C@H]4[C@H](C)CC[C@@H]1[C@@]24OO3] | (S)-3-hydroxy- 3-methyl-2- ((1R,4R)-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclo- hexane-1- carboxamido) butanoic acid | ¹H NMR (400 MHz, DMSO) δ 12.39 (s, 1H), 7.60 (d, J = 8.7 Hz, 1H), 5.32 (s, 1H), 4.77 (d, J = 3.5 Hz, 1H), 4.70 (s, 1H), 4.16 (d, J = 8.7 Hz, 1H), 3.80 (s, 1H), 2.44-2.27 (m, 2H), 2.18 (td, J = 13.9, 3.9 Hz, 1H), 1.99 (d, J = 14.2 Hz, 1H), 1.91-1.73 (m, 4H), 1.72-1.44 (m, 7H), 1.44-1.30 (m, 4H), 1.29-1.22 (m, | | | 20.0000 | >20 |
| | | | 4H), 1.16 (s, 3H), | | | | |
| | | | 1.13 (s, 3H), 0.90 (d, | | | | |
| | | | J = 6.2 Hz, 3H), .94- | | | | |
| | | | 0.87 (m, 4H), 0.85 | | | | |
| | | | (d, J = 7.3 Hz, 3H). | | | | |
| 250 | 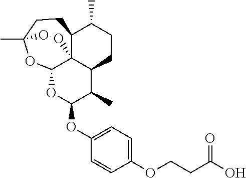 [SMILES: C[C@H]1[C@@H](Oc2ccc(OCCC(=O)O)cc2)O[C@@H]2O[C@@]3(C)CC[C@H]4[C@H](C)CC[C@@H]1[C@@]24OO3] | 3-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenoxy) propanoic acid | ¹H NMR (400 MHz, DMSO) δ 12.35 (s, 1H), 6.99 (d, J = 9.0 Hz, 2H), 6.91-6.84 (m, 2H), 5.46 (s, 1H), 5.37 (d, J = 3.4 Hz, 1H), 4.11 (t, J = 6.1 Hz, 2H), 2.66 (t, J = 6.1 Hz, 2H), 2.58-2.51 (m, 1H), 2.19 (dd, J = 14.1, 10.2 | | | 0.0882 | 17.60 |
| | | | Hz, 1H), 2.00 (d, J = | | | | |
| | | | 10.9 Hz, 1H), 1.90 | | | | |
| | | | (d, J = 12.0 Hz, 0H), | | | | |
| | | | 1.85-1.75 (s, 2H), | | | | |
| | | | 1.62 (d, J = 12.1 Hz, | | | | |
| | | | 1H), 1.55-1.46 (m, | | | | |
| | | | 1H), 1.45-1.32 (m, | | | | |
| | | | 2H), 1.28 (s, 3H), | | | | |
| | | | 1.25-1.12 (m, 2H), | | | | |
| | | | 0.97 (d, J = 7.3 Hz, | | | | |
| | | | 3H), 0.91 (d, J = 6.3 | | | | |
| | | | Hz, 3H). | | | | |
| 251 | 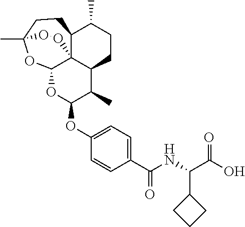 [SMILES: C[C@H]1[C@@H](Oc2ccc(C(=O)N[C@H](C(=O)O)C3CCC3)cc2)O[C@@H]2O[C@@]3(C)CC[C@H]4[C@H](C)CC[C@@H]1[C@@]24OO3] | (S)-2- cyclobutyl-2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) benzamido) acetic acid | | | | 0.0982 | 1.52 |
| 252 | 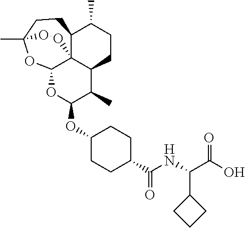 [SMILES: C[C@H]1[C@@H](O[C@H]2CC[C@@H](C(=O)N[C@H](C(=O)O)C3CCC3)CC2)O[C@@H]2O[C@@]3(C)CC[C@H]4[C@H](C)CC[C@@H]1[C@@]24OO3] | (S)-2- cyclobutyl-2- ((1R,4R)-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) cyclohexane-1- carboxamido) acetic acid | ¹H NMR (400 MHz, DMSO) δ 12.38 (s, 1H), 7.88 (d, J = 8.1 Hz, 1H), 5.32 (s, 1H), 4.77 (d, J = 3.5 Hz, 1H), 4.14 (t, J = 8.3 Hz, 1H), 3.80 (s, 1H), 2.57 (t, J = 8.2 Hz, 1H), 2.39 (dt, J = 7.9, 4.0 Hz, 1H), 2.25 (q, J = 10.5 Hz, 1H), 2.16 (dd, J = 13.8, 3.9 Hz, 1H), 1.99 (d, J = 13.7 Hz, 1H), 1.94- 1.86 (m, 1H), 1.87- | | | 0.2422 | >20 |
| | | | 1.73 (m, 7H), 1.72- | | | | |
| | | | 1.64 (m, 2H), 1.63- | | | | |
| | | | 1.53 (m, 3H), 1.53- | | | | |
| | | | 1.44 (m, 3H), 1.43- | | | | |
| | | | 1.34 (m, 3H), 1.34- | | | | |
| | | | 1.29 (m, 2H), 1.27 | | | | |
| | | | (s, 3H), 1.21-1.09 | | | | |
| | | | (m, 1H), 0.90 (d, J = | | | | |
| | | | 6.2 Hz, 3H), 0.85 | | | | |
| | | | (d, J = 7.3 Hz, 3H). | | | | |
| 253 | 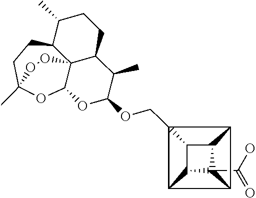 | (2S,3S,5S,6S,7S, 8S)-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cubane-1- carboxylic acid | ¹H NMR (400 MHz, DMSO) δ 12.25 (s, 1H), 5.28 (s, 1H), 4.71 (d, J = 3.4 Hz, 1H), 4.02 (dd, J = 5.6, 4.1 Hz, 3H), 3.82-3.73 (m, 4H), 3.56 (d, J = 11.5 Hz, 1H), 2.40 (ddd, J = 7.8, 5.9, 3.4 Hz, 1H), 2.18 (td, J = 14.0, 3.9 Hz, 1H), 1.99 (ddd, J = 14.6, | | | 0.2372 | >20 |
| | | | 4.9, 2.8 Hz, 1H), | | | | |
| | | | 1.86-1.77 (m, 1H), | | | | |
| | | | 1.76-1.61 (m, 2H), | | | | |
| | | | 1.56 (dd, J = 13.1, | | | | |
| | | | 3.3 Hz, 1H), 1.48- | | | | |
| | | | 1.30 (m, 3H), 1.28 | | | | |
| | | | (s, 3H), 1.14 (td, J = | | | | |
| | | | 11.4, 6.5 Hz, 1H), | | | | |
| | | | 0.95-0.80 (m, 7H). | | | | |
| 254 | 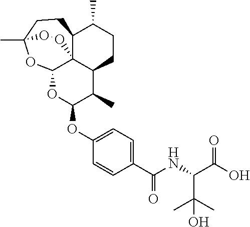 [SMILES: C[C@H]1[C@@H](Oc2ccc(C(=O)N[C@H](C(=O)O)C(C)(C)O)cc2)O[C@@H]2O[C@@]3(C)CC[C@H]4[C@H](C)CC[C@@H]1[C@@]24OO3] | (S)-3-hydroxy- 3-methyl-2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) benzamido) butanoic acid | | | | 4.5750 | >20 |
| 255 | 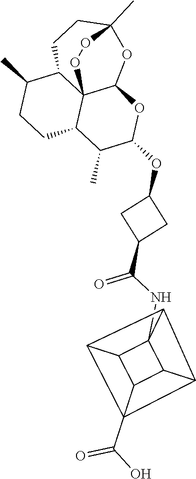 [SMILES: C[C@H]1[C@@H](O[C@H]2C[C@@H](C(=O)NC34C5C6C3C3C4C5C63C(=O)O)C2)O[C@@H]2O[C@@]3(C)CC[C@H]4[C@H](C)CC[C@@H]1[C@@]24OO3] | 4-((1R,3s)-3- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclo- butane-1- carboxamido) cubane-1- carboxylic acid | | | | 2.3186 | >20 |
| 256 | 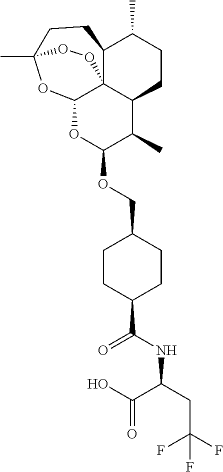 [SMILES: C[C@H]1[C@@H](OC[C@H]2CC[C@@H](C(=O)N[C@@H](CC(F)(F)F)C(=O)O)CC2)O[C@@H]2O[C@@]3(C)CC[C@H]4[C@H](C)CC[C@@H]1[C@@]24OO3] | (S)-4,4,4- trifluro-2- ((1R,4R)-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxamido) butanoic acid | | | | 3.0687 | 15.30 |
| 257 | 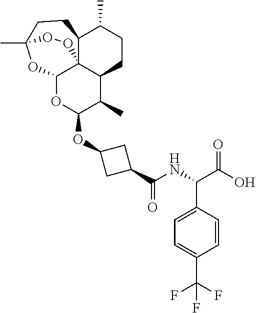 [SMILES: C[C@H]1[C@@H](O[C@H]2C[C@@H](C(=O)N[C@H](C(=O)O)c3ccc(C(F)(F)F)cc3)C2)O[C@@H]2O[C@@]3(C)CC[C@H]4[C@H](C)CC[C@@H]1[C@@]24OO3] | (S)-2-(4- (trifluoromethyl) phenyl)-2- ((1R,3R)-3- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclo- butane-1- carboxamido) acetic acid | | | | 3.2137 | 11.50 |
| 258 | 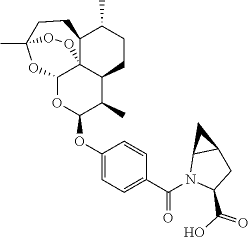 [SMILES: C[C@H]1[C@@H](Oc2ccc(C(=O)N3[C@H](C(=O)O)C[C@@H]4C[C@@H]43)cc2)O[C@@H]2O[C@@]3(C)CC[C@H]4[C@H](C)CC[C@@H]1[C@@]24OO3] | (1S,3S,5S)-2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzoyl)- 2-azabicyclo [3.1.0]hexane-3- carboxylic acid | ¹H NMR (400 MHz, DMSO) δ 12.69 (s, 1H), 7.70 (d, J = 8.4 Hz, 2H), 7.15 (d, J = 8.3 Hz, 2H), 5.65 (d, J = 3.4 Hz, 1H), 5.43 (s, 1H), 4.84 (dd, J = 11.7, 3.6 Hz, 1H), 2.76-2.56 (m, 2H), 2.20 (t, J = 13.6 Hz, 1H), 2.04- 1.88 (m, 3H), 1.87- 1.77 (m, 2H), 1.71 (d, J = 7.0 Hz, 1H), 1.64 (d, J = 12.4 Hz, 1H), 1.58-1.49 (m, 1H), 1.47-1.32 (m, | | | >20 | >20 |
| | | | 2H), 1.29 (s, 3H), | | | | |
| | | | 1.22 (dd, J = 11.3, | | | | |
| | | | 6.3 Hz, 1H), 1.04- | | | | |
| | | | 0.70 (m, 10H). | | | | |
| 259 | 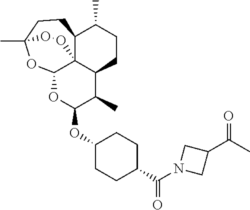 [SMILES: CC(=O)C1CN(C(=O)[C@H]2CC[C@@H](O[C@H]3O[C@@H]4O[C@@]5(C)CC[C@H]6[C@H](C)CC[C@@H]([C@H]3C)[C@@]46OO5)CC2)C1] | 1-((1R,4s)-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)cyclo- hexane-1- carbonyl) azetidine-3- carboxylic acid | ¹H NMR (400 MHz, DMSO) δ 12.74 (s, 1H), 5.32 (s, 1H), 4.77 (d, J = 3.5 Hz, 1H), 4.30 (t, J = 8.7 Hz, 1H), 4.17 (dd, J = 8.4, 5.8 Hz, 1H), 3.97 (t, J = 9.3 Hz, 1H), 3.86-3.77 (m, 2H), 2.39 (dt, J = 7.9, 4.2 Hz, 1H), 2.24-2.11 (m, 2H), 1.99 (d, J = 14.1 Hz, 1H), 1.91-1.74 (m, | | | 0.1718 | 10.76 |
| | | | 4H), 1.68 (dd, J = | | | | |
| | | | 14.1, 3.9 Hz, 1H), | | | | |
| | | | 1.61-1.49 (m, 4H), | | | | |
| | | | 1.47-1.30 (m, 6H), | | | | |
| | | | 1.27 (s, 3H), 1.23 (s, | | | | |
| | | | 1H), 1.15 (td, J = | | | | |
| | | | 11.4, 6.5 Hz, 1H), | | | | |
| | | | 0.94-0.82 (m, 7H). | | | | |
| 260 | 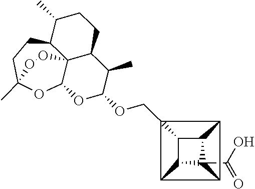 | (2R,3R,5R,6R,7R, 8R)-4- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) cubane-1- carboxylic acid | ¹H NMR (400 MHz, DMSO) δ 12.24 (s, 1H), 5.41 (s, 1H), 4.47 (d, J = 9.2 Hz, 1H), 4.03 (dd, J = 5.5, 4.2 Hz, 3H), 3.86-3.77 (m, 4H), 3.65 (d, J = 12.0 Hz, 1H), 2.24-2.05 (m, 2H), 1.98 (dt, J = 14.0, 3.9 Hz, 1H), 1.85-1.75 (m, 1H), 1.65-1.55 (m, 2H), | | | 0.4223 | >20 |
| | | | 1.42 (td, J = 13.4, | | | | |
| | | | 6.3 Hz, 2H), 1.38- | | | | |
| | | | 1.30 (m, 2H), 1.28 | | | | |
| | | | (s, 3H), 1.15 (td, J = | | | | |
| | | | 11.3, 6.5 Hz, 1H), | | | | |
| | | | 1.00-0.91 (m, 1H), | | | | |
| | | | 0.89 (d, J = 6.3 Hz, | | | | |
| | | | 3H), 0.79 (d, J = 7.1 | | | | |
| | | | Hz, 3H). | | | | |
| 261 | 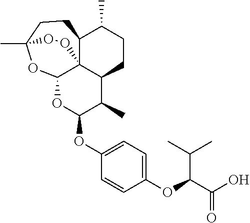 [SMILES: CC(C)[C@H](Oc1ccc(O[C@H]2O[C@@H]3O[C@@]4(C)CC[C@H]5[C@H](C)CC[C@@H]([C@H]2C)[C@@]35OO4)cc1)C(=O)O] | (S)-3-methyl-2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenoxy) butanoic acid | ¹H NMR (400 MHz, DMSO) δ 12.93 (s, 1H), 7.02-6.94 (m, 2H), 6.90-6.78 (m, 2H), 5.45 (s, 1H), 5.38 (t, J = 2.7 Hz, 1H), 4.37 (dd, J = 5.1, 1.9 Hz, 1H), 2.60-2.51 (m, 1H), 2.25-2.10 (m, 2H), 2.04-1.96 (m, 1H), 1.94-1.86 (m, 1H), 1.81 (q, J = 8.7 Hz, 2H), 1.62 (dd, J = 13.1, 3.4 Hz, 1H), 1.50 (dt, J = 13.2, | | | <0.01 | 0.36 |
| | | | 4.7 Hz, 1H), 1.45- | | | | |
| | | | 1.33 (m, 2H), 1.27 | | | | |
| | | | (s, 3H), 1.19 (td, J = | | | | |
| | | | 11.9, 6.8 Hz, 1H), | | | | |
| | | | 1.00 (d, J = 6.8 Hz, | | | | |
| | | | 3H), 0.98-0.86 (m, | | | | |
| | | | 7H). | | | | |
| 262 | 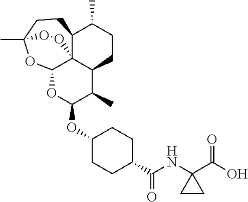 [SMILES: C[C@H]1[C@@H](O[C@H]2CC[C@@H](C(=O)NC3(C(=O)O)CC3)CC2)O[C@@H]2O[C@@]3(C)CC[C@H]4[C@H](C)CC[C@@H]1[C@@]24OO3] | 1-((1R,4s)-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) cyclohexane-1- carboxamido) cyclopropane-1- | ¹H NMR (400 MHz, DMSO) δ 12.23 (s, 1H), 8.25 (s, 1H), 5.32 (s, 1H), 4.76 (d, J = 3.5 Hz, 1H), 3.78 (s, 1H), 2.39 (dt, J = 7.9, 4.2 Hz, 1H), 2.18 (td, J = 14.0, 3.9 Hz, 1H), 2.12-2.04 (m, 1H), 1.98 (dd, J = 14.4, 4.2 Hz, 1H), 1.91- 1.72 (m, 4H), 1.69 | | | 11.3731 | >20 |
| | | carboxylic acid | (dd, J = 14.2, 3.9 | | | | |
| | | | Hz, 1H), 1.60 (td, | | | | |
| | | | J = 12.9, 3.2 Hz, | | | | |
| | | | 3H), 1.52-1.30 (m, | | | | |
| | | | 7H), 1.29-1.23 (m, | | | | |
| | | | 5H), 1.15 (tt, J = | | | | |
| | | | 11.5, 6.6 Hz, 1H), | | | | |
| | | | 0.99-0.76 (m, 9H). | | | | |
| 263 | 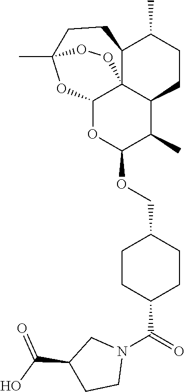 [SMILES: C[C@H]1[C@@H](OC[C@H]2CC[C@@H](C(=O)N3CC[C@@H](C(=O)O)C3)CC2)O[C@@H]2O[C@@]3(C)CC[C@H]4[C@H](C)CC[C@@H]1[C@@]24OO3] | (R)-1-((1R,4S)- 4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carbonyl) pyrrolidine-3- carboxylic acid | | | | 0.3738 | 16.79 |
| 264 | 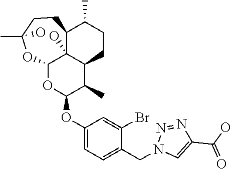 [SMILES: C[C@H]1[C@@H](Oc2ccc(Cn3cc(C(=O)O)nn3)c(Br)c2)O[C@@H]2O[C@@]3(C)CC[C@H]4[C@H](C)CC[C@@H]1[C@@]24OO3] | 1-(2-bromo-4- (((3R,5aS,6R,8aS,9R,10S,12R, 12aR)-3,6,9- trimethyldeca- hydro-12H-3,12- epoxy[1,2] dioxepino[4,3-i] isochromen-10- yl)oxy)benzyl)- 1H-1,2,3- | | | | 0.1586 | 1.43 |
| | | triazole-4- | | | | | |
| | | carboxylic acid | | | | | |
| 265 | 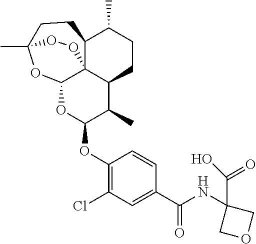 [SMILES: C[C@H]1[C@@H](Oc2ccc(C(=O)NC3(C(=O)O)COC3)cc2Cl)O[C@@H]2O[C@@]3(C)CC[C@H]4[C@H](C)CC[C@@H]1[C@@]24OO3] | 3-(3-chloro-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) benzamido) oxetane-3- carboxylic acid | | | | 6.4454 | 17.15 |
| 266 | 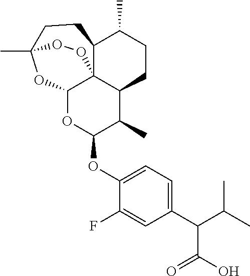 [SMILES: CC(C)C(C(=O)O)c1ccc(O[C@H]2O[C@@H]3O[C@@]4(C)CC[C@H]5[C@H](C)CC[C@@H]([C@H]2C)[C@@]35OO4)c(F)c1] | 2-(3-fluoro-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl)-3- methylbutanoic acid | ¹H NMR (400 MHz, MeOD) δ 12.39 (s, 1H), 7.28 (td, J = 8.6, 1.2 Hz, 1H), 7.17 (dd, J = 12.2, 2.1 Hz, 1H), 7.09 (dd, J = 8.5, 1.8 Hz, 1H), 5.47 (d, J = 1.9 Hz, 1H), 3.09 (d, J = 10.4 Hz, 1H), 2.66- 2.54 (m, 1H), 2.25- 2.08 (m, 2H), 2.00 (dt, J = 14.6, 4.1 Hz, 1H), 1.94-1.85 (m, 1H), 1.84-1.74 (m, 2H), 1.62 (dd, J = 13.1, 3.3 Hz, 1H), 1.53 (dt, J = 13.2, 4.7 Hz, 1H), | | | 0.0103 | 0.06 |
| | | | 1.44-1.30 | | | | |
| | | | (m, 2H), 1.28 (s, | | | | |
| | | | 3H), 1.22 (dd, J = | | | | |
| | | | 11.7, 5.9 Hz, 1H), | | | | |
| | | | 1.02-0.92 (m, 7H), | | | | |
| | | | 0.91 (d, J = 6.3 Hz, | | | | |
| | | | 3H), 0.65 (d, J = 6.6 | | | | |
| | | | Hz, 3H). | | | | |
| 267 | 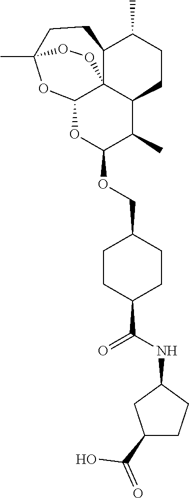 [SMILES: C[C@H]1[C@@H](OC[C@H]2CC[C@@H](C(=O)N[C@H]3CC[C@@H](C(=O)O)C3)CC2)O[C@@H]2O[C@@]3(C)CC[C@H]4[C@H](C)CC[C@@H]1[C@@]24OO3] | (1R,3S)-3- ((1R,4R)-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxamido) cyclopentane-1- carboxylic acid | ¹H NMR (400 MHz, MeOD) δ 12.08 (s, 1H), 7.65 (d, J = 7.4 Hz, 1H), 5.26 (s, 1H), 4.65 (d, J = 3.5 Hz, 1H), 3.98 (p, J = 7.3 Hz, 1H), 3.63 (dd, J = 9.5, 7.3 Hz, 1H), 3.20 (dd, J = 9.4, 6.7 Hz, 1H), 2.68 (p, J = 8.3 Hz, 1H), 2.39 (dt, J = 7.9, 4.1 Hz, 1H), 2.25-2.13 (m, 2H), 2.07 (dt, J = 12.7, 7.7 Hz, 1H), 2.02- 1.95 (m, 1H), 1.85- 1.75 (m, 4H), 1.75- 1.69 (m, 1H), 1.64 (dt, J = 7.9, 5.2 Hz, 3H), 1.60-1.52 (m, 2H), 1.52-1.33 (m, 9H), 1.29 (s, 3H), 1.26-1.23 (m, 1H), 1.14 (td, J = 11.4, 6.6 Hz, 1H), 0.93- 0.81 (m, 7H). | | | 0.1563 | 20.00 |
| 268 | 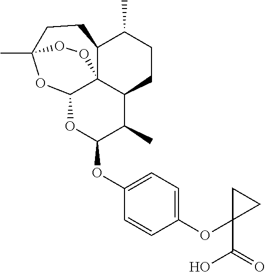 [SMILES: C[C@H]1[C@@H](Oc2ccc(OC3(C(=O)O)CC3)cc2)O[C@@H]2O[C@@]3(C)CC[C@H]4[C@H](C)CC[C@@H]1[C@@]24OO3] | 1-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenoxy) cyclopropane-1- carboxylic acid | ¹H NMR (400 MHz, MeOD) δ 6.99 (d, J = 9.1 Hz, 2H), 6.83 (d, J = 9.1 Hz, 2H), 5.44 (s, 1H), 5.39 (d, J = 3.4 Hz, 1H), 2.56 (q, J = 4.5 Hz, 1H), 2.24-2.13 (m, 1H), 2.03-1.96 (m, 1H), 1.92-1.75 (m, 3H), 1.62 (dd, J = 13.1, 3.3 Hz, 1H), 1.55-1.44 (m, 3H), 1.43-1.34 (m, 2H), 1.28 (s, 3H), 1.25-1.20 (m, 3H), 1.00-0.86 (m, 7H). | | | 0.0489 | 4.28 |
| 269 | 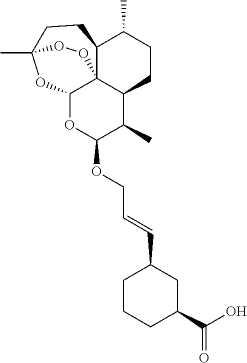 [SMILES: C[C@H]1[C@@H](OC/C=C/[C@@H]2CCC[C@H](C(=O)O)C2)O[C@@H]2O[C@@]3(C)CC[C@H]4[C@H](C)CC[C@@H]1[C@@]24OO3] | (1S,3R)-3-((E)- 3- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)prop-1- en-1-yl) cyclohexane-1- carboxylic acid | | | | 0.1431 | 7.33 |
| 270 | 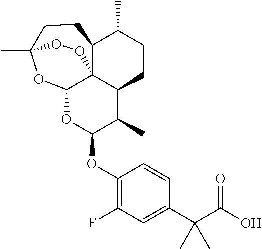 [SMILES: C[C@H]1[C@@H](Oc2ccc(C(C)(C)C(=O)O)cc2F)O[C@@H]2O[C@@]3(C)CC[C@H]4[C@H](C)CC[C@@H]1[C@@]24OO3] | 2-(3-fluoro-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl)- 2-methyl- propanoic acid | ¹H NMR (400 MHz, MeOD) δ 12.42 (s, 1H), 7.28 (t, J = 8.8 Hz, 1H), 7.17 (dd, J = 12.8, 2.3 Hz, 1H), 7.12 (dd, J = 8.6, 2.3 Hz, 1H), 5.55 (d, J = 3.4 Hz, 1H), 5.46 (s, 1H), 2.66-2.54 (m, 1H), 2.19 (td, J = 13.9, 3.9 Hz, 1H), 1.99 (dt, J = 14.3, 4.1 Hz, 1H), 1.90 (td, J = 13.6, 3.3 Hz, 1H), 1.85-1.76 (m, 2H), | | | 0.0286 | 0.70 |
| | | | 1.63 (dd, J = 13.0, | | | | |
| | | | 3.2 Hz, 1H), 1.53 | | | | |
| | | | (dt, J = 13.2, 4.7 | | | | |
| | | | Hz, 1H), 1.45 (s, | | | | |
| | | | 6H), 1.42-1.33 (m, | | | | |
| | | | 2H), 1.27 (s, 3H), | | | | |
| | | | 1.21 (dt, J = 10.8, | | | | |
| | | | 5.3 Hz, 1H), 0.99 | | | | |
| | | | (d, J = 7.3 Hz, 3H), | | | | |
| | | | 0.97-0.85 (m, 4H). | | | | |
| 271 | 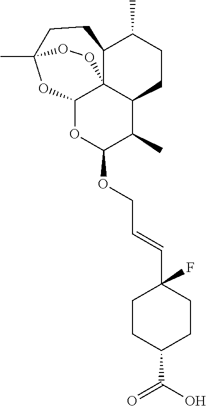 [SMILES: C[C@H]1[C@@H](OC/C=C/[C@]2(F)CC[C@H](C(=O)O)CC2)O[C@@H]2O[C@@]3(C)CC[C@H]4[C@H](C)CC[C@@H]1[C@@]24OO3] | (1S,4r)-4- fluoro-4-((E)-3- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)prop-1-en- 1-yl) cyclohexane-1- carboxylic acid | | | | 0.0924 | 19.05 |
| 272 | 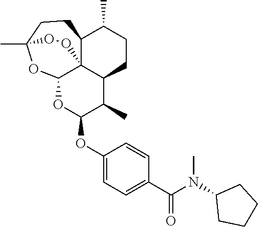 | (1R,3S)-3-(N- methyl-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) benzamido) cyclopentane-1- carboxylic acid | | | | 0.1052 | 9.27 |
| 273 | 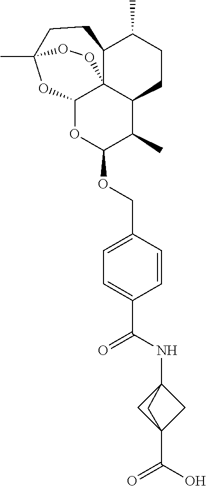 [SMILES: C[C@H]1[C@@H](OCc2ccc(C(=O)NC34CC(C(=O)O)(C3)C4)cc2)O[C@@H]2O[C@@]3(C)CC[C@H]4[C@H](C)CC[C@@H]1[C@@]24OO3] | 3-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamido) bicyclo[1.1.1] pentane-1- carboxylic acid | ¹H NMR (400 MHz, DMSO) δ 12.46 (s, 1H), 9.05 (s, 1H), 7.81 (d, J = 8.2 Hz, 2H), 7.39 (d, J = 8.1 Hz, 2H), 5.40 (s, 1H), 4.85-4.77 (m, 2H), 4.51 (t, J = 12.5 Hz, 1H), 2.45 (dq, J = 7.7, 3.8 Hz, 1H), 2.28 (s, 6H), 2.19 (td, J = 13.9, 4.0 Hz, 1H), 2.04- 1.96 (m, 1H), 1.84- 1.64 (m, 3H), 1.54 (dd, J = 13.0, 3.4 Hz, 1H), 1.46-1.32 (m, 3H), 1.30 (s, 3H), 1.20-1.11 (m, 1H), 0.92-0.81 (m, 7H). | | | 2.8866 | 19.41 |
| 274 | 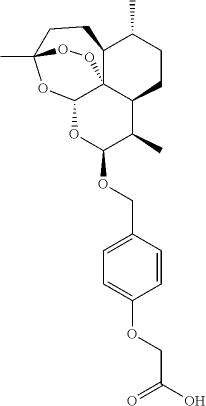 [SMILES: C[C@H]1[C@@H](OCc2ccc(OCC(=O)O)cc2)O[C@@H]2O[C@]3(C)CC[C@H]4[C@H](C)CC[C@@H]1[C@@]24OO3] | 2-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) phenoxy)acetic acid | | | | 0.8482 | >20 |
| 275 | 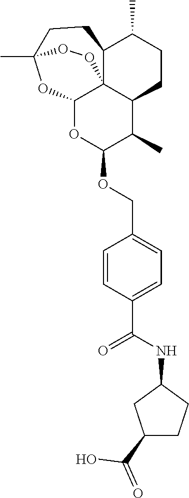 [SMILES: C[C@H]1[C@@H](OCc2ccc(C(=O)N[C@H]3CC[C@@H](C(=O)O)C3)cc2)O[C@@H]2O[C@@]3(C)CC[C@H]4[C@H](C)CC[C@@H]1[C@@]24OO3] | (1R,3S)-3-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamido) cyclopentane-1- carboxylic acid | ¹H NMR (400 MHz, DMSO) δ 12.14 (s, 1H), 8.37 (d, J = 7.3 Hz, 1H), 7.82 (d, J = 8.1 Hz, 2H), 7.39 (d, J = 8.1 Hz, 2H), 5.41 (s, 1H), 4.85- 4.77 (m, 2H), 4.49 (d, J = 12.8 Hz, 1H), 4.24 (p, J = 7.4 Hz, 1H), 2.75 (p, J = 8.5 Hz, 1H), 2.46 (dt, J = 7.9, 4.1 Hz, 1H), 2.25-2.11 (m, 2H), 2.01 (d, J = 14.4 Hz, 1H), 1.95-1.58 (m, 8H), 1.55 (dd, J = 13.1, 3.3 Hz, 1H), 1.46- 1.31 (m, 3H), 1.30 (s, 3H), 1.21-1.10 (m, 1H), 0.92-0.81 (m, 7H). | | | 0.2402 | 6.80 |
| 276 | 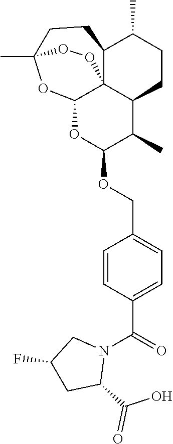 [SMILES: C[C@H]1[C@@H](OCc2ccc(C(=O)N3C[C@@H](F)C[C@H]3C(=O)O)cc2)O[C@@H]2O[C@@]3(C)CC[C@H]4[C@H](C)CC[C@@H]1[C@@]24OO3] | (2S,4S)-4- fluoro-1-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzoyl) pyrrolidine-2- carboxylic acid | | | | >20 | >20 |
| 277 | 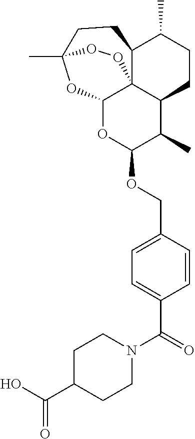 [SMILES: C[C@H]1[C@@H](OCc2ccc(C(=O)N3CCC(C(=O)O)CC3)cc2)O[C@@H]2O[C@@]3(C)CC[C@H]4[C@H](C)CC[C@@H]1[C@@]24OO3] | 1-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzoyl) piperidine-4- carboxylic acid | | | | 1.776 | >20 |
| 278 | 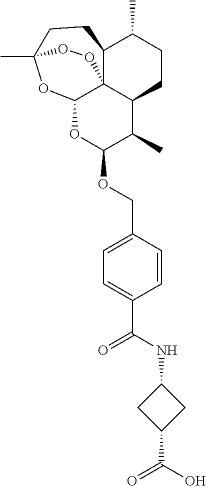 [SMILES: C[C@H]1[C@@H](OCc2ccc(C(=O)N[C@H]3C[C@@H](C(=O)O)C3)cc2)O[C@@H]2O[C@@]3(C)CC[C@H]4[C@H](C)CC[C@@H]1[C@@]24OO3] | (1R,3s)-3-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamido) cyclobutane-1- carboxylic acid | ¹H NMR (400 MHz, DMSO) δ 12.18 (s, 1H), 8.69 (d, J = 7.7 Hz, 1H), 7.85 (d, J = 8.0 Hz, 2H), 7.39 (d, J = 7.9 Hz, 2H), 5.40 (s, 1H), 4.85-4.77 (m, 2H), 4.36 (h, J = 8.3 Hz, 1H), 2.77 (tt, J = 9.9, 8.0 Hz, 1H), 2.50-2.38 (m, 3H), 2.30-2.12 (m, 3H), 2.04-1.96 (m, 1H), 1.85-1.64 (m, 3H), 1.58-1.50 (m, 1H), 1.45-1.32 (m, 3H), 1.30 (s, 3H), 1.16 (dt, J = 11.6, 5.4 Hz, 1H), 0.95-0.81 (m, 7H). | | | 0.3539 | >20 |
| 279 | 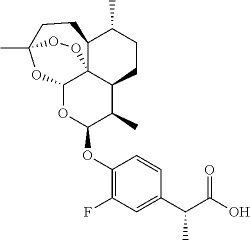 [SMILES: C[C@H]1[C@@H](Oc2ccc([C@@H](C)C(=O)O)cc2F)O[C@@H]2O[C@@]3(C)CC[C@H]4[C@H](C)CC[C@@H]1[C@@]24OO3] | (R)-2-(3-fluoro- 4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) propanoic acid | ¹H NMR (400 MHz, DMSO) δ 12.45 (s, 1H), 7.28 (t, J = 8.6 Hz, 1H), 7.15 (dd, J = 12.2, 2.1 Hz, 1H), 7.07 (dd, J = 8.6, 2.1 Hz, 1H), 5.53 (d, J = 3.4 Hz, 1H), 5.46 (s, 1H), 3.65 (q, J = 7.1 Hz, 1H), 2.64-2.54 (m, 1H), 2.19 (td, J = 13.9, 3.8 Hz, 1H), 2.04-1.95 (m, 1H), 1.91 (dd, J = 13.5, 3.3 Hz, 1H), 1.85- 1.77 (m, 2H), 1.63 | | | 0.0268 | 4.86 |
| | | | (dd, J = 13.1, 3.3 | | | | |
| | | | Hz, 1H), 1.53 (dt, | | | | |
| | | | J = 13.3, 4.6 Hz, | | | | |
| | | | 1H), 1.43-1.37 (m, | | | | |
| | | | 1H), 1.36-1.30 (m, | | | | |
| | | | 4H), 1.27 (s, 3H), | | | | |
| | | | 1.22 (dd, J = 11.5, | | | | |
| | | | 6.2 Hz, 1H), 0.99 (d, | | | | |
| | | | J = 7.3 Hz, 3H), | | | | |
| | | | 0.97-0.87 (m, 4H). | | | | |
| 280 | 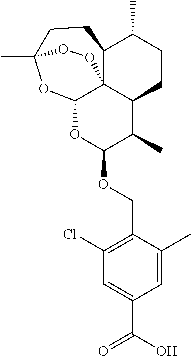 | 3-chloro-5- methyl-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzoic acid | | | | 0.2462 | 3.22 |
| 281 | 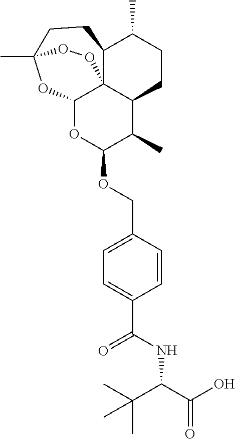 [SMILES: C[C@H]1[C@@H](OCc2ccc(C(=O)N[C@H](C(=O)O)C(C)(C)C)cc2)O[C@@H]2O[C@@]3(C)CC[C@H]4[C@H](C)CC[C@@H]1[C@@]24OO3] | (S)-3,3- dimethyl-2-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamido) butanoic acid | | | | 0.1123 | 0.43 |
| 282 | 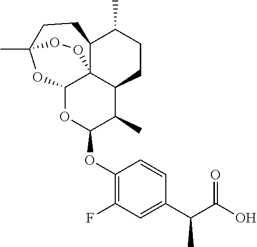 [SMILES: C[C@H]1[C@@H](Oc2ccc([C@H](C)C(=O)O)cc2F)O[C@@H]2O[C@@]3(C)CC[C@H]4[C@H](C)CC[C@@H]1[C@@]24OO3] | (S)-2-(3-fluoro- 4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) propanoic acid | ¹H NMR (400 MHz, DMSO) δ 12.41 (s, 1H), 7.28 (t, J = 8.6 Hz, 1H), 7.15 (dd, J = 12.2, 2.1 Hz, 1H), 7.10-7.02 (m, 1H), 5.53 (d, J = 3.4 Hz, 1H), 5.46 (s, 1H), 3.66 (q, J = 7.1 Hz, 1H), 2.60 (dt, J = 7.9, 4.2 Hz, 1H), 2.25-2.13 (m, 1H), 2.00 (d, J = 13.8 Hz, 1H), 1.94- 1.85 (m, 1H), 1.85- 1.76 (m, 2H), 1.63 (dd, J = 13.2, 3.3 | | | 0.0127 | 4.82 |
| | | | Hz, 1H), 1.58-1.49 | | | | |
| | | | (m, 1H), 1.44-1.36 | | | | |
| | | | (m, 1H), 1.36-1.30 | | | | |
| | | | (m, 4H), 1.27 (s, | | | | |
| | | | 3H), 1.23 (t, J = 5.6 | | | | |
| | | | Hz, 1H), 0.99 (d, J = | | | | |
| | | | 7.3 Hz, 3H), 0.96- | | | | |
| | | | 0.86 (m, 4H). | | | | |
| 283 | 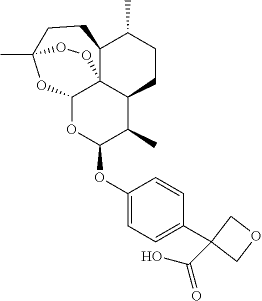 [SMILES: C[C@H]1[C@@H](Oc2ccc(C3(C(=O)O)COC3)cc2)O[C@@H]2O[C@@]3(C)CC[C@H]4[C@H](C)CC[C@@H]1[C@@]24OO3] | 3-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) oxetane-3- carboxylic acid | ¹H NMR (400 MHz, DMSO) δ 12.97 (s, 1H), 7.25-7.18 (m, 2H), 7.08 (d, J = 8.7 Hz, 2H), 5.57 (d, J = 3.4 Hz, 1H), 5.41 (s, 1H), 5.05 (d, J = 6.1 Hz, 2H), 4.83 (dd, J = 6.2, 1.5 Hz, 2H), 2.63- 2.57 (m, 1H), 2.24- 2.14 (m, 1H), 2.00 (d, J = 14.8 Hz, 1H), 1.91 (d, J = 13.0 Hz, 1H), 1.87-1.77 (d, J = 15.0 Hz, 2H), 1.63 (d, J = 13.3 Hz, 1H), 1.53 (dd, J = 11.2, 6.4 Hz, 1H), 1.46-1.33 (m, 2H), | | | 0.0403 | >20 |
| | | | 1.28 (s, 3H), 1.21 | | | | |
| | | | (dd, J = 11.3, 6.5 | | | | |
| | | | Hz, 1H), 1.00-0.87 | | | | |
| | | | (m, 7H). | | | | |
| 284 | 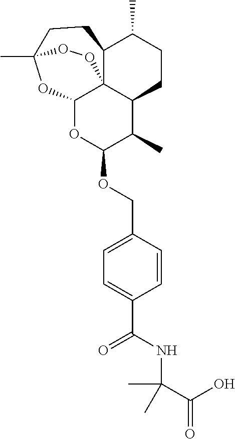 [SMILES: C[C@H]1[C@@H](OCc2ccc(C(=O)NC(C)(C)C(=O)O)cc2)O[C@@H]2O[C@@]3(C)CC[C@H]4[C@H](C)CC[C@@H]1[C@@]24OO3] | 2-methyl-2-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamido) propanoic acid | ¹H NMR (400 MHz, DMSO) δ 12.09 (s, 1H), 8.43 (s, 1H), 7.84 (d, J = 8.0 Hz, 2H), 7.41 (d, J = 8.0 Hz, 2H), 5.41 (s, 1H), 4.85- 4.77 (m, 2H), 4.50 (d, J = 12.8 Hz, 1H), 2.19 (td, J = 14.0, 3.8 Hz, 1H), 2.01 (d, J = 14.8 Hz, 1H), 1.86-1.73 (m, 2H), 1.73-1.65 (m, 1H), 1.55 (dd, J = 13.0, 3.3 Hz, 1H), 1.48- 1.33 (m, 9H), 1.32- 1.26 (m, 4H), 1.17 (dd, J = 11.4, 6.5 Hz, 1H), 0.95-0.81 (m, 7H). | | | 0.5236 | >20 |
| 285 | 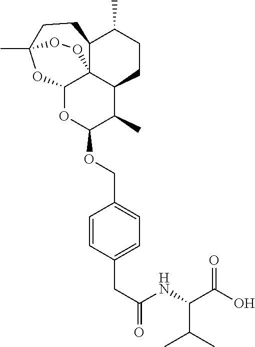 [SMILES: CC(C)[C@H](NC(=O)Cc1ccc(CO[C@H]2O[C@@H]3O[C@@]4(C)CC[C@H]5[C@H](C)CC[C@@H]([C@H]2C)[C@@]35OO4)cc1)C(=O)O] | (2-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) phenyl)acetyl)-L- valine | | | | | |
| 286 | 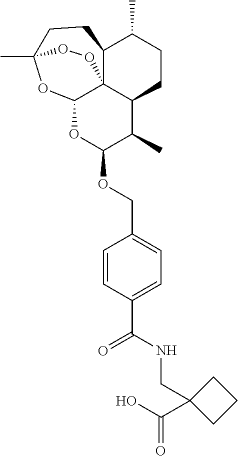 [SMILES: C[C@H]1[C@@H](OCc2ccc(C(=O)NCC3(C(=O)O)CCC3)cc2)O[C@@H]2O[C@@]3(C)CC[C@H]4[C@H](C)CC[C@@H]1[C@@]24OO3] | 1-((4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamido) methyl) cyclobutane-1- carboxylic acid | ¹H NMR (400 MHz, DMSO) δ 12.26 (s, 1H), 8.45 (t, J = 6.1 Hz, 1H), 7.85-7.79 (m, 2H), 7.41 (d, J = 8.1 Hz, 2H), 5.41 (s, 1H), 4.92-4.73 (m, 2H), 4.49 (d, J = 12.8 Hz, 1H), 3.61 (d, J = 6.0 Hz, 2H), 2.30-2.13 (m, 3H), 2.12-1.97 (m, 3H), 1.80 (dt, J = 19.6, 11.3 Hz, 4H), 1.73- 1.64 (m, 1H), 1.55 (dd, J = 13.0, 3.3 Hz, 1H), 1.46-1.32 (m, 3H), 1.31 (s, 3H), 1.26-1.11 (m, 2H), 0.95-0.83 (m, 7H). | | | 0.0180 | 1.53 |
| 287 | 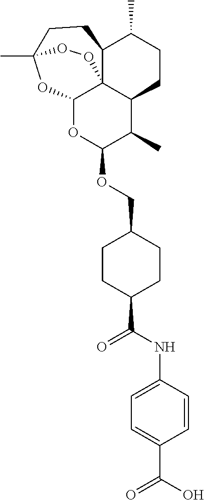 [SMILES: C[C@H]1[C@@H](OC[C@H]2CC[C@@H](C(=O)Nc3ccc(C(=O)O)cc3)CC2)O[C@@H]2O[C@@]3(C)CC[C@H]4[C@H](C)CC[C@@H]1[C@@]24OO3] | 4-((1R,4s)-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxamido) benzoic acid | ¹H NMR (400 MHz, DMSO) δ 12.66 (s, 1H), 10.08 (s, 1H), 7.90-7.82 (m, 2H), 7.75-7.67 (m, 2H), 5.28 (s, 1H), 4.67 (d, J = 3.4 Hz, 1H), 3.66 (dd, J = 9.5, 7.3 Hz, 1H), 3.24 (dd, J = 9.5, 6.6 Hz, 1H), 2.44-2.36 (m, 1H), 2.17 (td, J = 14.0, 3.8 Hz, 1H), 1.99 (d, J = 14.3 Hz, 1H), 1.84- 1.70 (m, 5H), 1.69- 1.47 (m, 8H), 1.43- 1.32 (m, 2H), 1.32- 1.20 (m, 5H), 1.18- 1.08 (m, 1H), 0.92- 0.79 (m, 7H). | | | 0.0608 | 1.00 |
| 288 | 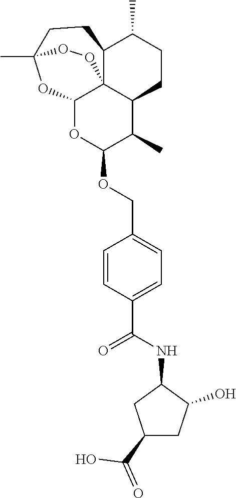 [SMILES: C[C@H]1[C@@H](OCc2ccc(C(=O)N[C@@H]3C[C@H](C(=O)O)C[C@H]3O)cc2)O[C@@H]2O[C@@]3(C)CC[C@H]4[C@H](C)CC[C@@H]1[C@@]24OO3] | (1S,3R,4R)-3- hydroxy-4-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamido) cyclopentane-1- carboxylic acid | | | | 5.5829 | >20 |
| 289 | 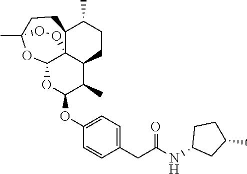 | (1S,3R)-3-(2- (4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetamido) cyclopentane-1- carboxylic acid | | | | 0.0276 | >20 |
| 290 | 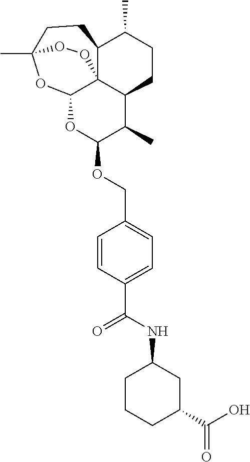 [SMILES: C[C@H]1[C@@H](OCc2ccc(C(=O)N[C@@H]3CCC[C@@H](C(=O)O)C3)cc2)O[C@@H]2O[C@@]3(C)CC[C@H]4[C@H](C)CC[C@@H]1[C@@]24OO3] | (1R,3R)-3-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamido) cyclohexane-1- carboxylic acid | ¹H NMR (400 MHz, DMSO) δ 12.16 (s, 1H), 8.11 (d, J = 7.5 Hz, 1H), 7.82 (d, J = 8.2 Hz, 2H), 7.40 (d, J = 8.1 Hz, 2H), 5.41 (s, 1H), 4.85-4.77 (m, 2H), 4.49 (d, J = 12.8 Hz, 1H), 4.05 (s, 1H), 2.74 (d, J = 5.1 Hz, 1H), 2.69 (s, 1H), 2.47-2.42 (m, 1H), 2.19 (td, J = 14.0, 3.9 Hz, 1H), 2.01 (d, J = 14.0 Hz, 1H), 1.94-1.86 (m, 1H), 1.85-1.74 (m, 2H), 1.73-1.60 (m, 4H), 1.55 (d, J = 10.1 Hz, 2H), 1.49-1.33 (m, 5H), 1.31 (s, 3H), 1.23 (s, 1H), 1.17 (dd, J = 11.4, 6.6 Hz, 1H), 0.90 (s, 3H), 0.88 (s, 3H). | | | 0.0834 | 11.29 |
| 291 | 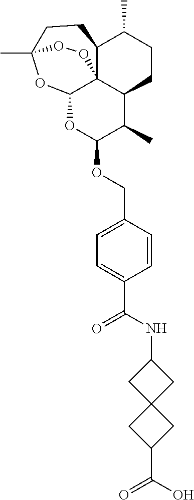 [SMILES: C[C@H]1[C@@H](OCc2ccc(C(=O)NC3CC4(C3)CC(C(=O)O)C4)cc2)O[C@@H]2O[C@@]3(C)CC[C@H]4[C@H](C)CC[C@@H]1[C@@]24OO3] | 6-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamido)spiro [3.3]heptane-2- carboxylic acid | | | | 0.0478 | 13.42 |
| 292 | 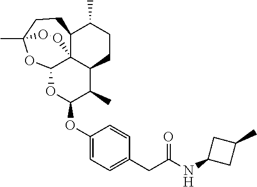 | (1R,3s)-3-(2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetamido) cyclobutane-1- | ¹H NMR (400 MHz, DMSO) δ 12.15 (s, 1H), 8.33 (d, J = 7.9 Hz, 1H), 7.16 (d, J = 8.6 Hz, 2H), 6.98 (d, J = 8.6 Hz, 2H), 5.49 (d, J = 3.4 Hz, 1H), 5.41 (s, 1H), 4.09 (h, J = 8.4 Hz, 1H), 3.28 (s, 2H), 2.70 (p, J = 8.8 Hz, 1H), 2.61-2.55 | | | 0.0429 | >20 |
| | | carboxylic acid | (m, 1H), 2.42-2.32 | | | | |
| | | | (m, 2H), 2.19 (td, | | | | |
| | | | J = 13.9, 3.8 | | | | |
| | | | Hz, 1H), 2.02 | | | | |
| | | | (qd, J = 9.6, | | | | |
| | | | 2.9 Hz, 3H), 1.94- | | | | |
| | | | 1.76 (m, 3H), 1.63 | | | | |
| | | | (dd, J = 13.0, 3.4 | | | | |
| | | | Hz, 1H), 1.51 (dt, | | | | |
| | | | J = 13.4, 4.7 Hz, | | | | |
| | | | 1H), 1.46-1.33 (m, | | | | |
| | | | 2H), 1.28 (s, 3H), | | | | |
| | | | 1.21 (dd, J = 11.4, | | | | |
| | | | 6.5 Hz, 1H), 1.00- | | | | |
| | | | 0.86 (m, 7H). | | | | |
| 293 | 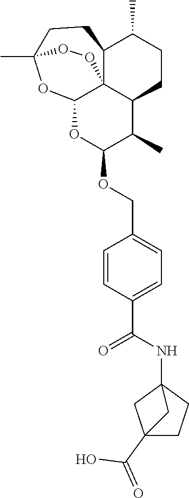 [SMILES: C[C@H]1[C@@H](OCc2ccc(C(=O)NC34CCC(C(=O)O)(C3)C4)cc2)O[C@@H]2O[C@@]3(C)CC[C@H]4[C@H](C)CC[C@@H]1[C@@]24OO3] | 4-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamido) bicyclo[2.1.1] hexane-1- carboxylic acid | ¹H NMR (400 MHz, DMSO) δ 12.27 (s, 1H), 8.80 (s, 1H), 7.83 (d, J = 8.3 Hz, 2H), 7.39 (d, J = 8.1 Hz, 2H), 5.41 (s, 1H), 4.82 (s, 1H), 4.80 (d, J = 8.6 Hz, 1H), 4.49 (d, J = 12.9 Hz, 1H), 2.48-2.41 (m, 1H), 2.27-2.08 (m, 3H), 2.00 (dd, J = 14.7, 4.5 Hz, 1H), 1.96- 1.90 (m, 2H), 1.87- 1.63 (m, 7H), 1.54 (dd, J = 13.1, 3.4 Hz, 1H), 1.45-1.31 (m, 3H), 1.31 (s, 3H), 1.16 (td, J = 11.3, 6.6 Hz, 1H), 0.97-0.77 (m, 7H). | | | 0.0432 | >20 |
| 294 | 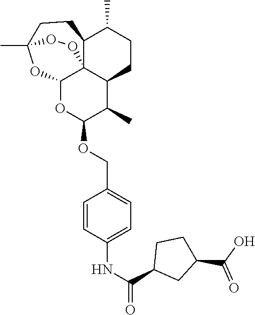 [SMILES: C[C@H]1[C@@H](OCc2ccc(NC(=O)[C@H]3CC[C@@H](C(=O)O)C3)cc2)O[C@@H]2O[C@@]3(C)CC[C@H]4[C@H](C)CC[C@@H]1[C@@]24OO3] | (1R,3S)-3-((4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) phenyl) carbamoyl) cyclopentane-1- carboxylic acid | | | | 0.0650 | 15.76 |
| 295 | 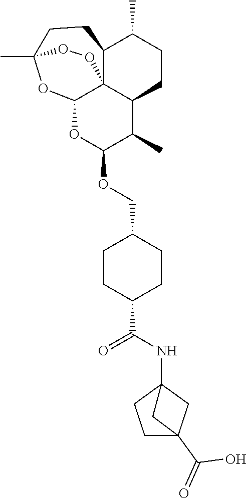 [SMILES: C[C@H]1[C@@H](OC[C@H]2CC[C@@H](C(=O)NC34CCC(C(=O)O)(C3)C4)CC2)O[C@@H]2O[C@@]3(C)CC[C@H]4[C@H](C)CC[C@@H]1[C@@]24OO3] | 4-((1R,4s)-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxamido) bicyclo[2.1.1] hexane-1- carboxylic acid | | | | 0.1966 | >20 |
| 296 | 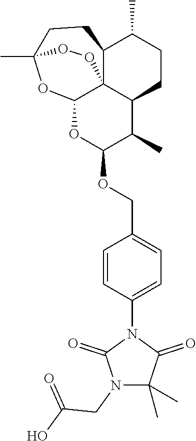 [SMILES: C[C@H]1[C@@H](OCc2ccc(N3C(=O)N(CC(=O)O)C(C)(C)C3=O)cc2)O[C@@H]2O[C@@]3(C)CC[C@H]4[C@H](C)CC[C@@H]1[C@@]24OO3] | 2-(5,5- dimethyl-2,4- dioxo-3-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) phenyl) imidazolidin-1- yl)acetic acid | | | | 2.9133 | >20 |
| 297 | 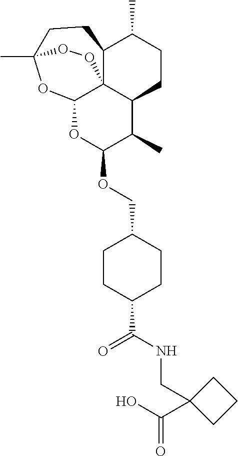 [SMILES: C[C@H]1[C@@H](OC[C@H]2CC[C@@H](C(=O)NCC3(C(=O)O)CCC3)CC2)O[C@@H]2O[C@@]3(C)CC[C@H]4[C@H](C)CC[C@@H]1[C@@]24OO3] | 1-(((1R,4s)-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) cyclohexane-1- carboxamido) methyl) cyclobutane-1- carboxylic acid | | | | 0.0317 | 17.69 |
| 298 | 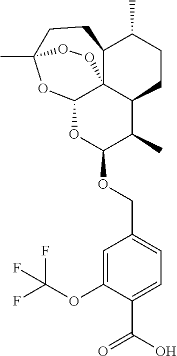 [SMILES: C[C@H]1[C@@H](OCc2ccc(C(=O)O)c(OC(F)(F)F)c2)O[C@@H]2O[C@@]3(C)CC[C@H]4[C@H](C)CC[C@@H]1[C@@]24OO3] | 2- (trifluoro- methoxy)-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzoic acid | | | | 0.0531 | >20 |
| 299 | 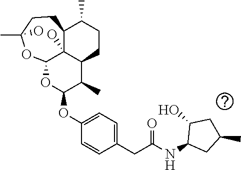 | (1S,3R,4R)-3- hydroxy-4-(2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetamido) cyclopentane-1- carboxylic acid | | 546.4 | | 0.1079 | 4.99 |
| 300 | 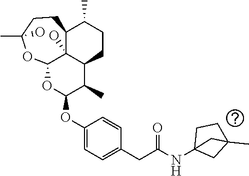 | 4-(2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetamido) bicyclo[2.1.1] hexane-1- carboxylic acid | | | | 0.0170 | 19.47 |
| 301 | 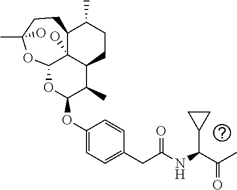 [SMILES: CC(=O)[C@@H](NC(=O)Cc1ccc(O[C@H]2O[C@@H]3O[C@@]4(C)CC[C@H]5[C@H](C)CC[C@@H]([C@H]2C)[C@@]35OO4)cc1)C1CC1] | (S)-2- cyclopropyl-2- (2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetamido) | | | | 0.0635 | >20 |
| | | acetic acid | | | | | |
| 302 | 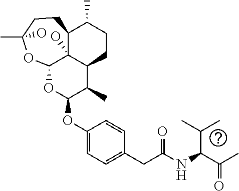 [SMILES: CC(=O)[C@@H](NC(=O)Cc1ccc(O[C@H]2O[C@@H]3O[C@@]4(C)CC[C@H]5[C@H](C)CC[C@@H]([C@H]2C)[C@@]35OO4)cc1)C(C)C] | (2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetyl)-L-valine | | | | 0.0162 | 20.00 |
| 303 | 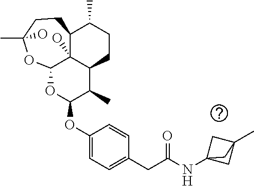 | 3-(2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetamido) bicyclo[1.1.1] | | | | 0.0464 | >20 |
| | | pentane-1- | | | | | |
| | | carboxylic acid | | | | | |
| 304 | 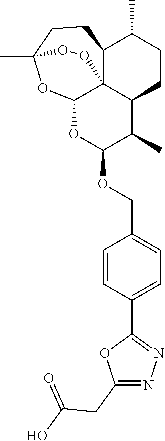 [SMILES: C[C@H]1[C@@H](OCc2ccc(-c3nnc(CC(=O)O)o3)cc2)O[C@@H]2O[C@@]3(C)CC[C@H]4[C@H](C)CC[C@@H]1[C@@]24OO3] | 2-(5-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) phenyl)-1,3,4- oxadiazol-2- yl)acetic acid | ¹H NMR (400 MHz, DMSO) δ 13.21 (s, 1H), 7.96 (d, J = 8.3 Hz, 2H), 7.58-7.52 (m, 2H), 5.42 (s, 1H), 4.90-4.82 (m, 2H), 4.54 (dd, J = 13.2, 4.6 Hz, 1H), 2.58 (s, 2H), 2.46 (d, J = 4.1 Hz, 1H), 2.19 (td, J = 14.1, 3.9 Hz, 1H), 2.01 (d, J = 14.1 Hz, 1H), 1.85-1.65 (m, 3H), 1.60-1.53 (m, 1H), 1.47-1.33 (m, 3H), 1.31 (s, 3H), 1.16 (td, J = 11.4, 6.6 Hz, 1H), 0.97- 0.81 (m, 7H). | 501.4 | | 0.0112 | 0.03 |
| 305 | 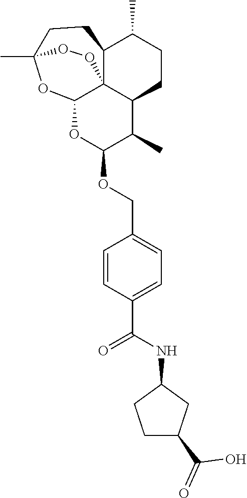 [SMILES: C[C@H]1[C@@H](OCc2ccc(C(=O)N[C@@H]3CC[C@H](C(=O)O)C3)cc2)O[C@@H]2O[C@@]3(C)CC[C@H]4[C@H](C)CC[C@@H]1[C@@]24OO3] | (1S,3R)-3-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamido) cyclopentane-1- carboxylic acid | ¹H NMR (400 MHz, DMSO) δ 12.13 (s, 1H), 8.38 (d, J = 7.3 Hz, 1H), 7.82 (d, J = 8.3 Hz, 2H), 7.39 (d, J = 8.1 Hz, 2H), 5.41 (s, 1H), 4.91-4.73 (m, 2H), 4.49 (d, J = 12.8 Hz, 1H), 4.36-4.13 (m, 1H), 2.76 (q, J = 8.6 Hz, 1H), 2.48-2.41 (m, 1H), 2.25-2.11 (m, 2H), 2.01 (d, J = 14.6 Hz, 1H), 1.95- 1.64 (m, 7H), 1.64- 1.51 (m, 2H), 1.46- 1.32 (m, 3H), 1.30 (s, 3H), 1.16 (td, J = 11.3, 6.5 Hz, 1H), 0.97-0.79 (m, 7H). | | | | |
| 306 | 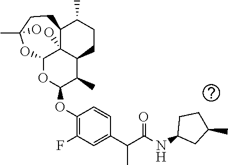 | (1R,3S)-3-(2- (3-fluoro-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) | | | | 0.0133 | 0.69 |
| | | propanamido) | | | | | |
| | | cyclopentane-1- | | | | | |
| | | carboxylic acid | | | | | |
| 307 | 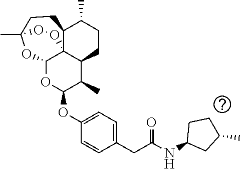 | (1S,3S)-3-(2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetamido) cyclopentane-1- | ¹H NMR (400 MHz, DMSO) δ 12.10 (s, 1H), 8.04 (d, J = 7.2 Hz, 1H), 7.17 (d, J = 8.6 Hz, 2H), 6.98 (d, J = 8.6 Hz, 2H), 5.49 (d, J = 3.4 Hz, 1H), 5.42 (s, 1H), 4.04 (h, J = 6.6 Hz, 1H), 3.30 (d, J = 8.2 Hz, 2H), 2.83 (h, J = 7.4 Hz, 1H), 2.62- | | | 0.0259 | 11.68 |
| | | carboxylic acid | 2.53 (m, 1H), 2.19 | | | | |
| | | | (td, J = 13.9, 3.9 Hz, | | | | |
| | | | 1H), 2.05-1.76 (m, | | | | |
| | | | 7H), 1.74-1.58 (m, | | | | |
| | | | 3H), 1.51 (dt, J = | | | | |
| | | | 13.2, 4.7 Hz, 1H), | | | | |
| | | | 1.46-1.33 (m, 3H), | | | | |
| | | | 1.28 (s, 3H), 1.20 | | | | |
| | | | (td, J = 11.4, 6.5 Hz, | | | | |
| | | | 1H), 0.99-0.86 (m, | | | | |
| | | | 7H). | | | | |
| 308 | 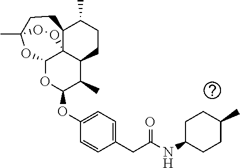 | (1R,4s)-4-(2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetamido) cyclohexane-1- | ¹H NMR (400 MHz, DMSO) δ 12.13 (s, 1H), 7.94 (d, J = 7.6 Hz, 1H), 7.17 (d, J = 8.6 Hz, 2H), 6.98 (d, J = 8.6 Hz, 2H), 5.49 (d, J = 3.4 Hz, 1H), 5.42 (s, 1H), 3.65 (s, 1H), 2.58 (dt, J = 7.7, 4.2 Hz, 1H), 2.43-2.30 (m, 1H), 2.19 (td, J = | | | 0.0121 | >20 |
| | | carboxylic acid | 13.9, 3.9 Hz, 1H), | | | | |
| | | | 2.04-1.88 (m, 2H), | | | | |
| | | | 1.87-1.75 (m, 5H), | | | | |
| | | | 1.63 (dd, J = 13.0, | | | | |
| | | | 3.3 Hz, 1H), 1.58- | | | | |
| | | | 1.47 (m, 5H), 1.47- | | | | |
| | | | 1.33 (m, 4H), 1.28 | | | | |
| | | | (s, 3H), 1.19 (dt, J = | | | | |
| | | | 11.5, 5.8 Hz, 1H), | | | | |
| | | | 1.0-0.83 (m, 7H). | | | | |
| 309 | 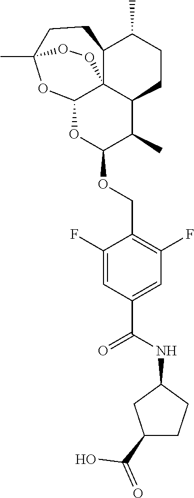 [SMILES: C[C@H]1[C@@H](OCc2c(F)cc(C(=O)N[C@H]3CC[C@@H](C(=O)O)C3)cc2F)O[C@@H]2O[C@@]3(C)CC[C@H]4[C@H](C)CC[C@@H]1[C@@]24OO3] | (1R,3S)-3-(3,5- difluoro-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamido) cyclopentane-1- carboxylic acid | | | | 0.0313 | 1.74 |
| 310 | 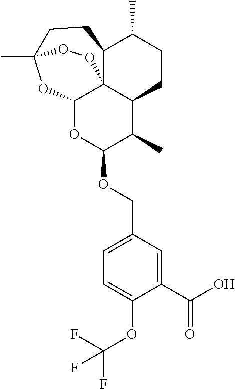 [SMILES: C[C@H]1[C@@H](OCc2ccc(OC(F)(F)F)c(C(=O)O)c2)O[C@@H]2O[C@@]3(C)CC[C@H]4[C@H](C)CC[C@@H]1[C@@]24OO3] | 2- (trifluoro- methoxy)-5- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzoic acid | ¹H NMR (400 MHz, DMSO) δ 7.88 (s, 1H), 7.64 (d, J = 8.5 Hz, 1H), 7.48 (d, J = 8.3 Hz, 1H), 5.40 (s, 1H), 4.85 (s, 1H), 4.83 (d, J = 9.9 Hz, 1H), 4.52 (d, J = 13.0 Hz, 1H), 2.45- 2.39 (m, 1H), 2.24- 2.13 (m, 1H), 2.00 (d, J = 14.6 Hz, 1H), 1.85-1.65 (m, 3H), 1.55 (d, J = 12.4 Hz, 1H), 1.45-1.31 (m, 3H), 1.30 (s, 3H), 1.20-1.10 (m, 1H), 0.95-0.80 (m, 7H). | | | | |
| 311 | 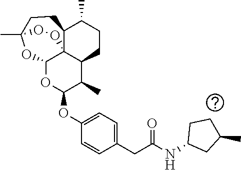 | (1R,3R)-3-(2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetemido) cyclopentane-1- | ¹H NMR (400 MHz, DMSO) δ 12.10 (s, 1H), 8.04 (d, J = 7.2 Hz, 1H), 7.17 (d, J = 8.6 Hz, 2H), 6.98 (d, J = 8.6 Hz, 2H), 5.49 (d, J = 3.4 Hz, 1H), 5.42 (s, 1H), 4.04 (q, J = 6.5 Hz, 1H), 3.29 (s, 2H), 2.83 (p, J = 8.1 Hz, 1H), 2.62-2.54 | | | | |
| | | carboxylic acid | (dt, J = 7.8, 4.3 Hz, | | | | |
| | | | 0H), 2.19 (td, J = | | | | |
| | | | 13.9, 3.7 Hz, 1H), | | | | |
| | | | 2.05-1.76 (m, 7H), | | | | |
| | | | 1.75-1.59 (m, 3H), | | | | |
| | | | 1.56-1.48 (m, 1H), | | | | |
| | | | 1.46-1.31 (m, 3H), | | | | |
| | | | 1.28 (s, 3H), 1.26- | | | | |
| | | | 1.14 (m, 1H), 1.01- | | | | |
| | | | 0.82 (m, 7H). | | | | |
| 312 | 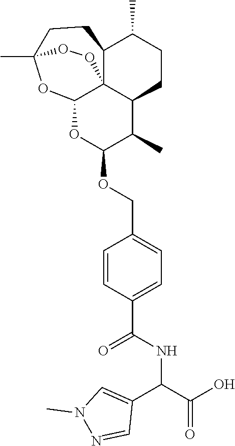 [SMILES: C[C@H]1[C@@H](OCc2ccc(C(=O)NC(C(=O)O)c3cnn(C)c3)cc2)O[C@@H]2O[C@@]3(C)CC[C@H]4[C@H](C)CC[C@@H]1[C@@]24OO3] | 2-(1-methyl- 1H-pyrazol-4- yl)-2-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamido) acetic acid | | | | | |
| 313 | 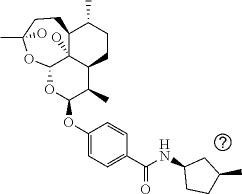 | (1S,3R)-3-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy) benzamido) cyclopentane-1- carboxylic acid | | | | | |
| 314 | 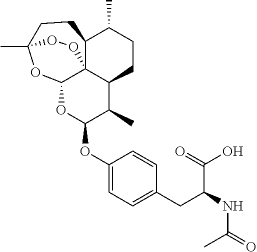 [SMILES: CC(=O)N[C@@H](Cc1ccc(O[C@H]2O[C@@H]3O[C@@]4(C)CC[C@H]5[C@H](C)CC[C@@H]([C@H]2C)[C@@]35OO4)cc1)C(=O)O] | (S)-2- acetamido)-3-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) propanoic acid | ¹H NMR (400 MHz, DMSO) δ 12.66 (s, 1H), 8.16 (d, J = 8.0 Hz, 1H), 7.15 (d, J = 8.6 Hz, 2H), 6.97 (d, J = 8.6 Hz, 2H), 5.50 (d, J = 3.4 Hz, 1H), 5.42 (s, 1H), 4.35 (ddd, J = 9.2, 7.9, 4.9 Hz, 1H), 2.97 (dd, J = 13.9, 5.0 Hz, 1H), 2.78 (dd, J = 13.9, 9.3 Hz, 1H), 2.58 (dt, J = 7.6, 4.1 Hz, 1H), 2.19 (td, J = | | | | |
| | | | 13.9, 3.9 Hz, 1H), | | | | |
| | | | 1.99 (dt, J = 14.6, | | | | |
| | | | 4.0 Hz, 1H), 1.89 | | | | |
| | | | (td, J = 13.4, 3.2 | | | | |
| | | | Hz, 1H), 1.85-1.82 | | | | |
| | | | (m, 1H), 1.81-1.74 | | | | |
| | | | (m, 4H), 1.62 (dd, | | | | |
| | | | J = 13.1, 3.4 Hz, | | | | |
| | | | 1H), 1.51 (dt, J = | | | | |
| | | | 13.2, 4.7 Hz, 1H), | | | | |
| | | | 1.37 (dq, J = 20.9, | | | | |
| | | | 7.2 Hz, 1H), 1.28 | | | | |
| | | | (s, 3H), 1.24-1.17 | | | | |
| | | | (m, 1H), 1.02-0.82 | | | | |
| | | | (m, 7H). | | | | |
| 315 | 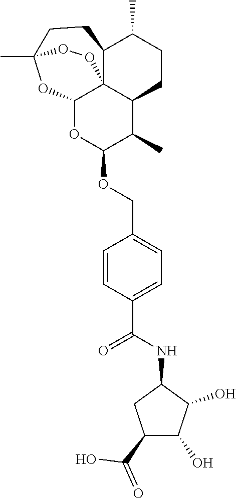 [SMILES: C[C@H]1[C@@H](OCc2ccc(C(=O)N[C@@H]3C[C@H](C(=O)O)[C@@H](O)[C@H]3O)cc2)O[C@@H]2O[C@@]3(C)CC[C@H]4[C@H](C)CC[C@@H]1[C@@]24OO3] | (1S,2R,3S,4R)- 2,3-dihydroxy- 4-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamido) cyclopentane-1- carboxylic acid | | | | | |
| 316 | 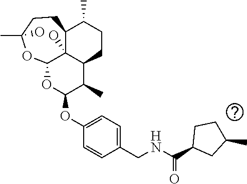 | (1R,3S)-3-((4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)benzyl) carbamoyl) cyclopentane-1- | ¹H NMR (400 MHz, DMSO) δ 12.08 (s, 1H), 8.27 (t, J = 5.9 Hz, 1H), 7.17 (d, J = 8.6 Hz, 2H), 7.01 (d, J = 8.6 Hz, 2H), 5.51 (d, J = 3.4 Hz, 1H), 5.41 (s, 1H), 4.19 (d, J = 5.8 Hz, 2H), 2.66 (ddd, J = 18.2, 10.0, 7.8 Hz, 1H), 2.61-2.55 (m, | | | | |
| | | carboxylic acid | 1H), 2.19 (td, J = | | | | |
| | | | 14.0, 3.9 Hz, 1H), | | | | |
| | | | 2.09-1.96 (m, 2H), | | | | |
| | | | 1.94-1.87 (m, 1H), | | | | |
| | | | 1.87-1.69 (m, 7H), | | | | |
| | | | 1.63 (dd, J = 13.1, | | | | |
| | | | 3.3 Hz, 1H), 1.51 | | | | |
| | | | (dt, J = 13.2, 4.6 Hz, | | | | |
| | | | 1H), 1.44-1.32 (m, | | | | |
| | | | 2H), 1.27 (s, 3H), | | | | |
| | | | 1.20 (dt, J = 11.3, | | | | |
| | | | 5.6 Hz, 1H), 1.00- | | | | |
| | | | 0.86 (m, 7H). | | | | |
| 317 | 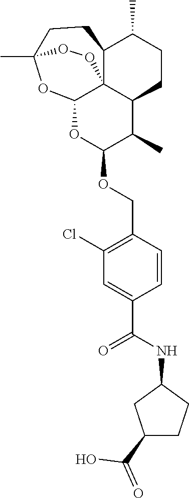 [SMILES: C[C@H]1[C@@H](OCc2ccc(C(=O)N[C@H]3CC[C@@H](C(=O)O)C3)cc2Cl)O[C@@H]2O[C@@]3(C)CC[C@H]4[C@H](C)CC[C@@H]1[C@@]24OO3] | (1R,3S)-3-(3- chloro-4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzamido) cyclopentane-1- carboxylic acid | | | | | |
| 318 | 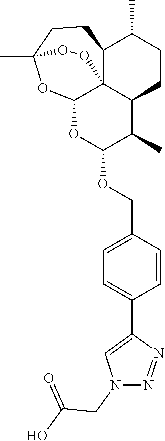 [SMILES: C[C@H]1[C@H](OCc2ccc(-c3cn(CC(=O)O)nn3)cc2)O[C@@H]2O[C@@]3(C)CC[C@H]4[C@H](C)CC[C@@H]1[C@@]24OO3] | 2-(4-(4- ((((3R,5aS,6R,8aS, 9R,10R,12R, 12aR)-3,6,9- trimethyldeca- hydro-12H-3,12- epoxy[1,2] dioxepino[4,3-i] isochromen-10- yl)oxy)methyl) phenyl)-1H- 1,2,3-triazol-1- yl)acetic acid | ¹H NMR (400 MHz, DMSO) δ 13.44 (s, 1H), 8.54 (s, 1H), 7.84 (d, J = 8.2 Hz, 2H), 7.44 (d, J = 8.1 Hz, 2H), 5.47 (s, 1H), 5.31 (s, 2H), 4.83 (d, J = 12.6 Hz, 1H), 4.65-4.54 (m, 2H), 2.19 (td, J = 14.1, 4.1 Hz, 2H), 2.00 (d, J = 14.5 Hz, 1H), 1.86- 1.77 (m, 1H), 1.65- 1.53 (m, 2H), 1.53- 1.43 (m, 1H), 1.43- 1.27 (m, 6H), 1.18 (dd, J = 11.4, 6.5 Hz, 1H), 1.00-0.86 (m, 4H), 0.84 (d, J = 7.1 Hz, 3H). | | | | |
| 319 | 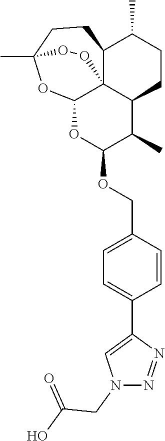 [SMILES: C[C@H]1[C@@H](OCc2ccc(-c3cn(CC(=O)O)nn3)cc2)O[C@@H]2O[C@@]3(C)CC[C@H]4[C@H](C)CC[C@@H]1[C@@]24OO3] | 2-(4-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) phenyl)-1H-1,2,3- triazol-1-yl) acetic acid | ¹H NMR (400 MHz, DMSO) δ 13.41 (s, 1H), 8.54 (s, 1H), 7.84 (d, J = 8.2 Hz, 2H), 7.41 (d, J = 8.0 Hz, 2H), 5.42 (s, 1H), 5.32 (s, 2H), 4.84 (d, J = 3.5 Hz, 1H), 4.80 (d, J = 12.5 Hz, 1H), 4.47 (d, J = 12.5 Hz, 1H), 2.47-2.42 (m, 1H), 2.19 (td, J = 13.9, 3.9 Hz, 1H), 2.05- 1.96 (m, 1H), 1.87- 1.65 (m, 3H), 1.60- 1.51 (m, 1H), 1.46- 1.28 (m, 6H), 1.17 (dd, J = 11.4, 6.5 Hz, 1H), 0.97-0.78 (m, 7H). | | | | |
| 320 | 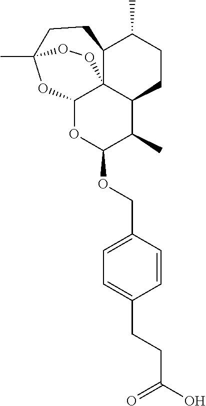 [SMILES: C[C@H]1[C@@H](OCc2ccc(CCC(=O)O)cc2)O[C@@H]2O[C@@]3(C)CC[C@H]4[C@H](C)CC[C@@H]1[C@@]24OO3] | 3-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) phenyl)propanoic acid | | | | | |
| 321 | 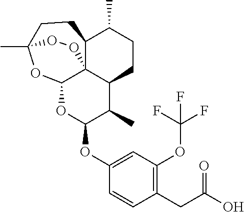 [SMILES: C[C@H]1[C@@H](Oc2ccc(CC(=O)O)c(OC(F)(F)F)c2)O[C@@H]2O[C@@]3(C)CC[C@H]4[C@H](C)CC[C@@H]1[C@@]24OO3] | 2-(2- (trifluoro- methoxy)-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 12.45 (s, 1H), 7.37 (d, J = 8.5 Hz, 1H), 7.09 (dd, J = 8.5, 2.4 Hz, 1H), 7.02 (s, 1H), 5.61 (d, J = 3.4 Hz, 1H), 5.45 (s, 1H), 3.58 (s, 2H), 2.60 (dt, J = 7.9, 4.2 Hz, 1H), 2.19 (td, J = 13.9, 3.8 Hz, 1H), 2.04- 1.95 (m, 1H), 1.82 | | | | |
| | | | (dq, J = 13.4, 5.9 | | | | |
| | | | Hz, 3H), 1.63 (dd, | | | | |
| | | | J = 13.2, 3.4 Hz, | | | | |
| | | | 1H), 1.52 (dt, J = | | | | |
| | | | 11.9, 5.1 Hz, 1H), | | | | |
| | | | 1.48-1.40 (m, 1H), | | | | |
| | | | 1.35 (dt, J = 13.5, | | | | |
| | | | 6.6 Hz, 0H), 1.29 | | | | |
| | | | (s, 3H), 1.19 (dt, J = | | | | |
| | | | 11.1, 5.7 Hz, 1H), | | | | |
| | | | 1.02-0.83 (m, 7H). | | | | |
| 322 | 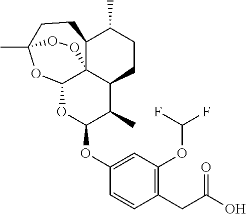 [SMILES: C[C@H]1[C@@H](Oc2ccc(CC(=O)O)c(OC(F)F)c2)O[C@@H]2O[C@@]3(C)CC[C@H]4[C@H](C)CC[C@@H]1[C@@]24OO3] | 2-(2- (difluoro- methoxy)-4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetic acid | ¹H NMR (400 MHz, DMSO) δ 12.34 (s, 1H), 7.42-7.14 (m, 2H), 7.00-6.91 (m, 1H), 6.86 (d, J = 2.3 Hz, 1H), 5.59 (d, J = 3.4 Hz, 1H), 5.43 (s, 1H), 3.53 (s, 2H), 2.64-2.56 (m, 1H), 2.19 (td, J = 13.8, 3.8 Hz, 1H), 2.00 (dt, J = 14.5, 4.0 Hz, 1H), 1.92-1.76 (m, 3H), 1.64 (dd, J = | | | | |
| | | | 13.1, 3.3 Hz, 1H), | | | | |
| | | | 1.53 (dt, J = 12.8, | | | | |
| | | | 4.9 Hz, 1H), 1.47- | | | | |
| | | | 1.34 (m, 2H), 1.29 | | | | |
| | | | (s, 3H), 1.25-1.16 | | | | |
| | | | (m, 1H), 1.01-0.80 | | | | |
| | | | (m, 7H). | | | | |
| 323 | 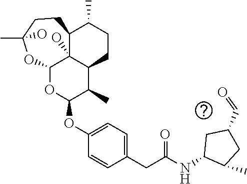 | (1R,2S,4R,5S)- 4-(2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetamido) bicyclo[3.1.0] | | | | | |
| | | hexane-2- | | | | | |
| | | carboxylic acid | | | | | |
| 324 | 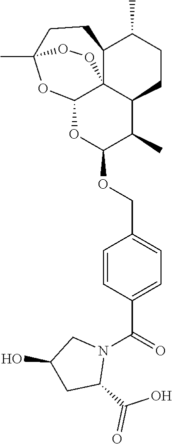 [SMILES: C[C@H]1[C@@H](OCc2ccc(C(=O)N3C[C@H](O)C[C@H]3C(=O)O)cc2)O[C@@H]2O[C@@]3(C)CC[C@H]4[C@H](C)CC[C@@H]1[C@@]24OO3] | (2S,4R)-4- hydroxy-1-(4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) benzoyl) pyrrolidine-2- carboxylic acid | ¹H NMR (400 MHz, DMSO) δ 12.57 (s, 1H), 7.53 (d, J = 8.2 Hz, 1H), 7.41 (d, J = 8.1 Hz, 2H), 5.40 (s, 1H), 4.91-4.73 (m, 2H), 4.57-4.43 (m, 2H), 4.27 (s, 1H), 3.73 (dd, J = 11.0, 3.7 Hz, 1H), 3.29 (d, J = 11.3 Hz, 2H), 2.48-2.41 (m, 1H), 2.19 (tt, J = 14.0, 3.7 Hz, 2H), 2.07- 1.89 (m, 2H), 1.76 (dddd, J = 31.6, 15.5, 8.9, 5.3 Hz, 3H), 1.56 (dd, J = 13.1, 3.4 Hz, 1H), 1.48-1.33 (m, 3H), 1.30 (s, 3H), 1.17 (dt, J = 11.2, 5.6 Hz, 1H), 0.99-0.79 (m, 7H). | | | | |
| 325 | 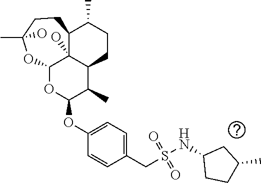 | (1R,3S)-3-(((4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) methyl) sulfonamido) | ¹H NMR (400 MHz, DMSO) δ 12.10 (s, 1H), 7.33-7.26 (m, 2H), 7.19 (d, J = 7.5 Hz, 1H), 7.12-7.02 (m, 2H), 5.57 (d, J = 3.4 Hz, 1H), 5.41 (s, 1H), 4.24 (s, 2H), 3.61-3.48 (m, 1H), 2.67-2.51 (m, 2H), 2.17 (ddd, J = 20.2, 16.4, 10.8 Hz, 2H), | | | | |
| | | cyclopentane-1- | 1.99 (dd, J = 14.9, | | | | |
| | | carboxylic acid | 4.5 Hz, 1H), 1.95- | | | | |
| | | | 1.70 (m, 6H), 1.68- | | | | |
| | | | 1.56 (m, 2H), 1.56- | | | | |
| | | | 1.45 (m, 2H), 1.39 | | | | |
| | | | (ddd, J = 25.7, 12.9, | | | | |
| | | | 4.1 Hz, 2H), 1.28 (s, | | | | |
| | | | 3H), 1.21 (dd, J = | | | | |
| | | | 11.0, 5.9 Hz, 1H), | | | | |
| | | | 1.02-0.82 (m, 7H). | | | | |
| 326 | 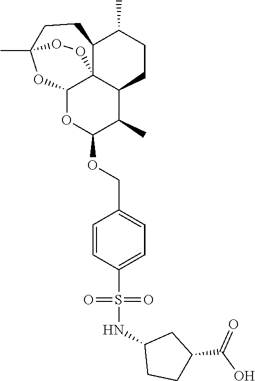 [SMILES: C[C@H]1[C@@H](OCc2ccc(S(=O)(=O)N[C@H]3CC[C@@H](C(=O)O)C3)cc2)O[C@@H]2O[C@@]3(C)CC[C@H]4[C@H](C)CC[C@@H]1[C@@]24OO3] | (1R,3S)-3-((4- ((((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)methyl) phenyl) sulfonamido) cyclopentane-1- carboxylic acid | | | | | |
| 327 | 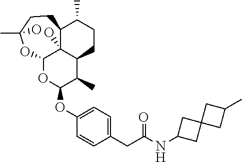 | 6-(2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetamido)spiro [3.3]heptane-2- | ¹H NMR (400 MHz, DMSO) δ 12.03 (s, 1H), 8.19 (d, J = 7.5 Hz, 1H), 7.15 (d, J = 8.6 Hz, 2H), 6.97 (d, J = 8.6 Hz, 2H), 5.49 (d, J = 3.4 Hz, 1H), 5.41 (s, 1H), 3.99 (h, J = 8.0 Hz, 1H), 3.26 (s, 2H), 2.91 (s, 1H), 2.62- 2.55 (m, 1H), 2.42- | | | | |
| | | carboxylic acid | 2.05 (m, 7H), 2.03- | | | | |
| | | | 1.75 (m, 6H), 1.67- | | | | |
| | | | 1.46 (m, 2H), 1.45- | | | | |
| | | | 1.33 (m, 2H), 1.28 | | | | |
| | | | (s, 3H), 1.19 (td, J = | | | | |
| | | | 11.4, 6.5 Hz, 1H), | | | | |
| | | | 1.02-0.82 (m, 7H). | | | | |
| 328 | 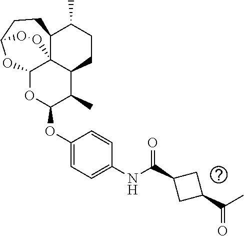 [SMILES: CC(=O)[C@H]1C[C@@H](C(=O)Nc2ccc(O[C@H]3O[C@@H]4O[C@H]5CC[C@H]6[C@H](C)CC[C@@H]([C@H]3C)[C@@]46OO5)cc2)C1] | (1R,3s)-3-((4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) carbamoyl) cyclobutane-1- carboxylic acid | | | | | |
| 329 | 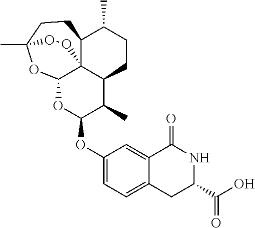 [SMILES: C[C@H]1[C@@H](Oc2ccc3c(c2)C(=O)N[C@H](C(=O)O)C3)O[C@@H]2O[C@@]3(C)CC[C@H]4[C@H](C)CC[C@@H]1[C@@]24OO3] | (S)-1-oxo-7- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)-1,2,3,4- tetrahydroiso- quinoline-3- carboxylic acid | ¹H NMR (400 MHz, DMSO) δ 8.06 (d, J = 4.2 Hz, 1H), 7.49 (d, J = 2.7 Hz, 1H), 7.25 (d, J = 8.3 Hz, 1H), 7.15 (dd, J = 8.2, 2.7 Hz, 1H), 5.55 (d, J = 3.5 Hz, 1H), 5.44 (s, 1H), 4.19 (dt, J = 7.2, 3.8 Hz, 1H), 3.24 (dd, J = 16.0, 6.6 Hz, 2H), 2.60 (dt, J = 7.6, 4.0 Hz, 1H), 2.19 (td, J = 14.0, | | | | |
| | | | 3.8 Hz, 1H), 2.04- | | | | |
| | | | 1.87 (m, 2H), 1.82 | | | | |
| | | | (dd, J = 18.3, 5.5 | | | | |
| | | | Hz, 2iH), 1.68-1.60 | | | | |
| | | | (m, 1H), 1.57-1.47 | | | | |
| | | | (m, 1H), 1.47-1.32 | | | | |
| | | | (m, 2H), 1.30 (s, | | | | |
| | | | 3H), 1.21 (dd, J = | | | | |
| | | | 11.3, 6.4 Hz, 1H), | | | | |
| | | | 0.97 (d, J = 7.3 Hz, | | | | |
| | | | 3H), 0.90 (d, J = 6.4 | | | | |
| | | | Hz, 3H). | | | | |
| 330 | 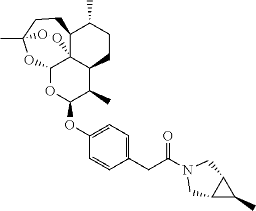 | (1R,5S,6S)-3- (2-(4- (((3R,5aS,6R,8aS, 9R,10S,12R, 12aR)-3,6,9- trimethyldeca- hydro-12H-3,12- epoxy[1,2] dioxepino[4,3-i] isochromen-10- yl)oxy)phenyl) acetyl)-3- azabicyclo[3.1.0] hexane-6- | ¹H NMR (400 MHz, DMSO) δ 12.27 (s, 1H), 7.12 (d, J = 8.5 Hz, 2H), 6.99 (d, J = 8.7 Hz, 2H), 5.50 (d, J = 3.4 Hz, 1H), 5.42 (s, 1H), 3.77 (d, J = 10.7 Hz, 1H), 3.66 (d, J = 12.1 Hz, 1H), 3.55 (dd, J = 10.7, 4.2 Hz, 1H), 3.51 (s, 2H), 2.58 (dt, J = 7.6, 4.2 Hz, 1H), | | | | |
| | | carboxylic acid | 2.19 (td, J = 14.0, | | | | |
| | | | 3.9 Hz, 1H), 2.10- | | | | |
| | | | 2.04 (m, 1H), 1.99 | | | | |
| | | | (dt, J = 7.3, 3.4 Hz, | | | | |
| | | | 2H), 1.86 (ddd, J = | | | | |
| | | | 39.6, 13.3, 3.8 Hz, | | | | |
| | | | 3H), 1.63 (dd, J = | | | | |
| | | | 13.1, 3.4 Hz, 1H), | | | | |
| | | | 1.51 (dt, J = 13.3, | | | | |
| | | | 4.7 Hz, 1H), 1.46- | | | | |
| | | | 1.33 (m, 2H), 1.32- | | | | |
| | | | 1.25 (m, 4H), 1.24- | | | | |
| | | | 1.13 (m, 1H), 1.01- | | | | |
| | | | 0.84 (m, 7H). | | | | |

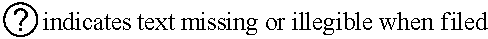

The following non-limiting examples are additional embodiments that further illustrate the present disclosure.

All endoperoxide compounds described in the present disclosure are obtained from commercial sources or are synthesized according to published procedures. See, e.g., M. Rudrapal et al. Drug Design, Development and Therapy 10 (2016) 3575-3590, D. M. Opsenica et al. J. Serb. Chem. Soc. 74(11) (2009) 1155-1193, and references cited therein.

MDS-L differentiation assay. Given a limited number of patient samples and the low yield from each biopsy, it is not tractable to conduct high-throughput screens with primary cells. We therefore obtained a cell line isolated from CD34+MDS HPCs termed MDS-L, which has been previously demonstrated to be differentiation competent when supplemented with appropriate cytokines (J. Fang et al., Nat. Med. 22(7) (2016) 727-734; K. Tohyama et al., Br. J. Haematol. 91(4) (1995) 795-799).

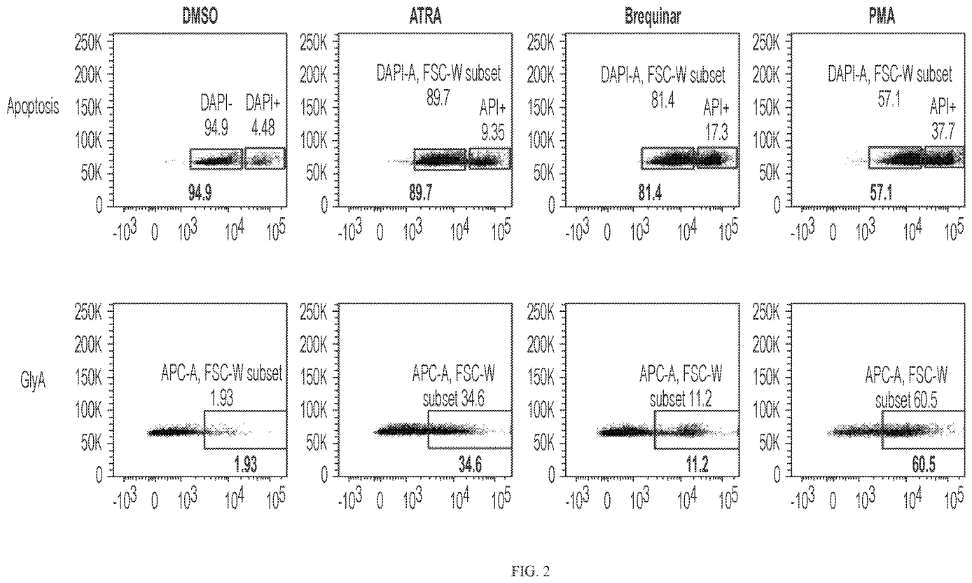

To establish a screening assay, we selected known differentiation inducers of acute myeloid leukemia cells including retinoic acid (ATRA), Brequinar, and phorbol 12-myristate 13-acetate (PMA). We found that all agents were efficacious at inducing differentiation to more terminal blood lineages, a measurement assessed by cellular immunopositivity to anti-GlyA directed antibodies in a 384-well FACS-based assay. Among these, we found ATRA to be highly efficacious at inducing differentiation while displaying minimal cytotoxicity as readout by live dead cell gating (FIG. 2). For these reasons, ATRA served as a suitable positive control for screening efforts. With this established miniaturized assay, we then screened the comprehensive drug repurposing library ReFRAME for previously unannotated agents that induce differentiation of MDS-L cells with minimal cytotoxicity. Among the top validated hits from this screen were molecules representative of known mechanisms for inducing cell cycle arrest in MDS cells including topoisomerase inhibitors, tubulin-targeting agents, DNA damaging chemotherapeutics, and a number of receptor tyrosine kinase inhibitors, among others.

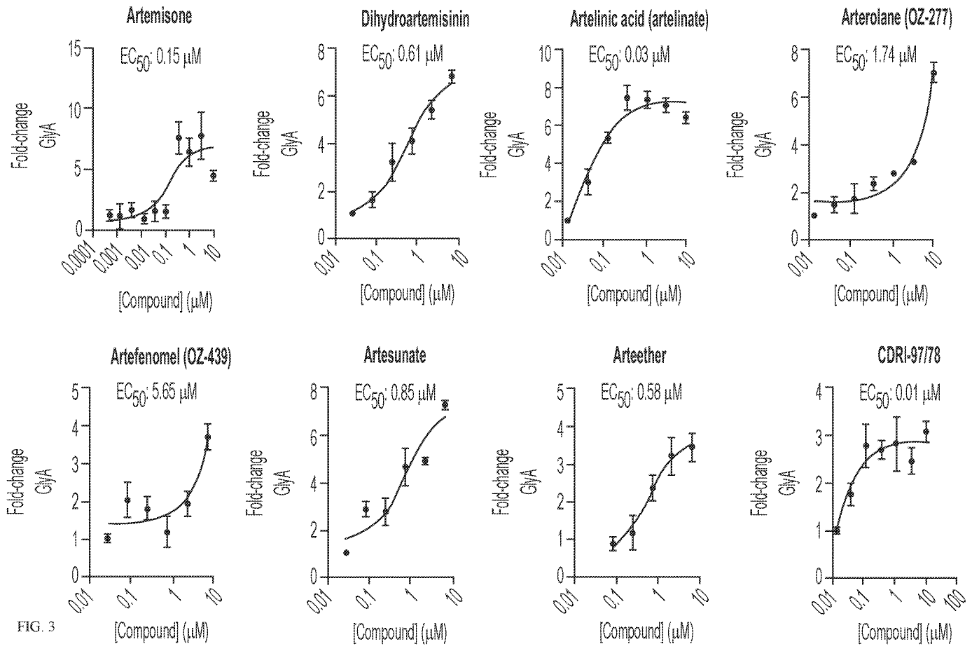

Surprisingly, screening hits with no previously reported activity in MDS biology were two semi-synthetic derivatives of artemisinin, a naturally occurring endoperoxide that is an established antimalarial drug. We therefore evaluated representative endoperoxides, including dihydroartemisinin, arteether, artemisone, artesunate, and the synthetic trioxolane artefenomel. MDS-L were cultured in RPMI (17-105-CV, Corning) without phenol red and supplemented with 2 mM glutamine and 10% FBS. Cells first expanded in IL-3 until 70% confluence then pre-conditioned for 14 days with IL-3 (200-03, PeproTech), GCSF (300-23, PeproTech), and EPO (287-TC, R&D Systems) to promote erythroid and myeloid differentiation. A density of 10,000 cells per well were used in phenotypic assays. MDSL cells were spun and treatment media was replaced with media containing APC Anti-CD235a (GlyA) (551336, BD Biosciences) (1.25 μL per 50 L test) and incubated for 1 hour at 4° C. Cells were washed then counter stained with SYTOX-Green 1 M per 100 L prior to flow cytometry analysis. Dose response and pharmacological assays were analyzed using a ZE5 Cell Analyzer (BioRad) equipped with Everest software. Single, live, cells were analyzed for GlyA expression using FlowJo v10.7.2 software. Results are shown in Table 5. All the tested compounds were efficacious at inducing GlyA positivity induction in MDS-L cells; the half maximal potencies of these agents varied over two orders of magnitude (FIG. 3).

TABLE 2
MDS-L differentiation assay of antimalarial endoperoxide compounds.

| | | MDS-L GlyA | Maximal |
|---|---|---|---|
| | | induction EC₅₀ | MDS-LGlyA |
| Compound name | Structure | (μM) | fold induction |
| Artelinic acid (artelinate) | 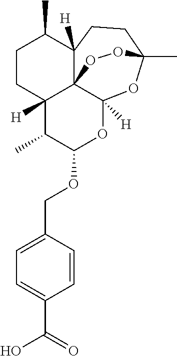 [SMILES: C[C@H]1[C@@H](OCc2ccc(C(=O)O)cc2)O[C@@H]2O[C@@]3(C)CC[C@H]4[C@H](C)CC[C@@H]1[C@@]24OO3] | 0.03 | 7.26 |
| Artemisone | 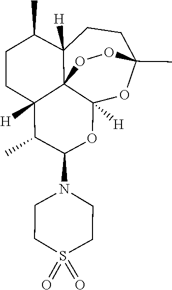 [SMILES: C[C@H]1[C@H](N2CCS(=O)(=O)CC2)O[C@@H]2O[C@@]3(C)CC[C@H]4[C@H](C)CC[C@@H]1[C@@]24OO3] | 0.15 | 6.84 |
| Arteether | 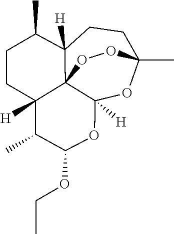 [SMILES: CCO[C@H]1O[C@@H]2O[C@@]3(C)CC[C@H]4[C@H](C)CC[C@@H]([C@H]1C)[C@@]24OO3] | 0.58 | 3.82 |
| Dihydroartemisin | 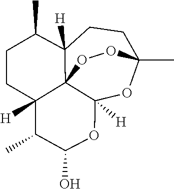 [SMILES: C[C@H]1[C@@H](O)O[C@@H]2O[C@@]3(C)CC[C@H]4[C@H](C)CC[C@@H]1[C@@]24OO3] | 0.61 | 7.09 |
| Artesunate | 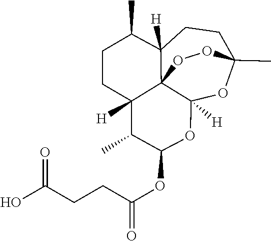 [SMILES: C[C@H]1[C@H](OC(=O)CCC(=O)O)O[C@@H]2O[C@@]3(C)CC[C@H]4[C@H](C)CC[C@@H]1[C@@]24OO3] | 0.85 | 7.51 |
| Arterolane (OZ-277) | 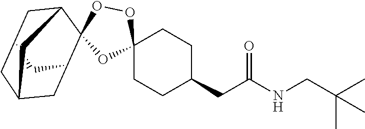 [SMILES: CC(C)(C)CNC(=O)C[C@H]1CC[C@]2(CC1)OO[C@]1(O2)[C@@H]2C[C@H]3C[C@@H](C2)C[C@@H]1C3] | 1.74 | 7.02 |
| Artefenomel (OZ-439) | 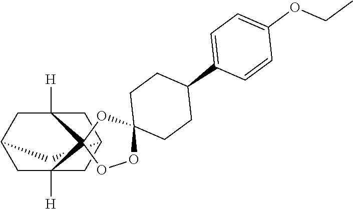 | 5.65 | 3.71 |
| CDRI-97/78 | 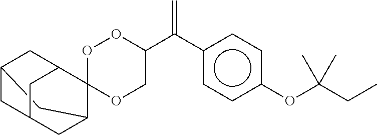 | 0.01253 | 3.09 |

Likewise, we demonstrated that the exemplary artemisinin compounds aretmisone and arteether efficaciously induced terminal differentiation of MDS-L cells to CFU—Es while decreasing the number of BFU-E, CFU-GM and CFU-GEMM type colonies in CFU assays. Additionally, artemisone and arteether efficaciously promoted the loss of CD71 and displayed no significant induction in apoptosis below 20 μM. Together, these results indicate a functional and non-toxic impact on multi-lineage differentiation in MDS blastic cells. In contrast, the non-peroxide containing molecule 2-deoxy-artemisinin was inactive in differentiation assays.

Heme depletion induces terminal differentiation of MDS-L cells. The purpose of this example is to establish a pharmacological understanding of how endoperoxides, which have no known therapeutic activity in MDS, are surprisingly efficacious for inducing the differentiation of MDS blasts. Because 2-deoxy-artemisinin is inactive for this purpose, we reasoned that the endoperoxide core of the active molecules identified in the screen corresponds to their activity, indicating a similar mechanism to how artemisinins induce compromise of the malarial parasite. In this therapeutically distinct context, artemisinins are known to be activated by heme-iron, a catalytic event which catalyzes cleavage of the endoperoxide, generating free radicals and forming reactive species that can inactivate essential proteins required for parasite survival.

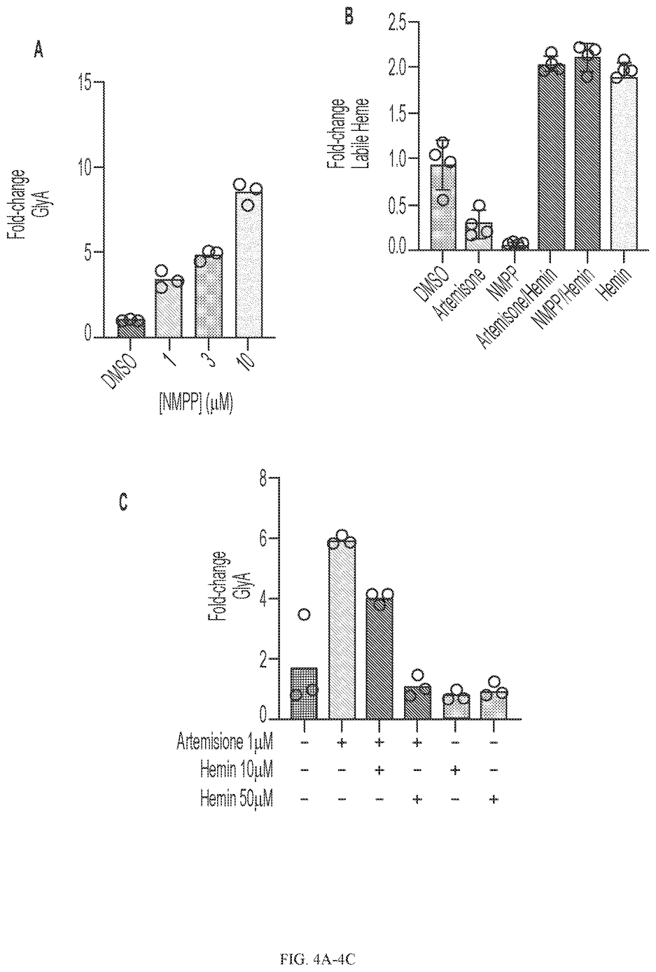

An additional consequence of this mechanism is the alkylation of artemisinin to heme, resulting in its functional inactivation. Because we found no other reactive oxygen species (ROS)-inducers or alkylators as active hits from the screen, we reasoned that heme depletion is a way in which artemisinins promote differentiation of this cellular population. To illustrate further, we found that inhibition of heme biosynthesis effectively induced MDS differentiation, as treatment with the ferrochelatase inhibitor NMPP (N-methylprotoporphyrin) was found to increase GlyA positivity in MDS-L cells (FIG. 4A). Similarly, we found that treatment with NMPP and representative artemisinin derivative artemisone efficiently reduced levels of labile heme in MDS-L cells over relevant time periods (FIG. 4B). Further, we found that the addition of exogenous heme in the form of hemin suppressed the potential of artemisone to induce differentiation of MDS-L cells (FIG. 4C). These results position pharmacological heme depletion, such as by direct conjugation or by inhibition of its biosynthesis, as a means to induce differentiation of an MDS relevant cell type and a means for differentiation-inducing therapy for MDS.

### Pharmaceutical Composition

The disclosure also provides, optionally for use in combination with the methods described herein, a pharmaceutical composition comprising a therapeutically effective amount of one or more compounds or a pharmaceutically acceptable salt described herein, in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Tables 1 to 4, or a pharmaceutically acceptable salt thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound or a pharmaceutically acceptable salt thereof that is administered is governed by such considerations, and is the minimum amount necessary to restore normal erythropoiesis in patients suffering from MDS, to induce differentiation of MDS-L cells, to inhibit heme biosynthesis, or any combination thereof. Such amount may be below the amount that is toxic to normal cells, or the subject as a whole. Generally, the initial therapeutically effective amount of a compound of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of a compound of the present disclosure. In another embodiment, such dosage forms contain from about 50 mg to about 500 mg of a compound of the present disclosure. In yet another embodiment, such dosage forms contain from about 25 mg to about 200 mg of a compound of the present disclosure. In still another embodiment, such dosage forms contain from about 10 mg to about 100 mg of a compound of the present disclosure. In a further embodiment, such dosage forms contain from about 5 mg to about 50 mg of a compound of the present disclosure. In any of the foregoing embodiments the dosage form can be administered once a day or twice per day.

The compositions of the present disclosure can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

In an embodiment, also encompassed are pharmaceutical compositions suitable for single unit dosages that comprise a compound of the disclosure or its pharmaceutically acceptable salt, and a pharmaceutically acceptable carrier.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of a compound of the present disclosure.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydroxpropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents.

The pharmaceutical composition may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compound as described herein may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### EXAMPLES

### Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), 1,3-Diisopropylcarbodiimide (DIC), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), 0-(7-azabenzotriazole-1-yl)-N, N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

The following scheme is a representative method f preparing the compounds in Table 1. One of skill in the art would be able to extrapolate this scheme in order to prepare each of the compounds in Table 1.

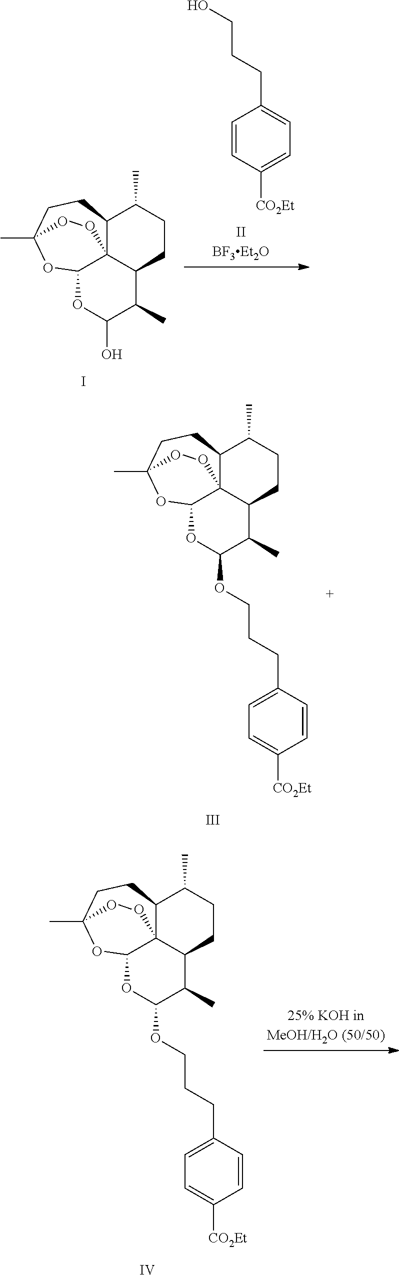

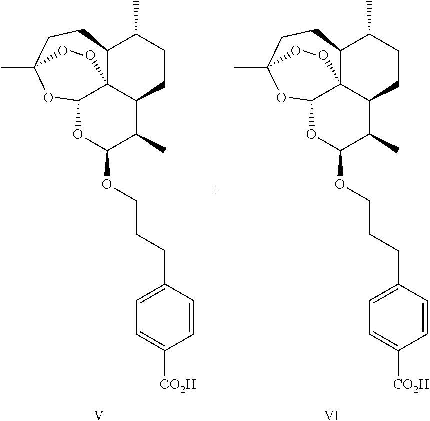

Dihydroartemisinin (I, 500 mg, 1.75 mmol, 1.0 eq) and ethyl 4-(3-hydroxypropyl) benzoate (II, 549 mg, 2.6 mmol, 1.5 eq) were stirred in 20 mL anhydrous DCM under N₂ atmosphere at 0° C., then BF₃·Et₂O (125 mg, 0.88 mmol, 0.5 eq) was added drop-wise. The solution was stirred for 2 h at room temperature. TLC showed the reaction completed and quenched with saturated NaHCO₃ solution, extracted with DCM 3 times. The organic phase was combined and dried with Na₂SO₄. The organic solvent was removed in vacuum and the crude product was purified via silica column chromatography (EtOAc/Hexane, 0:1-1:2) slowly. The epimers III and IV can be separated as colorless oil which were confirmed by HNMR and LCMS. Cis-3 was the major product.

Compound III, 1H NMR (400 MHz, CDCl3) δ 7.96 (d, J=8.3 Hz, 2H), 7.24 (d, J=8.3 Hz, OH), 5.39 (s, 1H), 4.78 (d, J=3.5 Hz, 1H), 4.36 (q, J=7.1 Hz, 2H), 3.86 (dt, J=9.8, 6.2 Hz, 1H), 3.39 (dt, J=9.8, 6.3 Hz, 1H), 2.73 (dd, J=8.8, 6.7 Hz, 2H), 2.63 (ddq, J=7.3, 3.8, 2.2 Hz, 1H), 2.37 (ddd, J=14.7, 13.5, 4.0 Hz, 1H), 2.03 (ddd, J=14.6, 5.0, 3.0 Hz, 1H), 1.94-1.84 (m, 3H), 1.84-1.73 (m, 2H), 1.64 (dq, J=13.2, 3.4 Hz, 2H), 1.55-1.44 (m, 2H), 1.43 (s, 3H), 1.38 (d, J=7.1 Hz, 3H), 1.32 (ddd, J=11.5, 6.2, 3.4 Hz, 2H), 1.29-1.17 (m, 2H), 0.94 (dd, J=12.1, 6.8 Hz, 7H).

Compound IV, 1H NMR (400 MHz, CDCl3) δ 7.95 (d, J=8.3 Hz, 2H), 7.47-7.01 (m, 2H), 5.32 (s, 1H), 4.40 (d, J=9.3 Hz, 1H), 4.35 (q, J=7.1 Hz, 2H), 3.99 (dt, J=9.7, 6.0 Hz, 1H), 3.40 (ddd, J=9.6, 7.5, 5.8 Hz, 1H), 2.89-2.66 (m, 2H), 2.50-2.31 (m, 2H), 2.02 (ddd, J=14.6, 4.9, 3.0 Hz, 1H), 1.98-1.91 (m, 1H), 1.88 (dtd, J=16.7, 6.1, 2.8 Hz, 2H), 1.76 (dq, J=13.5, 3.7 Hz, 1H), 1.70 (q, J=3.3 Hz, 1H), 1.67 (d, J=3.3 Hz, 1H), 1.55 (dt, J=14.0, 4.7 Hz, 1H), 1.50-1.45 (m, 1H), 1.44 (s, 2H), 1.38 (t, J=7.1 Hz, 3H), 1.34-1.21 (m, 3H), 1.07-0.98 (m, 1H), 0.952 (d, J=7.2 Hz, 3H). 0.91 (d, J=7.2 Hz, 3H).

25% KOH MeOH/H₂O (50/50 in volume) was prepared and then added to compound III, the solution was stirred for 2 h at room temperature, TLC showed the substrate consumed. The solution was acidized with acetic acid to PH neutral. The solution was extracted with ethyl acetate 3 times, and the organic phase were combined, dried with Na₂SO₄. The organic solvent was removed in vacuum and the crude product was purified via silica column chromatography (EtOAc/Hexane, 1:1-1:0) to give product V.

Compound V, 1H NMR (400 MHz, CDCl3) δ 8.03 (d, J=8.3 Hz, 1H), 7.29 (d, J=8.5 Hz, 1H), 5.40 (s, 1H), 4.80 (d, J=3.5 Hz, 1H), 3.88 (dt, J=9.8, 6.2 Hz, 1H), 3.41 (dt, J=9.9, 6.3 Hz, 1H), 2.95-2.71 (m, 2H), 2.64 (dt, J=7.9, 4.2 Hz, 1H), 2.37 (ddd, J=14.6, 13.4, 4.0 Hz, 1H), 2.09-1.99 (m, 1H), 1.97-1.86 (m, 3H), 1.84-1.74 (m, 2H), 1.71-1.60 (m, 1H), 1.57-1.45 (m, 2H), 1.43 (s, 3H), 1.32 (ddd, J=11.4, 6.1, 3.4 Hz, 1H), 1.29-1.17 (m, 2H), 0.95 (dd, J=9.7, 6.8 Hz, 7H).

Compound VI was prepared using the same method. 1H NMR (400 MHz, CDCl3) δ 8.01 (d, J=8.3 Hz, 2H), 7.30 (d, J=8.6 Hz, 2H), 5.33 (s, 1H), 4.41 (d, J=9.2 Hz, 1H), 4.00 (dt, J=9.6, 5.9 Hz, 1H), 3.42 (ddd, J=9.7, 7.4, 5.8 Hz, 1H), 2.78 (tq, J=14.1, 7.3 Hz, 2H), 2.50-2.30 (m, 2zH), 2.09-1.84 (m, 4H), 1.76 (dt, J=13.3, 3.8 Hz, 1H), 1.69 (dd, J=13.2, 3.2 Hz, 1H), 1.56 (dt, J=13.8, 4.6 Hz, 1H), 1.52-1.46 (m, 1H), 1.44 (s, 3H), 1.37-1.19 (m, 4H), 1.07-0.98 (m, 1H), 0.96 (d, J=6.0 Hz, 3H), 0.92 (d, J=7.1 Hz, 3H).

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various issued patents, published patent applications, journal articles, and other publications, each of which are incorporated herein by reference.

What is claimed is:

1. A compound of any one of Formulae I-V 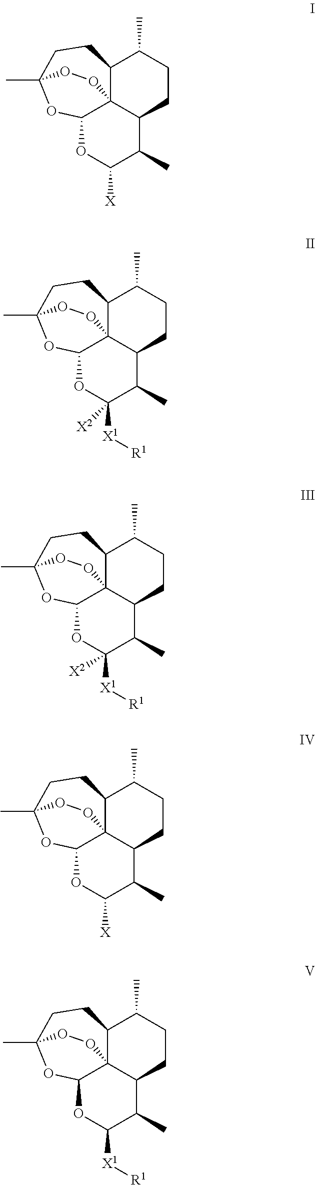
wherein
X is X¹R¹ or Y;
X¹ is —O—, —OC(═O)—, —OCH₂—, —OCH₂C(═O)—, —OCH(CH₃)—, —OCH₂C(═O)NH—, —CH₂—, —N(CH₃)—, —NH—, —NHCH₂—, —N(CH₃)CH₂—, —NHCH(CH₃)— or —S—;
R¹ is —(C₁-C₆) alkyl, —(C₁-C₆) heteroalkyl, —(C₁-C₆) hydroxyalkyl, —(C₆-C₁₀) aryl, —(C₁-C₆) alkyl (C₆-C₁₀) aryl, —(C₆-C₁₀) aryl-(C₁-C₆) alkyl, —NH(C₆-C₁₀) aryl, —(C₅-C₁₀) heteroaryl, —(C₁-C₆) alkyl (C₅-C₁₀) heteroaryl, —(C₃-C₁₀) cycloalkyl, —(C₁-C₆) alkyl (C₃-C₁₀) cycloalkyl, —(C₂-C₆) alkenyl (C₃-C₁₀) cycloalkyl, spiro —(C₃-C₁₀) cycloalkyl, —(C₃-C₁₀) heterocycloalkyl, —(C₁-C₆) alkyl (C₃-C₁₀) heterocycloalkyl, —S(═O)₂(C₁-C₆) alkyl, —O—S(═O)₂(C₁-C₆) alkyl, —(C₁-C₆) alkyl-S(═O)₂(C₁-C₆) alkyl, —(C₁-C₆) alkyl-S(═O)₂NH(C₁-C₆) alkyl), —(C₁-C₆) alkyl-S(═O)₂N((C₁-C₆) alkyl)₂, —(C₁-C₆) alkyl-S(═O)₂NH₂, —(C₁-C₆) alkyl-NHS(═O)₂(C₁-C₆) alkyl, —(C₁-C₆) alkyl) C(═O)NH(C₁-C₆) alkyl, —(C₁-C₆) alkyl) C(═O)NH₂, —C(═O)OH, —(C₁-C₆) alkyl-C(═O)OH, 2,3-dihydrobenzofuran, 2,3-dihydro-1H-indene, 3,4-dihydroisoquinolin-1(2H)-one, —B(OH)₂, —(C₁-C₆) alkyl-C(═O)O(C₁-C₆) alkyl, or partially unsaturated fused bicyclic (C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, —(C₁-C₆) heteroalkyl, or partially unsaturated fused bicyclic (C₃-C₁₀) heterocycloalkyl may optionally be substituted with one or more R¹′;
each R¹′ is independently —OH, halo, oxo, —CN, —NH₂, —(C₁-C₆) alkyl, —(C₁-C₆) halo alkyl, —O—(C₁-C₆) halo alkyl, —(C₁-C₆) alkyl-OH, —CH₂C(═O)O(C₁-C₆) alkyl, —C(═O)O(C₁-C₆) alkyl, —(C₁-C₆) alkyl-C(═O)OH, —CH(CH₃)C(═O)OH, —C(═O)OH, —C(═O)C(═O)OH, —(C₁-C₆) heteroalkyl, —O—(C₁-C₆) alkyl, —S(═O)₂(C₁-C₆) alkyl, —C(═O)NHOH, —C(═O)NH(C₁-C₆) alkyl, —(C₁-C₆) alkyl-C(═O)NH(C₁-C₆) alkyl, —NHS(═O)₂(C₁-C₆) alkyl, —S(═O)₂NH₂, —S(═O)₂NH(C₁-C₆) alkyl, —S(═O)₂N((C₁-C₆) alkyl)₂, —S(═O)₂NH—(C₃-C₁₀) cycloalkyl, —(C₁-C₆) alkyl-S(═O)₂NH—(C₃-C₁₀) cycloalkyl, —S(═O)₂CF₃, —C(═O)NH₂, —(C₃-C₁₀) cycloalkyl, —O—(C₃-C₁₀) cycloalkyl, —(C₃-C₁₀) heterocycloalkyl, —(C₅-C₁₀) heteroaryl, —(C₁-C₆) alkyl (C₅-C₁₀) heteroaryl, —C(═O)NH—(C₃-C₁₀) cycloalkyl, —NHC(═O)(C₃-C₁₀) cycloalkyl, —(C₁-C₆) alkyl-NHC(═O)(C₃-C₁₀) cycloalkyl, —(C₁-C₆) alkyl-C(═O)(C₃-C₁₀) heterocycloalkyl, —C(═O)N(CH₃)—(C₃-C₁₀) cycloalkyl, —(C₁-C₆) alkyl-C(═O)NH—(C₃-C₁₀) cycloalkyl, —C(═O)NH(C₁-C₆) alkyl-(C₃-C₁₀) cycloalkyl, —(C₁-C₆) alkyl-C(═O)NH(C₁-C₆) alkyl-(C₃-C₈) cycloalkyl, —C(═O)NH—(C₃-C₁₀) heterocycloalkyl, —C(═O)NH(C₅-C₁₀) heteroaryl, —(C₁-C₆) alkyl C(═O)NH(C₅-C₁₀) heteroaryl, C(═O)NH-thietanyl 1,1-dioxide, —(C₁-C₆) alkyl-C(═O)OH, —(C₂-C₆) alkenyl-C(═O)OH, —(C₂-C₆) alkynyl-C(═O)OH, —CH₂C(═O)NH₂, —C(═O)NH(C₁-C₆)alkyl), —C(═O)N((C₁-C₆)alkyl)₂, —(C₁-C₆) alkyl-C(═O)OH, —(C₆-C₁₀) aryl, —C(═O)NH(C₆-C₁₀) aryl, —C(═O)NH—(C₁-C₆) alkyl(C₆-C₁₀) aryl, —C(═O)NH—(C₁-C₆) alkyl-P⁺(Ph)₃, —P⁺(Ph)₃, —B(OH)₂, —NHC(═O)—C(═O)OH, or —C(═O)(C₃-C₁₀) heterocycloalkyl, wherein any —(C₆-C₁₀) aryl, —(C₅-C₁₀) heteroaryl, —(C₃-C₁₀) cycloalkyl, —(C₃-C₁₀) heterocycloalkyl, —(C₁-C₆) alkyl, —O—(C₁-C₆) alkyl, or —(C₁-C₆) heteroalkyl may optionally be substituted with one or more R¹″;
or two R¹′ together form —(C₃-C₁₀) cycloalkyl or (C₃-C₁₀) heterocycloalkyl, optionally substituted with one or more R¹″;
each R¹″ is independently —OH, —NH₂, —(C₁-C₆) alkyl, —C(═O)OH, —(C₁-C₆) alkyl-C(═O)OH, —C(═O)O(C₁-C₆) alkyl, —CH₂C(═O)O(C₁-C₆) alkyl, —O(C₁-C₆) alkyl, —(C₁-C₆) halo alkyl, —O—(C₁-C₆) halo alkyl, —NHC(═O)OH, —NHC(═O)—(C₁-C₆) alkyl, —S(═O)₂NH₂, —S(═O)₂NH(C₁-C₆) alkyl, —S(═O)₂N((C₁-C₆) alkyl)₂, —S(═O)₂CF₃, —C(═O)NH₂, —(C₃-C₁₀) cycloalkyl, —(C₃-C₁₀) heterocycloalkyl, —(C₅-C₁₀) heteroaryl, oxo, or halo; and
Y is —(C₃-C₈) heterocycloalkyl, optionally substituted with one or more R¹′;
including enantiomers, racemic and scalemic mixtures, and pharmaceutically acceptable salts thereof.

2. The compound of claim 1, wherein X² is H.

3. The compound of claim 2, wherein X is X¹R¹.

4. The compound of claim 3, wherein X¹ is —O—, —O—CH₂—, —O—CH₂C(═O)—, or —O—CH₂C(═O)NH—.

5. The compound of claim 4, wherein R¹ is Ph, —(C₅-C₆) heteroaryl, or —(C₃-C₇) heterocycloalkyl optionally substituted with one or more R¹′.

6. The compound of claim 5, wherein at least one R¹′ is —C(═O)OCH₃, —(C₁-C₆) alkyl-C(═O)OH, or —C(═O)OH.

7. The compound of claim 5, wherein at least one R¹′ is (C₁-C₆) alkyl or is —(C₁-C₆) heteroalkyl.

8. The compound of claim 5, wherein at least one R¹′ is —C(═O)NH(C₁-C₆) alkyl.

9. The compound of claim 5, wherein at least one R¹′ is —OH, halo (C₁-C₆) alkyl, or CN.

10. The compound of claim 5, wherein at least one R¹′ is —(C₃-C₁₀) cycloalkyl, —(C₃-C₁₀) heterocycloalkyl, or —(C₅-C₁₀) heteroaryl.

11. The compound of claim 5, wherein at least one R¹′ is —C(═O)OH or —(C₁-C₆) alkyl-C(═O)OH and at least one R¹′ is halo, halo (C₁-C₆) alkyl, or —(C₁-C₆) alkyl.

12. The compound of claim 5, wherein at least one R¹″ is —C(═O)OH.

13. A compound selected from the group consisting of:
(3R,5aS,6R,8aS,9R,10S,12R,12aR)-10-((2,2-difluorobenzo[d][1,3]dioxol-5-yl)methoxy)-3,6,9-trimethyldecahydro-12H-3,12-epoxy[1,2]dioxepino[4,3-i]isochromene;
5-(4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)phenyl)-2H-tetrazole;
2-(3-methoxy-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
3-(methylsulfonyl)-1-((3R,5aS,6R,8aS,9R,10R,12R,12aR)-3,6,9-trimethyldecahydro-12H-3,12-epoxy[1,2]dioxepino[4,3-i]isochromen-10-yl)azetidine;
N-hydroxy-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamide;
(3R,5aS,6R,8aS,9R,10R,12R,12aR)-10-((2,2-difluorobenzo[d][1,3]dioxol-5-yl)methoxy)-3,6,9-trimethyldecahydro-12H-3,12-epoxy[1,2]dioxepino[4,3-i]isochromene;
4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzonitrile;
2-(3-methyl-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
2-(3-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
4-(3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)propyl)benzoic acid;
(1R,4s)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
2-(4-(((3R,5aS,6R,8aS,9R,10R,12R,12aR)-3,6,9-trimethyldecahydro-12H-3,12-epoxy[1,2]dioxepino[4,3-i]isochromen-10-yl)oxy)phenyl)acetic acid;
5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)picolinic acid;
2-(2,6-difluoro-4-(((3R,5aS,6R,8aS,9R,10R,12R,12aR)-3,6,9-trimethyldecahydro-12H-3,12-epoxy[1,2]dioxepino[4,3-i]isochromen-10-yl)oxy)phenyl)acetic acid;
4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)picolinic acid;
2-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
(3R,5aS,6R,8aS,9R,10R,12R,12aR)—N,3,6,9-tetramethyl-N-(2-(methylsulfonyl)ethyl)decahydro-12H-3,12-epoxy[1,2]dioxepino[4,3-i]isochromen-10-amine;
4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzenesulfonamide;
4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzonitrile;
2-(3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
4-(3-(((3R,5aS,6R,8aS,9R,10R,12R,12aR)-3,6,9-trimethyldecahydro-12H-3,12-epoxy[1,2]dioxepino[4,3-i]isochromen-10-yl)oxy)propyl)benzoic acid;
2-(2,6-difluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
2-(2,3-difluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
2-fluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
2-fluoro-4-(((3R,5aS,6R,8aS,9R,10S,12S,12aR)-3,6,9-trimethyldecahydro-12H-3,12-epoxy[1,2]dioxepino[4,3-i]isochromen-10-yl)oxy)benzoic acid;
2-(2-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
2-(2-fluoro-5-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
(3R,5aS,6R,8aS,9R,10R,12R,12aR)-3,6,9-trimethyl-N-(2-((trifluoromethyl)sulfonyl)ethyl)decahydro-12H-3,12-epoxy[1,2]dioxepino[4,3-i]isochromen-10-amine;
2,6-difluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamide;
2,3-difluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)furan-2-carboxylic acid;
2-chloro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
2-(3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)acetic acid;
2-methoxy-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
4-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)ethyl)benzoic acid;
(3R,5aS,6R,8aS,9R,10R,12R,12aR)—N-((2,6-dimethylpyridin-4-yl)methyl)-3,6,9-trimethyldecahydro-12H-3,12-epoxy[1,2]dioxepino[4,3-i]isochromen-10-amine;
5-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)-2H-tetrazole;
2-fluoro-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
(1R,4r)-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)cyclohexane-1-carboxylic acid;
2-methyl-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
(1S,4s)-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)cyclohexane-1-carboxylic acid;
(1S,4r)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
3-methyl-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
3-fluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
(3R,5aS,6R,8aS,9R,10R,12R,12aR)-3,6,9-trimethyl-N-((1-methyl-1H-pyrazol-4-yl)methyl)decahydro-12H-3,12-epoxy[1,2]dioxepino[4,3-i]isochromen-10-amine;
2-((diethylamino)methyl)-5-(1-(((3R,5aS,6R,8aS,9R,10R,12R,12aR)-3,6,9-trimethyldecahydro-12H-3,12-epoxy[1,2]dioxepino[4,3-i]isochromen-10-yl)oxy)ethyl)phenol;
(3R,5aS,6R,8aS,9R,10R,12R,12aR)—N-((2-fluoropyridin-4-yl)methyl)-3,6,9-trimethyldecahydro-12H-3,12-epoxy[1,2]dioxepino[4,3-i]isochromen-10-amine;
4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzenesulfonamide;
(3R,5aS,6R,8aS,9R,10R,12R,12aR)—N-((2-methoxypyridin-4-yl)methyl)-3,6,9-trimethyldecahydro-12H-3,12-epoxy[1,2]dioxepino[4,3-i]isochromen-10-amine;
2-methyl-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
3-(((3R,5aS,6R,8aS,9R,10R,12R,12aR)-3,6,9-trimethyldecahydro-12H-3,12-epoxy[1,2]dioxepino[4,3-i]isochromen-10-yl)amino)thietane 1,1-dioxide;
(3R,5aS,6R,8aS,9R,10R,12R,12aR)-3,6,9-trimethyl-10-(2-(methylsulfonyl)ethoxy)decahydro-12H-3,12-epoxy[1,2]dioxepino[4,3-i]isochromene;
2-methoxy-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)bicyclo[1.1.1]pentane-1-carboxylic acid;
(3R,5aS,6R,8aS,9R,10S,12R,12aR)—N-(3-fluorophenyl)-3,6,9-trimethyldecahydro-12H-3,12-epoxy[1,2]dioxepino[4,3-i]isochromen-10-amine;
(3R,5aS,6R,8aS,9R,10R,12R,12aR)-3,6,9-trimethyl-N—((S)-1-(4-(methylsulfonyl)phenyl)ethyl)decahydro-12H-3,12-epoxy[1,2]dioxepino[4,3-i]isochromen-10-amine;
(3R,5aS,6R,8aS,9R,10R,12R,12aR)-3,6,9-trimethyl-N-(4-(methylsulfonyl)benzyl)decahydro-12H-3,12-epoxy[1,2]dioxepino[4,3-i]isochromen-10-amine;
N,N-dimethyl-2-(((3R,5aS,6R,8aS,9R,10R,12R,12aR)-3,6,9-trimethyldecahydro-12H-3,12-epoxy[1,2]dioxepino[4,3-i]isochromen-10-yl)amino)ethane-1-sulfonamide;
N-(oxetan-3-yl)-1-((3R,5aS,6R,8aS,9R,10R,12R,12aR)-3,6,9-trimethyldecahydro-12H-3,12-epoxy[1,2]dioxepino[4,3-i]isochromen-10-yl)piperidine-4-carboxamide;
5-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)furan-2-carboxylic acid;
N-(2-((3R,5aS,6R,8aS,9R,10S,12R,12aR)-3,6,9-trimethyldecahydro-12H-3,12-epoxy[1,2]dioxepino[4,3-i]isochromen-10-yl)ethyl)methanesulfonamide;
1-((3R,5aS,6R,8aS,9R,10R,12R,12aR)-3,6,9-trimethyldecahydro-12H-3,12-epoxy[1,2]dioxepino[4,3-i]isochromen-10-yl)piperidine;
1-((3R,5aS,6R,8aS,9R,10R,12R,12aR)-3,6,9-trimethyldecahydro-12H-3,12-epoxy[1,2]dioxepino[4,3-i]isochromen-10-yl)piperidine-4-carboxylic acid;
N-(1,1-dioxidothietan-3-yl)-1-((3R,5aS,6R,8aS,9R,10R,12R,12aR)-3,6,9-trimethyldecahydro-12H-3,12-epoxy[1,2]dioxepino[4,3-i]isochromen-10-yl)piperidine-4-carboxamide;
1-(2-((3R,5aS,6R,8aS,9R,10R,12R,12aR)-3,6,9-trimethyldecahydro-12H-3,12-epoxy[1,2]dioxepino[4,3-i]isochromen-10-yl)ethyl)piperidine-4-carboxylic acid;
N-methyl-4-((3R,5aS,6R,8aS,9R,10R,12R,12aR)-3,6,9-trimethyldecahydro-12H-3,12-epoxy[1,2]dioxepino[4,3-i]isochromen-10-yl)piperazine-1-carboxamide;
1-(2-((3R,5aS,6R,8aS,9R,10R,12R,12aR)-3,6,9-trimethyldecahydro-12H-3,12-epoxy[1,2]dioxepino[4,3-i]isochromen-10-yl)ethyl)azetidine-3-carboxylic acid;
(R)-1-((3R,5aS,6R,8aS,9R,10R,12R,12aR)-3,6,9-trimethyldecahydro-12H-3,12-epoxy[1,2]dioxepino[4,3-i]isochromen-10-yl)pyrrolidine-3-carboxylic acid;
methyl (R)-1-(2-((3R,5aS,6R,8aS,9R,10R,12R,12aR)-3,6,9-trimethyldecahydro-12H-3,12-epoxy[1,2]dioxepino[4,3-i]isochromen-10-yl)ethyl)pyrrolidine-3-carboxylate;
(1R,4s)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxylic acid;
5-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)pentanoic acid;
5-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)-2H-tetrazole;
3-methyl-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)bicyclo[2.2.2]octane-1-carboxylic acid;
3-methoxy-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
3-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanoic acid;
2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamide;
2-((1S,4s)-4-(((3R,5aS,6R,8aS,9R,10R,12R,12aR)-3,6,9-trimethyldecahydro-12H-3,12-epoxy[1,2]dioxepino[4,3-i]isochromen-10-yl)oxy)cyclohexyl)acetic acid;
6-(((3R,5aS,6R,8aS,9R,10R,12R,12aR)-3,6,9-trimethyldecahydro-12H-3,12-epoxy[1,2]dioxepino[4,3-i]isochromen-10-yl)oxy)spiro[3.3]heptane-2-carboxylic acid;
6-(((3R,5aS,6R,8aS,9R,10S,12R,12aR)-3,6,9-trimethyldecahydro-12H-3,12-epoxy[1,2]dioxepino[4,3-i]isochromen-10-yl)oxy)spiro[3.3]heptane-2-carboxylic acid;
2-((1R,4s)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexyl)acetic acid;
(3R,5aS,6R,8aS,9R,10R,12R,12aR)-3,6,9-trimethyldecahydro-12H-3,12-epoxy[1,2]dioxepino[4,3-i]isochromen-10-yl 4-(dimethylcarbamoyl)benzoate;
(R)-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanoic acid;
(3R,5aS,6R,8aS,9R,10S,12R,12aR)-10-(4-fluorophenoxy)-3,6,9-trimethyldecahydro-12H-3,12-epoxy[1,2]dioxepino[4,3-i]isochromene;
2-((1R,3S)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexyl)acetic acid;
2-fluoro-4-(3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)propyl)benzoic acid;
2-fluoro-4-(3-(((3R,5aS,6R,8aS,9R,10R,12R,12aR)-3,6,9-trimethyldecahydro-12H-3,12-epoxy[1,2]dioxepino[4,3-i]isochromen-10-yl)oxy)propyl)benzoic acid;
2-methoxy-4-(3-(((3R,5aS,6R,8aS,9R,10R,12R,12aR)-3,6,9-trimethyldecahydro-12H-3,12-epoxy[1,2]dioxepino[4,3-i]isochromen-10-yl)oxy)propyl)benzoic acid;
(3R,5aS,6R,8aS,9R,10S,12R,12aR)-3,6,9-trimethyl-10-(2-(methylsulfonyl)ethoxy)decahydro-12H-3,12-epoxy[1,2]dioxepino[4,3-i]isochromene;
3-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
2-chloro-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
(3R,5aS,6R,8aS,9R,10R,12R,12aR)-3,6,9-trimethyldecahydro-12H-3,12-epoxy[1,2]dioxepino[4,3-i]isochromen-10-yl 4-(4-chlorophenyl)-4-hydroxybutanoate;
(3R,5aS,6R,8aS,9R,10R,12R,12aR)-3,6,9-trimethyl-N-phenethyldecahydro-12H-3,12-epoxy[1,2]dioxepino[4,3-i]isochromen-10-amine;
N-methyl-3-(((3R,5aS,6R,8aS,9R,10R,12R,12aR)-3,6,9-trimethyldecahydro-12H-3,12-epoxy[1,2]dioxepino[4,3-i]isochromen-10-yl)amino)propenamide;
3-(((3R,5aS,6R,8aS,9R,10R,12R,12aR)-3,6,9-trimethyldecahydro-12H-3,12-epoxy[1,2]dioxepino[4,3-i]isochromen-10-yl)amino)propenamide;
N-methyl-2-(((3R,5aS,6R,8aS,9R,10R,12R,12aR)-3,6,9-trimethyldecahydro-12H-3,12-epoxy[1,2]dioxepino[4,3-i]isochromen-10-yl)amino)ethane-1-sulfonamide;
(3R,5aS,6R,8aS,9R,10R,12R,12aR)-3,6,9-trimethyl-N-(pyridin-4-ylmethyl)decahydro-12H-3,12-epoxy[1,2]dioxepino[4,3-i]isochromen-10-amine;
(3R,5aS,6R,8aS,9R,10R,12R,12aR)-3,6,9-trimethyl-N-(2-(methylsulfonyl)ethyl)decahydro-12H-3,12-epoxy[1,2]dioxepino[4,3-i]isochromen-10-amine;
(3R,5aS,6R,8aS,9R,10R,12R,12aR)-3,6,9-trimethyl-N-(3-(methylsulfonyl)propyl)decahydro-12H-3,12-epoxy[1,2]dioxepino[4,3-i]isochromen-10-amine;
(3R,5aS,6R,8aS,9R,10R,12R,12aR)—N-benzyl-3,6,9-trimethyldecahydro-12H-3,12-epoxy[1,2]dioxepino[4,3-i]isochromen-10-amine;
4-((methyl((3R,5aS,6R,8aS,9R,10R,12R,12aR)-3,6,9-trimethyldecahydro-12H-3,12-epoxy[1,2]dioxepino[4,3-i]isochromen-10-yl)amino)methyl)benzoic acid;
3-(methyl((3R,5aS,6R,8aS,9R,10R,12R,12aR)-3,6,9-trimethyldecahydro-12H-3,12-epoxy[1,2]dioxepino[4,3-i]isochromen-10-yl)amino)bicyclo[1.1.1]pentane-1-carboxylic acid;
N-(tert-butyl)-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamide;
(3R,5aS,6R,8aS,9R,10R,12R,12aR)-10-((4,4-difluorocyclohexyl)methoxy)-3,6,9-trimethyldecahydro-12H-3,12-epoxy[1,2]dioxepino[4,3-i]isochromene;
(3R,5aS,6R,8aS,9R,10S,12R,12aR)-10-((4,4-difluorocyclohexyl)methoxy)-3,6,9-trimethyldecahydro-12H-3,12-epoxy[1,2]dioxepino[4,3-i]isochromene;
4′-(((3R,5aS,6R,8aS,9R,10S,12R,12aR)-3,6,9-trimethyldecahydro-12H-3,12-epoxy[1,2]dioxepino[4,3-i]isochromen-10-yl)oxy)-[1,1′-biphenyl]-4-carboxylic acid;
((R)-3-fluoropyrrolidin-1-yl)(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)methanone;
3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)bicyclo[1.1.1]pentane-1-carboxylic acid;
(1R,3s)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclobutane-1-carboxylic acid;
2-(3-fluoro-4-(((3R,5aS,6R,8aS,9R,10R,12R,12aR)-3,6,9-trimethyldecahydro-12H-3,12-epoxy[1,2]dioxepino[4,3-i]isochromen-10-yl)oxy)phenyl)propanoic acid;
(3R,5aS,6R,8aS,9R,10S,12R,12aR)-10-((1,3-dihydroisobenzofuran-5-yl)oxy)-3,6,9-trimethyldecahydro-12H-3,12-epoxy[1,2]dioxepino[4,3-i]isochromene;
2-(3-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanoic acid;
(1S,3r)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclobutane-1-carboxylic acid;
3-methyl-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
3-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)bicyclo[1.1.1]pentane-1-carboxylic acid;
4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzonitrile;
2-methyl-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)pyridine;
3-chloro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
2-methyl-4-(3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)propyl)benzoic acid;
2-methoxy-4-(3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)propyl)benzoic acid;
2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propan-2-ol;
4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)bicyclo[2.2.1]heptane-1-carboxylic acid;
1-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)cyclopropane-1-carboxylic acid;
1-(4-(((3R,5aS,6R,8aS,9R,10R,12R,12aR)-3,6,9-trimethyldecahydro-12H-3,12-epoxy[1,2]dioxepino[4,3-i]isochromen-10-yl)oxy)phenyl)cyclopropane-1-carboxylic acid;
2-methyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanoic acid;
1-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclobutane-1-carboxylic acid;
2-oxo-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
2-oxo-2-(4-(((3R,5aS,6R,8aS,9R,10R,12R,12aR)-3,6,9-trimethyldecahydro-12H-3,12-epoxy[1,2]dioxepino[4,3-i]isochromen-10-yl)oxy)phenyl)acetic acid;
2-((1R,3S)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclopentyl)acetic acid;
(1R,4s)-4-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)propan-2-yl)cyclohexane-1-carboxylic acid;
tert-butyl 2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetate;
3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propiolic acid;
(1R,3S)-3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)cyclopentane-1-carboxylic acid;
(1R,3S)-3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
(1R,3S)-3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclopentane-1-carboxylic acid;
3,5-difluoro-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
(1S,3R)-3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
3-(4-(((3R,5aS,6R,8aS,9R,10R,12R,12aR)-3,6,9-trimethyldecahydro-12H-3,12-epoxy[1,2]dioxepino[4,3-i]isochromen-10-yl)oxy)phenyl)propiolic acid;
(1S,4r)-4-fluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)isophthalic acid;
(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)glycine;
(1R,4s)-N-cyclopropyl-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamide;
(2S,5R)-1-methyl-5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)pyrrolidine-2-carboxylic acid;
(3R,5aS,6R,8aS,9R,10R,12R,12aR)-3,6,9-trimethyldecahydro-12H-3,12-epoxy[1,2]dioxepino[4,3-i]isochromen-10-yl 2-(3-(trifluoromethyl)-1H-pyrazol-1-yl)acetate;
3-methyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)butanoic acid;
(1R,3S,5r)-5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1,3-dicarboxylic acid;
1-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetyl)piperidine-4-carboxylic acid;
1-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)azetidine-3-carboxylic acid;
(2S,4R)-4-fluoro-1-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetyl)pyrrolidine-2-carboxylic acid;
(1R,2S,5S)-6,6-dimethyl-3-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetyl)-3-azabicyclo[3.1.0]hexane-2-carboxylic acid;
(1R,3S)-3-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetamido)cyclopentane-1-carboxylic acid;
(1R,4s)-N-(tert-butyl)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamide;
6-(((3R,5aS,6R,8aS,9R,10S,12R,12aR)-3,6,9-trimethyldecahydro-12H-3,12-epoxy[1,2]dioxepino[4,3-i]isochromen-10-yl)oxy)-2,3-dihydrobenzofuran-2-carboxylic acid;
2-(trifluoromethyl)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
((1R,4s)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexyl)methanol;
(1R,5R)-6-(((3R,5aS,6R,8aS,9R,10S,12R,12aR)-3,6,9-trimethyldecahydro-12H-3,12-epoxy[1,2]dioxepino[4,3-i]isochromen-10-yl)oxy)bicyclo[3.2.0]heptane-3-carboxylic acid;
6-(((3R,5aS,6R,8aS,9R,10S,12R,12aR)-3,6,9-trimethyldecahydro-12H-3,12-epoxy[1,2]dioxepino[4,3-i]isochromen-10-yl)oxy)bicyclo[3.1.0]hexane-3-carboxylic acid;
(2S,4R)-4-fluoro-1-(2-(((3R,5aS,6R,8aS,9R,10R,12R,12aR)-3,6,9-trimethyldecahydro-12H-3,12-epoxy[1,2]dioxepino[4,3-i]isochromen-10-yl)oxy)acetyl)pyrrolidine-2-carboxylic acid;
(2S,4S)-4-hydroxy-1-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetyl)pyrrolidine-2-carboxylic acid;
(R)-1-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetyl)pyrrolidine-3-carboxylic acid;
(1R,2S,5S)-6,6-dimethyl-3-(2-(((3R,5aS,6R,8aS,9R,10R,12R,12aR)-3,6,9-trimethyldecahydro-12H-3,12-epoxy[1,2]dioxepino[4,3-i]isochromen-10-yl)oxy)acetyl)-3-azabicyclo[3.1.0]hexane-2-carboxylic acid;
(R)-1-(2-(((3R,5aS,6R,8aS,9R,10R,12R,12aR)-3,6,9-trimethyldecahydro-12H-3,12-epoxy[1,2]dioxepino[4,3-i]isochromen-10-yl)oxy)acetyl)pyrrolidine-3-carboxylic acid;
1-(2-(((3R,5aS,6R,8aS,9R,10R,12R,12aR)-3,6,9-trimethyldecahydro-12H-3,12-epoxy[1,2]dioxepino[4,3-i]isochromen-10-yl)oxy)acetyl)piperidine-4-carboxylic acid;
3-fluoro-4-(3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)prop-1-yn-1-yl)benzoic acid;
3-fluoro-4-(3-(((3R,5aS,6R,8aS,9R,10R,12R,12aR)-3,6,9-trimethyldecahydro-12H-3,12-epoxy[1,2]dioxepino[4,3-i]isochromen-10-yl)oxy)prop-1-yn-1-yl)benzoic acid;
4-chloro-3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
4-chloro-3-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)boronic acid;
1-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)-2-oxabicyclo[2.1.1]hexane-4-carboxylic acid;
2,2-difluoro-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
2-phenyl-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)propanoic acid;
(1R,3S)-3-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)cyclohexane-1-carboxylic acid;
3-fluoro-4-(3-(((3R,5aS,6R,8aS,9R,10R,12R,12aR)-3,6,9-trimethyldecahydro-12H-3,12-epoxy[1,2]dioxepino[4,3-i]isochromen-10-yl)oxy)propyl)benzoic acid;
3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)cyclobutane-1-carboxylic acid;
(4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)phenyl)boronic acid;
3,5-dichloro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
3-chloro-5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
3-chloro-4-(1-(((3R,5aS,6R,8aS,9R,10S,12R,12aR)-3,6,9-trimethyldecahydro-12H-3,12-epoxy[1,2]dioxepino[4,3-i]isochromen-10-yl)oxy)ethyl)benzoic acid;
2-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)-2-methylpropanoic acid;
3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)bicyclo[3.1.0]hexane-6-carboxylic acid;
(1R,3s)-3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)cyclobutane-1-carboxylic acid;
2-(3-(trifluoromethyl)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
(S)-3,3-dimethyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)butanoic acid;
(S)-2-cyclobutyl-2-((1R,4R)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)acetic acid;
2-(3-chloro-5-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
(1R,2S)-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)cyclopentane-1-carboxylic acid;
(S)-3,3-dimethyl-2-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetamido)butanoic acid;
(S)-2-phenyl-2-((1R,4R)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)acetic acid;
(1R,2S,5S)-6,6-dimethyl-3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)-3-azabicyclo[3.1.0]hexane-2-carboxylic acid;
(1S,3aR,6aS)-2-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetyl)octahydrocyclopenta[c]pyrrole-1-carboxylic acid;
2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)cyclopropane-1-carboxylic acid;
(1R,4s)-4-methoxy-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
(S)-2-amino-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
3-(trifluoromethyl)-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
3-chloro-4-((((3R,5aS,6R,8aS,9R,10R,12R,12aR)-3,6,9-trimethyl-10-(trifluoromethyl)decahydro-12H-3,12-epoxy[1,2]dioxepino[4,3-i]isochromen-10-yl)oxy)methyl)benzoic acid;
3-(trifluoromethyl)-4-((((3R,5aS,6R,8aS,9R,12R,12aR)-3,6,9-trimethyldecahydro-12H-3,12-epoxy[1,2]dioxepino[4,3-i]isochromen-10-yl)oxy)methyl)benzoic acid;
N—((S)-2,6-dioxopiperidin-3-yl)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamide;
(1S,4r)-4-methoxy-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
(1R,4s)-4-fluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
(1R,3S)-3-((1R,3R)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclobutane-1-carboxamido)cyclopentane-1-carboxylic acid;
(1R,3S)-3-((1R,4R)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)cyclopentane-1-carboxylic acid;
2-oxo-2-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)piperidin-1-yl)acetic acid;
(1S,4s)-4-((((3R,5aS,6R,8aS,9R,10R,12R,12aR)-3,6,9-trimethyl-10-(trifluoromethyl)decahydro-12H-3,12-epoxy[1,2]dioxepino[4,3-i]isochromen-10-yl)oxy)methyl)cyclohexane-1-carboxylic acid;
(S)-3-cyclobutyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)propanoic acid;
(1R,3s)-3-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)propan-2-yl)cyclobutane-1-carboxylic acid;
(2S,3aS,6aS)-1-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)octahydrocyclopenta[b]pyrrole-2-carboxylic acid;
5-(((3R,5aS,6R,8aS,9R,10S,12R,12aR)-3,6,9-trimethyldecahydro-12H-3,12-epoxy[1,2]dioxepino[4,3-i]isochromen-10-yl)oxy)-1H-indole-2-carboxylic acid;
5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)tetrahydrofuran-2-carboxylic acid;
(3R)-3-fluoro-3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclopentane-1-carboxylic acid;
4-hydroxy-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
(6R)-6-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)tetrahydro-2H-pyran-3-carboxylic acid;
4-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)acetamido)benzoic acid;
6-(((3R,5aS,6R,8aS,9R,10S,12R,12aR)-3,6,9-trimethyldecahydro-12H-3,12-epoxy[1,2]dioxepino[4,3-i]isochromen-10-yl)oxy)-2-naphthoic acid;
3-chloro-5-fluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
3-chloro-5-(methylsulfonyl)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
3-chloro-5-(methylsulfonyl)-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)benzoic acid;
2-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)acetic acid;
2-((4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzyl)oxy)acetic acid;
2-(3-fluoro-5-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)acetic acid;
2-methyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)propanoic acid;
3-fluoro-3-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxylic acid;
2-(3-chloro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenoxy)acetic acid;
2-(((1R,4s)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexyl)oxy)acetic acid;
1-(((3R,5aS,6R,8aS,9R,10R,12R,12aR)-3,6,9-trimethyldecahydro-12H-3,12-epoxy[1,2]dioxepino[4,3-i]isochromen-10-yl)oxy)-2,3-dihydro-1H-indene-5-carboxylic acid;
1-(((3R,5aS,6R,8aS,9R,10S,12R,12aR)-3,6,9-trimethyldecahydro-12H-3,12-epoxy[1,2]dioxepino[4,3-i]isochromen-10-yl)oxy)-2,3-dihydro-1H-indene-5-carboxylic acid;
3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)cyclopentane-1-carboxylic acid;
(1S,3s)-3-((1R,4S)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)cyclobutane-1-carboxylic acid;
(S)-3,3-dimethyl-2-((1R,4R)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)butanoic acid;
2-(2-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)acetic acid;
(S)-2-cyclohexyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)acetic acid;
(1R,3S)-3-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)cyclopentane-1-carboxylic acid;
(1R,3s)-3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)cyclobutane-1-carboxylic acid;
(S)-2-phenyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)acetic acid;
3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoic acid;
(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)-L-valine;
(S)-4,4,4-trifluoro-2-((1R,4R)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)butanoic acid;
2-(3,5-difluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)acetic acid;
(1R,3S)-3-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)cyclopentane-1-carboxylic acid;
(S)-4,4,4-trifluoro-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)butanoic acid;
(1S,3s)-3-((1R,4S)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)cyclobutane-1-carboxylic acid;
(S)-3,3-dimethyl-2-((1R,4R)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)butanoic acid;
(1R,4s)-4-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)cyclohexane-1-carboxylic acid;
1-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)azetidine-3-carboxylic acid;
1-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzyl)-1H-1,2,3-triazole-4-carboxylic acid;
4-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)benzoic acid;
(R)-1-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)pyrrolidine-3-carboxylic acid;
O-methyl-N-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)-L-threonine;
(S)-3-hydroxy-3-methyl-2-((1R,4R)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)butanoic acid;
3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)propanoic acid;
(S)-2-cyclobutyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)acetic acid;
(S)-2-cyclobutyl-2-((1R,4R)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)acetic acid;
(2S,3S,5S,6S,7S,8S)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cubane-1-carboxylic acid;
(S)-3-hydroxy-3-methyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)butanoic acid;
4-((1R,3s)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclobutane-1-carboxamido)cubane-1-carboxylic acid;
(S)-4,4,4-trifluoro-2-((1R,4R)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)butanoic acid;
(S)-2-(4-(trifluoromethyl)phenyl)-2-((1R,3R)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclobutane-1-carboxamido)acetic acid;
(1S,3S,5S)-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzoyl)-2-azabicyclo[3.1.0]hexane-3-carboxylic acid;
1-((1R,4s)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carbonyl)azetidine-3-carboxylic acid;
(2R,3R,5R,6R,7R,8R)-4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)cubane-1-carboxylic acid;
(S)-3-methyl-2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)butanoic acid;
1-((1R,4s)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)cyclohexane-1-carboxamido)cyclopropane-1-carboxylic acid;
(R)-1-((1R,4S)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carbonyl)pyrrolidine-3-carboxylic acid;
1-(2-bromo-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzyl)-1H-1,2,3-triazole-4-carboxylic acid;
3-(3-chloro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)oxetane-3-carboxylic acid;
2-(3-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)-3-methylbutanoic acid;
(1R,3S)-3-((1R,4R)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)cyclopentane-1-carboxylic acid;
1-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenoxy)cyclopropane-1-carboxylic acid;
(1S,3R)-3-((E)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)prop-1-en-1-yl)cyclohexane-1-carboxylic acid;
2-(3-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)-2-methylpropanoic acid;
(1S,4r)-4-fluoro-4-((E)-3-(((3R,5aS,6R,8aS,9R,10S,12R,12aR)-3,6,9-trimethyldecahydro-12H-3,12-epoxy[1,2]dioxepino[4,3-i]isochromen-10-yl)oxy)prop-1-en-1-yl)cyclohexane-1-carboxylic acid;
(1R,3S)-3-(N-methyl-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)cyclopentane-1-carboxylic acid;
3-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)bicyclo[1.1.1]pentane-1-carboxylic acid;
2-(4-((((3S,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenoxy)acetic acid;
(1R,3S)-3-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclopentane-1-carboxylic acid;
(2S,4S)-4-fluoro-1-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoyl)pyrrolidine-2-carboxylic acid;
1-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoyl)piperidine-4-carboxylic acid;
(1R,3s)-3-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclobutane-1-carboxylic acid;
(R)-2-(3-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanoic acid;
3-chloro-5-methyl-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
(S)-3,3-dimethyl-2-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)butanoic acid;
(S)-2-(3-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanoic acid;
3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)oxetane-3-carboxylic acid;
2-methyl-2-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)propanoic acid;
(2-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)acetyl)-L-valine;
1-((4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)methyl)cyclobutane-1-carboxylic acid;
4-((1R,4s)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)benzoic acid;
(1S,3R,4R)-3-hydroxy-4-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclopentane-1-carboxylic acid;
(1S,3R)-3-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)cyclopentane-1-carboxylic acid;
(1R,3R)-3-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclohexane-1-carboxylic acid;
6-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)spiro[3.3]heptane-2-carboxylic acid;
(1R,3s)-3-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)cyclobutane-1-carboxylic acid;
4-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)bicyclo[2.1.1]hexane-1-carboxylic acid;
(1R,3S)-3-((4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)carbamoyl)cyclopentane-1-carboxylic acid;
4-((1R,4s)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)bicyclo[2.1.1]hexane-1-carboxylic acid;
2-(5,5-dimethyl-2,4-dioxo-3-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)imidazolidin-1-yl)acetic acid;
1-(((1R,4s)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)cyclohexane-1-carboxamido)methyl)cyclobutane-1-carboxylic acid;
2-(trifluoromethoxy)-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
(1S,3R,4R)-3-hydroxy-4-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)cyclopentane-1-carboxylic acid;
4-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)bicyclo[2.1.1]hexane-1-carboxylic acid;
(S)-2-cyclopropyl-2-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)acetic acid;
(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetyl)-L-valine;
3-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)bicyclo[1.1.1]pentane-1-carboxylic acid;
2-(5-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)-1,3,4-oxadiazol-2-yl)acetic acid;
(1S,3R)-3-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclopentane-1-carboxylic acid;
(1R,3S)-3-(2-(3-fluoro-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanamido)cyclopentane-1-carboxylic acid;
(1S,3S)-3-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)cyclopentane-1-carboxylic acid;
(1R,4s)-4-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)cyclohexane-1-carboxylic acid;
(1R,3S)-3-(3,5-difluoro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclopentane-1-carboxylic acid;
2-(trifluoromethoxy)-5-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoic acid;
(1R,3R)-3-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)cyclopentane-1-carboxylic acid;
2-(1-methyl-1H-pyrazol-4-yl)-2-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)acetic acid;
(1S,3R)-3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzamido)cyclopentane-1-carboxylic acid;
(S)-2-acetamido-3-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)propanoic acid;
(1S,2R,3S,4R)-2,3-dihydroxy-4-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclopentane-1-carboxylic acid;
(1R,3S)-3-((4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)benzyl)carbamoyl)cyclopentane-1-carboxylic acid;
(1R,3S)-3-(3-chloro-4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzamido)cyclopentane-1-carboxylic acid;
2-(4-(4-((((3R,5aS,6R,8aS,9R,10R,12R,12aR)-3,6,9-trimethyldecahydro-12H-3,12-epoxy[1,2]dioxepino[4,3-i]isochromen-10-yl)oxy)methyl)phenyl)-1H-1,2,3-triazol-1-yl)acetic acid;
2-(4-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)-1H-1,2,3-triazol-1-yl)acetic acid;
3-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)propanoic acid;
2-(2-(trifluoromethoxy)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
2-(2-(difluoromethoxy)-4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetic acid;
(1R,2S,4R,5S)-4-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)bicyclo[3.1.0]hexane-2-carboxylic acid;
(2S,4R)-4-hydroxy-1-(4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)benzoyl)pyrrolidine-2-carboxylic acid;
(1R,3S)-3-(((4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)methyl)sulfonamido)cyclopentane-1-carboxylic acid;
(1R,3S)-3-((4-((((3R,5aS,6R,8aS,9R,10S,12R,12aR)-3,6,9-trimethyldecahydro-12H-3,12-epoxy[1,2]dioxepino[4,3-i]isochromen-10-yl)oxy)methyl)phenyl)sulfonamido)cyclopentane-1-carboxylic acid;
6-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetamido)spiro[3.3]heptane-2-carboxylic acid;
(1R,3s)-3-((4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)carbamoyl)cyclobutane-1-carboxylic acid;
(S)-1-oxo-7-(((3R,5aS,6R,8aS,9R,10S,12R,12aR)-3,6,9-trimethyldecahydro-12H-3,12-epoxy[1,2]dioxepino[4,3-i]isochromen-10-yl)oxy)-1,2,3,4-tetrahydroisoquinoline-3-carboxylic acid; and
(1R,5S,6S)-3-(2-(4-(((3R,5aS,6R,8aS,9R,10S,12R,12aR)-3,6,9-trimethyldecahydro-12H-3,12-epoxy[1,2]dioxepino[4,3-i]isochromen-10-yl)oxy)phenyl)acetyl)-3-azabicyclo[3.1.0]hexane-6-carboxylic acid.

14. A pharmaceutical composition comprising the compound of any one of claims 1-13 admixed with a pharmaceutically acceptable carrier, diluent, or excipient.

15. The pharmaceutical composition of claim 14, further comprising one or more therapeutic compounds or compositions.

16. The pharmaceutical composition of claim 15, wherein the one or more therapeutic compounds or compositions is a second anticancer compound or composition.

17. A method of preventing, ameliorating, or treating a myelodysplastic syndrome (MDS), comprising administering to a subject in need thereof a therapeutically effective amount of the compound of either claim 1 or claim 13.

18. A method of preventing, ameliorating, or treating a myelodysplastic syndrome (MDS), comprising administering to a subject in need thereof a therapeutically effective amount of the pharmaceutical composition of claim 14.

19. A method of preventing, ameliorating, or treating a myelodysplastic syndrome (MDS), comprising administering to a subject in need thereof a therapeutically effective amount of the pharmaceutical composition of claim 15.

20. A method of preventing, ameliorating, or treating a myelodysplastic syndrome (MDS), comprising administering to a subject in need thereof a therapeutically effective amount of the pharmaceutical composition of claim 16.

21. A method for slowing or preventing the development of leukemia in a subject, comprising administering to the subject a therapeutically effective amount of the compound of either claim 1 or claim 13.

22. A method for slowing or preventing the development of leukemia in a subject, comprising administering to the subject a therapeutically effective amount of the pharmaceutical composition of claim 14.

23. A method for slowing or preventing the development of leukemia in a subject, comprising administering to the subject a therapeutically effective amount of the pharmaceutical composition of claim 15.

24. A method for slowing or preventing the development of leukemia in a subject, comprising administering to the subject a therapeutically effective amount of the pharmaceutical composition of claim 16.

25. The method of claim 17, wherein the MDS is primary MDS.

26. The method of claim 17, wherein the MDS is secondary MDS.

27. The method of claim 17, wherein the MDS is one or more subtypes chosen from refractory anemia (RA), refractory anemia with ringed sideroblasts (RARS), refractory cytopenia with multilineage dysplasia (RCMD), refractory cytopenia with multilineage dysplasia and ringed sideroblasts (RCMD-RS), refractory anemia with excess blasts (RAEB), myelodysplastic syndrome unclassified (MDS-U), and MDS associated with isolated del(5q).
